(19) United States
(12) Patent Application Publication
(10) Pub. No.: US 2020/0144508 A1
(43) Pub. Date: May 7, 2020

(54) ORGANIC OPTOELECTRONIC DEVICE AND DISPLAY DEVICE

(71) Applicant: SAMSUNG SDI CO., LTD., Yongin-si (KR)

(72) Inventors: Kipo JANG, Yongin-si (KR); Byungku KIM, Yongin-si (KR); Youngmok SON, Hwaseong-si (KR); Eun Sun YU, Yongin-si (KR); Bang-Lin LEE, Suwon-si (KR); Byoungkwan LEE, Yongin-si (KR); Suyong LIM, Yongin-si (KR); Youngkyoung JO, Yongin-si (KR)

(21) Appl. No.: 16/672,880

(22) Filed: Nov. 4, 2019

(30) Foreign Application Priority Data
Nov. 2, 2018 (KR) 10-2018-0133747

(51) Int. Cl. H01L 51/00; C07D 209/88; C09K 11/06; C07D 405/04; C07D 405/14; C07D 495/04; C07D 251/24; C07D 333/76; C07D 307/91; C07D 409/12

(52) U.S. Cl. CPC H01L 51/0061; H01L 51/5064; C09K 11/06; H01L 51/006; C07D 405/04; H01L 51/0067; H01L 51/0073; H01L 51/0052; C07D 405/14; C07D 495/04; H01L 51/0071; H01L 51/0072; C07D 251/24; H01L 51/0054; C07D 333/76; C07D 307/91; C07D 409/12; C07D 209/88

(57) ABSTRACT

An organic optoelectronic device and a display device including the organic optoelectronic device, the organic optoelectronic device including an anode and a cathode facing each other, a light emitting layer between the anode and the cathode, a hole transport layer between the anode and the light emitting layer, and a hole transport auxiliary layer between the light emitting layer and the hole transport layer, wherein the light emitting layer includes a first compound represented by Chemical Formula 1 and a second compound represented by Chemical Formula 2, and the hole transport auxiliary layer includes a third compound represented by Chemical Formula 3,

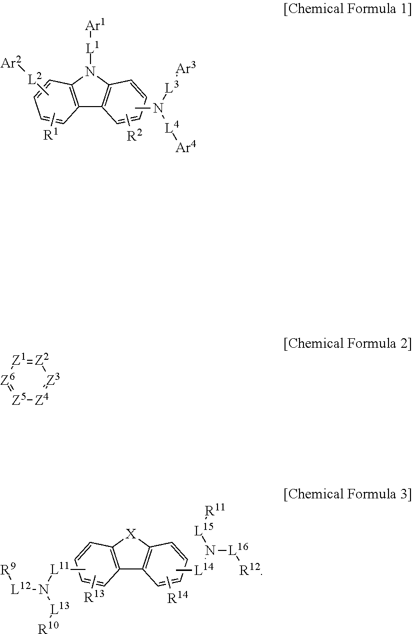

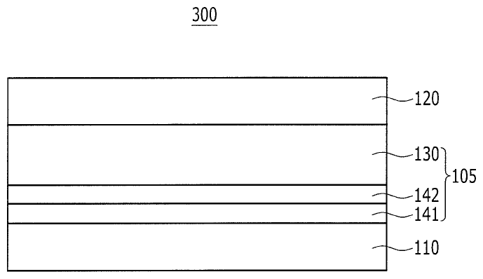

### CROSS-REFERENCE TO RELATED APPLICATION

Korean Patent Application No. 10-2018-0133747, filed on Nov. 2, 2018, in the Korean Intellectual Property Office, and entitled: “Organic Optoelectronic Device and Display Device,” is incorporated by reference herein in its entirety.

### BACKGROUND

### 1. Field

Embodiments relate to an organic optoelectronic device and a display device.

### 2. Description of the Related Art

An organic optoelectronic device (e.g., an organic optoelectronic diode) is a device that converts electrical energy into photoenergy, and vice versa.

An organic optoelectronic device may be classified as follows in accordance with its driving principles. One is a photoelectric device in which excitons generated by photoenergy are separated into electrons and holes and the electrons and holes are transferred to different electrodes respectively and electrical energy is generated, and the other is a light emitting device to generate photoenergy from electrical energy by supplying a voltage or a current to electrodes.

Examples of the organic optoelectronic device include an organic photoelectric device, an organic light emitting diode, an organic solar cell, and an organic photo conductor drum.

Among them, the organic light emitting diode (OLED) has recently drawn attention due to an increase in demand for flat panel displays. The organic light emitting diode converts electrical energy into light, and the performance of organic light emitting diode is greatly influenced by the organic materials between electrodes.

### SUMMARY

The embodiments may be realized by providing an organic optoelectronic device including an anode and a cathode facing each other, a light emitting layer between the anode and the cathode, a hole transport layer between the anode and the light emitting layer, and a hole transport auxiliary layer between the light emitting layer and the hole transport layer, wherein the light emitting layer includes a first compound represented by Chemical Formula 1 and a second compound represented by Chemical Formula 2, and the hole transport auxiliary layer includes a third compound represented by Chemical Formula 3,

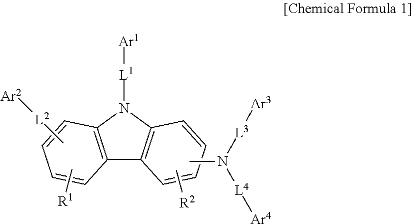

wherein, in Chemical Formula 1, Ar¹ and Ar² are each independently a substituted or unsubstituted C6 to C12 aryl group. Ar³ and Ar⁴ are each independently a substituted or unsubstituted C6 to C30 aryl group, L¹ is a single bond or a substituted or unsubstituted phenylene group, L² to L⁴ are each independently a single bond or a substituted or unsubstituted C6 to C12 arylene group, and R¹ and R² are each independently hydrogen, deuterium, a cyano group, or a substituted or unsubstituted C1 to C10 alkyl group;

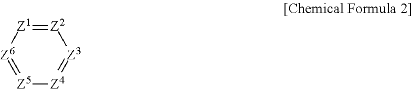

wherein, in Chemical Formula 2, Z¹ is N or C-L⁵-R³, Z² is N or C-L⁶-R⁴, Z³ is N or C-L⁷-R⁵, Z⁴ is N or C-L⁸-R⁶, Z⁵ is N or C-L⁹-R⁷, Z⁶ is N or C-L¹⁰-R⁸, at least two of Z¹ to Z⁶ are N, L⁵ to L¹⁰ are each independently a single bond, a substituted or unsubstituted C6 to C20 arylene group, a substituted or unsubstituted divalent C2 to C20 heterocyclic group, or a combination thereof, R³ to R⁸ are each independently hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted silyl group, a substituted or unsubstituted amine group, a halogen, a cyano group, or a combination thereof, at least one of R³ to R⁸ is a substituted or unsubstituted dibenzofuranyl group, a substituted or unsubstituted dibenzothiophenyl group, a substituted or unsubstituted carbazolyl group, a substituted or unsubstituted benzocarbazolyl group, a substituted or unsubstituted dibenzocarbazolyl group, or a substituted or unsubstituted triphenylene group, and R³ to R⁸ are each separately present or adjacent groups thereof are linked with each other to form a substituted or unsubstituted aliphatic monocyclic or polycyclic ring, a substituted or unsubstituted aromatic monocyclic or polycyclic ring, or a substituted or unsubstituted heteroaromatic monocyclic or polycyclic ring;

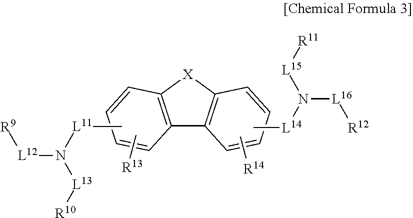

wherein, in Chemical Formula 3, X is O or S, L¹¹ to L¹⁶ are each independently a single bond, a substituted or unsubstituted C6 to C20 arylene group, a substituted or unsubstituted divalent C2 to C20 heterocyclic group, or a combination thereof, R⁹ to R¹² are each independently a substituted or unsubstituted C6 to C30 aryl group, a substituted or unsubstituted C2 to C30 heterocyclic group, or a combination thereof, and R¹³ and R¹⁴ are each independently hydrogen, deuterium, a cyano group, or a substituted or unsubstituted C1 to C10 alkyl group.

The first compound may be represented by Chemical Formula 1-2 or Chemical Formula 1-3:

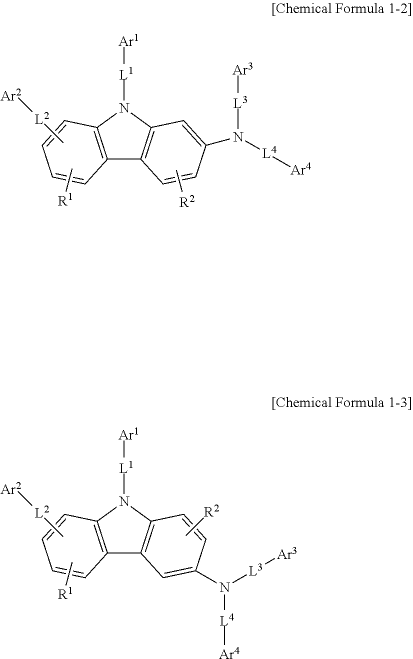

wherein, in Chemical Formula 1-2 and Chemical Formula 1-3, Ar¹ and Ar² are each independently a substituted or unsubstituted C6 to C12 aryl group, Ar³ and Ar⁴ are each independently a substituted or unsubstituted C6 to C30 aryl group, L¹ is a single bond or a substituted or unsubstituted phenylene group, L² to L⁴ are each independently a single bond or a substituted or unsubstituted C6 to C12 arylene group, and R¹ and R² are each independently hydrogen, deuterium, a cyano group, or a substituted or unsubstituted C1 to C10 alkyl group.

The first compound may be represented by Chemical Formula 1-2b or Chemical Formula 1-3b:

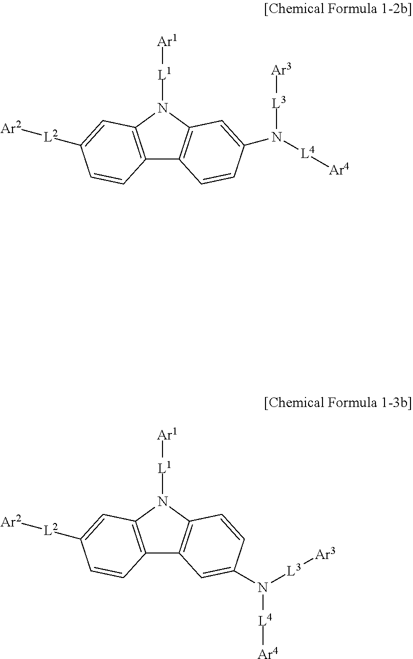

wherein, in Chemical Formula 1-2b and Chemical Formula 1-3b, Ar¹ and Ar² are each independently a substituted or unsubstituted C6 to C12 aryl group, Ar³ and Ar⁴ are each independently a substituted or unsubstituted C6 to C30 aryl group, L¹ is a single bond or a substituted or unsubstituted phenylene group, and L² to L⁴ are each independently a single bond or a substituted or unsubstituted C6 to C12 arylene group.

Ar¹ and Ar² may each independently be a substituted or unsubstituted phenyl group, a substituted or unsubstituted biphenyl group, or a substituted or unsubstituted naphthyl group.

Ar³ and Ar⁴ may each independently be a substituted or unsubstituted phenyl group, a substituted or unsubstituted biphenyl group, a substituted or unsubstituted naphthyl group, a substituted or unsubstituted terphenyl group, a substituted or unsubstituted anthracenyl group, or a substituted or unsubstituted phenanthrenyl group.

The second compound may be represented by Chemical Formula 2-1 to Chemical Formula 2-3:

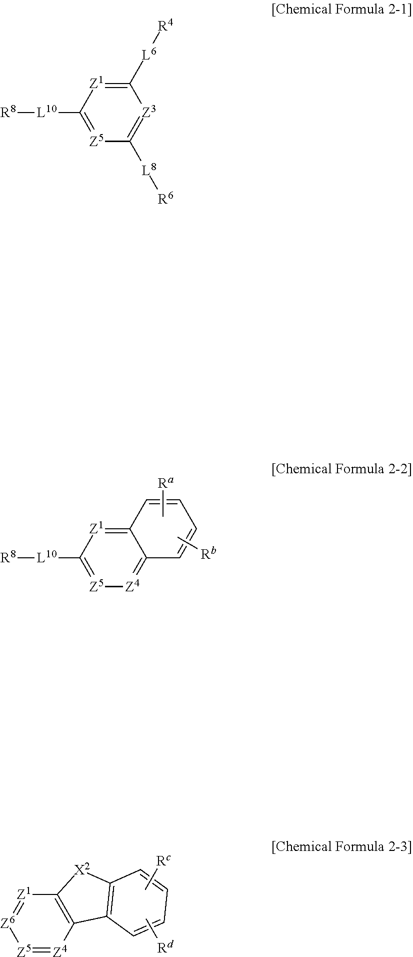

wherein, in Chemical Formula 2-1 to Chemical Formula 2-3, Z¹ is N or C-L¹⁰-R³, Z³ is N or C-L⁷-R⁵, Z⁴ is N or C-L⁸-R⁶, Z⁵ is N or C-L⁹-R⁷, Z⁶ is N or C-L¹⁰-R⁸, at least two of Z¹ and Z³ to Z⁶ are N, L⁵ to L¹⁰ are each independently a single bond, a substituted or unsubstituted C6 to C20 arylene group, a substituted or unsubstituted divalent C2 to C20 heterocyclic group, or a combination thereof, R³ to R⁸ are each independently hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted silyl group, a substituted or unsubstituted amine group, a halogen, a cyano group, or a combination thereof, at least one of R³ to R⁸ is a substituted or unsubstituted dibenzofuranyl group, a substituted or unsubstituted dibenzothiophenyl group, a substituted or unsubstituted carbazolyl group, a substituted or unsubstituted benzocarbazolyl group, a substituted or unsubstituted dibenzocarbazolyl group, or a substituted or unsubstituted triphenylene group, and R^{a}, R^{b}, R^{c}, and R^{d} are each independently hydrogen, deuterium, a halogen, a cyano group, a C1 to C20 alkyl group, a C6 to C30 aryl group, a C2 to C30 heteroaryl group, or a combination thereof.

At least one of R³ to R⁸ may be a group of the following Group II:

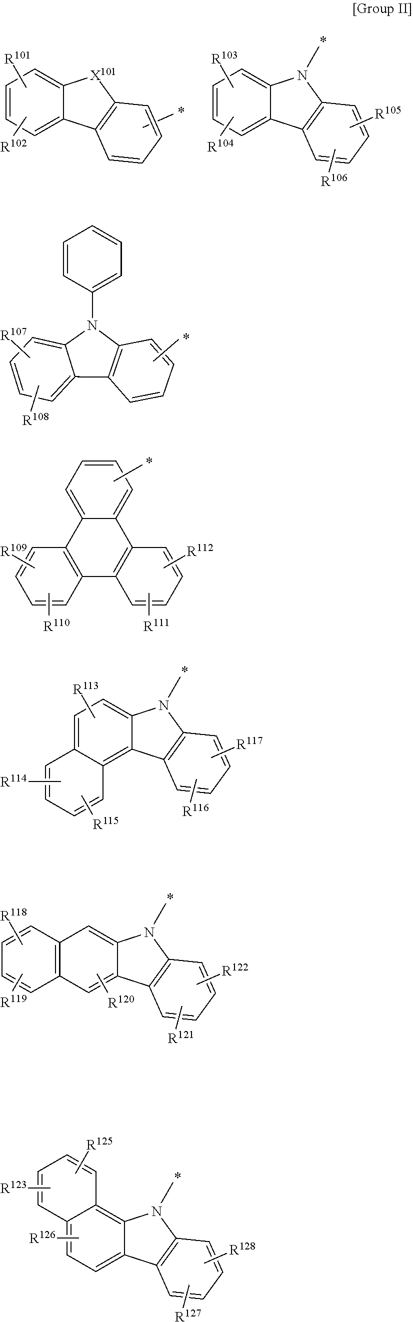

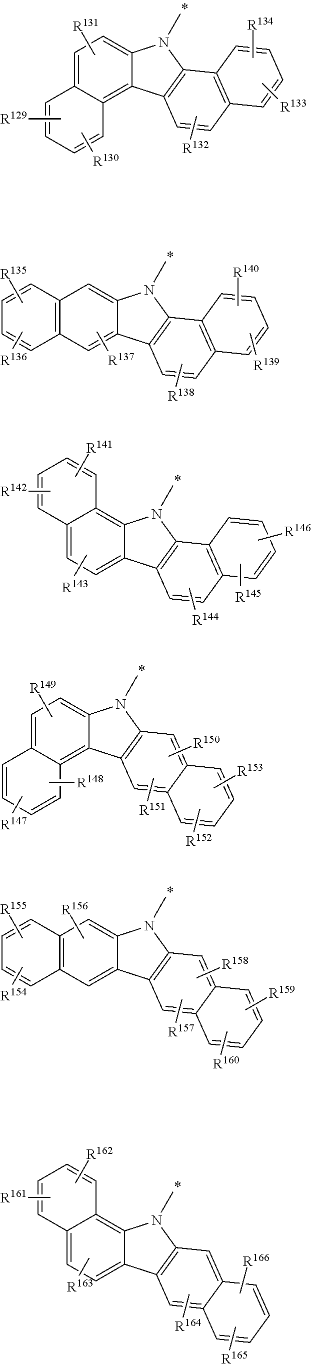

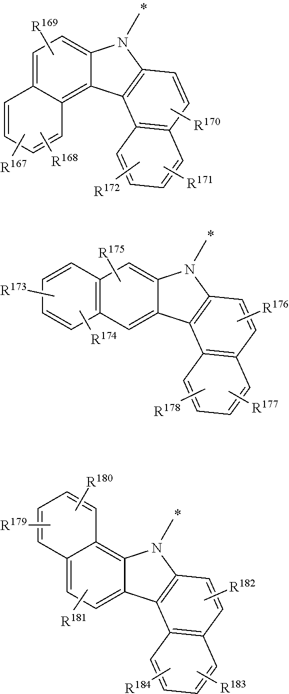

wherein, in Group II, X¹⁰¹ is O or S, R¹⁰¹ to R¹²³ and R¹²⁵ to R¹⁸⁴ are each independently hydrogen, deuterium, a halogen, a cyano group, a C1 to C20 alkyl group, a C6 to C30 aryl group, a C2 to C30 heteroaryl group, or a combination thereof, and * is a linking point.

The second compound may be represented by Chemical Formula 2-1a or Chemical Formula 2-3a:

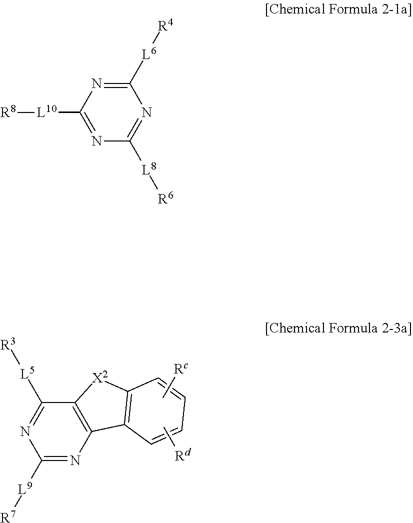

wherein, in Chemical Formula 2-1a, L⁶, L⁸, and L¹⁰ are each independently a single bond or a substituted or unsubstituted phenylene group, R⁴, R⁶, and R⁸ are each independently a substituted or unsubstituted phenyl group, a substituted or unsubstituted biphenyl group, a substituted or unsubstituted terphenyl group, a substituted or unsubstituted naphthyl group, a substituted or unsubstituted triphenylene group, a substituted or unsubstituted dibenzofuranyl group, a substituted or unsubstituted dibenzothiophenyl group, or a substituted or unsubstituted carbazolyl group, at least one of R⁴, R⁶, and R⁸ is a substituted or unsubstituted triphenylene group, a substituted or unsubstituted carbazolyl group, a substituted or unsubstituted dibenzofuranyl group, or a substituted or unsubstituted dibenzothiophenyl group; wherein, in Chemical Formula 2-3a, X² is O or S. L⁵ and L⁹ are each independently a single bond or a substituted or unsubstituted phenylene group, R³ and R⁷ are each independently a substituted or unsubstituted phenyl group, a substituted or unsubstituted biphenyl group, a substituted or unsubstituted terphenyl group, a substituted or unsubstituted naphthyl group, a substituted or unsubstituted triphenylene group, a substituted or unsubstituted dibenzofuranyl group, a substituted or unsubstituted dibenzothiophenyl group, or a substituted or unsubstituted carbazolyl group, at least one of R³ and R⁷ is a substituted or unsubstituted carbazolyl group, a substituted or unsubstituted dibenzofuranyl group, or a substituted or unsubstituted dibenzothiophenyl group, and R^{c} and R^{d} are each independently hydrogen, deuterium, a C1 to C10 alkyl group, a C6 to C12 aryl group, or a combination thereof.

The first compound may be represented by Chemical Formula 1-2b:

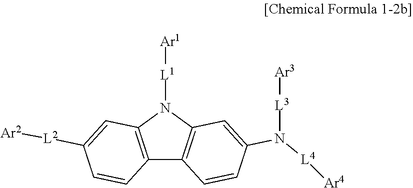

wherein, in Chemical Formula 1-2b, L¹ to L⁴ are each independently a single bond or a substituted or unsubstituted phenylene group, and Ar¹ to Ar⁴ are each independently a substituted or unsubstituted phenyl group, a substituted or unsubstituted biphenyl group, or a substituted or unsubstituted naphthyl group.

The third compound is represented by one of Chemical Formula 3-1 to Chemical Formula 3-4:

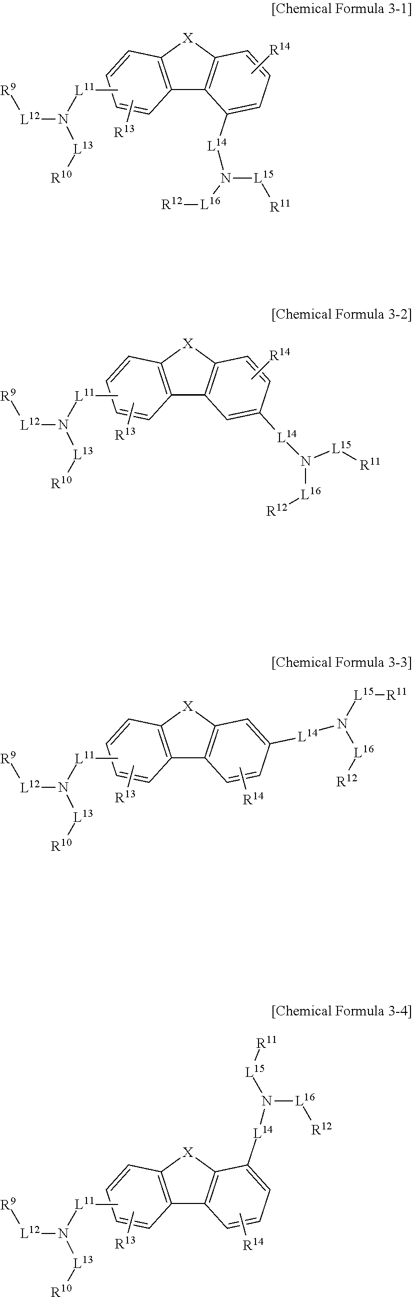

wherein, in Chemical Formula 3-1 to Chemical Formula 3-4, X is O or S, L¹ to L¹⁶ are each independently a single bond, a substituted or unsubstituted C6 to C20 arylene group, a substituted or unsubstituted divalent C2 to C20 heterocyclic group, or a combination thereof, R⁹ to R¹² are each independently a substituted or unsubstituted C6 to C30 aryl group, a substituted or unsubstituted C2 to C30 heterocyclic group, or a combination thereof, and R¹³ and R¹⁴ are each independently hydrogen, deuterium, a cyano group, or a substituted or unsubstituted C1 to C10 alkyl group.

The third compound may be represented by one of Chemical Formula 3-1b, Chemical Formula 3-2b, Chemical Formula 3-2c, Chemical Formula 3-3b, Chemical Formula 3-3c, and Chemical Formula 3-3d:

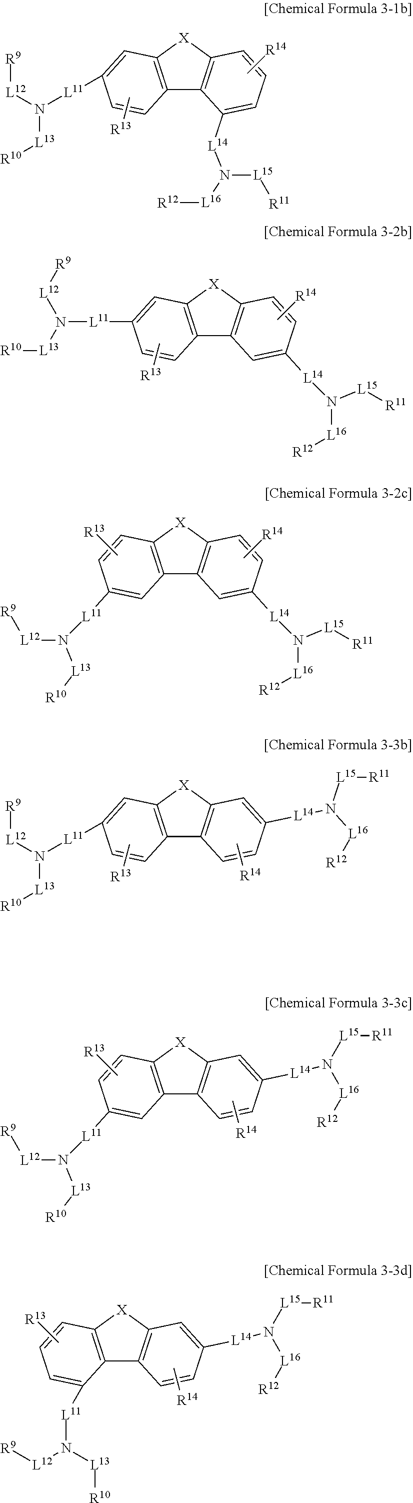

wherein, in Chemical Formula 3-1b, Chemical Formula 3-2b, Chemical Formula 3-2c, Chemical Formula 3-3b, Chemical Formula 3-3c, and Chemical Formula 3-3d, X is O or S, L¹¹ to L¹⁶ are each independently a single bond, a substituted or unsubstituted C6 to C20 arylene group, a substituted or unsubstituted divalent C2 to C20 heterocyclic group, or a combination thereof, R⁹ to R¹² are each independently a substituted or unsubstituted C6 to C30 aryl group, a substituted or unsubstituted C2 to C30 heterocyclic group, or a combination thereof, and R¹³ and R¹⁴ are each independently hydrogen, deuterium, a cyano group, or a substituted or unsubstituted C1 to C10 alkyl group.

The first compound may be represented by Chemical Formula 1-2b, and the second compound may be represented by Chemical Formula 2-1a or Chemical Formula 2-3a,

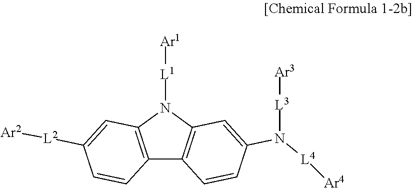

wherein, in Chemical Formula 1-2b, L¹ to L⁴ are each independently a single bond or a substituted or unsubstituted phenylene group, and Ar¹ to Ar⁴ are each independently a substituted or unsubstituted phenyl group, a substituted or unsubstituted biphenyl group, or a substituted or unsubstituted naphthyl group;

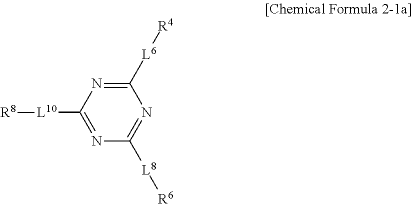

wherein, in Chemical Formula 2-1a, L⁶, L⁸, and L¹⁰ are each independently a single bond or a substituted or unsubstituted phenylene group. R⁴, R⁶, and R⁸ are each independently a substituted or unsubstituted phenyl group, a substituted or unsubstituted biphenyl group, a substituted or unsubstituted terphenyl group, a substituted or unsubstituted naphthyl group, a substituted or unsubstituted triphenylene group, a substituted or unsubstituted dibenzofuranyl group, a substituted or unsubstituted dibenzothiophenyl group, or a substituted or unsubstituted carbazolyl group, and at least one of R⁴, R⁶, and R⁸ is a substituted or unsubstituted triphenylene group, a substituted or unsubstituted carbazolyl group, a substituted or unsubstituted dibenzofuranyl group, or a substituted or unsubstituted dibenzothiophenyl group;

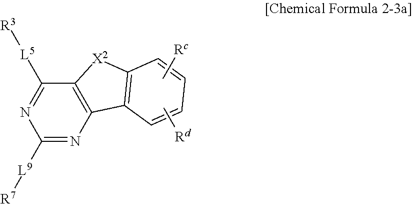

wherein, in Chemical Formula 2-3a, X² is O or S, L⁵ and L⁹ are each independently a single bond or a substituted or unsubstituted phenylene group, R³ and R⁷ are each independently substituted or unsubstituted phenyl group, a substituted or unsubstituted biphenyl group, a substituted or unsubstituted terphenyl group, a substituted or unsubstituted naphthyl group, a substituted or unsubstituted triphenylene group, a substituted or unsubstituted dibenzofuranyl group, a substituted or unsubstituted dibenzothiophenyl group, or a substituted or unsubstituted carbazolyl group, at least one of R³ and R⁷ is a substituted or unsubstituted carbazolyl group, a substituted or unsubstituted dibenzofuranyl group, or a substituted or unsubstituted dibenzothiophenyl group, and R^{c} and R^{d} are each independently hydrogen, deuterium, a C1 to C10 alkyl group, a C6 to C12 aryl group, or a combination thereof.

At least one of R⁹ to R¹² of Chemical Formula 3 may be a substituted or unsubstituted dibenzofuranyl group or a substituted or unsubstituted dibenzothiophenyl group.

The first compound and the second compound may be phosphorescent hosts of the light emitting layer.

The light emitting layer may further include a dopant.

The embodiments may be realized by providing a display device comprising the organic optoelectronic device according to an embodiment.

### BRIEF DESCRIPTION OF THE DRAWING

Features will be apparent to those of skill in the art by describing in detail exemplary embodiments with reference to the attached drawing in which:

The FIGURE illustrates a schematic cross-sectional view of an organic optoelectronic device according to an embodiment.

### DETAILED DESCRIPTION

Example embodiments will now be described more fully hereinafter with reference to the accompanying drawing; however, they may be embodied in different forms and should not be construed as limited to the embodiments set forth herein. Rather, these embodiments are provided so that this disclosure will be thorough and complete, and will fully convey exemplary implementations to those skilled in the art.

In the drawing FIGURE, the dimensions of layers and regions may be exaggerated for clarity of illustration. It will also be understood that when a layer or element is referred to as being “on” another layer or element, it can be directly on the other layer or element, or intervening layers may also be present. In addition, it will also be understood that when a layer is referred to as being “between” two layers, it can be the only layer between the two layers, or one or more intervening layers may also be present. Like reference numerals refer to like elements throughout. As used herein, the term “or” is not an exclusive term, e.g., “A or B” would include A, B, or A and B.

As used herein, when a definition is not otherwise provided, “substituted” refers to replacement of at least one hydrogen of a substituent or a compound by deuterium, a halogen, a hydroxyl group, an amino group, a substituted or unsubstituted C1 to C30 amine group, a nitro group, a silane group, a substituted or unsubstituted C1 to C40 silyl group, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, a C1 to C20 alkoxy group, a C1 to C10 trifluoroalkyl group, a cyano group, or a combination thereof.

In one example, “substituted” refers to replacement of at least one hydrogen of a substituent or a compound by deuterium, a halogen, a silane group, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, or a cyano group. In addition, in specific examples, “substituted” refers to replacement of at least one hydrogen of a substituent or a compound by deuterium, a halogen, a silane group, a C1 to C20 alkyl group, a C6 to C30 aryl group, a C2 to C30 heteroaryl group, or a cyano group. In addition, in specific examples, “substituted” refers to replacement of at least one hydrogen of a substituent or a compound by deuterium, a fluoro group, a silane group, a C1 to C5 alkyl group, a C6 to C18 aryl group, a pyridinyl group, a quinolinyl group, an isoquinolinyl group, a dibenzofuranyl group, a dibenzothiophenyl group, a carbazolyl group, or a cyano group. In addition, in specific examples, “substituted” refers to replacement of at least one hydrogen of a substituent or a compound by deuterium, a fluoro group, a silane group, a C1 to C5 alkyl group, a C6 to C18 aryl group, or a cyano group. In addition, in specific examples, “substituted” refers to replacement of at least one hydrogen of a substituent or a compound by deuterium, a fluoro group, a silane group, a methyl group, an ethyl group, a propanyl group, a butyl group, a phenyl group, a biphenyl group, a naphthyl group, or a cyano group.

As used herein, when a definition is not otherwise provided, “hetero” refers to one including one to three heteroatoms selected from N, O, S, P, and Si, and remaining carbons in one functional group.

In the present specification, “aryl group” refers to a group including at least one hydrocarbon aromatic moiety, and may include a group in which all elements of the hydrocarbon aromatic moiety have p-orbitals which form conjugation, for example a phenyl group, a naphthyl group, and the like, a group in which two or more hydrocarbon aromatic moieties may be linked by a sigma bond, for example a biphenyl group, a terphenyl group, a quarterphenyl group, and the like, and a group in which two or more hydrocarbon aromatic moieties are fused directly or indirectly to provide a non-aromatic fused ring, for example a fluorenyl group, and the like.

The aryl group may include a monocyclic, polycyclic, or fused ring polycyclic (i.e., rings sharing adjacent pairs of carbon atoms) functional group.

In the present specification, “heterocyclic group” is a generic concept of a heteroaryl group, and may include at least one heteroatom selected from N, O, S, P, and Si instead of carbon (C) in a cyclic compound such as an aryl group, a cycloalkyl group, a fused ring thereof, or a combination thereof. When the heterocyclic group is a fused ring, the entire ring or each ring of the heterocyclic group may include one or more heteroatoms.

For example, “heteroaryl group” may refer to an aryl group including at least one heteroatom selected from N, O, S, P, and Si. Two or more heteroaryl groups are linked by a sigma bond directly, or when the heteroaryl group includes two or more rings, the two or more rings may be fused. When the heteroaryl group is a fused ring, each ring may include one to three heteroatoms.

More specifically, the substituted or unsubstituted C6 to C30 aryl group may be a substituted or unsubstituted phenyl group, a substituted or unsubstituted naphthyl group, a substituted or unsubstituted anthracenyl group, a substituted or unsubstituted phenanthrenyl group, a substituted or unsubstituted naphthacenyl group, a substituted or unsubstituted pyrenyl group, a substituted or unsubstituted biphenyl group, a substituted or unsubstituted p-terphenyl group, a substituted or unsubstituted m-terphenyl group, a substituted or unsubstituted o-terphenyl group, a substituted or unsubstituted chrysenyl group, a substituted or unsubstituted triphenylene group, a substituted or unsubstituted perylenyl group, a substituted or unsubstituted fluorenyl group, a substituted or unsubstituted indenyl group, or a combination thereof.

More specifically, the substituted or unsubstituted C2 to C30 heterocyclic group may be a substituted or unsubstituted furanyl group, a substituted or unsubstituted thiophenyl group, a substituted or unsubstituted pyrrolyl group, a substituted or unsubstituted pyrazolyl group, a substituted or unsubstituted imidazolyl group, a substituted or unsubstituted triazolyl group, a substituted or unsubstituted oxazolyl group, a substituted or unsubstituted thiazolyl group, a substituted or unsubstituted oxadiazolyl group, a substituted or unsubstituted thiadiazolyl group, a substituted or unsubstituted pyridyl group, a substituted or unsubstituted pyrimidinyl group, a substituted or unsubstituted pyrazinyl group, a substituted or unsubstituted triazinyl group, a substituted or unsubstituted benzofuranyl group, a substituted or unsubstituted benzothiophenyl group, a substituted or unsubstituted benzimidazolyl group, a substituted or unsubstituted indolyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted naphthyridinyl group, a substituted or unsubstituted benzoxazinyl group, a substituted or unsubstituted benzthiazinyl group, a substituted or unsubstituted acridinyl group, a substituted or unsubstituted phenazinyl group, a substituted or unsubstituted phenothiazinyl group, a substituted or unsubstituted phenoxazinyl group, a substituted or unsubstituted dibenzofuranyl group, or a substituted or unsubstituted dibenzothiophenyl group, or a combination thereof.

As used herein, the description of adjacent groups being linked with each other to form a substituted or unsubstituted aromatic monocyclic or polycyclic ring, or a substituted or unsubstituted heteroaromatic monocyclic or polycyclic ring means that any two adjacent substituents directly substituting an aromatic ring or an heteroaromatic ring with a single bond without a linking group are linked to form an additional ring.

For example, adjacent groups may be linked with each other to form a substituted or unsubstituted aromatic monocyclic or polycyclic ring and examples may be a substituted or unsubstituted aromatic monocyclic ring.

For example, any two substituents directly substituting the pyrimidine ring may be linked with each other to form an additional ring, and thereby a substituted or unsubstituted quinazolinyl group may be formed together with the pyrimidine ring.

In the present specification, hole characteristics refer to an ability to donate an electron to form a hole when an electric field is applied and that a hole formed in the anode may be easily injected into the light emitting layer and transported in the light emitting layer due to conductive characteristics according to a highest occupied molecular orbital (HOMO) level.

In addition, electron characteristics refer to an ability to accept an electron when an electric field is applied and that electron formed in the cathode may be easily injected into the light emitting layer and transported in the light emitting layer due to conductive characteristics according to a lowest unoccupied molecular orbital (LUMO) level.

Hereinafter, an organic optoelectronic device according to an embodiment is described.

The organic optoelectronic device may be a device to convert electrical energy into photoenergy and vice versa, and may include, e.g., an organic photoelectric device, an organic light emitting diode, an organic solar cell, or an organic photo-conductor drum.

Herein, an organic light emitting diode as one examples of an organic optoelectronic device is described, and may be applied to other organic optoelectronic devices in the same manner.

The FIGURE illustrates a cross-sectional view of an organic light emitting diode according to an embodiment.

Referring to the FIGURE an organic light emitting diode 300 according to an embodiment may include an anode 110 and a cathode 120 facing each other and an organic layer 105 between the anode 110 and the cathode 120. The organic layer 105 may include, e.g., a light emitting layer 130, a hole transport auxiliary layer 142, and a hole transport layer 141.

The anode 110 may be made of a conductor having a large work function to facilitate hole injection, and may include, e.g., a metal, a metal oxide, or a conductive polymer. In an implementation, the anode 110 may include, e.g., a metal nickel, platinum, vanadium, chromium, copper, zinc, gold, and the like or an alloy thereof; metal oxide such as zinc oxide, indium oxide, indium tin oxide (ITO), indium zinc oxide (IZO), and the like; a combination of metal and oxide such as ZnO and Al or SnO₂ and Sb; a conductive polymer such as poly(3-methylthiophene), poly(3,4-(ethylene-1,2-dioxy)thiophene) (PEDOT), polypyrrole, or polyaniline.

The cathode 120 may be made of a conductor having a small work function to facilitate electron injection, and may include, e.g., a metal, a metal oxide, or a conductive polymer. In an implementation, the cathode 120 may include, e.g., a metal or an alloy thereof such as magnesium, calcium, sodium, potassium, titanium, indium, yttrium, lithium, gadolinium, aluminum silver, tin, lead, cesium, barium, and the like; or a multi-layer structure material such as LiF/Al, LiO₂/Al, LiF/Ca, LiF/Al and BaF₂/Ca.

The light emitting layer 130 may be between the anode 110 and the cathode 120 and may include a plurality of hosts and at least one type of a dopant.

In an implementation, the light emitting layer 130 may include a host including, e.g., a first compound having relatively strong hole characteristics and a second compound having relatively strong electron characteristics.

The first compound may be, e.g., a compound having relatively strong hole characteristics and may be represented by Chemical Formula 1.

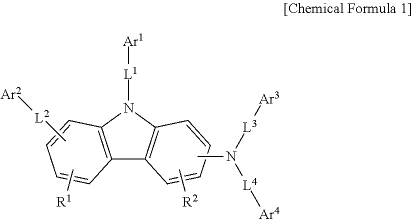

In Chemical Formula 1,

Ar¹ and Ar² may each independently be or include, e.g., a substituted or unsubstituted C6 to C12 aryl group,

Ar³ and Ar⁴ may each independently be or include, e.g., a substituted or unsubstituted C6 to C30 aryl group,

L¹ may be or may include, e.g., a single bond or a substituted or unsubstituted phenylene group,

L² to L⁴ may each independently be or include, e.g., a single bond or a substituted or unsubstituted C6 to C12 arylene group, and

R¹ and R² may each independently be or include, e.g., hydrogen, deuterium, a cyano group, a substituted or unsubstituted C1 to C10 alkyl group. For example, in the chemical formulae, other bonding positions (e.g., not bound to groups identified above) are bound to hydrogen.

The first compound may have a structure in which one benzene ring of carbazole is substituted with an aryl group, the other benzene ring of carbazole is directly substituted with an arylamine group, and the carbazole is substituted with an aryl group having 18 carbon atoms or less in the N-direction of carbazole (e.g., on the nitrogen atom of the carbazole).

The carbazole core may be directly substituted with the amine group, planarity of the molecular structure may increase, and driving and life-span characteristics of the organic optoelectronic device using the first compound may be improved.

In addition, the amine group may be substituted with an aryl group, the amine group may have an appropriate HOMO energy level, and driving and life-span characteristics may be further improved.

By including the aryl group having 18 carbon atoms or less on the nitrogen atom of the carbazole, it may be easy to secure thermal stability during deposition through a low molecular weight material, compared with a structure having more than 18 carbon atoms. The first compound has a higher HOMO energy level than a structure including a heteroaryl group, and a driving voltage of the device may be improved by enhancing hole injection characteristics and hole transport characteristics of the hole transport material.

For example, in the device to which the first compound is applied, long life-span characteristics with improved driving voltage may be realized.

In an implementation, the first compound may be, e.g., represented by one of Chemical Formula 1-1 to Chemical Formula 1-4, depending on a specific substitution position of the arylamine group.

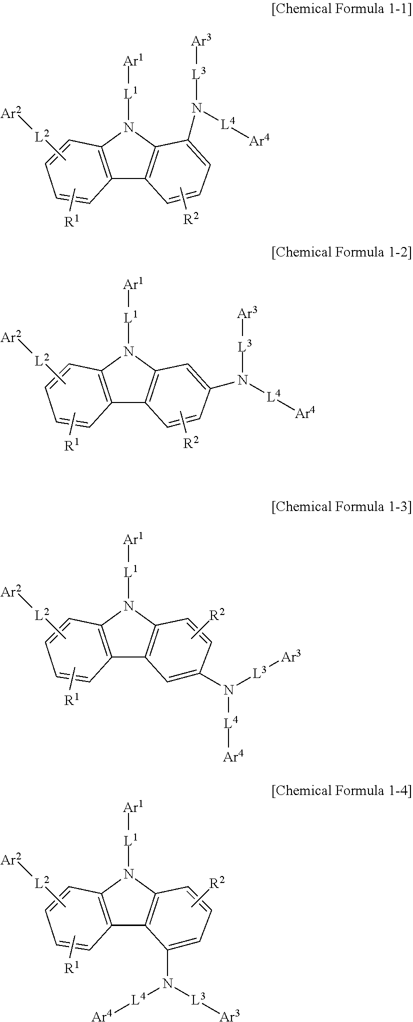

In Chemical Formula 1-1 to Chemical Formula 1-4, Ar¹ to Ar⁴, L¹ to L⁴, and R¹ and R² may be defined the same as those of Chemical Formula 1.

In an implementation, the first compound may be presented by, e.g., Chemical Formula 1-2 or Chemical Formula 1-3.

In an implementation, the first compound may be, e.g., represented by one of Chemical Formula 1a to Chemical Formula 1 d, depending on a specific substitution position of *-L²-Ar².

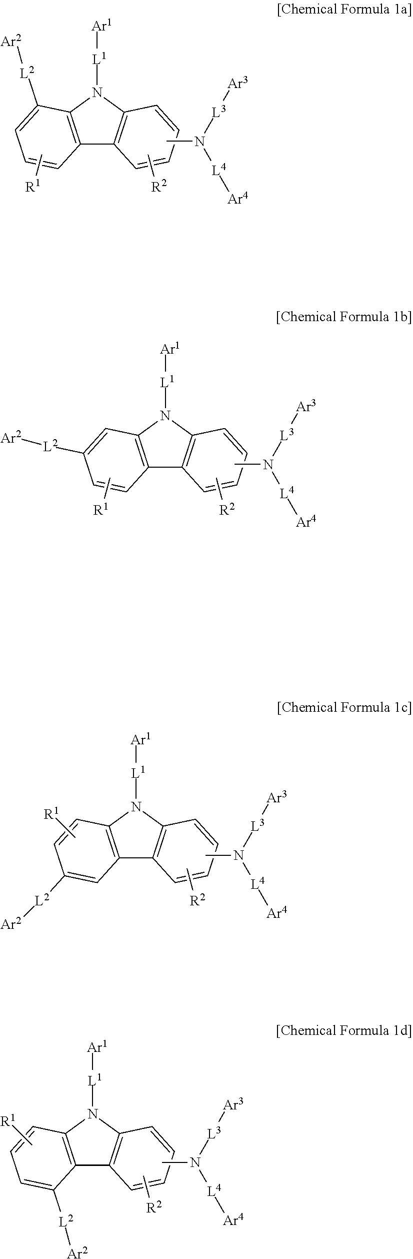

In Chemical Formula 1a to Chemical Formula 1d, Ar¹ to Ar⁴, L¹ to L⁴, and R¹ and R² may be defined the same as those of Chemical Formula 1.

In an implementation, Ar¹ and Ar² may each independently be, e.g., a substituted or unsubstituted phenyl group, a substituted or unsubstituted biphenyl group, or a substituted or unsubstituted naphthyl group.

In an implementation, L² may be, e.g., a single bond or a substituted or unsubstituted phenylene group.

In an implementation, Ar¹ and Ar² may each independently be, e.g., a substituted or unsubstituted phenyl group, or a substituted or unsubstituted naphthyl group, and L² may be, e.g., a single bond.

In an implementation, Ar³ and Ar⁴ may each independently be, e.g., a substituted or unsubstituted phenyl group, a substituted or unsubstituted biphenyl group, a substituted or unsubstituted naphthyl group, a substituted or unsubstituted terphenyl group, a substituted or unsubstituted anthracenyl group, or a substituted or unsubstituted phenanthrenyl group.

In an implementation, L³ and L⁴ may each independently be, e.g., a single bond, a substituted or unsubstituted phenylene group, or a substituted or unsubstituted naphthylene group.

In an implementation, Ar³ and Ar⁴ may each independently be, e.g., a substituted or unsubstituted phenyl group, a substituted or unsubstituted biphenyl group, or a substituted or unsubstituted naphthyl group and L³ and L⁴ may each independently be, e.g., a single bond or a substituted or unsubstituted phenylene group.

In an implementation, R¹ and R² may each independently be, e.g., hydrogen or a substituted or unsubstituted C1 to C5 alkyl group.

In an implementation, R¹ and R² may each be, e.g., hydrogen.

In an implementation, the first compound may be represented by Chemical Formula 1-2b or Chemical Formula 1-3b.

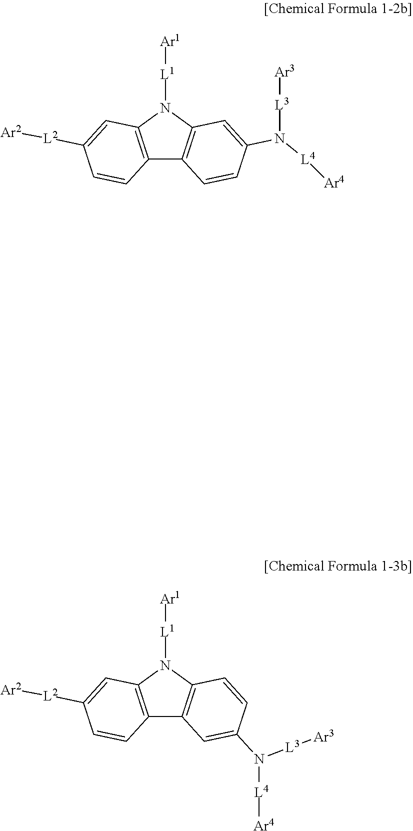

In Chemical Formula 1-2b and Chemical Formula 1-3b, Ar¹ to Ar⁴ and L¹ to L⁴ may be defined the same as those of Chemical Formula 1.

The first compound in which the amine is on the 2 or 3 position of one benzene ring of carbazole may have an improved effect on a driving voltage and life-span of the device, as compared with a structure in which the amine is on another position. By substituting the amine of the other benzene ring of carbazole with *-L²-Ar² at the 2 position, a T1 energy level may be lowered and may be particularly effective as a red host material having hole characteristics.

In an implementation, the first compound may be, e.g., represented by Chemical Formula 1-2b.

In an implementation, the first compound may be, e.g., a compound of the following Group 1.

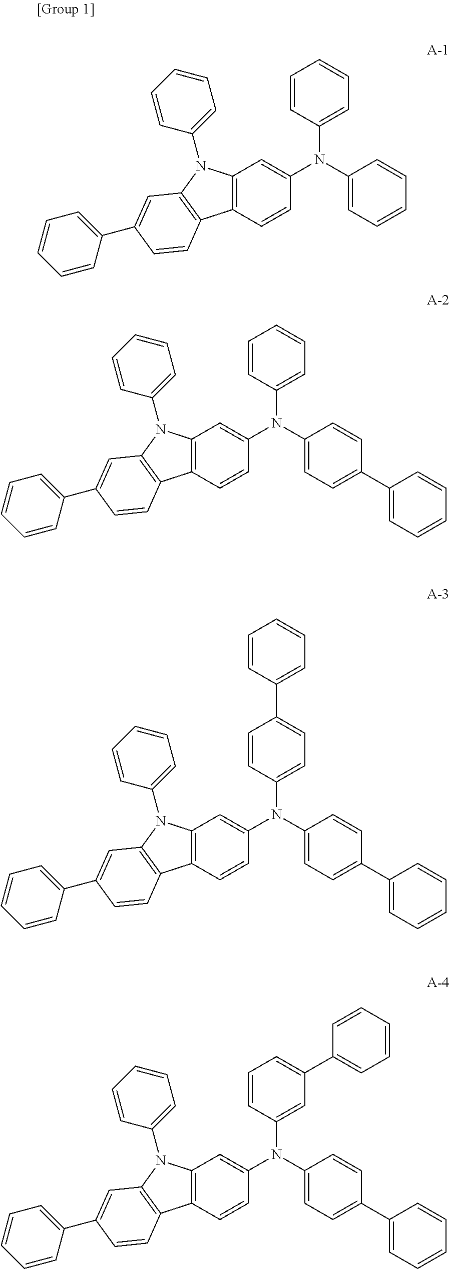 [SMILES: c1ccc(-c2ccc3c4ccc(N(c5ccccc5)c5ccccc5)cc4n(-c4ccccc4)c3c2)cc1 ; c1ccc(-c2ccc(N(c3ccccc3)c3ccc4c5ccc(-c6ccccc6)cc5n(-c5ccccc5)c4c3)cc2)cc1 ; c1ccc(-c2ccc(N(c3ccc(-c4ccccc4)cc3)c3ccc4c5ccc(-c6ccccc6)cc5n(-c5ccccc5)c4c3)cc2)cc1 ; c1ccc(-c2ccc(N(c3cccc(-c4ccccc4)c3)c3ccc4c5ccc(-c6ccccc6)cc5n(-c5ccccc5)c4c3)cc2)cc1]

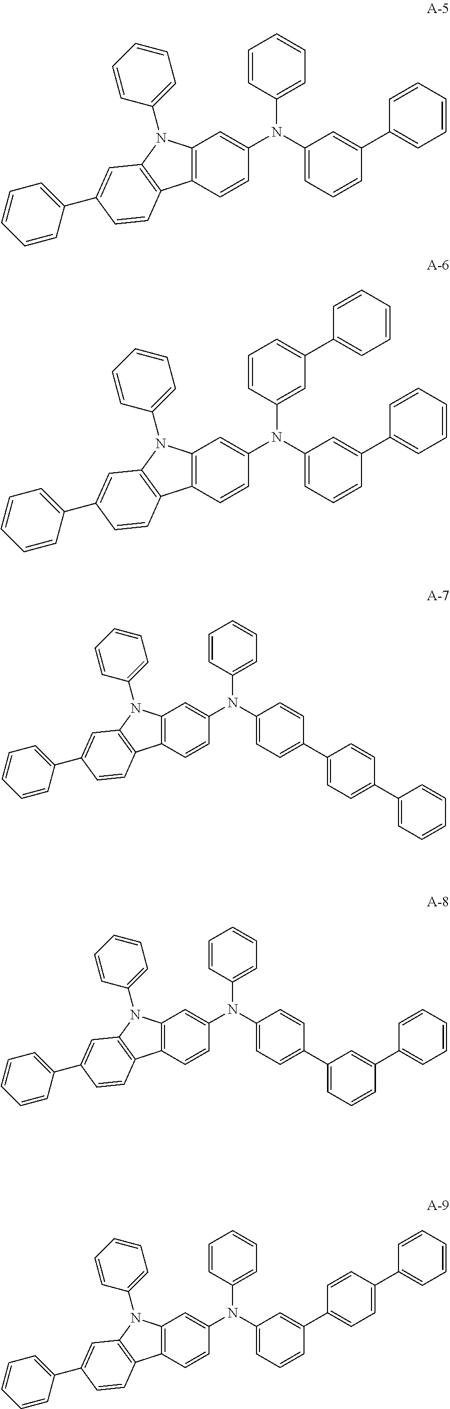 [SMILES: c1ccc(-c2cccc(N(c3ccccc3)c3ccc4c5ccc(-c6ccccc6)cc5n(-c5ccccc5)c4c3)c2)cc1 ; c1ccc(-c2cccc(N(c3cccc(-c4ccccc4)c3)c3ccc4c5ccc(-c6ccccc6)cc5n(-c5ccccc5)c4c3)c2)cc1 ; c1ccc(-c2ccc(-c3ccc(N(c4ccccc4)c4ccc5c6ccc(-c7ccccc7)cc6n(-c6ccccc6)c5c4)cc3)cc2)cc1 ; c1ccc(-c2cccc(-c3ccc(N(c4ccccc4)c4ccc5c6ccc(-c7ccccc7)cc6n(-c6ccccc6)c5c4)cc3)c2)cc1 ; c1ccc(-c2ccc(-c3cccc(N(c4ccccc4)c4ccc5c6ccc(-c7ccccc7)cc6n(-c6ccccc6)c5c4)c3)cc2)cc1]

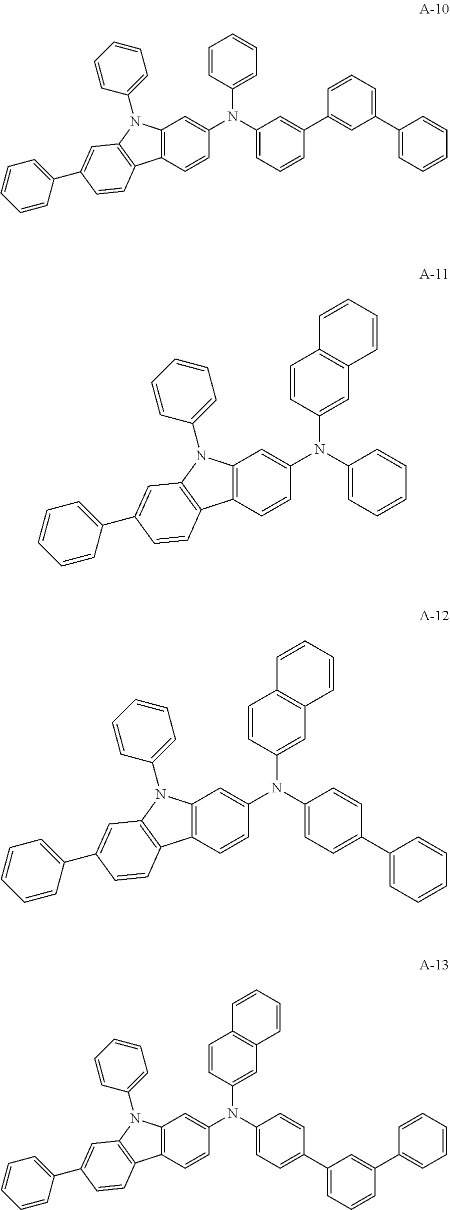 [SMILES: c1ccc(-c2cccc(-c3cccc(N(c4ccccc4)c4ccc5c6ccc(-c7ccccc7)cc6n(-c6ccccc6)c5c4)c3)c2)cc1 ; c1ccc(-c2ccc3c4ccc(N(c5ccccc5)c5ccc6ccccc6c5)cc4n(-c4ccccc4)c3c2)cc1 ; c1ccc(-c2ccc(N(c3ccc4ccccc4c3)c3ccc4c5ccc(-c6ccccc6)cc5n(-c5ccccc5)c4c3)cc2)cc1 ; c1ccc(-c2cccc(-c3ccc(N(c4ccc5ccccc5c4)c4ccc5c6ccc(-c7ccccc7)cc6n(-c6ccccc6)c5c4)cc3)c2)cc1]

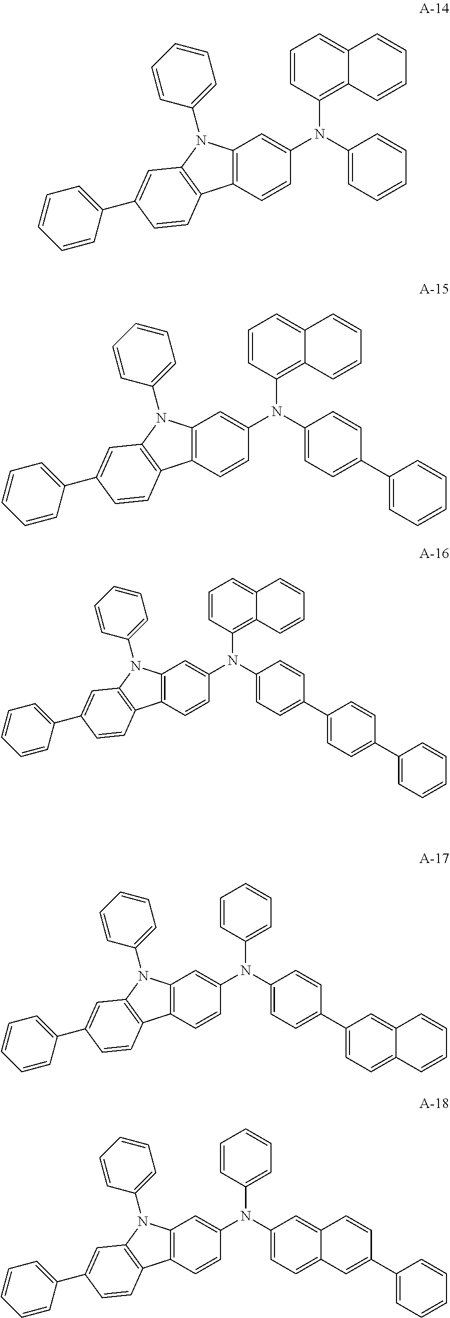 [SMILES: c1ccc(-c2ccc3c4ccc(N(c5ccccc5)c5cccc6ccccc56)cc4n(-c4ccccc4)c3c2)cc1 ; c1ccc(-c2ccc(N(c3ccc4c5ccc(-c6ccccc6)cc5n(-c5ccccc5)c4c3)c3cccc4ccccc34)cc2)cc1 ; c1ccc(-c2ccc(-c3ccc(N(c4ccc5c6ccc(-c7ccccc7)cc6n(-c6ccccc6)c5c4)c4cccc5ccccc45)cc3)cc2)cc1 ; c1ccc(-c2ccc3c4ccc(N(c5ccccc5)c5ccc(-c6ccc7ccccc7c6)cc5)cc4n(-c4ccccc4)c3c2)cc1 ; c1ccc(-c2ccc3cc(N(c4ccccc4)c4ccc5c6ccc(-c7ccccc7)cc6n(-c6ccccc6)c5c4)ccc3c2)cc1]

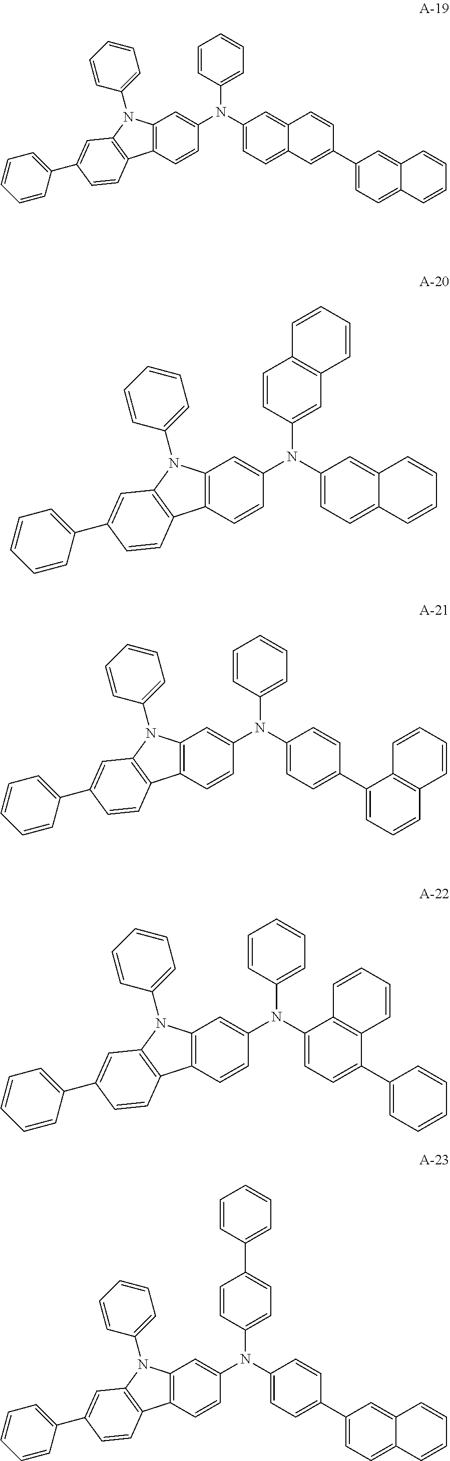 [SMILES: c1ccc(-c2ccc3c4ccc(N(c5ccccc5)c5ccc6cc(-c7ccc8ccccc8c7)ccc6c5)cc4n(-c4ccccc4)c3c2)cc1 ; c1ccc(-c2ccc3c4ccc(N(c5ccc6ccccc6c5)c5ccc6ccccc6c5)cc4n(-c4ccccc4)c3c2)cc1 ; c1ccc(-c2ccc3c4ccc(N(c5ccccc5)c5ccc(-c6cccc7ccccc67)cc5)cc4n(-c4ccccc4)c3c2)cc1 ; c1ccc(-c2ccc3c4ccc(N(c5ccccc5)c5ccc(-c6ccccc6)c6ccccc56)cc4n(-c4ccccc4)c3c2)cc1 ; c1ccc(-c2ccc(N(c3ccc(-c4ccc5ccccc5c4)cc3)c3ccc4c5ccc(-c6ccccc6)cc5n(-c5ccccc5)c4c3)cc2)cc1]

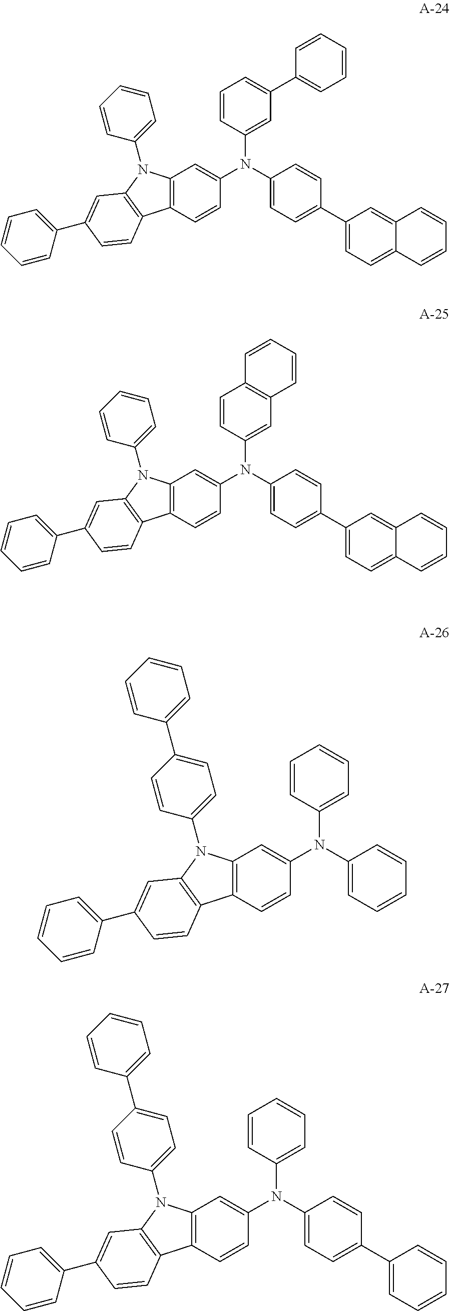 [SMILES: c1ccc(-c2cccc(N(c3ccc(-c4ccc5ccccc5c4)cc3)c3ccc4c5ccc(-c6ccccc6)cc5n(-c5ccccc5)c4c3)c2)cc1 ; c1ccc(-c2ccc3c4ccc(N(c5ccc(-c6ccc7ccccc7c6)cc5)c5ccc6ccccc6c5)cc4n(-c4ccccc4)c3c2)cc1 ; c1ccc(-c2ccc(-n3c4cc(-c5ccccc5)ccc4c4ccc(N(c5ccccc5)c5ccccc5)cc43)cc2)cc1 ; c1ccc(-c2ccc(N(c3ccccc3)c3ccc4c5ccc(-c6ccccc6)cc5n(-c5ccc(-c6ccccc6)cc5)c4c3)cc2)cc1]

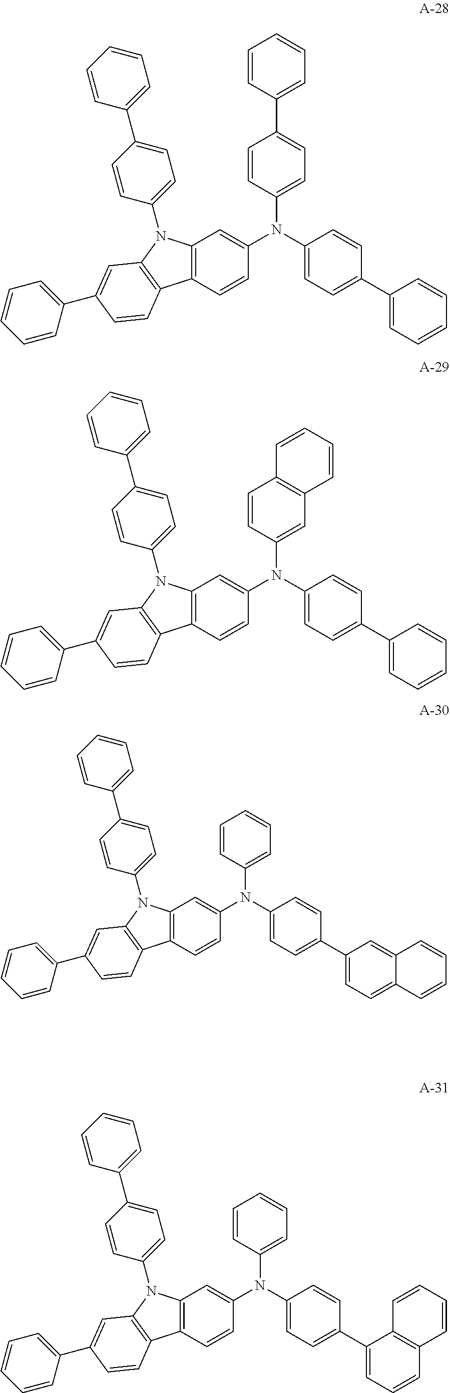 [SMILES: c1ccc(-c2ccc(N(c3ccc(-c4ccccc4)cc3)c3ccc4c5ccc(-c6ccccc6)cc5n(-c5ccc(-c6ccccc6)cc5)c4c3)cc2)cc1 ; c1ccc(-c2ccc(N(c3ccc4ccccc4c3)c3ccc4c5ccc(-c6ccccc6)cc5n(-c5ccc(-c6ccccc6)cc5)c4c3)cc2)cc1 ; c1ccc(-c2ccc(-n3c4cc(-c5ccccc5)ccc4c4ccc(N(c5ccccc5)c5ccc(-c6ccc7ccccc7c6)cc5)cc43)cc2)cc1 ; c1ccc(-c2ccc(-n3c4cc(-c5ccccc5)ccc4c4ccc(N(c5ccccc5)c5ccc(-c6cccc7ccccc67)cc5)cc43)cc2)cc1]

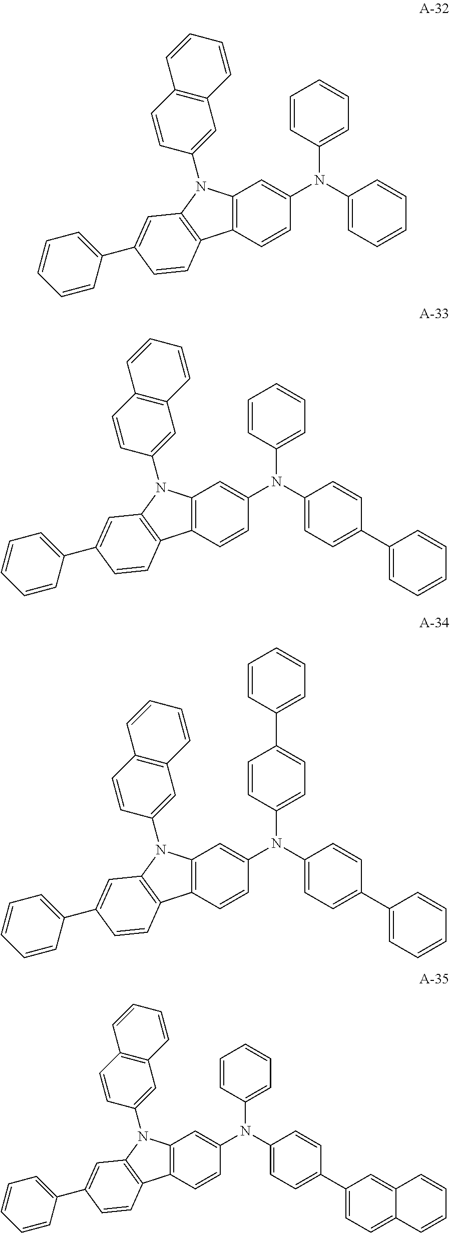 [SMILES: c1ccc(-c2ccc3c4ccc(N(c5ccccc5)c5ccccc5)cc4n(-c4ccc5ccccc5c4)c3c2)cc1 ; c1ccc(-c2ccc(N(c3ccccc3)c3ccc4c5ccc(-c6ccccc6)cc5n(-c5ccc6ccccc6c5)c4c3)cc2)cc1 ; c1ccc(-c2ccc(N(c3ccc(-c4ccccc4)cc3)c3ccc4c5ccc(-c6ccccc6)cc5n(-c5ccc6ccccc6c5)c4c3)cc2)cc1 ; c1ccc(-c2ccc3c4ccc(N(c5ccccc5)c5ccc(-c6ccc7ccccc7c6)cc5)cc4n(-c4ccc5ccccc5c4)c3c2)cc1]

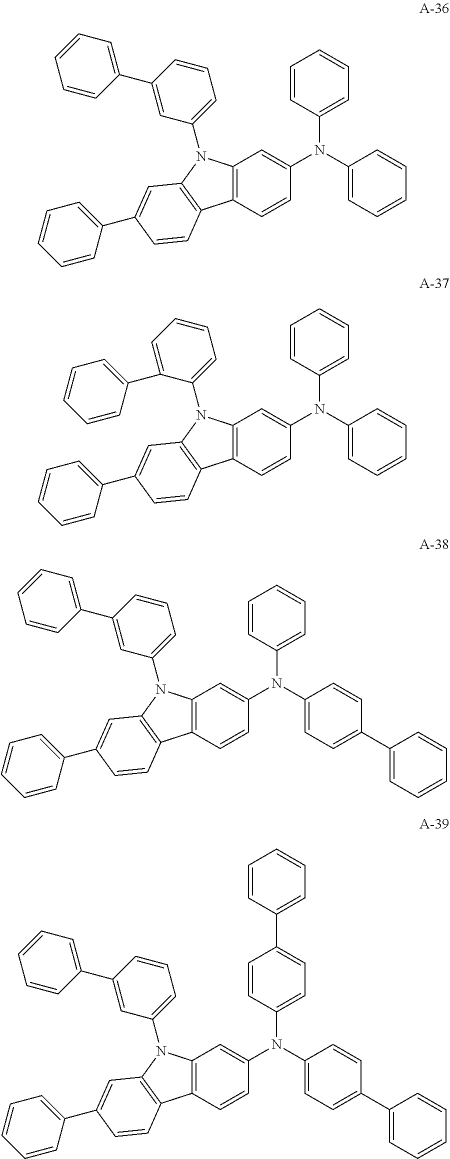 [SMILES: c1ccc(-c2cccc(-n3c4cc(-c5ccccc5)ccc4c4ccc(N(c5ccccc5)c5ccccc5)cc43)c2)cc1 ; c1ccc(-c2ccc3c4ccc(N(c5ccccc5)c5ccccc5)cc4n(-c4ccccc4-c4ccccc4)c3c2)cc1 ; c1ccc(-c2ccc(N(c3ccccc3)c3ccc4c5ccc(-c6ccccc6)cc5n(-c5cccc(-c6ccccc6)c5)c4c3)cc2)cc1 ; c1ccc(-c2ccc(N(c3ccc(-c4ccccc4)cc3)c3ccc4c5ccc(-c6ccccc6)cc5n(-c5cccc(-c6ccccc6)c5)c4c3)cc2)cc1]

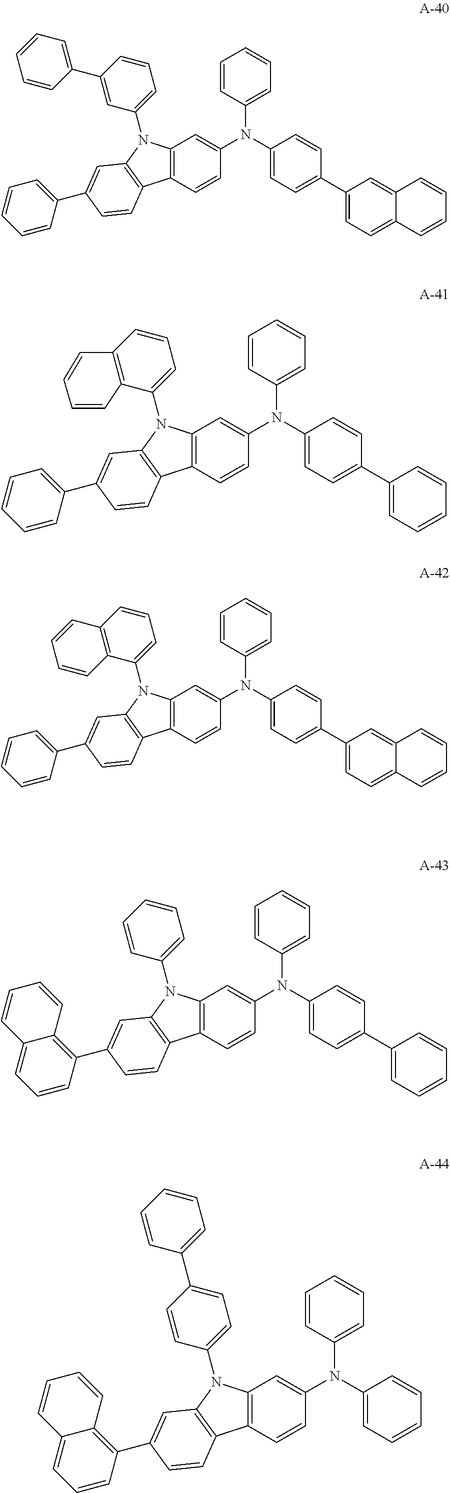 [SMILES: c1ccc(-c2cccc(-n3c4cc(-c5ccccc5)ccc4c4ccc(N(c5ccccc5)c5ccc(-c6ccc7ccccc7c6)cc5)cc43)c2)cc1 ; c1ccc(-c2ccc(N(c3ccccc3)c3ccc4c5ccc(-c6ccccc6)cc5n(-c5cccc6ccccc56)c4c3)cc2)cc1 ; c1ccc(-c2ccc3c4ccc(N(c5ccccc5)c5ccc(-c6ccc7ccccc7c6)cc5)cc4n(-c4cccc5ccccc45)c3c2)cc1 ; c1ccc(-c2ccc(N(c3ccccc3)c3ccc4c5ccc(-c6cccc7ccccc67)cc5n(-c5ccccc5)c4c3)cc2)cc1 ; c1ccc(-c2ccc(-n3c4cc(-c5cccc6ccccc56)ccc4c4ccc(N(c5ccccc5)c5ccccc5)cc43)cc2)cc1]

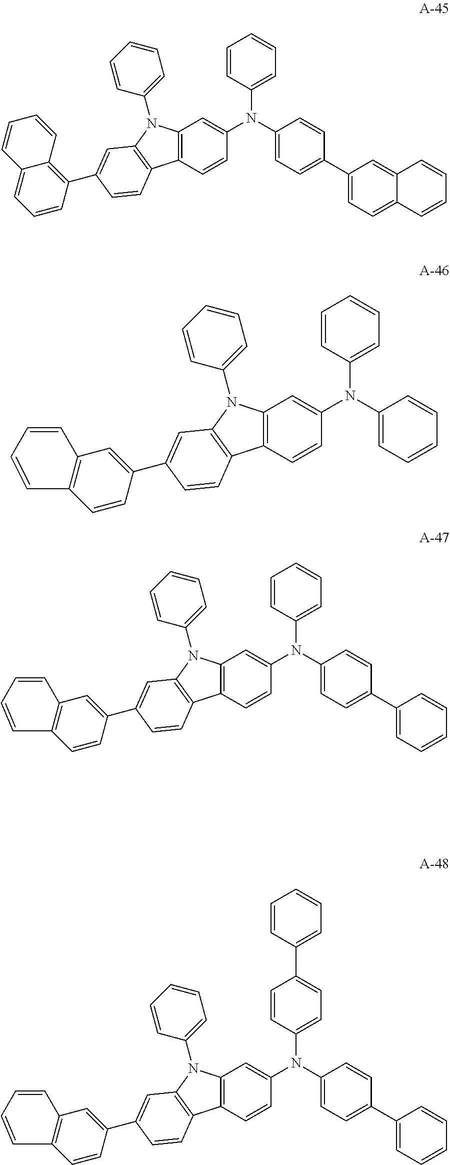 [SMILES: c1ccc(N(c2ccc(-c3ccc4ccccc4c3)cc2)c2ccc3c4ccc(-c5cccc6ccccc56)cc4n(-c4ccccc4)c3c2)cc1 ; c1ccc(N(c2ccccc2)c2ccc3c4ccc(-c5ccc6ccccc6c5)cc4n(-c4ccccc4)c3c2)cc1 ; c1ccc(-c2ccc(N(c3ccccc3)c3ccc4c5ccc(-c6ccc7ccccc7c6)cc5n(-c5ccccc5)c4c3)cc2)cc1 ; c1ccc(-c2ccc(N(c3ccc(-c4ccccc4)cc3)c3ccc4c5ccc(-c6ccc7ccccc7c6)cc5n(-c5ccccc5)c4c3)cc2)cc1]

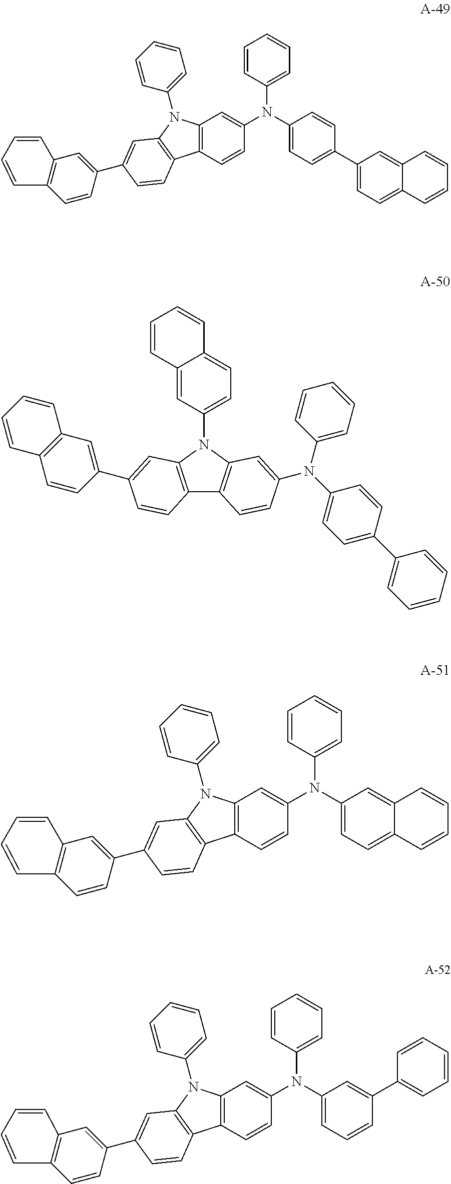 [SMILES: c1ccc(N(c2ccc(-c3ccc4ccccc4c3)cc2)c2ccc3c4ccc(-c5ccc6ccccc6c5)cc4n(-c4ccccc4)c3c2)cc1 ; c1ccc(-c2ccc(N(c3ccccc3)c3ccc4c5ccc(-c6ccc7ccccc7c6)cc5n(-c5ccc6ccccc6c5)c4c3)cc2)cc1 ; c1ccc(N(c2ccc3ccccc3c2)c2ccc3c4ccc(-c5ccc6ccccc6c5)cc4n(-c4ccccc4)c3c2)cc1 ; c1ccc(-c2cccc(N(c3ccccc3)c3ccc4c5ccc(-c6ccc7ccccc7c6)cc5n(-c5ccccc5)c4c3)c2)cc1]

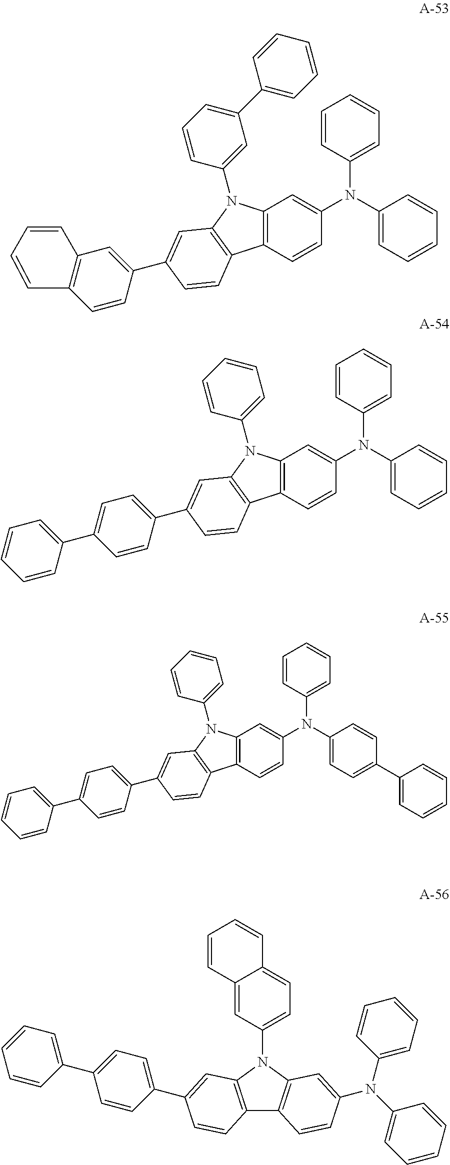 [SMILES: c1ccc(-c2cccc(-n3c4cc(-c5ccc6ccccc6c5)ccc4c4ccc(N(c5ccccc5)c5ccccc5)cc43)c2)cc1 ; c1ccc(-c2ccc(-c3ccc4c5ccc(N(c6ccccc6)c6ccccc6)cc5n(-c5ccccc5)c4c3)cc2)cc1 ; c1ccc(-c2ccc(-c3ccc4c5ccc(N(c6ccccc6)c6ccc(-c7ccccc7)cc6)cc5n(-c5ccccc5)c4c3)cc2)cc1 ; c1ccc(-c2ccc(-c3ccc4c5ccc(N(c6ccccc6)c6ccccc6)cc5n(-c5ccc6ccccc6c5)c4c3)cc2)cc1]

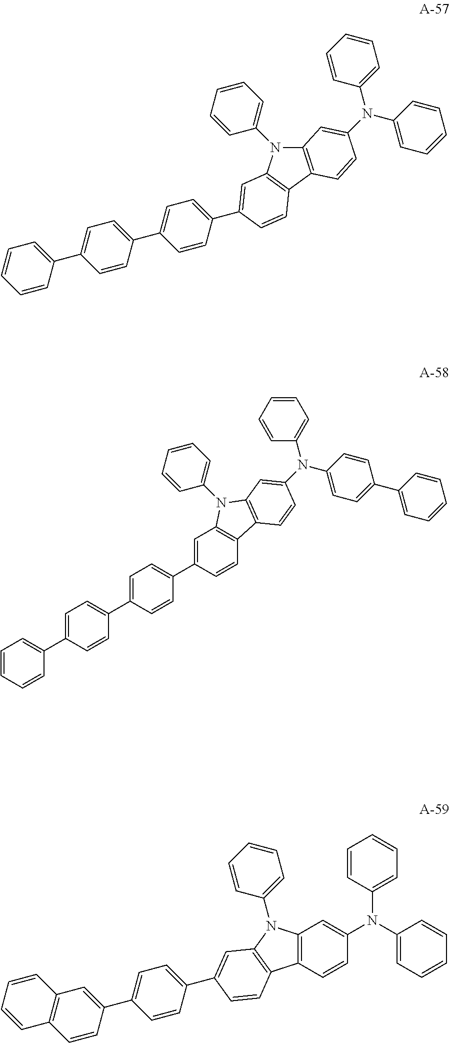 [SMILES: c1ccc(-c2ccc(-c3ccc(-c4ccc5c6ccc(N(c7ccccc7)c7ccccc7)cc6n(-c6ccccc6)c5c4)cc3)cc2)cc1 ; c1ccc(-c2ccc(-c3ccc(-c4ccc5c6ccc(N(c7ccccc7)c7ccc(-c8ccccc8)cc7)cc6n(-c6ccccc6)c5c4)cc3)cc2)cc1 ; c1ccc(N(c2ccccc2)c2ccc3c4ccc(-c5ccc(-c6ccc7ccccc7c6)cc5)cc4n(-c4ccccc4)c3c2)cc1]

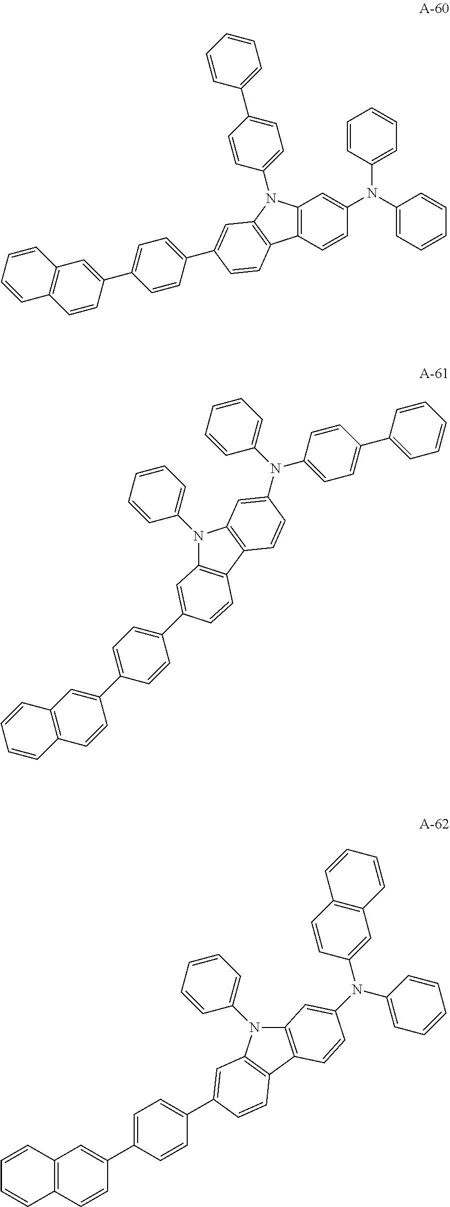 [SMILES: c1ccc(-c2ccc(-n3c4cc(-c5ccc(-c6ccc7ccccc7c6)cc5)ccc4c4ccc(N(c5ccccc5)c5ccccc5)cc43)cc2)cc1 ; c1ccc(-c2ccc(N(c3ccccc3)c3ccc4c5ccc(-c6ccc(-c7ccc8ccccc8c7)cc6)cc5n(-c5ccccc5)c4c3)cc2)cc1 ; c1ccc(N(c2ccc3ccccc3c2)c2ccc3c4ccc(-c5ccc(-c6ccc7ccccc7c6)cc5)cc4n(-c4ccccc4)c3c2)cc1]

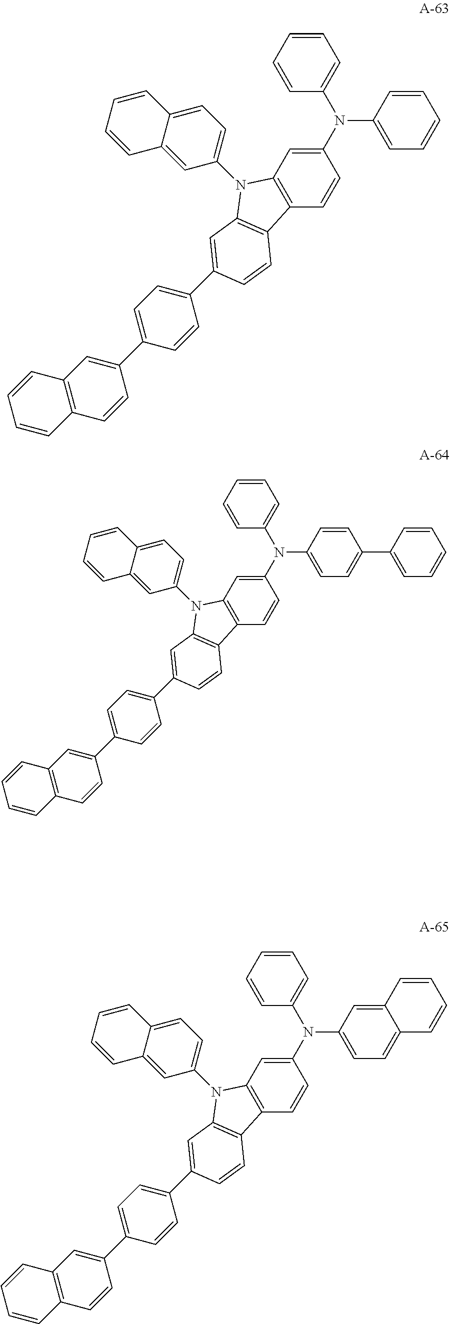 [SMILES: c1ccc(N(c2ccccc2)c2ccc3c4ccc(-c5ccc(-c6ccc7ccccc7c6)cc5)cc4n(-c4ccc5ccccc5c4)c3c2)cc1 ; c1ccc(-c2ccc(N(c3ccccc3)c3ccc4c5ccc(-c6ccc(-c7ccc8ccccc8c7)cc6)cc5n(-c5ccc6ccccc6c5)c4c3)cc2)cc1 ; c1ccc(N(c2ccc3ccccc3c2)c2ccc3c4ccc(-c5ccc(-c6ccc7ccccc7c6)cc5)cc4n(-c4ccc5ccccc5c4)c3c2)cc1]

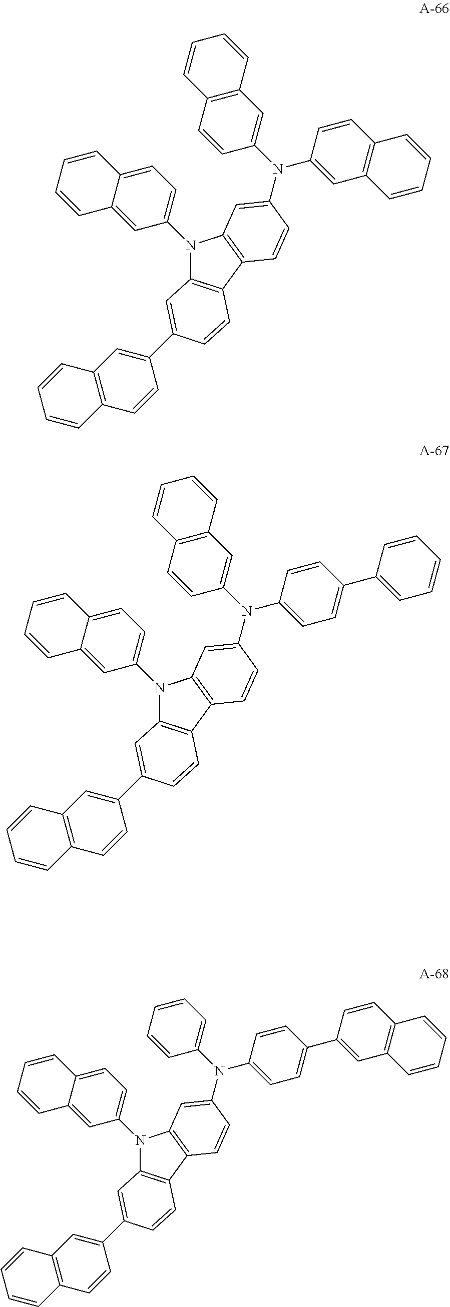 [SMILES: c1ccc2cc(-c3ccc4c5ccc(N(c6ccc7ccccc7c6)c6ccc7ccccc7c6)cc5n(-c5ccc6ccccc6c5)c4c3)ccc2c1 ; c1ccc(-c2ccc(N(c3ccc4ccccc4c3)c3ccc4c5ccc(-c6ccc7ccccc7c6)cc5n(-c5ccc6ccccc6c5)c4c3)cc2)cc1 ; c1ccc(N(c2ccc(-c3ccc4ccccc4c3)cc2)c2ccc3c4ccc(-c5ccc6ccccc6c5)cc4n(-c4ccc5ccccc5c4)c3c2)cc1]

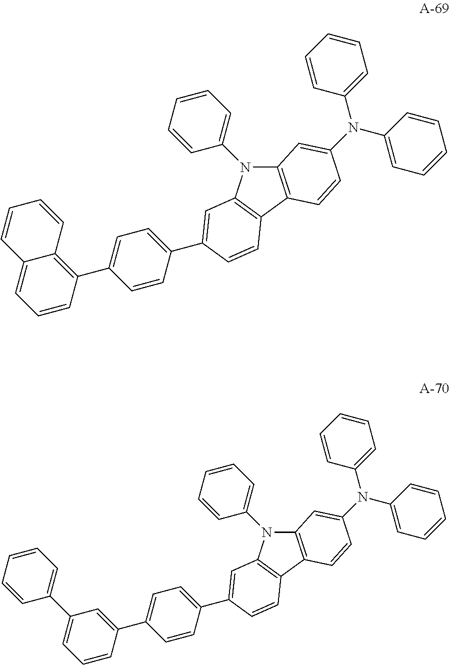 [SMILES: c1ccc(N(c2ccccc2)c2ccc3c4ccc(-c5ccc(-c6cccc7ccccc67)cc5)cc4n(-c4ccccc4)c3c2)cc1 ; c1ccc(-c2cccc(-c3ccc(-c4ccc5c6ccc(N(c7ccccc7)c7ccccc7)cc6n(-c6ccccc6)c5c4)cc3)c2)cc1]

In an implementation, the second compound may be, e.g., represented by Chemical Formula 2 as a compound having relatively strong electron characteristics.

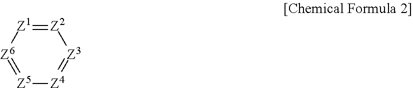

In Chemical Formula 2,

Z¹ may be, e.g., N or C-L⁵-R³,

Z² may be, e.g., N or C-L⁶-R⁴,

Z³ may be, e.g., N or C-L⁷-R⁵,

Z⁴ may be, e.g., N or C-L⁸-R⁶,

Z⁵ may be, e.g., N or C-L⁹-R⁷, and

Z⁶ may be, e.g., N or C-L¹⁰-R⁸.

In an implementation, at least two of Z¹ to Z⁶ are N.

L⁵ to L¹⁰ may each independently be or include, e.g., a single bond, a substituted or unsubstituted C6 to C20 arylene group, a substituted or unsubstituted divalent C2 to C20 heterocyclic group, or a combination thereof, and

R³ to R⁸ may each independently be or include, e.g., hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted silyl group, a substituted or unsubstituted amine group, a halogen, a cyano group, or a combination thereof.

In an implementation, at least one of R³ to R⁸ may be, e.g., a substituted or unsubstituted dibenzofuranyl group, a substituted or unsubstituted dibenzothiophenyl group, a substituted or unsubstituted carbazolyl group, a substituted or unsubstituted benzocarbazolyl group, a substituted or unsubstituted dibenzocarbazolyl group, or a substituted or unsubstituted triphenylene group.

In an implementation, R³ to R⁸ may be separate, or adjacent groups thereof may be linked with each other to form a substituted or unsubstituted aliphatic monocyclic or polycyclic ring, a substituted or unsubstituted aromatic monocyclic or polycyclic ring, or a substituted or unsubstituted heteroaromatic monocyclic or polycyclic ring.

The second compound may effectively expand the LUMO energy band by including the nitrogen-containing hexagonal (e.g., six-membered) ring moiety, and may be included along with the aforementioned first compound to improve a balance of holes and electrons, greatly improving life-span characteristics of the device including the same.

In an implementation, two of Z¹ to Z⁶ may be nitrogen (N) and remaining ones of Z¹ to Z⁶ may be CRⁿ.

Rⁿ refers to any substituent selected from R³ to R⁸.

In an implementation, Z¹ and Z³ may be nitrogen, Z² may be N or C-L⁶-R⁴, Z⁴ may be N or C-L⁸-R⁶, Z⁵ may be N or C-L⁹-R⁷, and Z⁶ may be N or C-L¹⁰-R⁸.

In this case, at least one of R⁴, R⁶ to R⁸ may be, e.g., a substituted or unsubstituted dibenzofuranyl group, a substituted or unsubstituted dibenzothiophenyl group, a substituted or unsubstituted carbazolyl group, a substituted or unsubstituted benzocarbazolyl group, a substituted or unsubstituted dibenzocarbazolyl group, or a substituted or unsubstituted triphenylene group.

In an implementation, three of Z¹ to Z⁶ may be nitrogen (N) and remaining ones of Z¹ to Z⁶ may be CR.

In an implementation, Z¹, Z³, and Z⁵ may be nitrogen, Z² may be N or C-L⁶-R⁴, Z⁴ may be N or C-L⁸-R⁶, and Z⁶ may be N or C-L¹⁰-R⁸.

In this case, at least one of R⁴, R⁶, and R⁸ may be, e.g., a substituted or unsubstituted dibenzofuranyl group, a substituted or unsubstituted dibenzothiophenyl group, a substituted or unsubstituted carbazolyl group, a substituted or unsubstituted benzocarbazolyl group, a substituted or unsubstituted dibenzocarbazolyl group, or a substituted or unsubstituted triphenylene group.

In an implementation, when R³ to R⁸ are each independently present, e.g., separately present, the second compound may be represented by Chemical Formula 2-1.

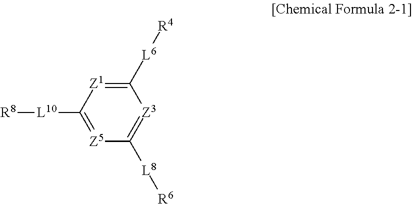

In Chemical Formula 2-1, Z¹, Z³, and Z⁵ may each independently be, e.g., N or CH, at least two of Z¹, Z³, and Z⁵ are N, L⁶, L⁸, and L¹⁰ and R⁴, R⁶, and R⁸ are the same as described above, and at least one of R⁴, R⁶, and R⁸ may be, e.g., a substituted or unsubstituted dibenzofuranyl group, a substituted or unsubstituted dibenzothiophenyl group, a substituted or unsubstituted carbazolyl group, a substituted or unsubstituted benzocarbazolyl group, a substituted or unsubstituted dibenzocarbazolyl group, or a substituted or unsubstituted triphenylene group.

In an implementation, the compound represented by Chemical Formula 2-1 may be represented by Chemical Formula 2-1a or Chemical Formula 2-1b.

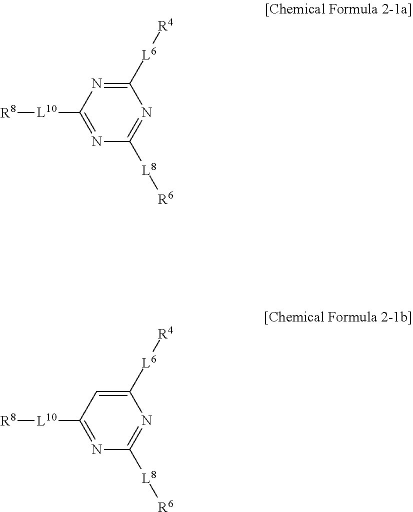

In Chemical Formula 2-1a and Chemical Formula 2-1b, L⁶, L⁸ and L¹⁰ and R⁴, R⁶, and R⁸ may be defined the same as those of Chemical Formula 2.

In an implementation, adjacent groups of R³ to R⁸ may be linked with each other to form, e.g., a substituted or unsubstituted aliphatic monocyclic or polycyclic ring, a substituted or unsubstituted aromatic monocyclic or polycyclic ring, or a substituted or unsubstituted heteroaromatic monocyclic or polycyclic ring, and at least one of R³ to R⁸ that do not form a ring may be a substituted or unsubstituted dibenzofuranyl group, a substituted or unsubstituted dibenzothiophenyl group, a substituted or unsubstituted carbazolyl group, a substituted or unsubstituted benzocarbazolyl group, a substituted or unsubstituted dibenzocarbazolyl group, or a substituted or unsubstituted triphenylene group.

As noted above, in the present specification, the description of adjacent groups thereof are linked with each other to form a substituted or unsubstituted aliphatic monocyclic or polycyclic ring, a substituted or unsubstituted aromatic monocyclic or polycyclic ring, or a substituted or unsubstituted heteroaromatic monocyclic or polycyclic ring means that any adjacent two substituents may be fused to form a ring. For example, adjacent R³ and R⁴, R⁴ and R⁵, R⁵ and R⁶, R⁶ and R⁷, or R⁷ and R⁸ in Chemical Formula 2 may be fused and may form a heteroaromatic polycyclic ring together with the nitrogen-containing hexagonal ring moiety substituted with these groups. Herein, examples of the formed heteroaromatic polycyclic ring may be a substituted or unsubstituted quinazolinyl group, a substituted or unsubstituted quinoxalinyl group, a substituted or unsubstituted benzofuranpyrimidinyl group, a substituted or unsubstituted benzothiophenepyrimidinyl group, and the like and for example R⁴ and R⁵ of Chemical Formula 2 are fused to form a heteroaromatic polycyclic ring together with the nitrogen-containing hexagonal ring moiety substituted with these groups. They may be represented by Chemical Formula 2-2 or Chemical Formula 2-3.

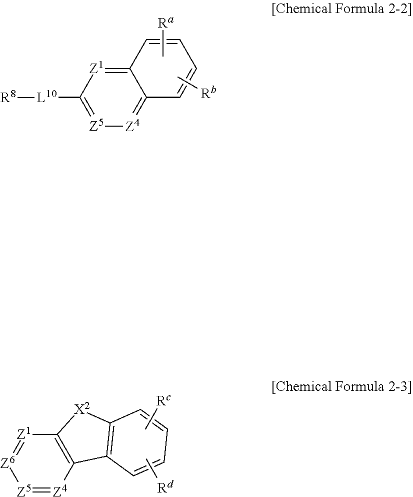

In Chemical Formula 2-2 and Chemical Formula 2-3, Z¹, Z⁴, Z⁵, Z⁶, L¹⁰, and R⁸ may be defined the same as those of Chemical Formula 2, X² may be, e.g., O or S, and R^{a}, R^{b}, R^{c}, and R^{d} may each independently be, e.g., hydrogen, deuterium, a halogen, a cyano group, a C1 to C20 alkyl group, a C6 to C30 aryl group, a C2 to C30 heteroaryl group, or a combination thereof.

In an implementation, Z¹ and Z⁵ of Chemical Formula 2-2 may be each N.

In an implementation, Z¹ and Z⁴ of Chemical Formula 2-2 may be each N.

In an implementation, the compound represented by Chemical Formula 2-2 may be represented by, e.g., Chemical Formula 2-2a or Chemical Formula 2-2b.

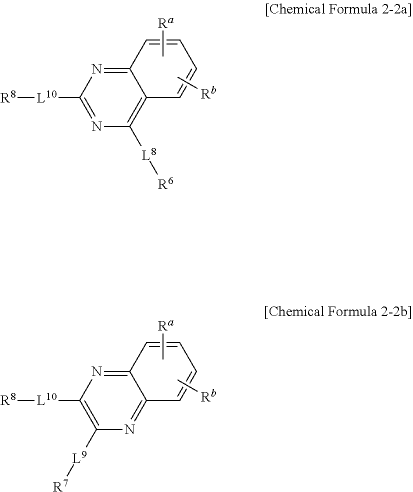

In Chemical Formula 2-2a and Chemical Formula 2-2b, L⁸, L¹⁰, R⁶, R⁸, R^{a}, and R^{b} may be defined the same as described above.

In an implementation, Z¹ and Z⁵ of Chemical Formula 2-3 may be each N.

In an implementation, Z¹ and Z⁴ of Chemical Formula 2-3 may be each N.

In an implementation, the compound represented by Chemical Formula 2-3 may be represented by, e.g., Chemical Formula 2-3a or Chemical Formula 2-3b.

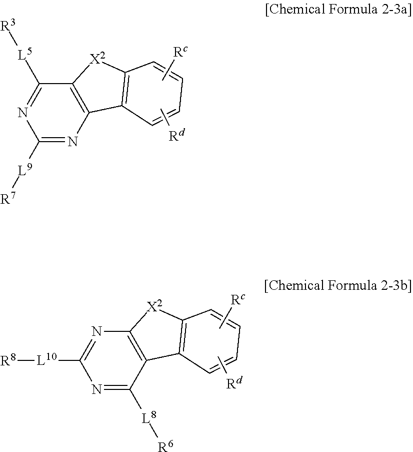

In Chemical Formula 2-3a and Chemical Formula 2-3b, X², L⁵, L⁸, L⁹, L¹⁰, R³, R⁶, R⁷, R⁸, R^{c}, and R^{d} may be defined the same as described above.

In an implementation, R³ to R⁸ of Chemical Formula 2 may each independently be, e.g., hydrogen, a substituted or unsubstituted C6 to C30 aryl group, or a substituted or unsubstituted C2 to C30 heterocyclic group.

In an implementation, R³ to R⁸ may each independently be, e.g., hydrogen, a substituted or unsubstituted phenyl group, a substituted or unsubstituted biphenyl group, a substituted or unsubstituted terphenyl group, a substituted or unsubstituted quaterphenyl group, a substituted or unsubstituted naphthyl group, a substituted or unsubstituted anthracenyl group, a substituted or unsubstituted phenanthrenyl group, a substituted or unsubstituted triphenylene group, a substituted or unsubstituted dibenzofuranyl group, a substituted or unsubstituted dibenzothiophenyl group, a substituted or unsubstituted carbazolyl group, a substituted or unsubstituted benzocarbazolyl group, a substituted or unsubstituted dibenzocarbazolyl group, a substituted or unsubstituted pyrimidinyl group, a substituted or unsubstituted triazinyl group, a substituted or unsubstituted benzofuranpyrimidinyl group, or a substituted or unsubstituted benzothiophenepyrimidinyl group.

In an implementation, at least one of R³ to R⁸ may be, e.g., a substituted or unsubstituted dibenzofuranyl group, a substituted or unsubstituted dibenzothiophenyl group, a substituted or unsubstituted carbazolyl group, a substituted or unsubstituted benzocarbazolyl group, a substituted or unsubstituted dibenzocarbazolyl group, or a substituted or unsubstituted triphenylene group.

Herein “substituted” refers to replacement of at least one hydrogen by at least one of a phenyl group, a biphenyl group, a terphenyl group, a naphthyl group, a dibenzofuranyl group, and a dibenzothiophenyl group.

In an implementation, R³ to R⁸ may each independently be, e.g., a group of the following Group I or II. In an implementation, and at least one of R³ to R⁸ may be, e.g., a group of the following Group II.

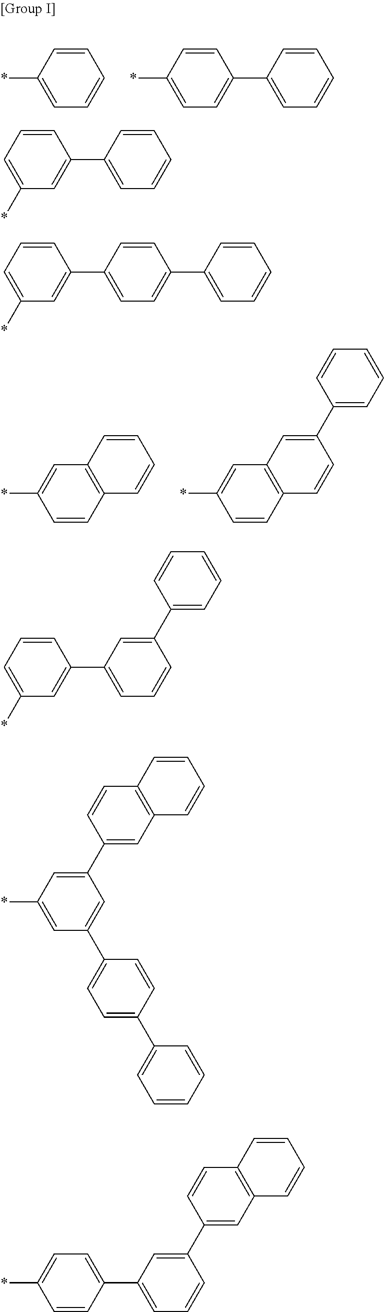

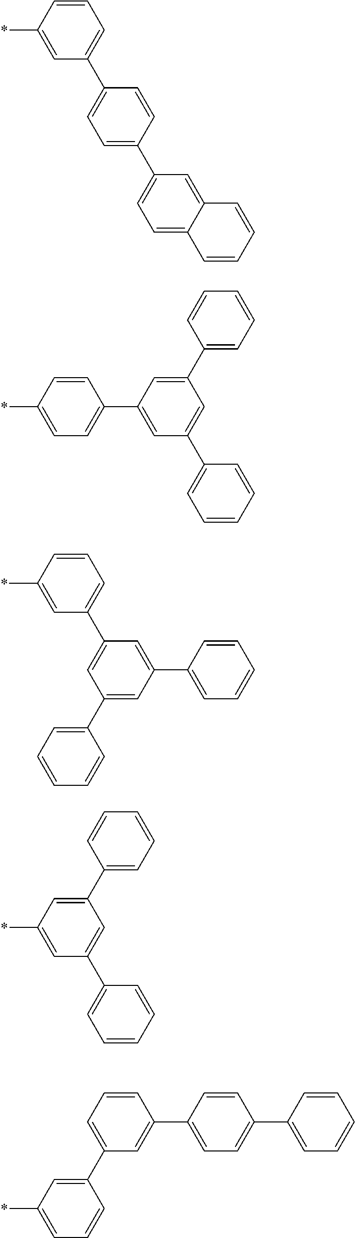

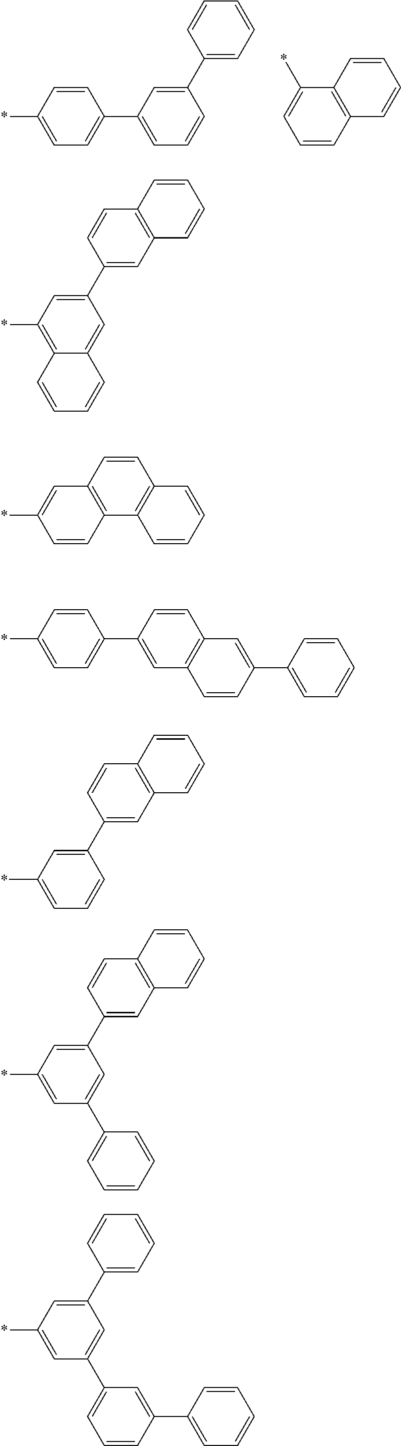

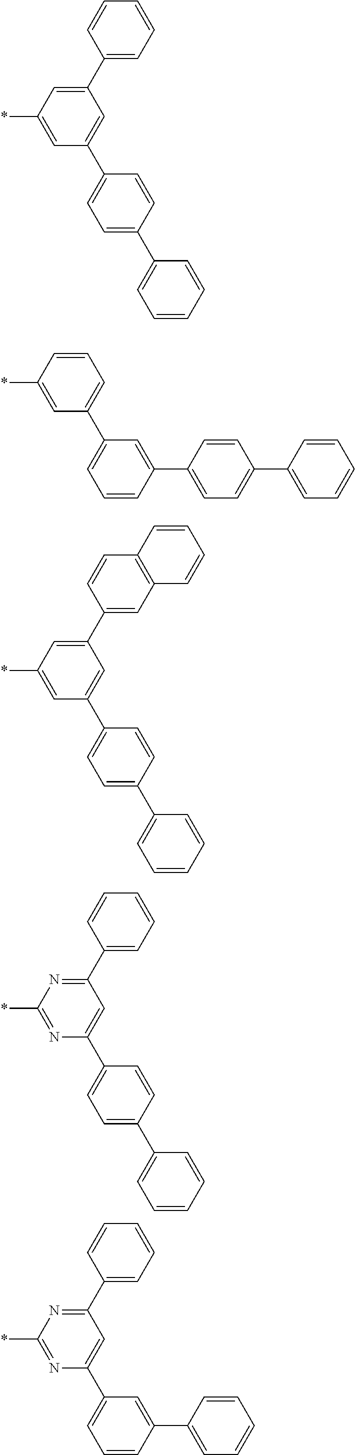

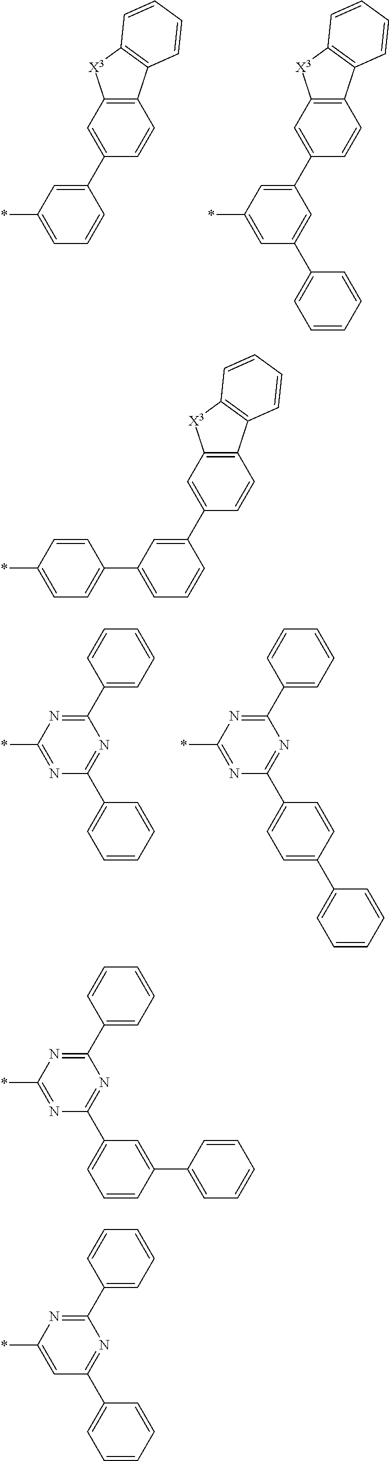

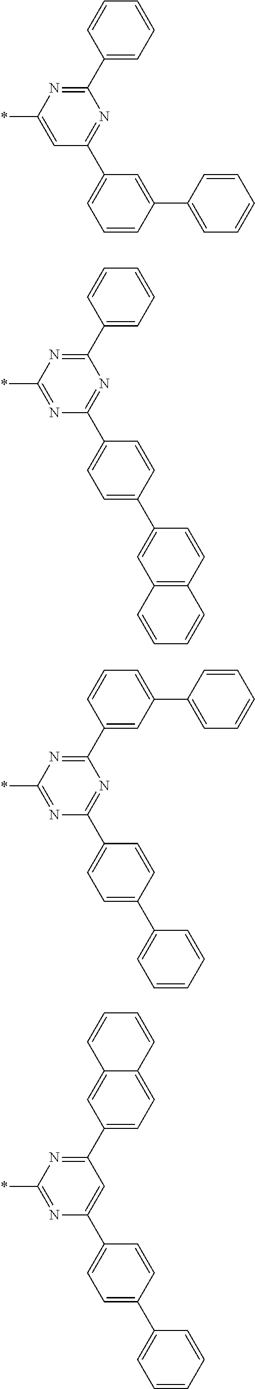

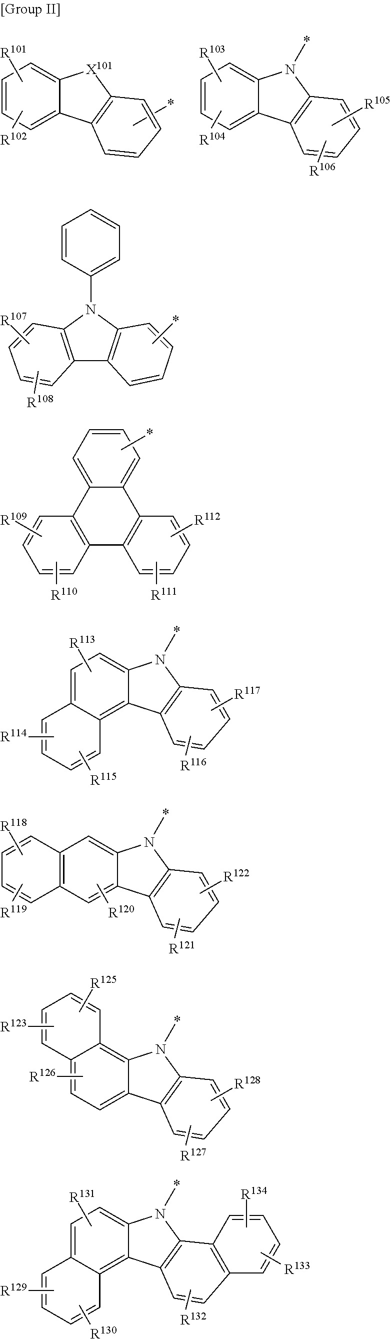

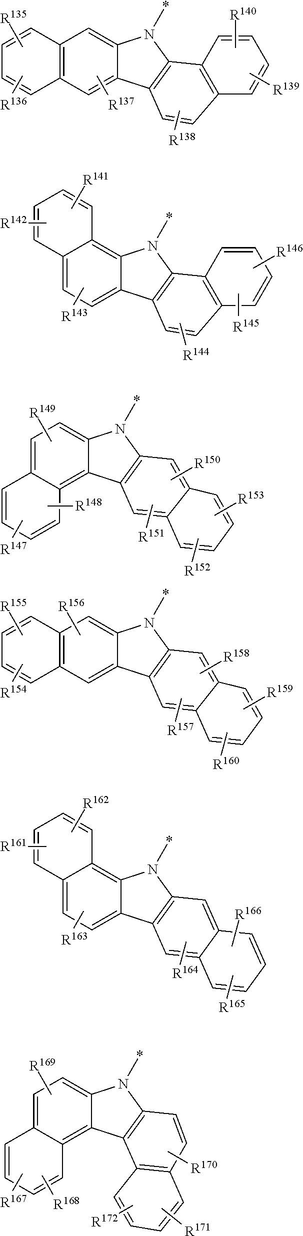

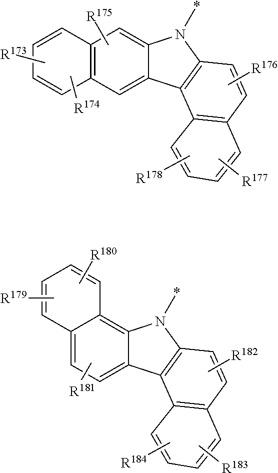

In Group I and Group II,

X³ and X¹⁰¹ may be, e.g., O or S,

R¹⁰¹ to R¹²³ and R¹²⁵ to R¹⁸⁴ may each independently be, e.g., hydrogen, deuterium, a halogen, a cyano group, a C1 to C20 alkyl group, a C6 to C30 aryl group, a C2 to C30 heteroaryl group, or a combination thereof, and

* is a linking point.

In an implementation, the compound represented by Chemical Formula 2 may be, e.g., represented by Chemical Formula 2-1a or Chemical Formula 2-3a.

In an implementation, in Chemical Formula 2-1a, L⁶, L⁸, and L¹⁰ may each independently be, e.g., a single bond or a substituted or unsubstituted phenylene group, R⁴, R⁶, and R⁸ may each independently be, e.g., a substituted or unsubstituted phenyl group, a substituted or unsubstituted biphenyl group, a substituted or unsubstituted terphenyl group, a substituted or unsubstituted naphthyl group, a substituted or unsubstituted triphenylene group, a substituted or unsubstituted dibenzofuranyl group, a substituted or unsubstituted dibenzothiophenyl group, or a substituted or unsubstituted carbazolyl group, and at least one of R⁴, R⁶ and R⁸ may be, e.g., a substituted or unsubstituted triphenylene group, a substituted or unsubstituted carbazolyl group, a substituted or unsubstituted dibenzofuranyl group, or a substituted or unsubstituted dibenzothiophenyl group.

In an implementation, in Chemical Formula 2-3a, L⁵ and L⁹ may each independently be, e.g., a single bond or a substituted or unsubstituted phenylene group, R³ and R⁷ may each independently be, e.g., a substituted or unsubstituted phenyl group, a substituted or unsubstituted biphenyl group, a substituted or unsubstituted terphenyl group, a substituted or unsubstituted naphthyl group, a substituted or unsubstituted triphenylene group, a substituted or unsubstituted dibenzofuranyl group, a substituted or unsubstituted dibenzothiophenyl group, or a substituted or unsubstituted carbazolyl group, at least one of R³ and R⁷ may be, e.g., a substituted or unsubstituted carbazolyl group, a substituted or unsubstituted dibenzofuranyl group, or a substituted or unsubstituted dibenzothiophenyl group, and R^{c} and R^{d} may each independently be, e.g., hydrogen, deuterium, a C1 to C10 alkyl group, a C6 to C12 aryl group, or a combination thereof.

In an implementation, the second compound may be, e.g., a compound of the following Group 2.

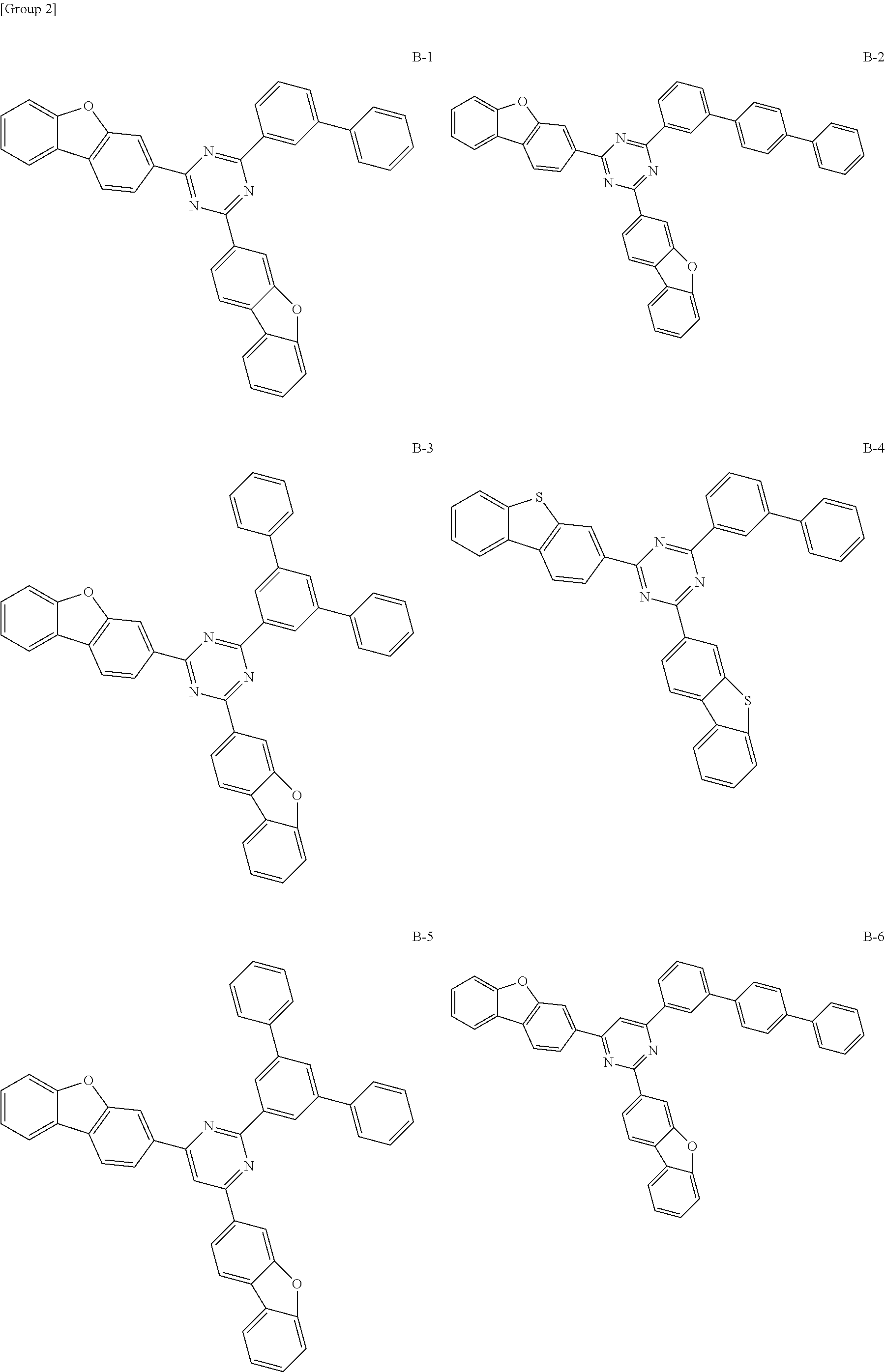

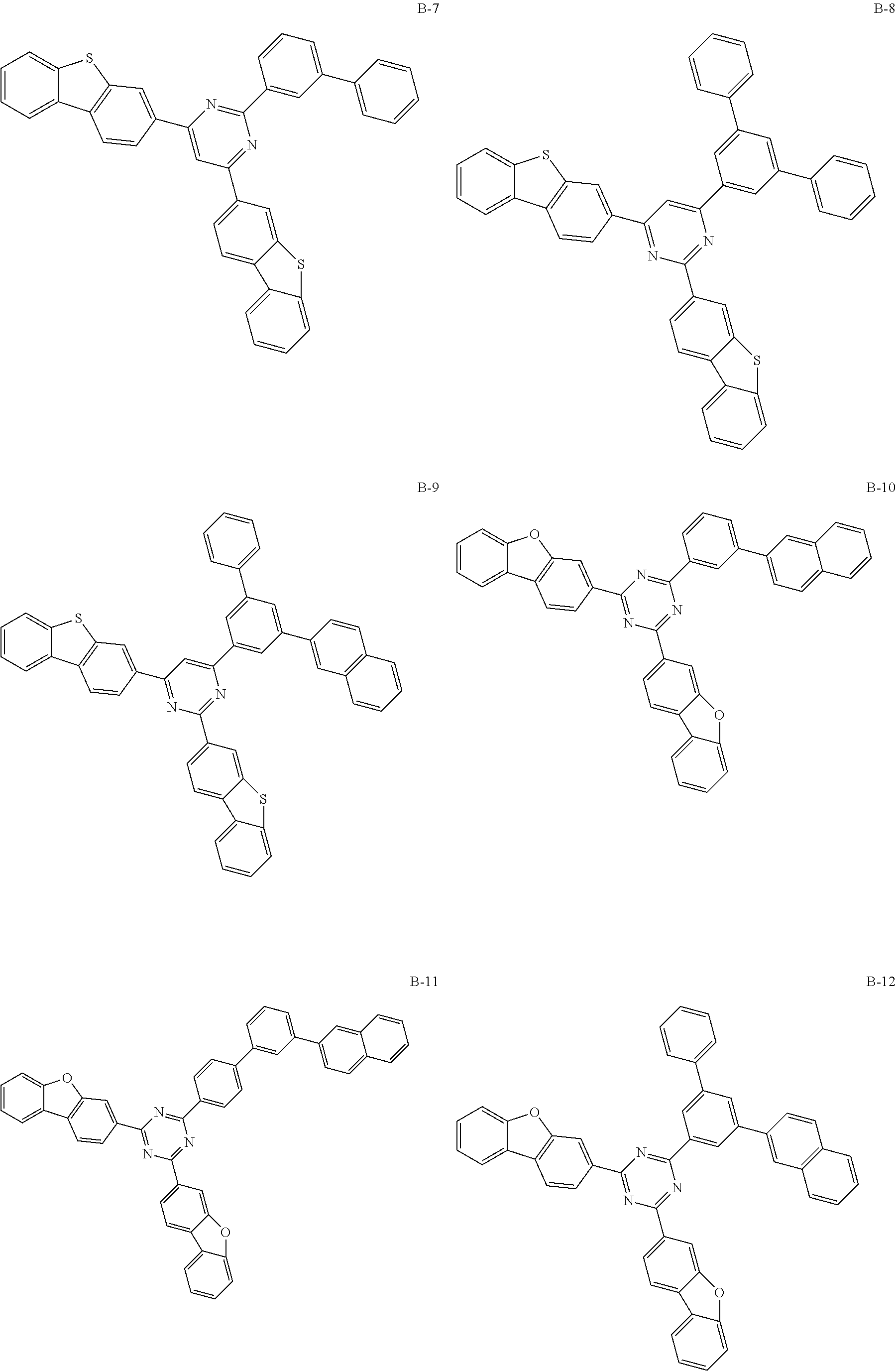

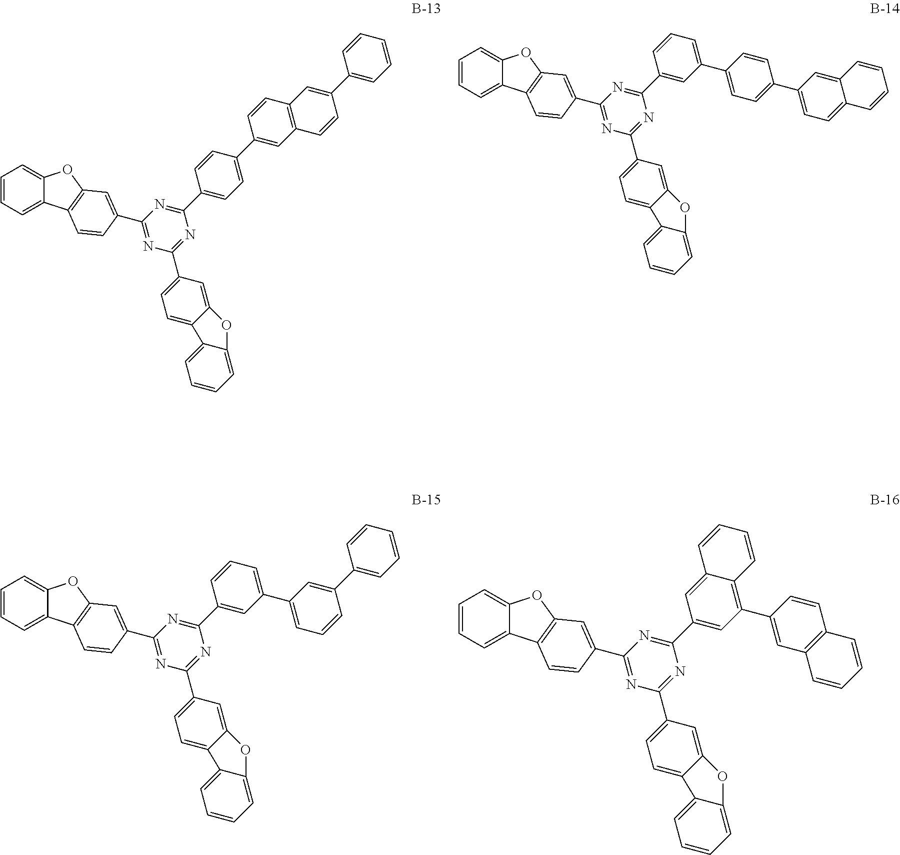 [SMILES: c1ccc(-c2ccc3cc(-c4ccc(-c5nc(-c6ccc7c(c6)oc6ccccc67)nc(-c6ccc7c(c6)oc6ccccc67)n5)cc4)ccc3c2)cc1 ; c1cc(-c2ccc(-c3ccc4ccccc4c3)cc2)cc(-c2nc(-c3ccc4c(c3)oc3ccccc34)nc(-c3ccc4c(c3)oc3ccccc34)n2)c1 ; c1ccc(-c2cccc(-c3cccc(-c4nc(-c5ccc6c(c5)oc5ccccc56)nc(-c5ccc6c(c5)oc5ccccc56)n4)c3)c2)cc1 ; c1ccc2cc(-c3cc(-c4nc(-c5ccc6c(c5)oc5ccccc56)nc(-c5ccc6c(c5)oc5ccccc56)n4)cc4ccccc34)ccc2c1]

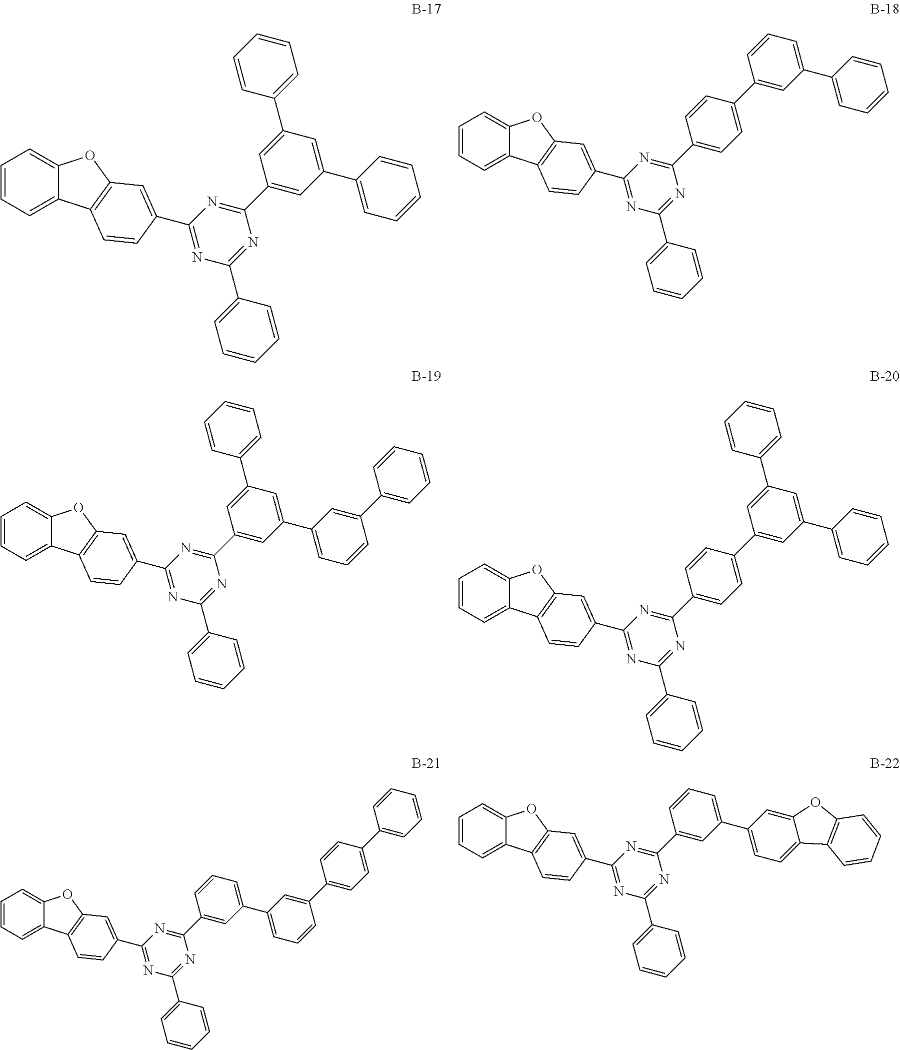

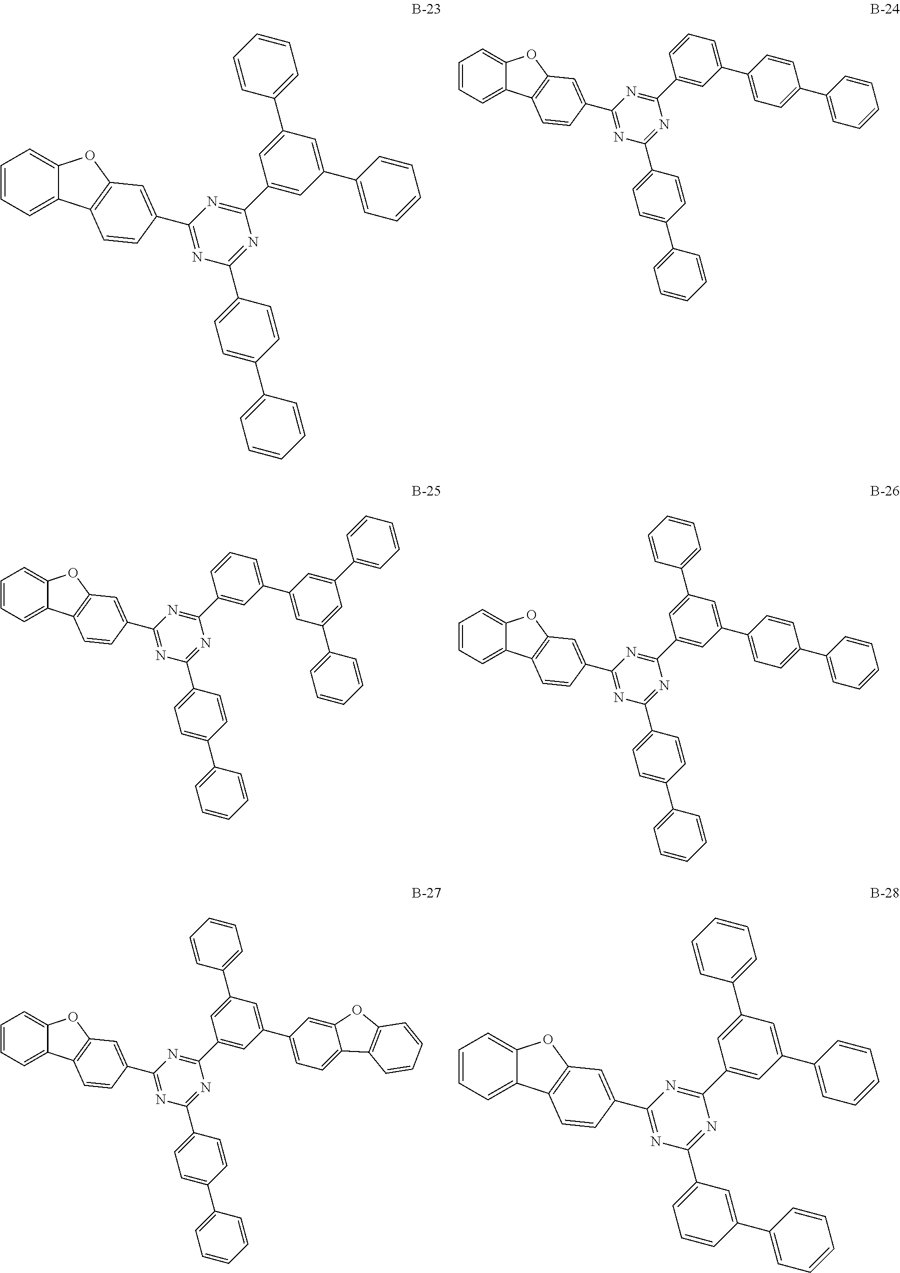

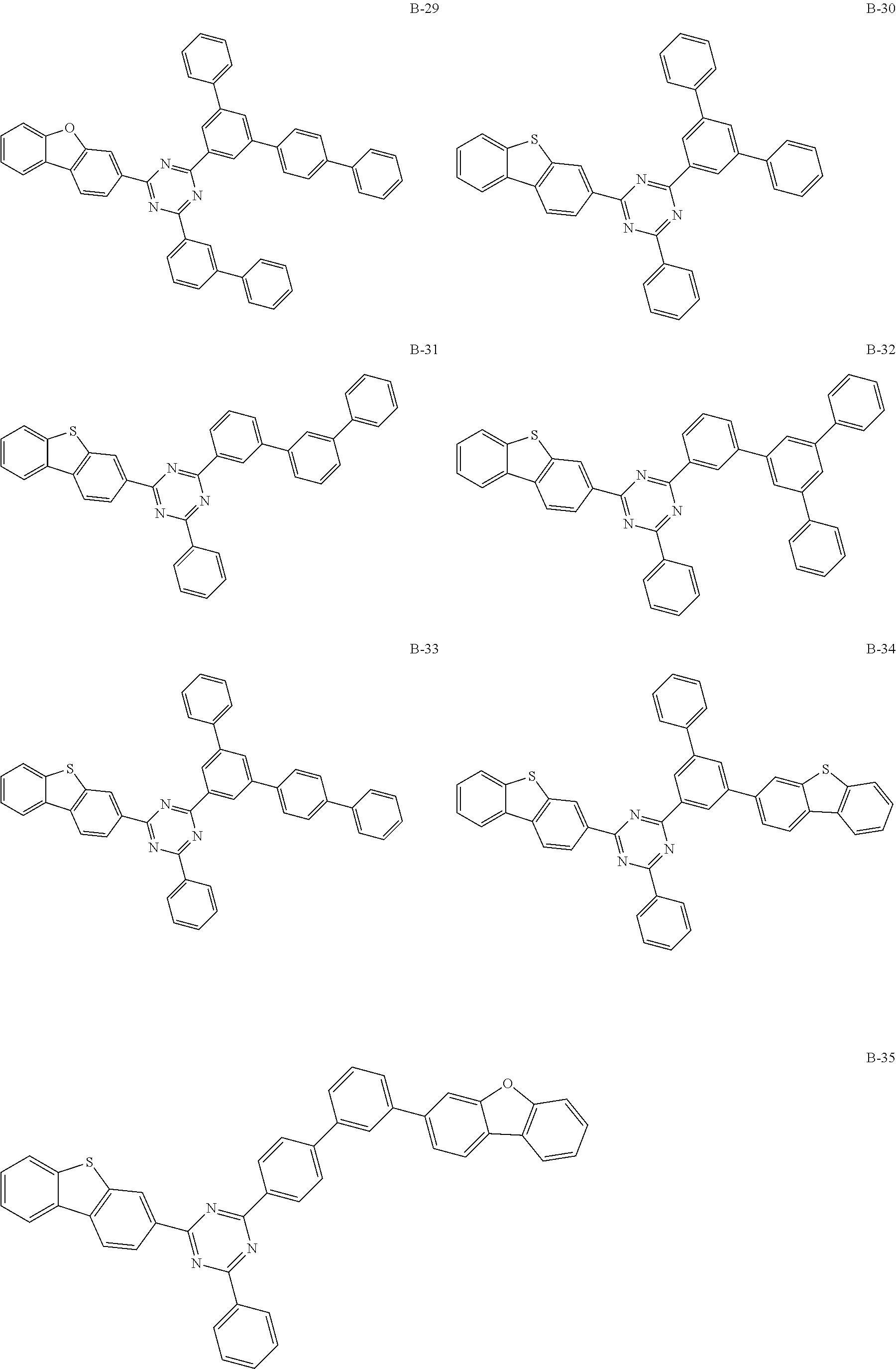

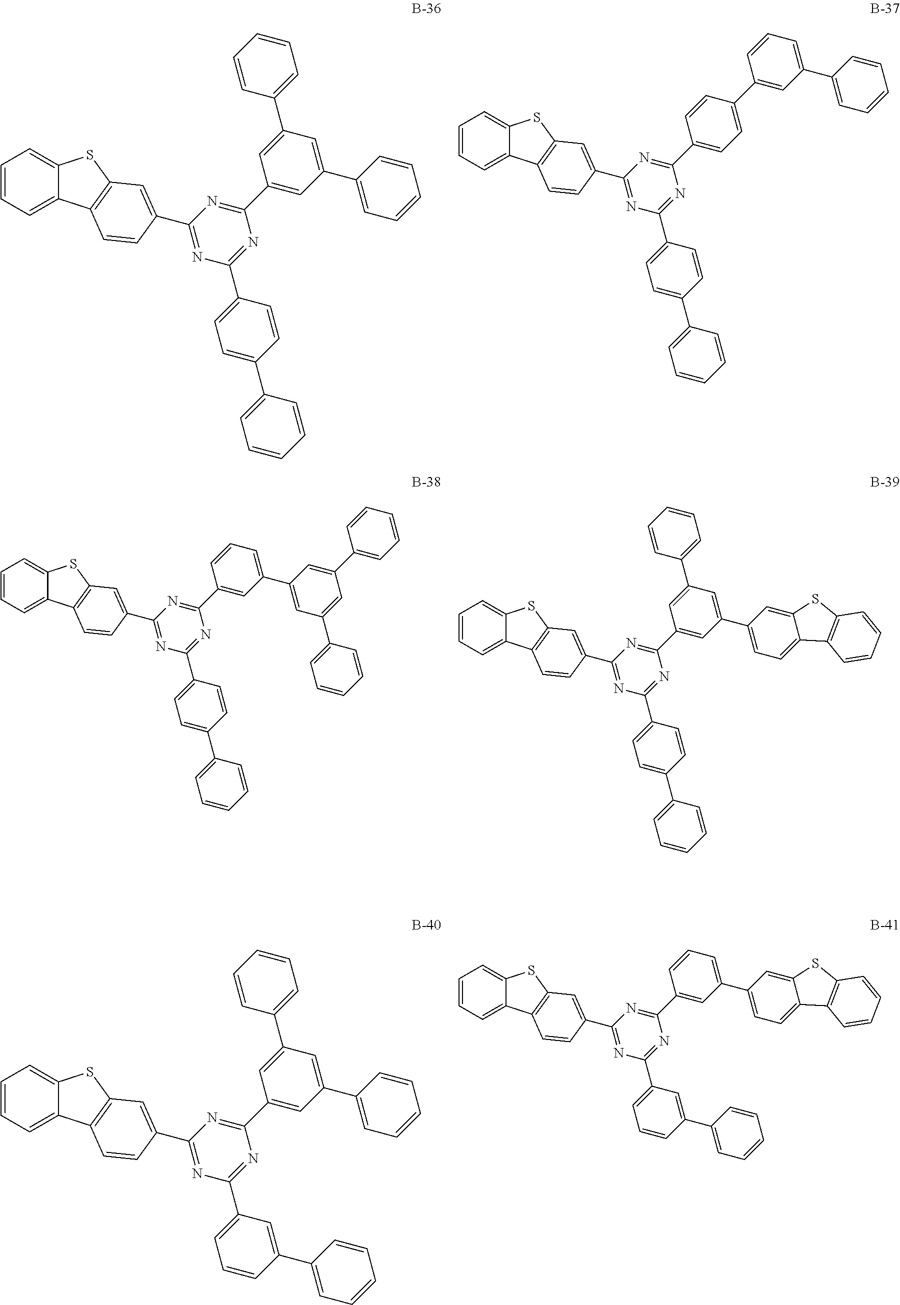

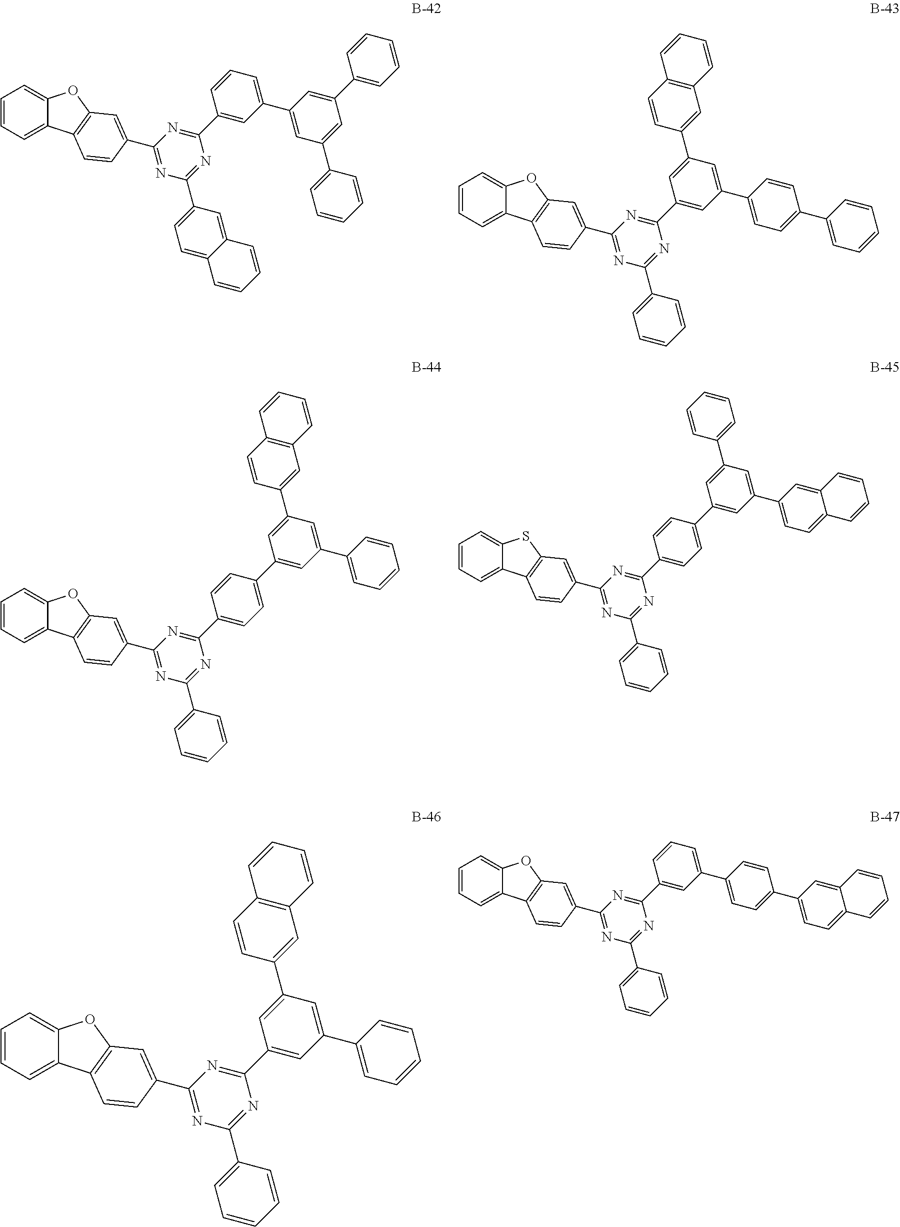

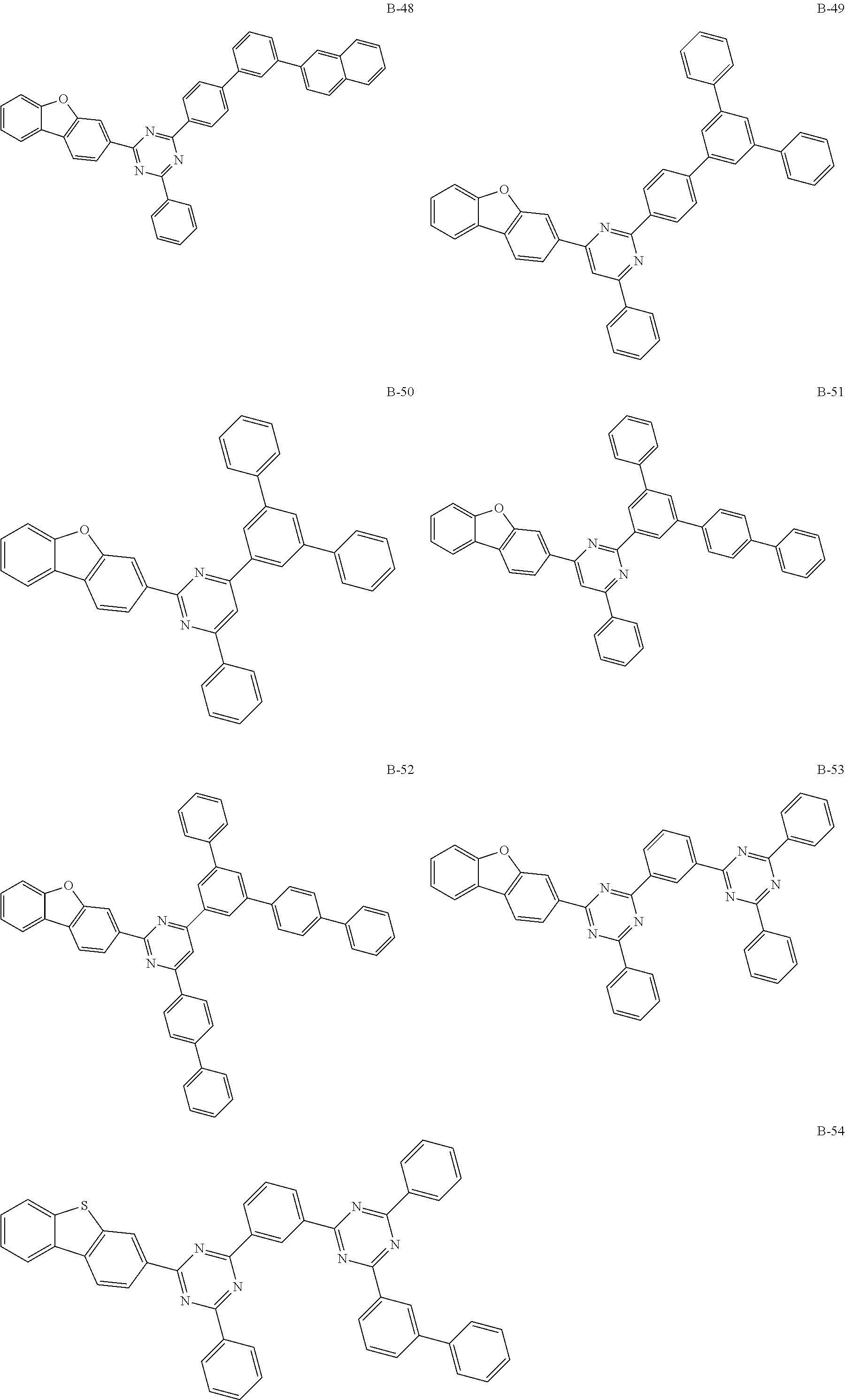

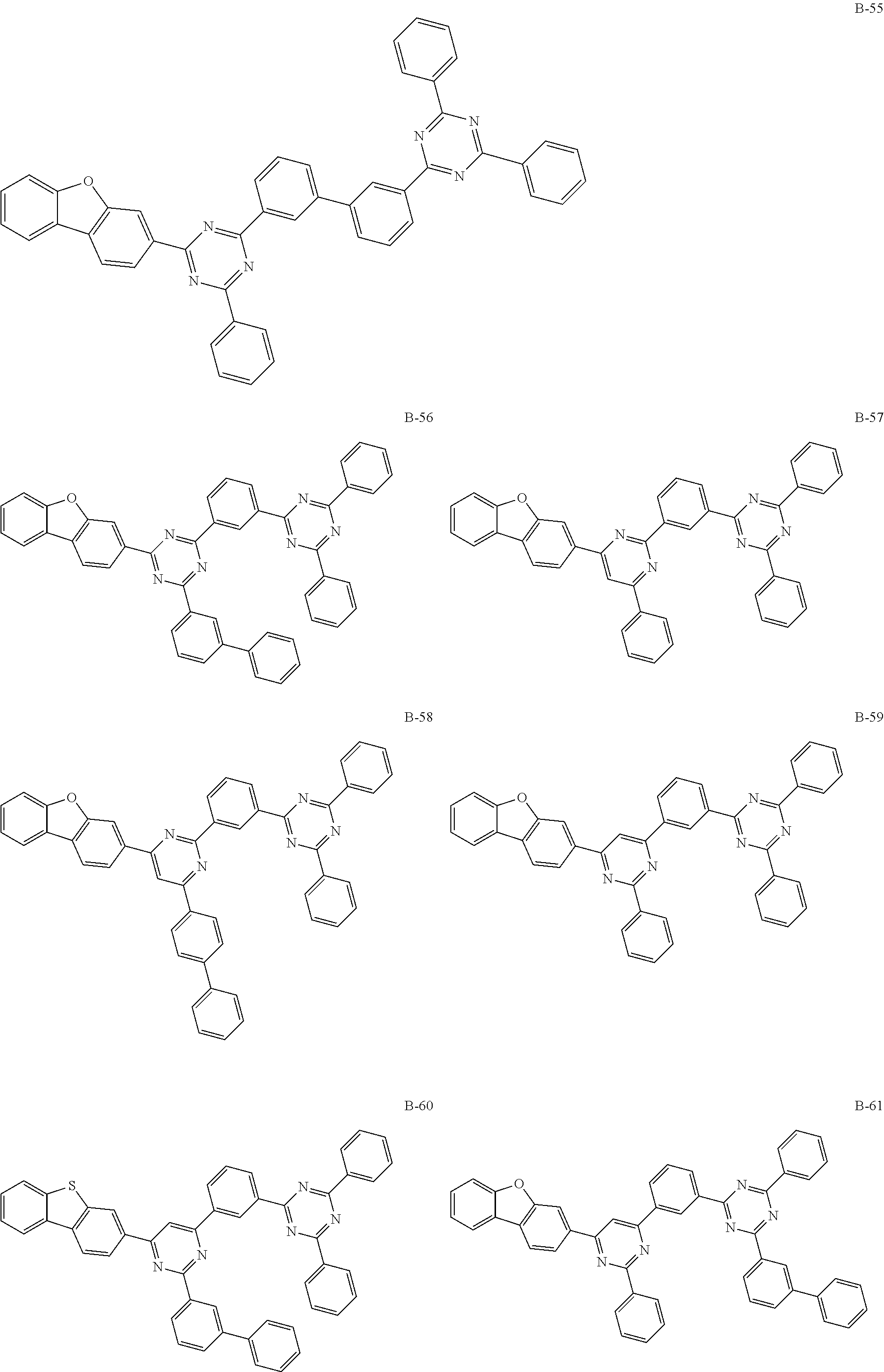

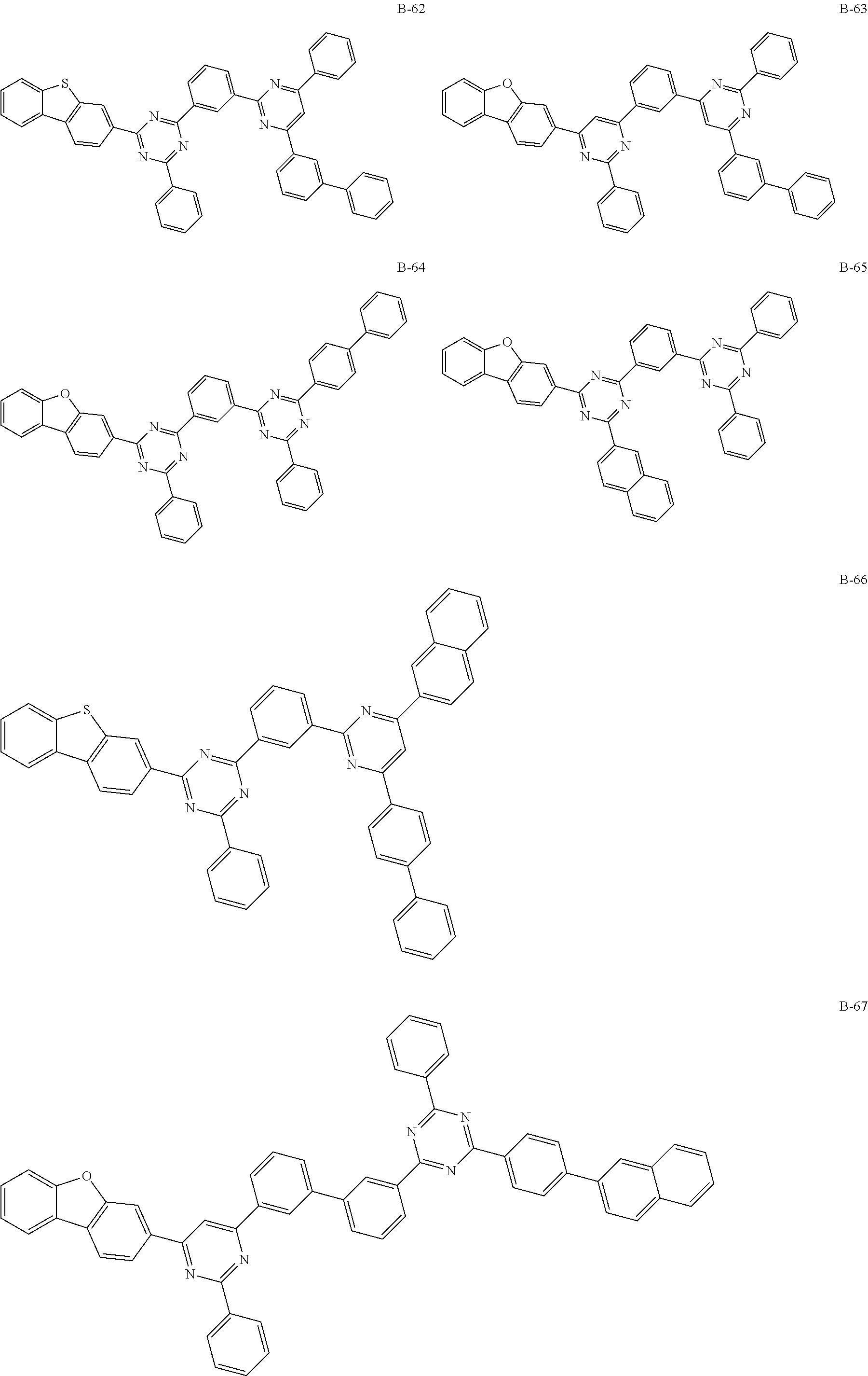

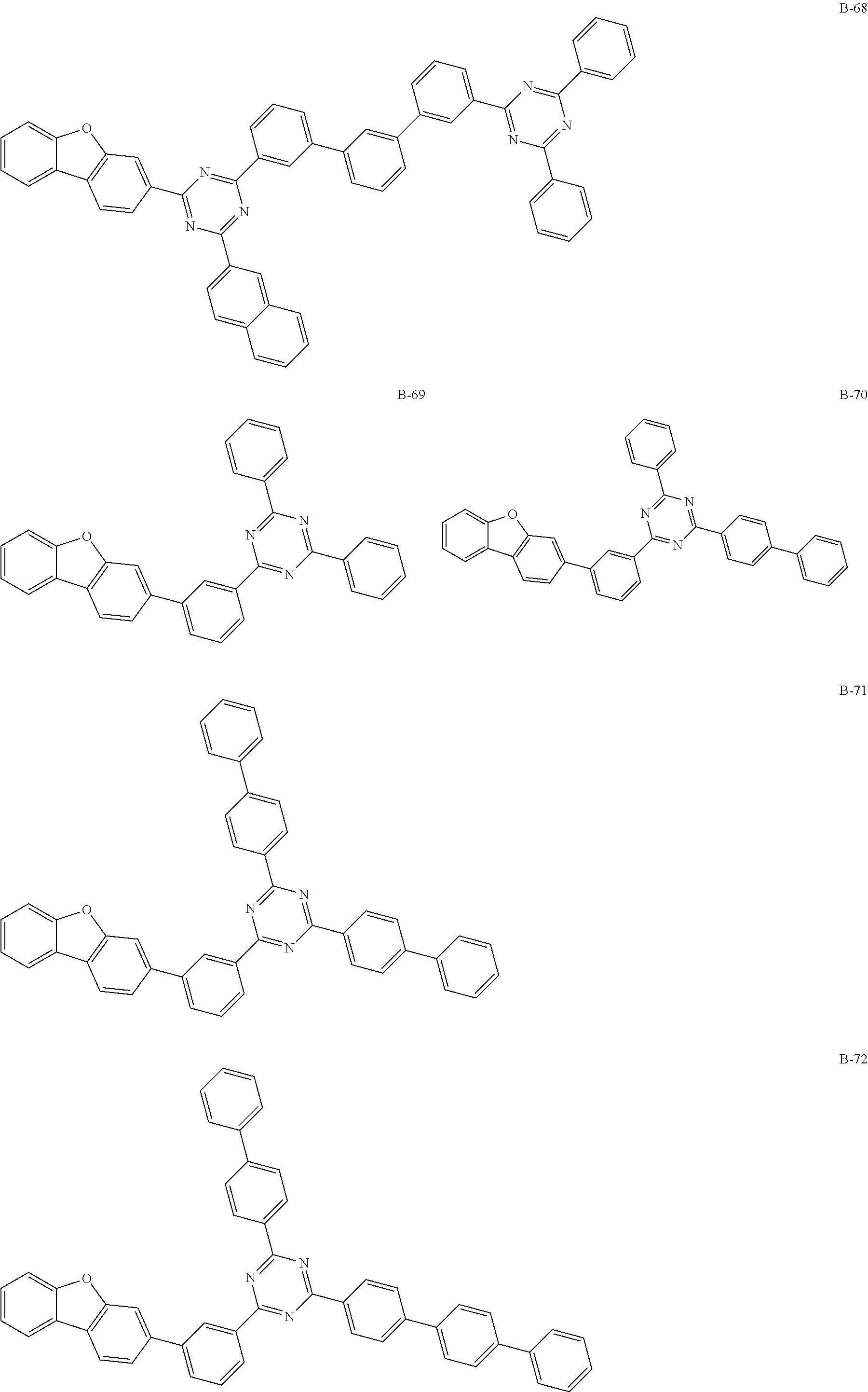 [SMILES: c1ccc(-c2nc(-c3ccccc3)nc(-c3cccc(-c4cccc(-c5cccc(-c6nc(-c7ccc8ccccc8c7)nc(-c7ccc8c(c7)oc7ccccc78)n6)c5)c4)c3)n2)cc1 ; c1ccc(-c2nc(-c3ccccc3)nc(-c3cccc(-c4ccc5c(c4)oc4ccccc45)c3)n2)cc1 ; c1ccc(-c2ccc(-c3nc(-c4ccccc4)nc(-c4cccc(-c5ccc6c(c5)oc5ccccc56)c4)n3)cc2)cc1 ; c1ccc(-c2ccc(-c3nc(-c4ccc(-c5ccccc5)cc4)nc(-c4cccc(-c5ccc6c(c5)oc5ccccc56)c4)n3)cc2)cc1 ; c1ccc(-c2ccc(-c3ccc(-c4nc(-c5ccc(-c6ccccc6)cc5)nc(-c5cccc(-c6ccc7c(c6)oc6ccccc67)c5)n4)cc3)cc2)cc1]

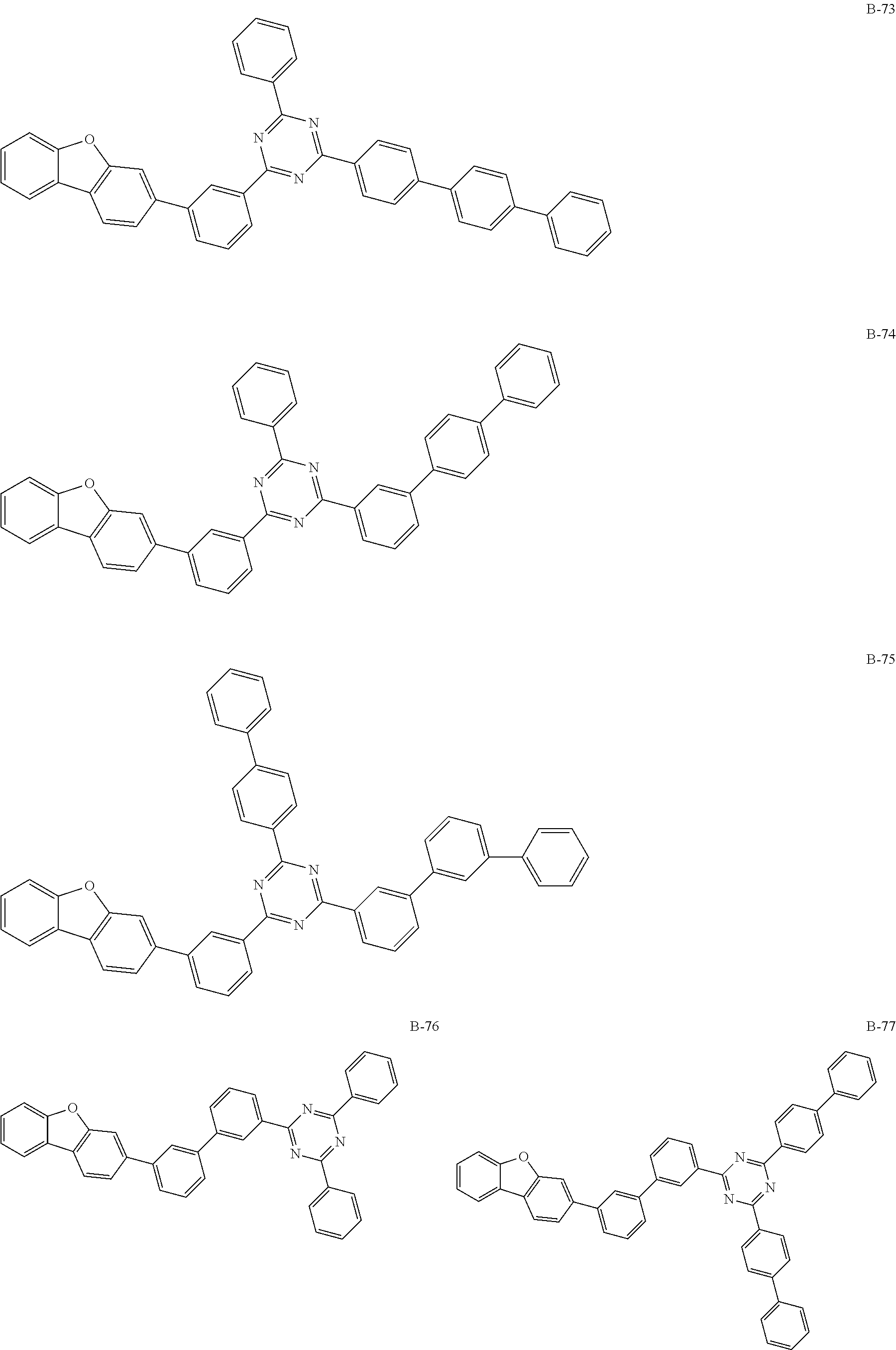 [SMILES: c1ccc(-c2ccc(-c3ccc(-c4nc(-c5ccccc5)nc(-c5cccc(-c6ccc7c(c6)oc6ccccc67)c5)n4)cc3)cc2)cc1 ; c1ccc(-c2ccc(-c3cccc(-c4nc(-c5ccccc5)nc(-c5cccc(-c6ccc7c(c6)oc6ccccc67)c5)n4)c3)cc2)cc1 ; c1ccc(-c2ccc(-c3nc(-c4cccc(-c5cccc(-c6ccccc6)c5)c4)nc(-c4cccc(-c5ccc6c(c5)oc5ccccc56)c4)n3)cc2)cc1 ; c1ccc(-c2nc(-c3ccccc3)nc(-c3cccc(-c4cccc(-c5ccc6c(c5)oc5ccccc56)c4)c3)n2)cc1 ; c1ccc(-c2ccc(-c3nc(-c4ccc(-c5ccccc5)cc4)nc(-c4cccc(-c5cccc(-c6ccc7c(c6)oc6ccccc67)c5)c4)n3)cc2)cc1]

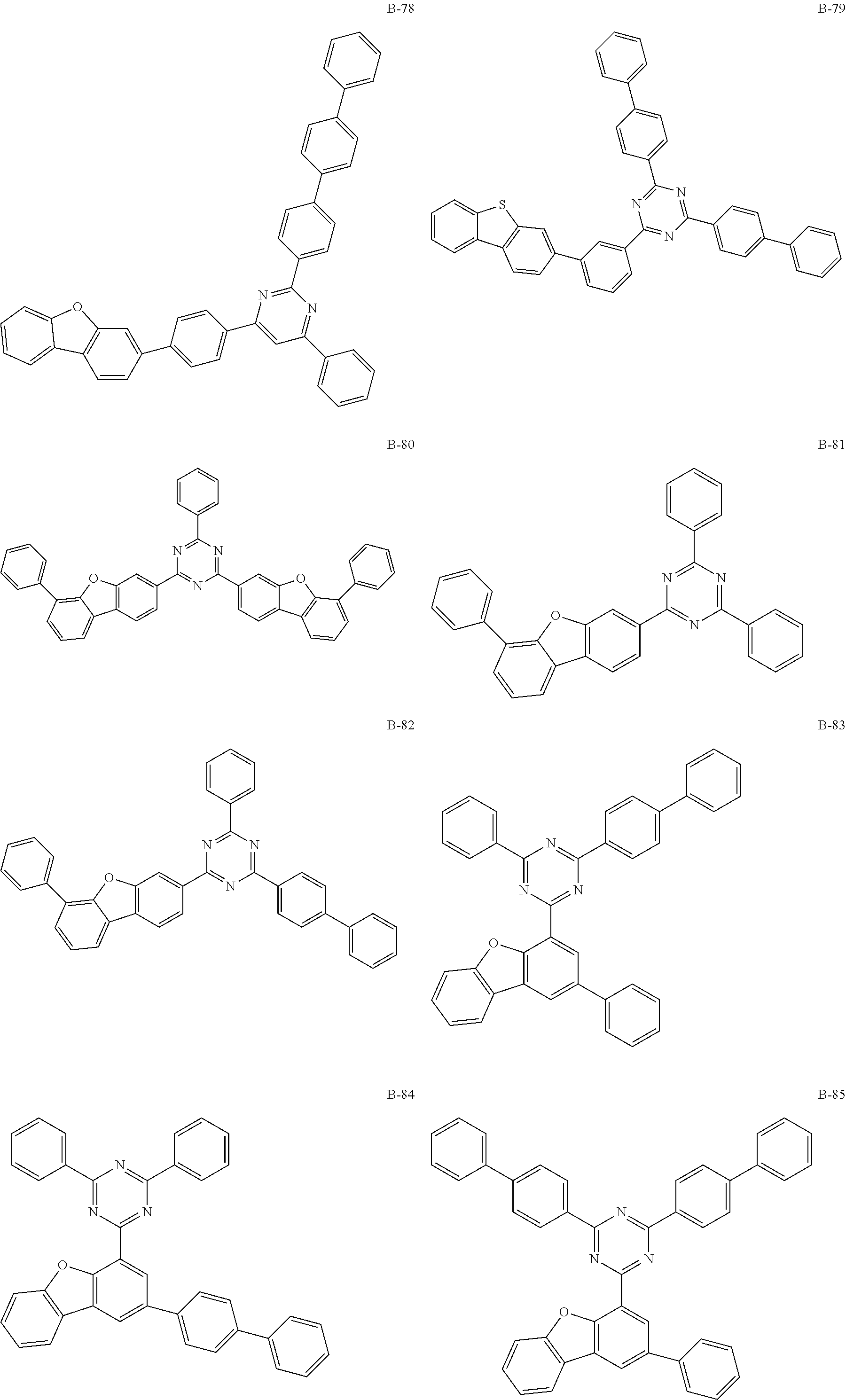

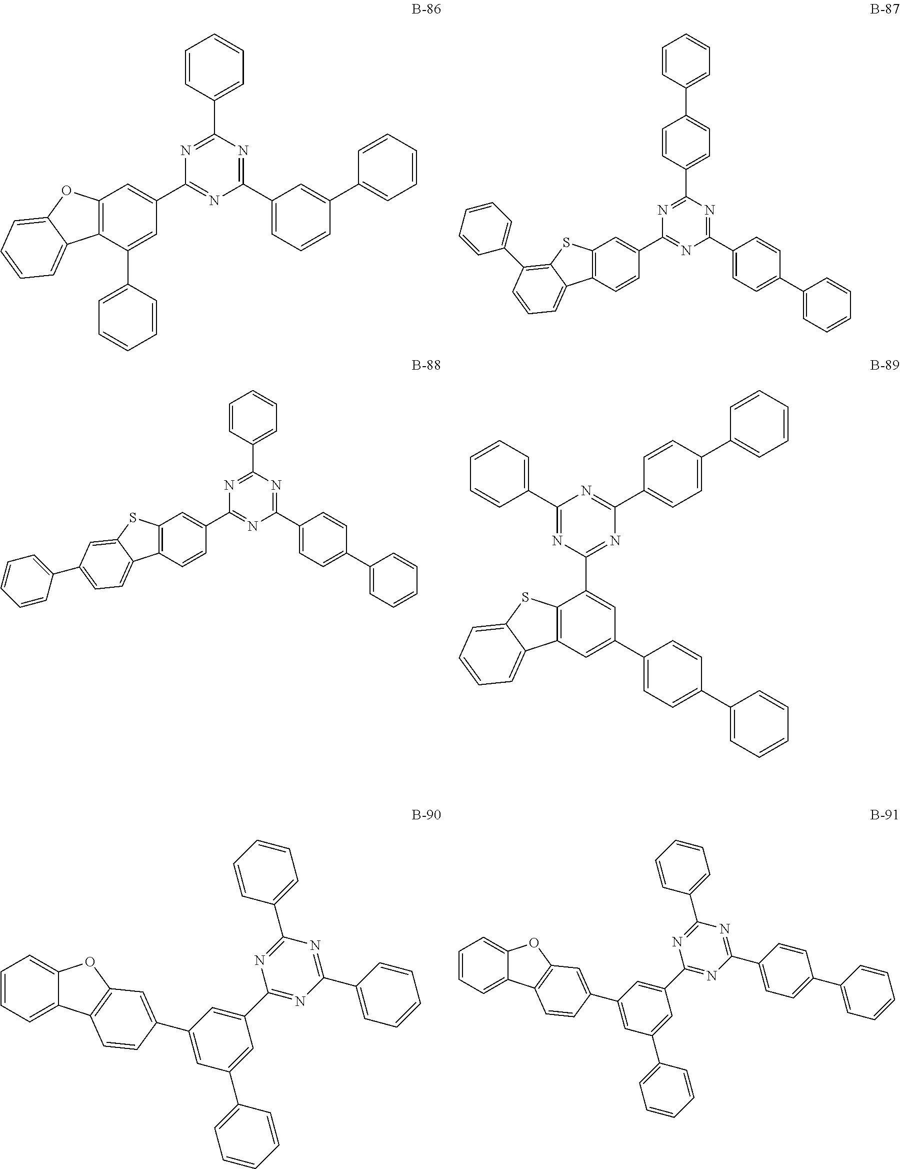

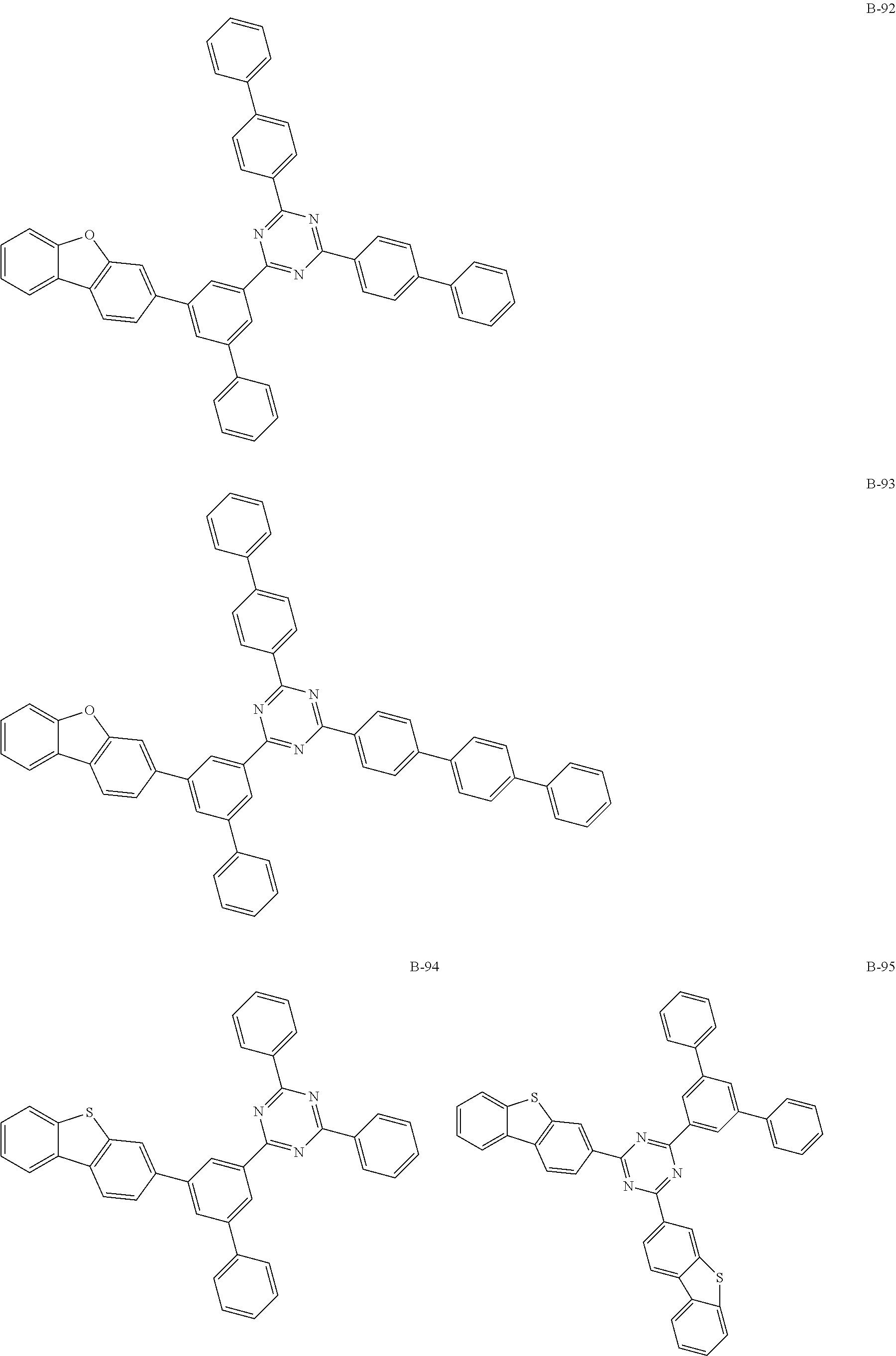

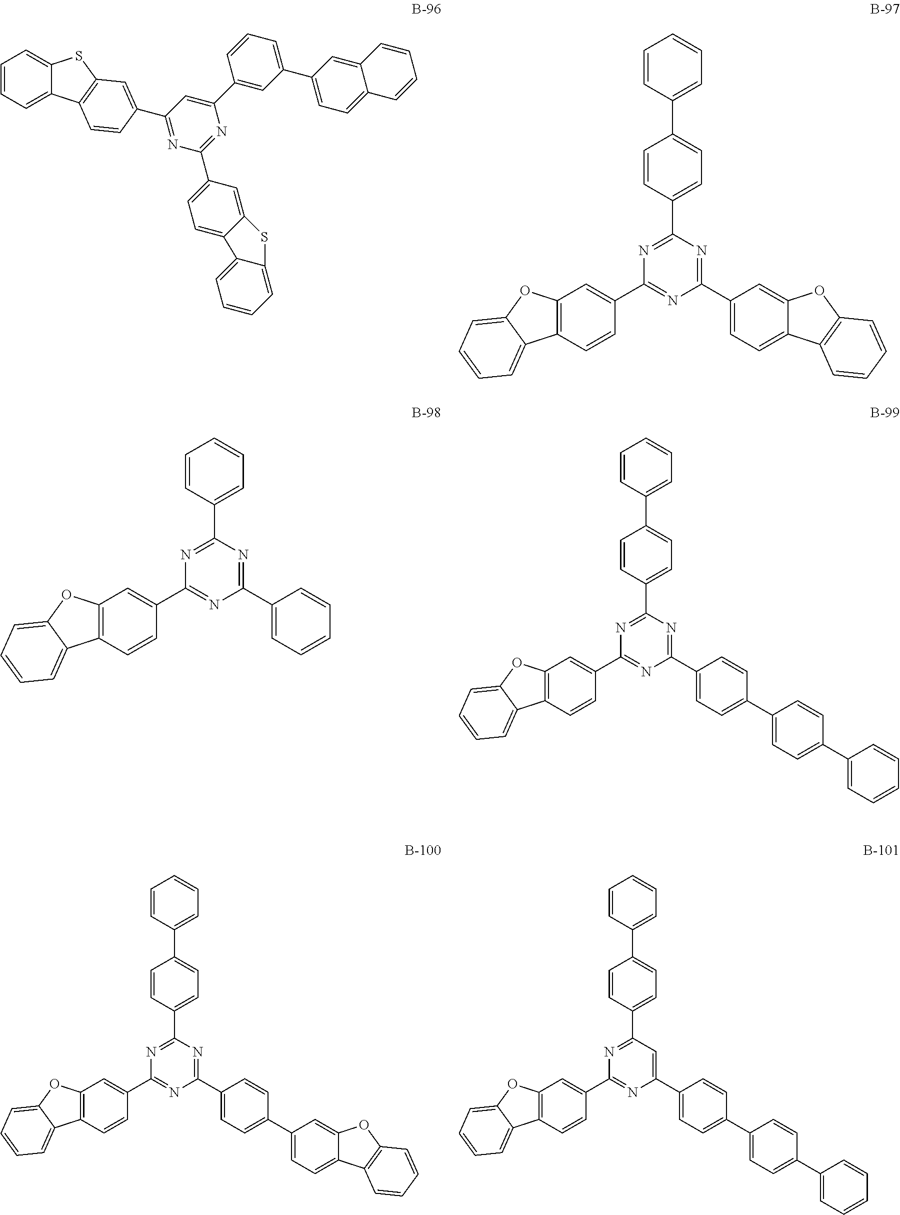 [SMILES: c1cc(-c2ccc3ccccc3c2)cc(-c2cc(-c3ccc4c(c3)sc3ccccc34)nc(-c3ccc4c(c3)sc3ccccc34)n2)c1 ; c1ccc(-c2ccc(-c3nc(-c4ccc5c(c4)oc4ccccc45)nc(-c4ccc5c(c4)oc4ccccc45)n3)cc2)cc1 ; c1ccc(-c2nc(-c3ccccc3)nc(-c3ccc4c(c3)oc3ccccc34)n2)cc1 ; c1ccc(-c2ccc(-c3ccc(-c4nc(-c5ccc(-c6ccccc6)cc5)nc(-c5ccc6c(c5)oc5ccccc56)n4)cc3)cc2)cc1 ; c1ccc(-c2ccc(-c3nc(-c4ccc(-c5ccc6c(c5)oc5ccccc56)cc4)nc(-c4ccc5c(c4)oc4ccccc45)n3)cc2)cc1 ; c1ccc(-c2ccc(-c3ccc(-c4cc(-c5ccc(-c6ccccc6)cc5)nc(-c5ccc6c(c5)oc5ccccc56)n4)cc3)cc2)cc1]

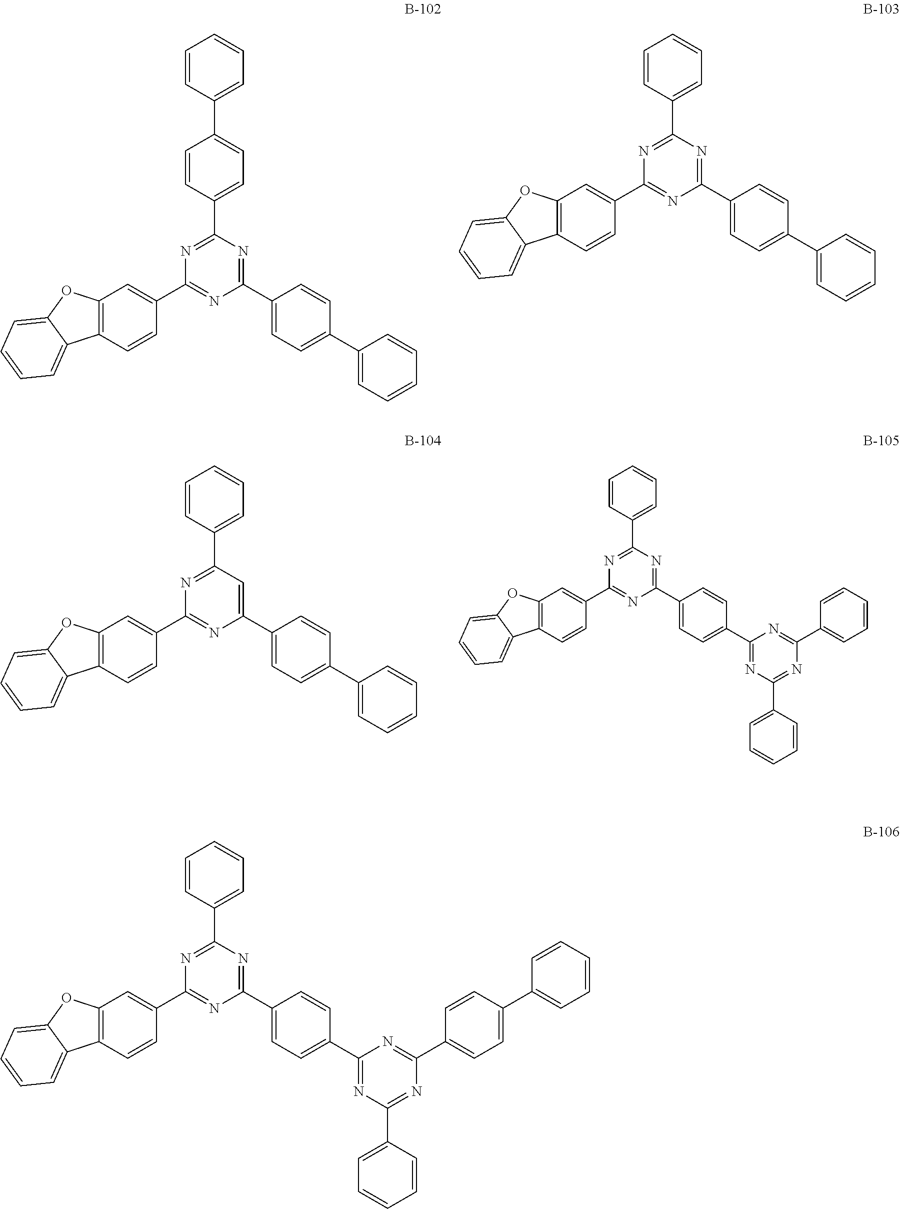 [SMILES: c1ccc(-c2ccc(-c3nc(-c4ccc(-c5ccccc5)cc4)nc(-c4ccc5c(c4)oc4ccccc45)n3)cc2)cc1 ; c1ccc(-c2ccc(-c3nc(-c4ccccc4)nc(-c4ccc5c(c4)oc4ccccc45)n3)cc2)cc1 ; c1ccc(-c2ccc(-c3cc(-c4ccccc4)nc(-c4ccc5c(c4)oc4ccccc45)n3)cc2)cc1 ; c1ccc(-c2nc(-c3ccccc3)nc(-c3ccc(-c4nc(-c5ccccc5)nc(-c5ccc6c(c5)oc5ccccc56)n4)cc3)n2)cc1 ; c1ccc(-c2ccc(-c3nc(-c4ccccc4)nc(-c4ccc(-c5nc(-c6ccccc6)nc(-c6ccc7c(c6)oc6ccccc67)n5)cc4)n3)cc2)cc1]

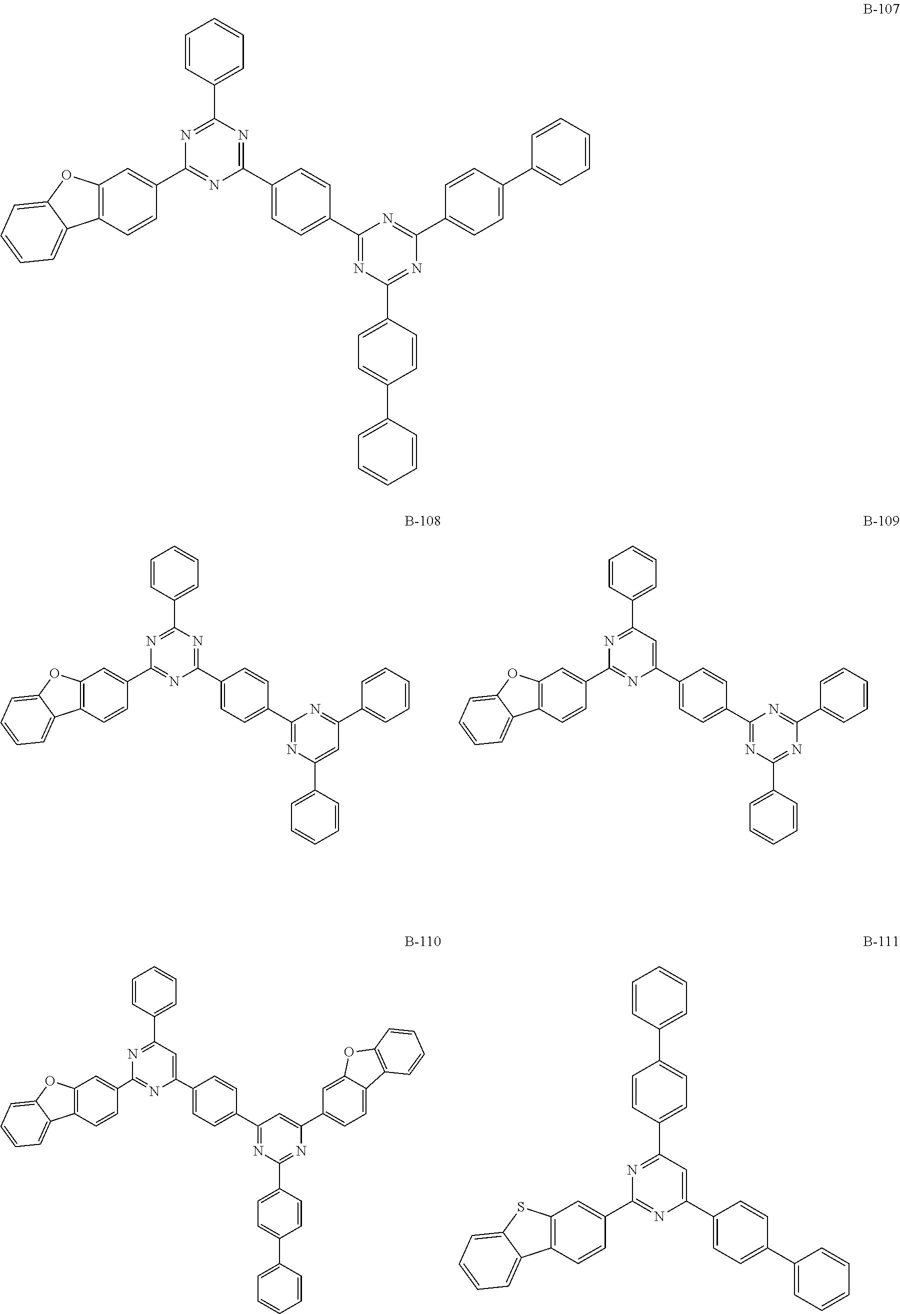 [SMILES: c1ccc(-c2ccc(-c3nc(-c4ccc(-c5ccccc5)cc4)nc(-c4ccc(-c5nc(-c6ccccc6)nc(-c6ccc7c(c6)oc6ccccc67)n5)cc4)n3)cc2)cc1 ; c1ccc(-c2cc(-c3ccccc3)nc(-c3ccc(-c4nc(-c5ccccc5)nc(-c5ccc6c(c5)oc5ccccc56)n4)cc3)n2)cc1 ; c1ccc(-c2cc(-c3ccc(-c4nc(-c5ccccc5)nc(-c5ccccc5)n4)cc3)nc(-c3ccc4c(c3)oc3ccccc34)n2)cc1 ; c1ccc(-c2ccc(-c3nc(-c4ccc(-c5cc(-c6ccccc6)nc(-c6ccc7c(c6)oc6ccccc67)n5)cc4)cc(-c4ccc5c(c4)oc4ccccc45)n3)cc2)cc1 ; c1ccc(-c2ccc(-c3cc(-c4ccc(-c5ccccc5)cc4)nc(-c4ccc5c(c4)sc4ccccc45)n3)cc2)cc1]

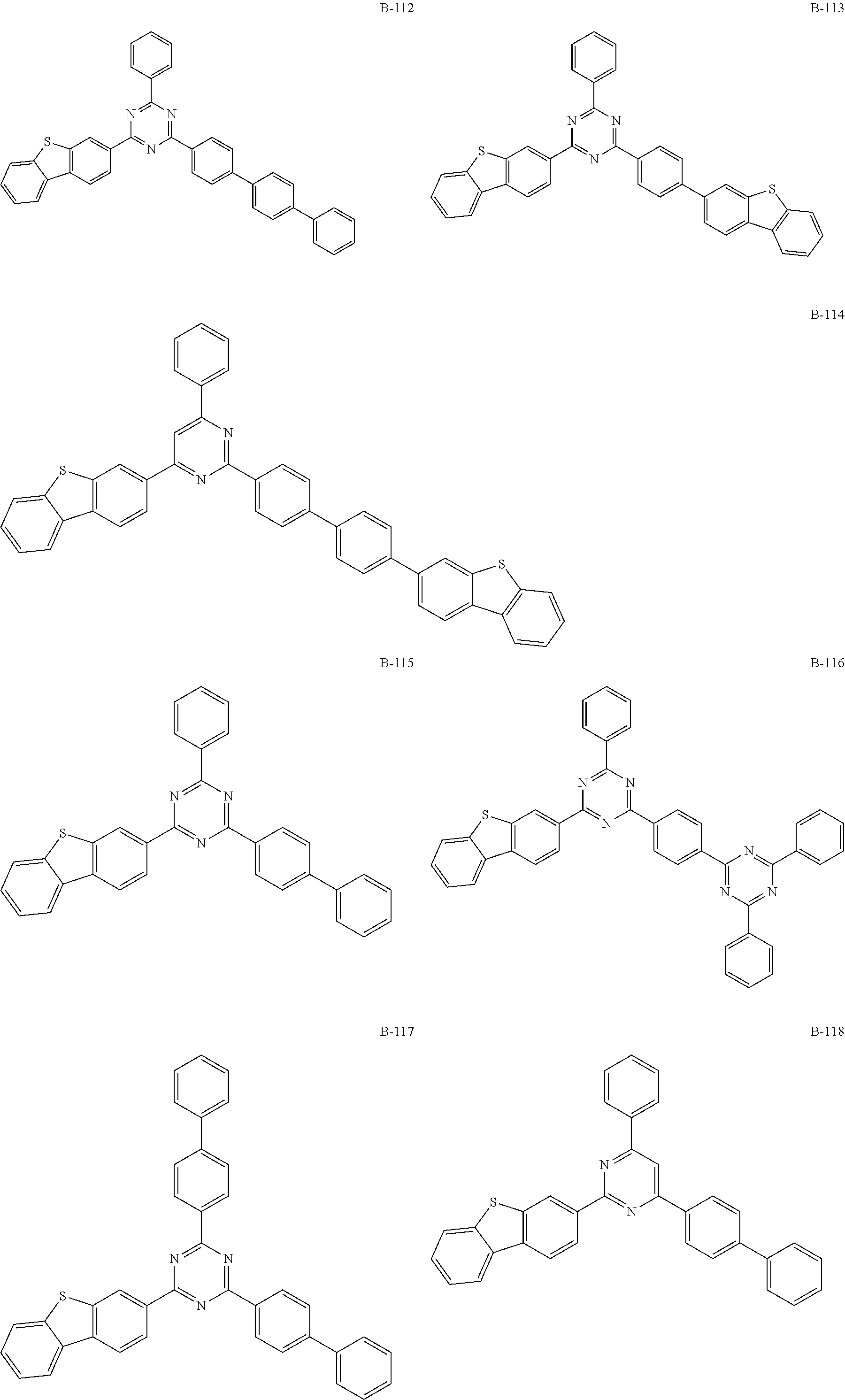

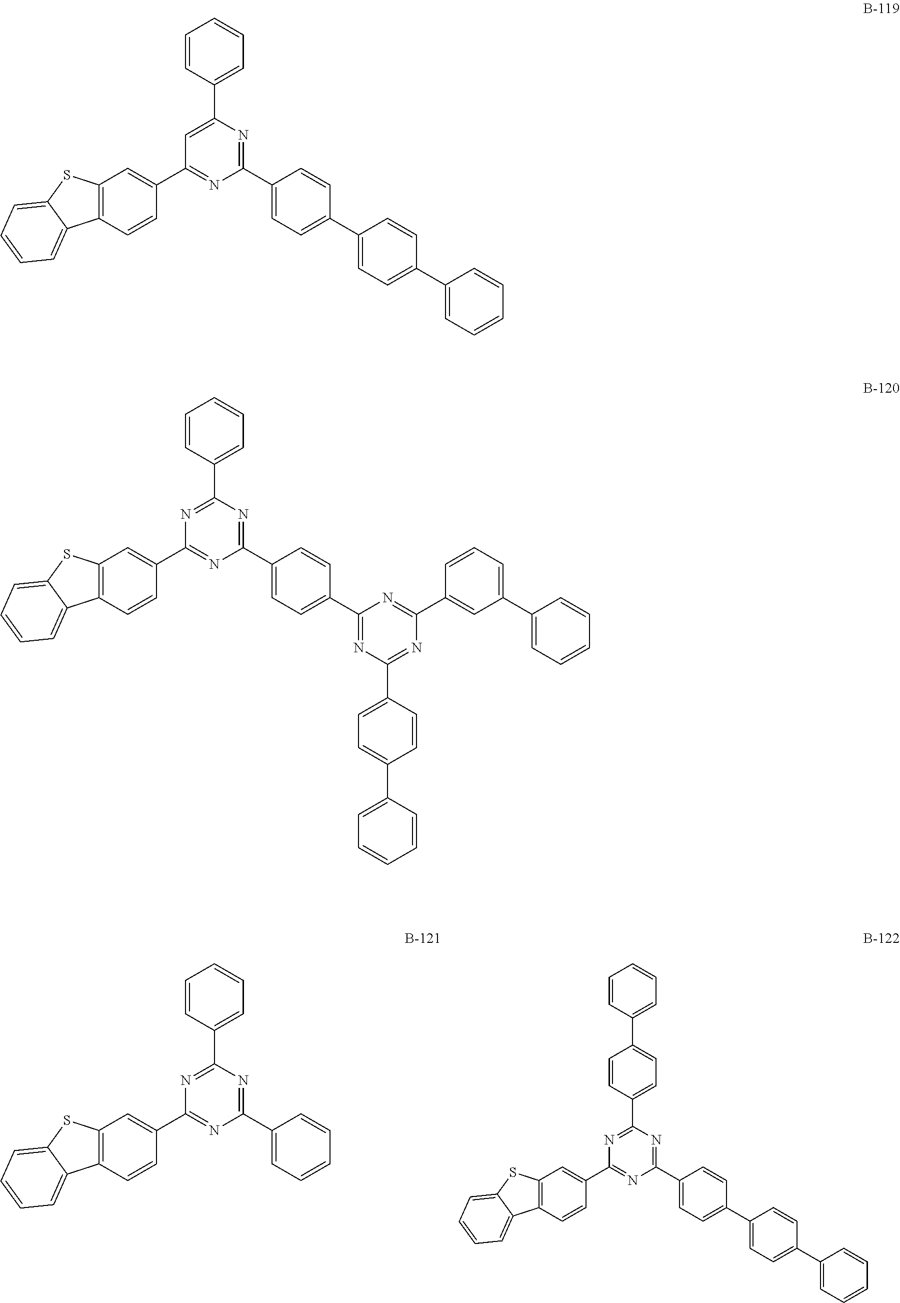 [SMILES: c1ccc(-c2ccc(-c3ccc(-c4nc(-c5ccccc5)cc(-c5ccc6c(c5)sc5ccccc56)n4)cc3)cc2)cc1 ; c1ccc(-c2ccc(-c3nc(-c4ccc(-c5nc(-c6ccccc6)nc(-c6ccc7c(c6)sc6ccccc67)n5)cc4)nc(-c4cccc(-c5ccccc5)c4)n3)cc2)cc1 ; c1ccc(-c2nc(-c3ccccc3)nc(-c3ccc4c(c3)sc3ccccc34)n2)cc1 ; c1ccc(-c2ccc(-c3ccc(-c4nc(-c5ccc(-c6ccccc6)cc5)nc(-c5ccc6c(c5)sc5ccccc56)n4)cc3)cc2)cc1]

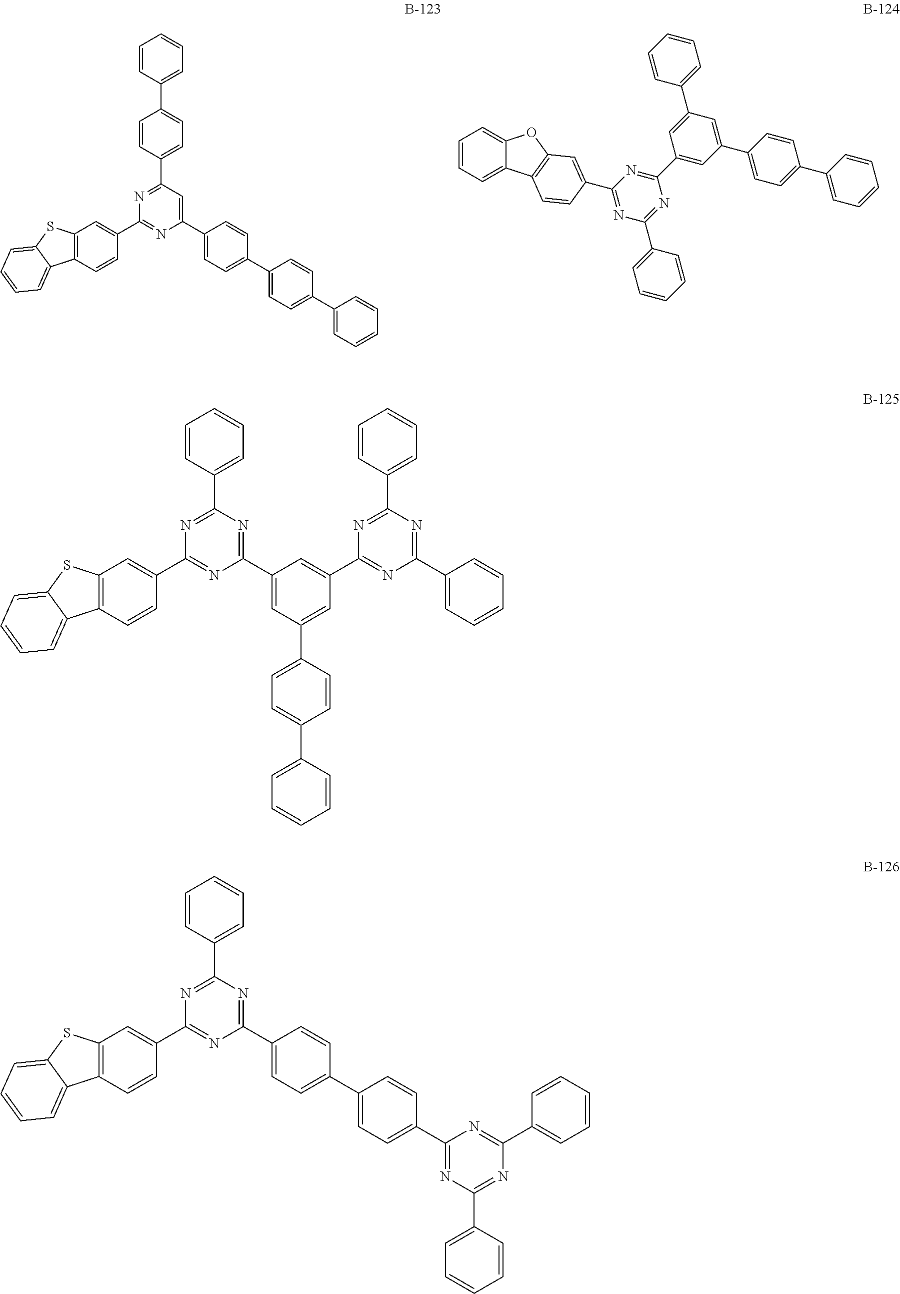 [SMILES: c1ccc(-c2ccc(-c3ccc(-c4cc(-c5ccc(-c6ccccc6)cc5)nc(-c5ccc6c(c5)sc5ccccc56)n4)cc3)cc2)cc1 ; c1ccc(-c2ccc(-c3cc(-c4ccccc4)cc(-c4nc(-c5ccccc5)nc(-c5ccc6c(c5)oc5ccccc56)n4)c3)cc2)cc1 ; c1ccc(-c2ccc(-c3cc(-c4nc(-c5ccccc5)nc(-c5ccccc5)n4)cc(-c4nc(-c5ccccc5)nc(-c5ccc6c(c5)sc5ccccc56)n4)c3)cc2)cc1 ; c1ccc(-c2nc(-c3ccccc3)nc(-c3ccc(-c4ccc(-c5nc(-c6ccccc6)nc(-c6ccc7c(c6)sc6ccccc67)n5)cc4)cc3)n2)cc1]

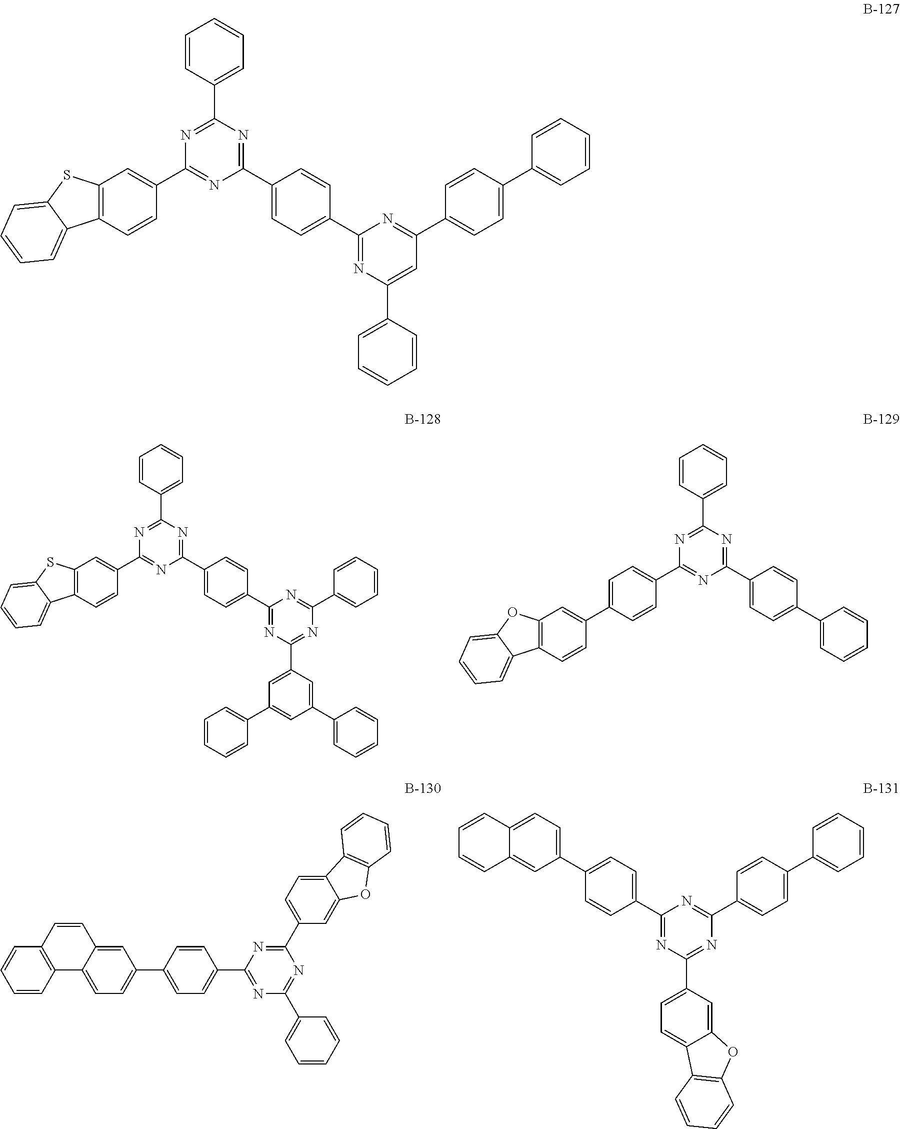 [SMILES: c1ccc(-c2ccc(-c3cc(-c4ccccc4)nc(-c4ccc(-c5nc(-c6ccccc6)nc(-c6ccc7c(c6)sc6ccccc67)n5)cc4)n3)cc2)cc1 ; c1ccc(-c2cc(-c3ccccc3)cc(-c3nc(-c4ccccc4)nc(-c4ccc(-c5nc(-c6ccccc6)nc(-c6ccc7c(c6)sc6ccccc67)n5)cc4)n3)c2)cc1 ; c1ccc(-c2ccc(-c3nc(-c4ccccc4)nc(-c4ccc(-c5ccc6c(c5)oc5ccccc56)cc4)n3)cc2)cc1 ; c1ccc(-c2nc(-c3ccc(-c4ccc5c(ccc6ccccc65)c4)cc3)nc(-c3ccc4c(c3)oc3ccccc34)n2)cc1 ; c1ccc(-c2ccc(-c3nc(-c4ccc(-c5ccc6ccccc6c5)cc4)nc(-c4ccc5c(c4)oc4ccccc45)n3)cc2)cc1]

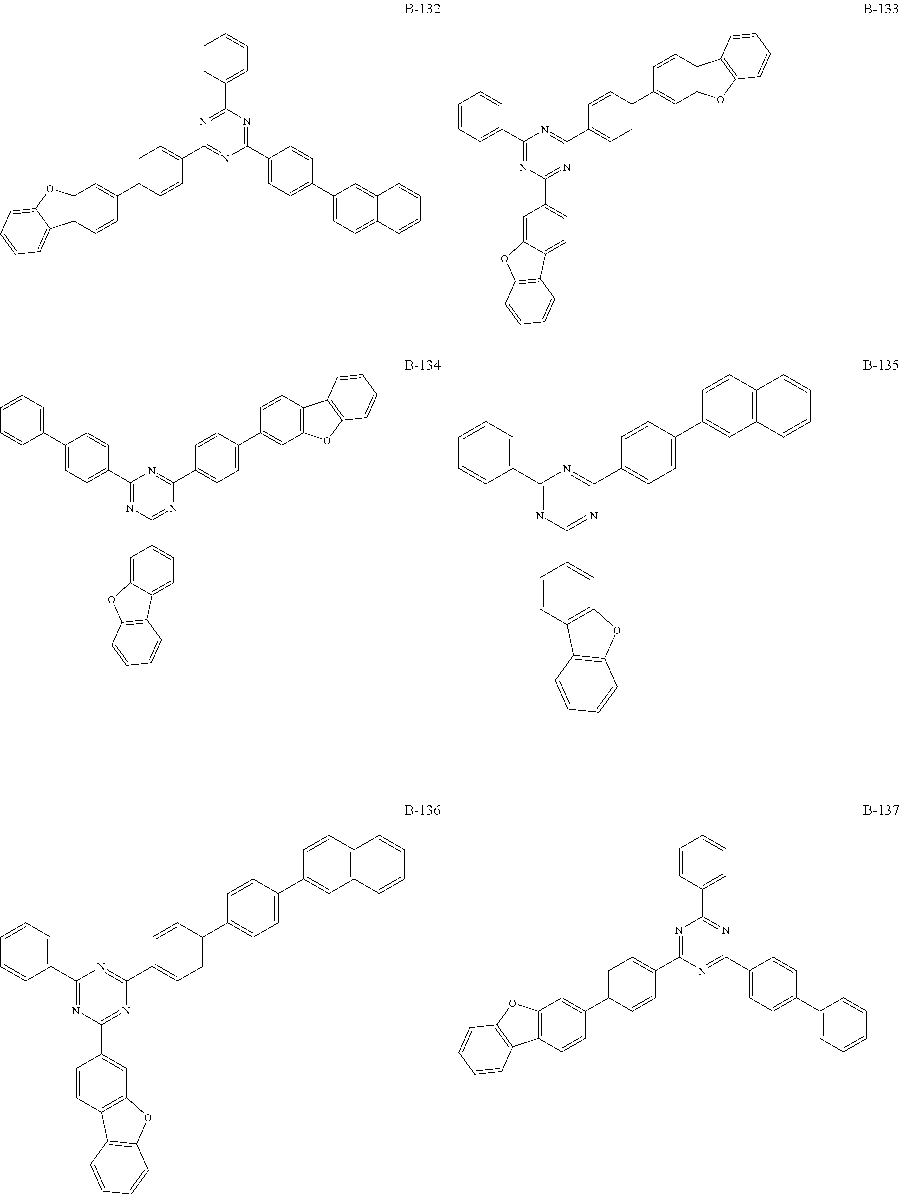 [SMILES: c1ccc(-c2nc(-c3ccc(-c4ccc5ccccc5c4)cc3)nc(-c3ccc(-c4ccc5c(c4)oc4ccccc45)cc3)n2)cc1 ; c1ccc(-c2nc(-c3ccc(-c4ccc5c(c4)oc4ccccc45)cc3)nc(-c3ccc4c(c3)oc3ccccc34)n2)cc1 ; c1ccc(-c2ccc(-c3nc(-c4ccc(-c5ccc6c(c5)oc5ccccc56)cc4)nc(-c4ccc5c(c4)oc4ccccc45)n3)cc2)cc1 ; c1ccc(-c2nc(-c3ccc(-c4ccc5ccccc5c4)cc3)nc(-c3ccc4c(c3)oc3ccccc34)n2)cc1 ; c1ccc(-c2nc(-c3ccc(-c4ccc(-c5ccc6ccccc6c5)cc4)cc3)nc(-c3ccc4c(c3)oc3ccccc34)n2)cc1 ; c1ccc(-c2ccc(-c3nc(-c4ccccc4)nc(-c4ccc(-c5ccc6c(c5)oc5ccccc56)cc4)n3)cc2)cc1]

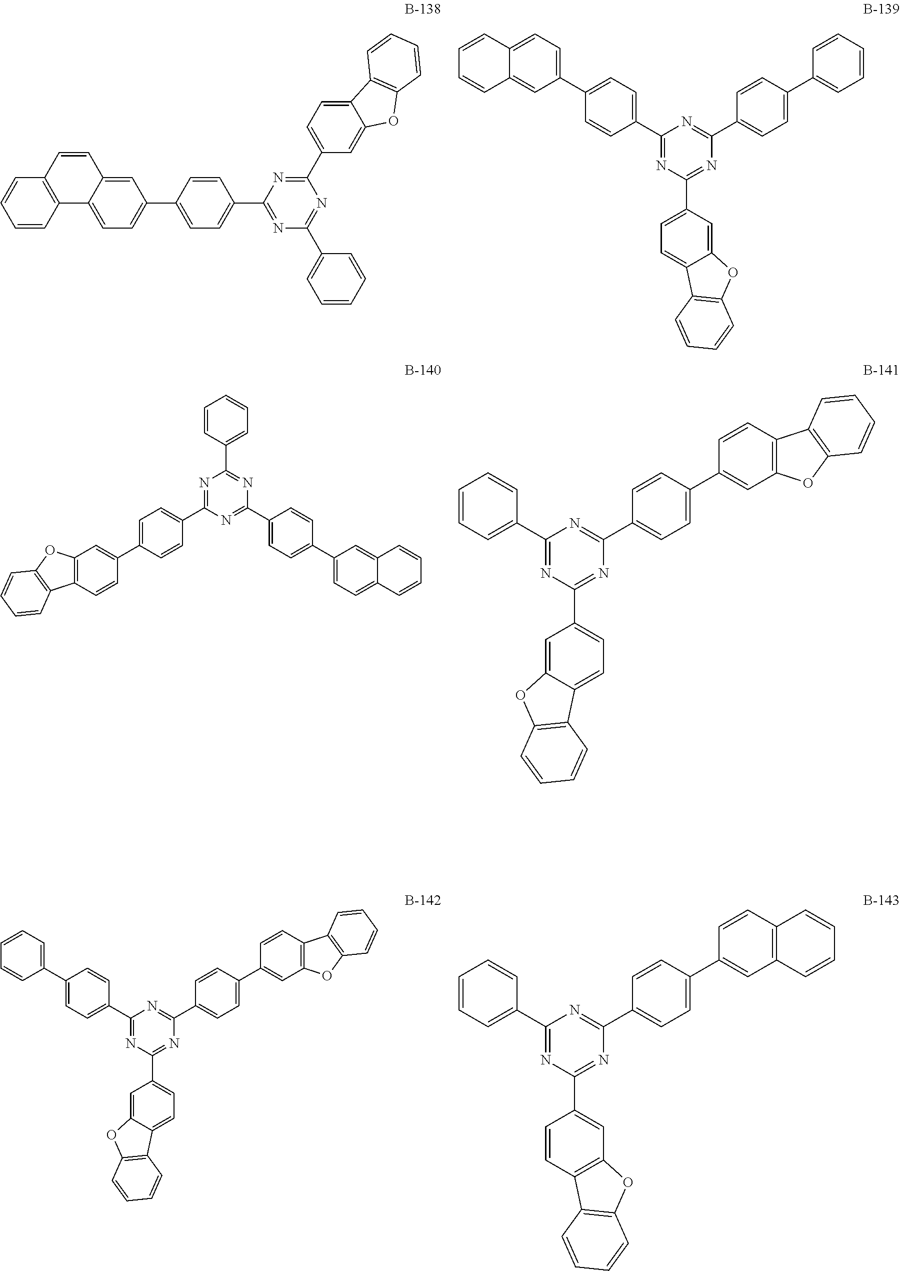

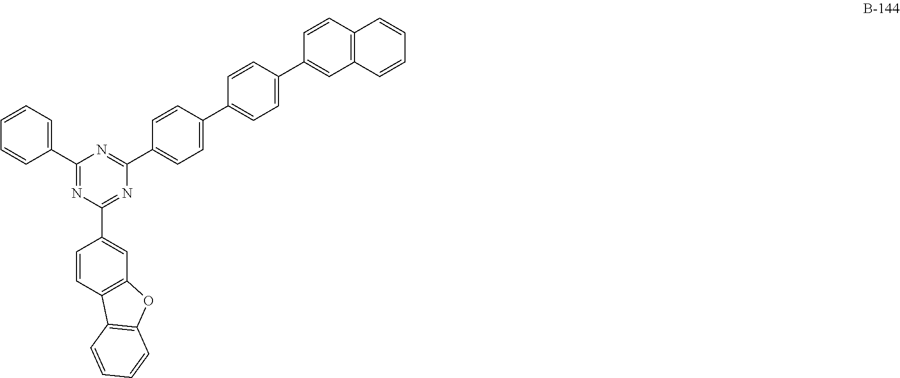 [SMILES: c1ccc(-c2nc(-c3ccc(-c4ccc(-c5ccc6ccccc6c5)cc4)cc3)nc(-c3ccc4c(c3)oc3ccccc34)n2)cc1]

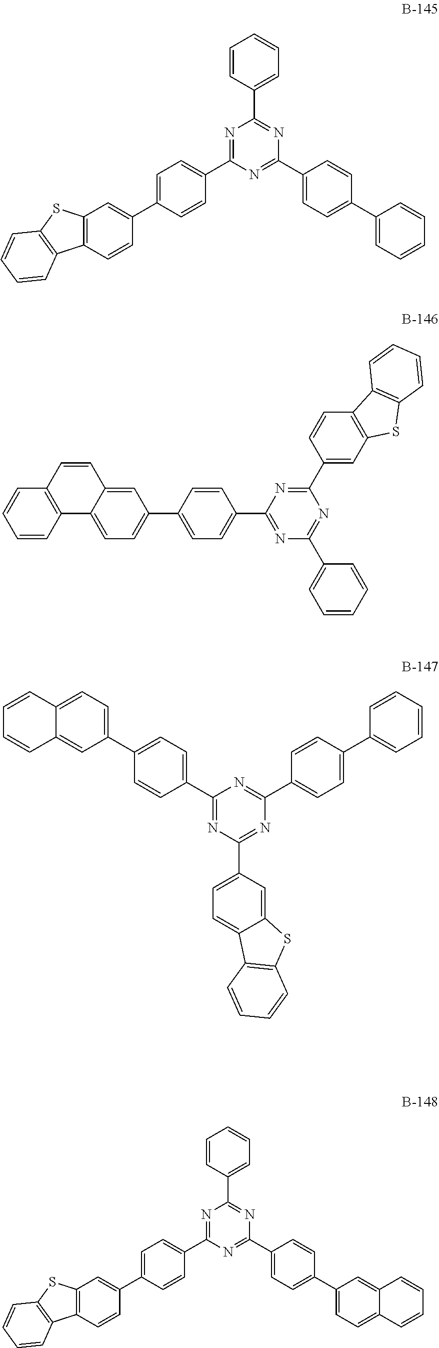 [SMILES: c1ccc(-c2ccc(-c3nc(-c4ccccc4)nc(-c4ccc(-c5ccc6c(c5)sc5ccccc56)cc4)n3)cc2)cc1 ; c1ccc(-c2nc(-c3ccc(-c4ccc5c(ccc6ccccc65)c4)cc3)nc(-c3ccc4c(c3)sc3ccccc34)n2)cc1 ; c1ccc(-c2ccc(-c3nc(-c4ccc(-c5ccc6ccccc6c5)cc4)nc(-c4ccc5c(c4)sc4ccccc45)n3)cc2)cc1 ; c1ccc(-c2nc(-c3ccc(-c4ccc5ccccc5c4)cc3)nc(-c3ccc(-c4ccc5c(c4)sc4ccccc45)cc3)n2)cc1]

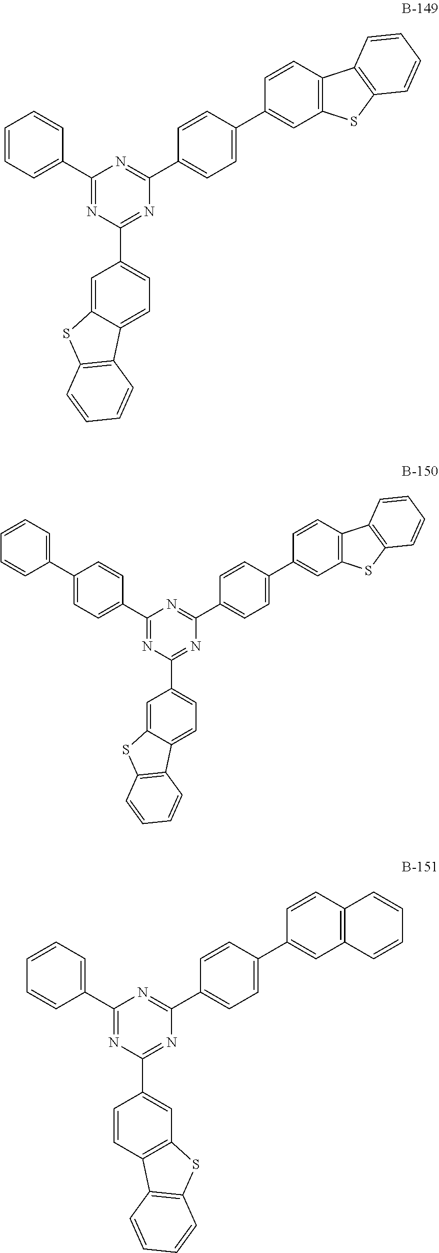 [SMILES: c1ccc(-c2nc(-c3ccc(-c4ccc5c(c4)sc4ccccc45)cc3)nc(-c3ccc4c(c3)sc3ccccc34)n2)cc1 ; c1ccc(-c2ccc(-c3nc(-c4ccc(-c5ccc6c(c5)sc5ccccc56)cc4)nc(-c4ccc5c(c4)sc4ccccc45)n3)cc2)cc1 ; c1ccc(-c2nc(-c3ccc(-c4ccc5ccccc5c4)cc3)nc(-c3ccc4c(c3)sc3ccccc34)n2)cc1]

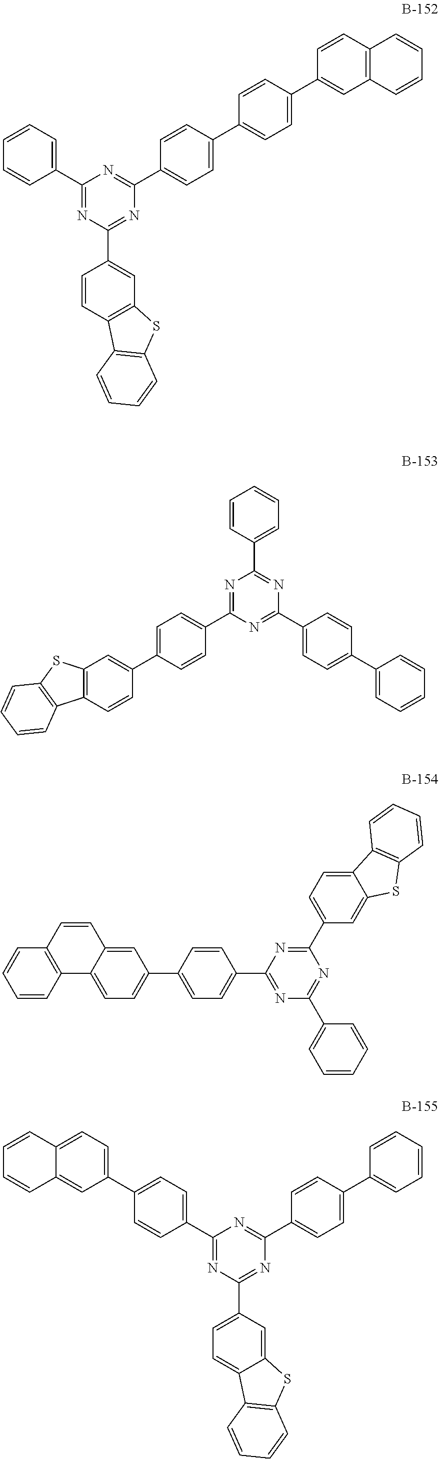 [SMILES: c1ccc(-c2nc(-c3ccc(-c4ccc(-c5ccc6ccccc6c5)cc4)cc3)nc(-c3ccc4c(c3)sc3ccccc34)n2)cc1 ; c1ccc(-c2ccc(-c3nc(-c4ccccc4)nc(-c4ccc(-c5ccc6c(c5)sc5ccccc56)cc4)n3)cc2)cc1 ; c1ccc(-c2nc(-c3ccc(-c4ccc5c(ccc6ccccc65)c4)cc3)nc(-c3ccc4c(c3)sc3ccccc34)n2)cc1 ; c1ccc(-c2ccc(-c3nc(-c4ccc(-c5ccc6ccccc6c5)cc4)nc(-c4ccc5c(c4)sc4ccccc45)n3)cc2)cc1]

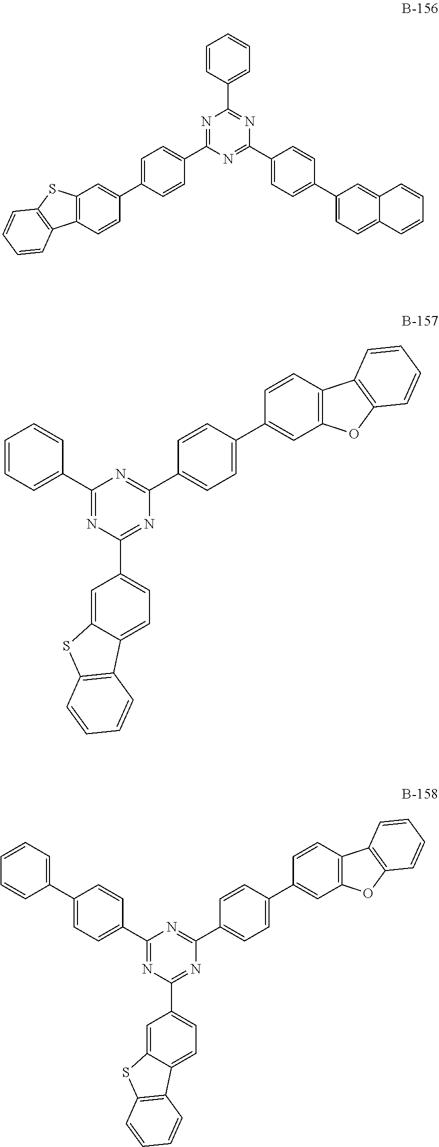 [SMILES: c1ccc(-c2nc(-c3ccc(-c4ccc5ccccc5c4)cc3)nc(-c3ccc(-c4ccc5c(c4)sc4ccccc45)cc3)n2)cc1 ; c1ccc(-c2nc(-c3ccc(-c4ccc5c(c4)oc4ccccc45)cc3)nc(-c3ccc4c(c3)sc3ccccc34)n2)cc1 ; c1ccc(-c2ccc(-c3nc(-c4ccc(-c5ccc6c(c5)oc5ccccc56)cc4)nc(-c4ccc5c(c4)sc4ccccc45)n3)cc2)cc1]

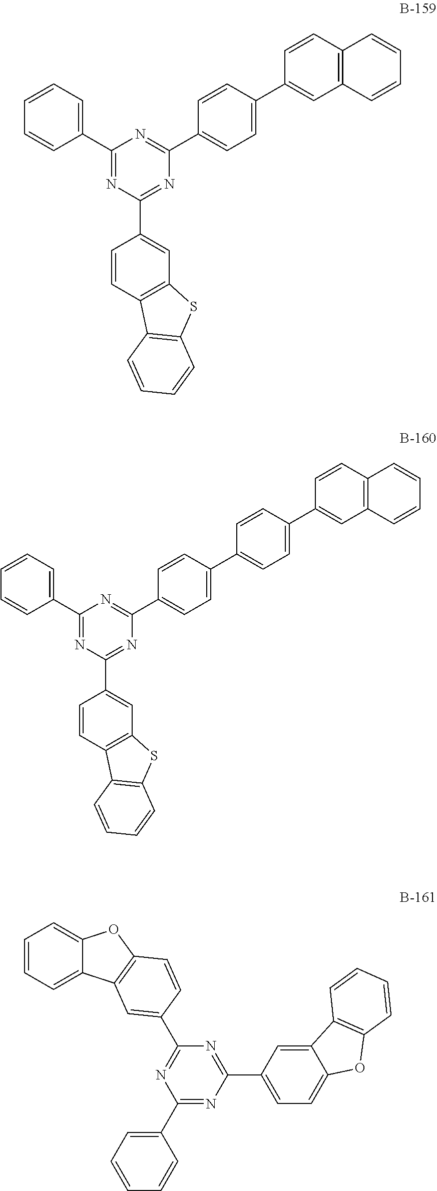 [SMILES: c1ccc(-c2nc(-c3ccc(-c4ccc5ccccc5c4)cc3)nc(-c3ccc4c(c3)sc3ccccc34)n2)cc1 ; c1ccc(-c2nc(-c3ccc(-c4ccc(-c5ccc6ccccc6c5)cc4)cc3)nc(-c3ccc4c(c3)sc3ccccc34)n2)cc1 ; c1ccc(-c2nc(-c3ccc4oc5ccccc5c4c3)nc(-c3ccc4oc5ccccc5c4c3)n2)cc1]

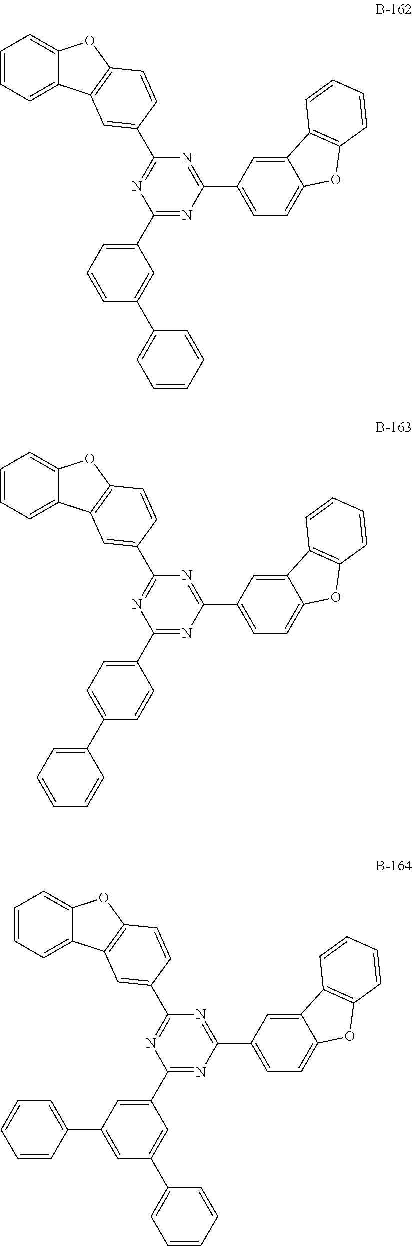 [SMILES: c1ccc(-c2cccc(-c3nc(-c4ccc5oc6ccccc6c5c4)nc(-c4ccc5oc6ccccc6c5c4)n3)c2)cc1 ; c1ccc(-c2ccc(-c3nc(-c4ccc5oc6ccccc6c5c4)nc(-c4ccc5oc6ccccc6c5c4)n3)cc2)cc1 ; c1ccc(-c2cc(-c3ccccc3)cc(-c3nc(-c4ccc5oc6ccccc6c5c4)nc(-c4ccc5oc6ccccc6c5c4)n3)c2)cc1]

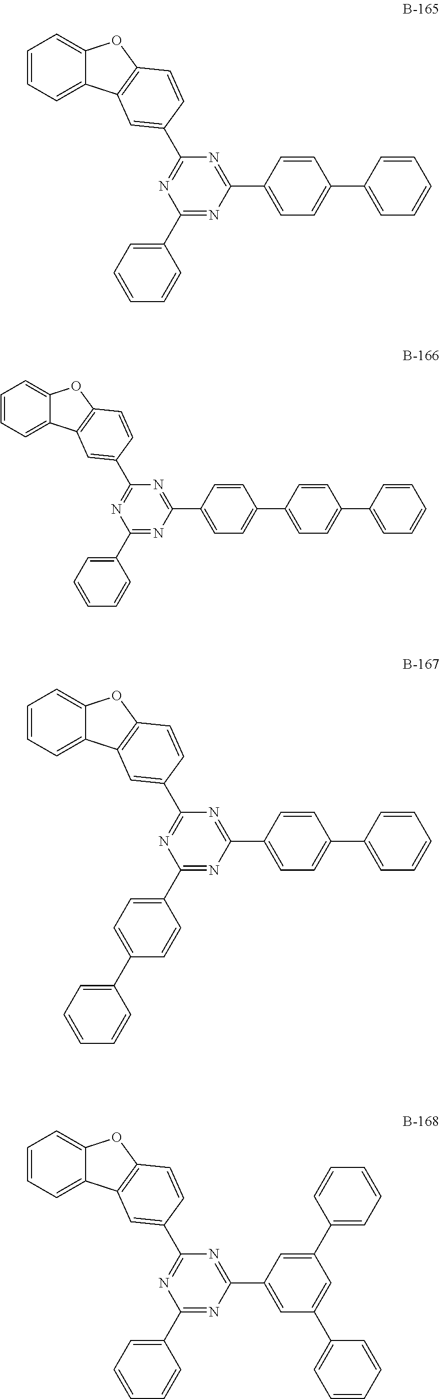 [SMILES: c1ccc(-c2ccc(-c3nc(-c4ccccc4)nc(-c4ccc5oc6ccccc6c5c4)n3)cc2)cc1 ; c1ccc(-c2ccc(-c3ccc(-c4nc(-c5ccccc5)nc(-c5ccc6oc7ccccc7c6c5)n4)cc3)cc2)cc1 ; c1ccc(-c2ccc(-c3nc(-c4ccc(-c5ccccc5)cc4)nc(-c4ccc5oc6ccccc6c5c4)n3)cc2)cc1 ; c1ccc(-c2cc(-c3ccccc3)cc(-c3nc(-c4ccccc4)nc(-c4ccc5oc6ccccc6c5c4)n3)c2)cc1]

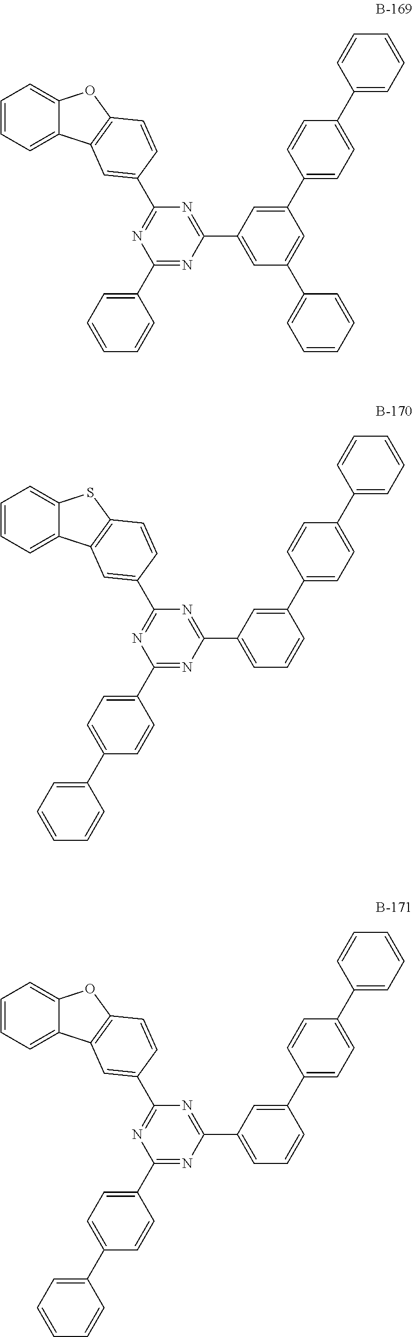 [SMILES: c1ccc(-c2ccc(-c3cc(-c4ccccc4)cc(-c4nc(-c5ccccc5)nc(-c5ccc6oc7ccccc7c6c5)n4)c3)cc2)cc1 ; c1ccc(-c2ccc(-c3cccc(-c4nc(-c5ccc(-c6ccccc6)cc5)nc(-c5ccc6sc7ccccc7c6c5)n4)c3)cc2)cc1 ; c1ccc(-c2ccc(-c3cccc(-c4nc(-c5ccc(-c6ccccc6)cc5)nc(-c5ccc6oc7ccccc7c6c5)n4)c3)cc2)cc1]

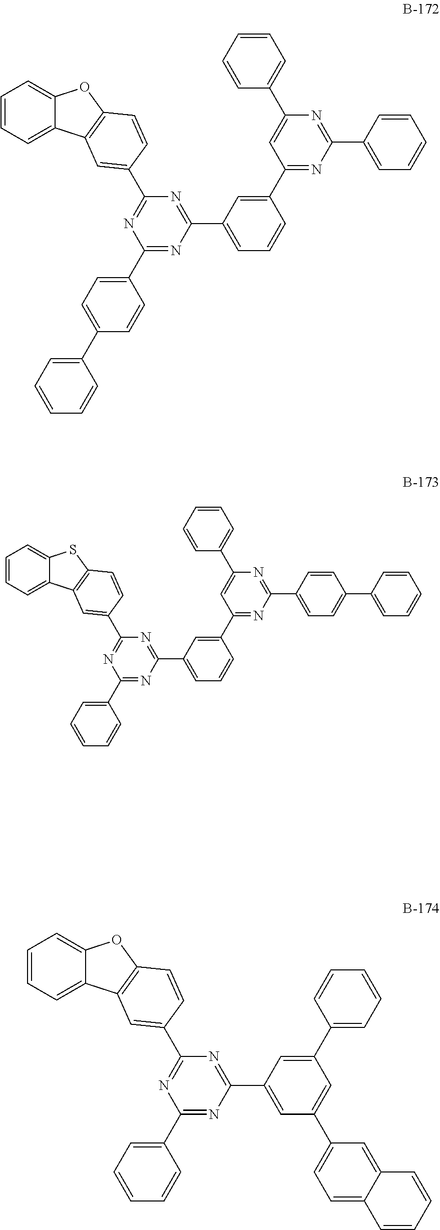 [SMILES: c1ccc(-c2ccc(-c3nc(-c4cccc(-c5cc(-c6ccccc6)nc(-c6ccccc6)n5)c4)nc(-c4ccc5oc6ccccc6c5c4)n3)cc2)cc1 ; c1ccc(-c2ccc(-c3nc(-c4ccccc4)cc(-c4cccc(-c5nc(-c6ccccc6)nc(-c6ccc7sc8ccccc8c7c6)n5)c4)n3)cc2)cc1 ; c1ccc(-c2cc(-c3ccc4ccccc4c3)cc(-c3nc(-c4ccccc4)nc(-c4ccc5oc6ccccc6c5c4)n3)c2)cc1]

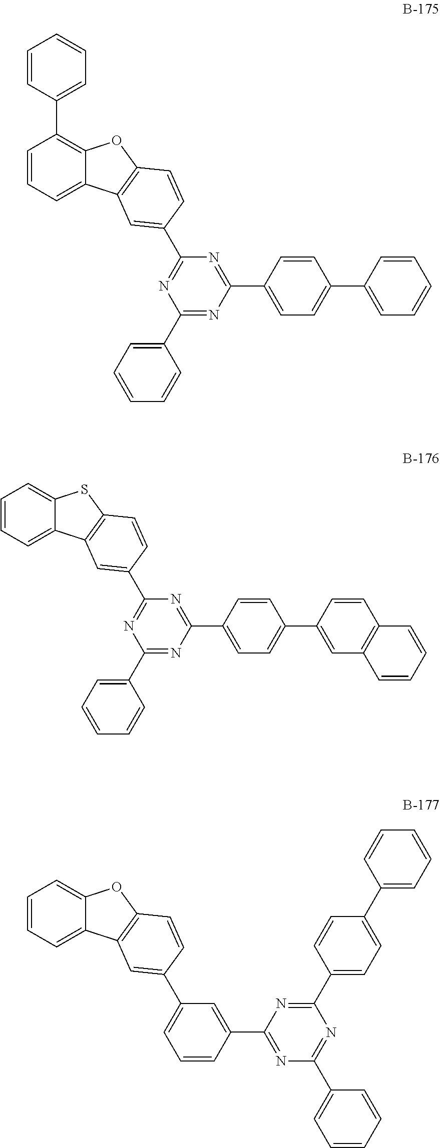 [SMILES: c1ccc(-c2ccc(-c3nc(-c4ccccc4)nc(-c4ccc5oc6c(-c7ccccc7)cccc6c5c4)n3)cc2)cc1 ; c1ccc(-c2nc(-c3ccc(-c4ccc5ccccc5c4)cc3)nc(-c3ccc4sc5ccccc5c4c3)n2)cc1 ; c1ccc(-c2ccc(-c3nc(-c4ccccc4)nc(-c4cccc(-c5ccc6oc7ccccc7c6c5)c4)n3)cc2)cc1]

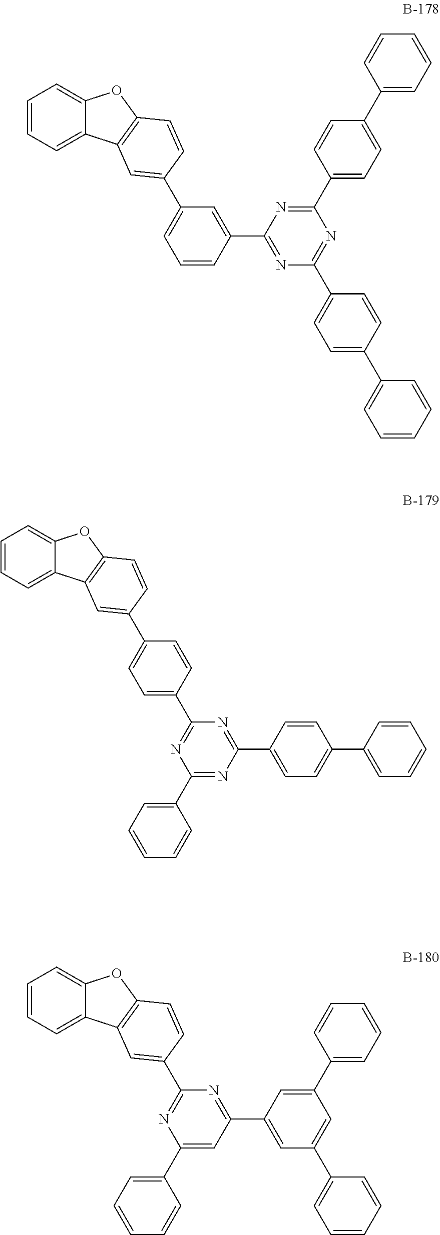 [SMILES: c1ccc(-c2ccc(-c3nc(-c4ccc(-c5ccccc5)cc4)nc(-c4cccc(-c5ccc6oc7ccccc7c6c5)c4)n3)cc2)cc1 ; c1ccc(-c2ccc(-c3nc(-c4ccccc4)nc(-c4ccc(-c5ccc6oc7ccccc7c6c5)cc4)n3)cc2)cc1 ; c1ccc(-c2cc(-c3ccccc3)cc(-c3cc(-c4ccccc4)nc(-c4ccc5oc6ccccc6c5c4)n3)c2)cc1]

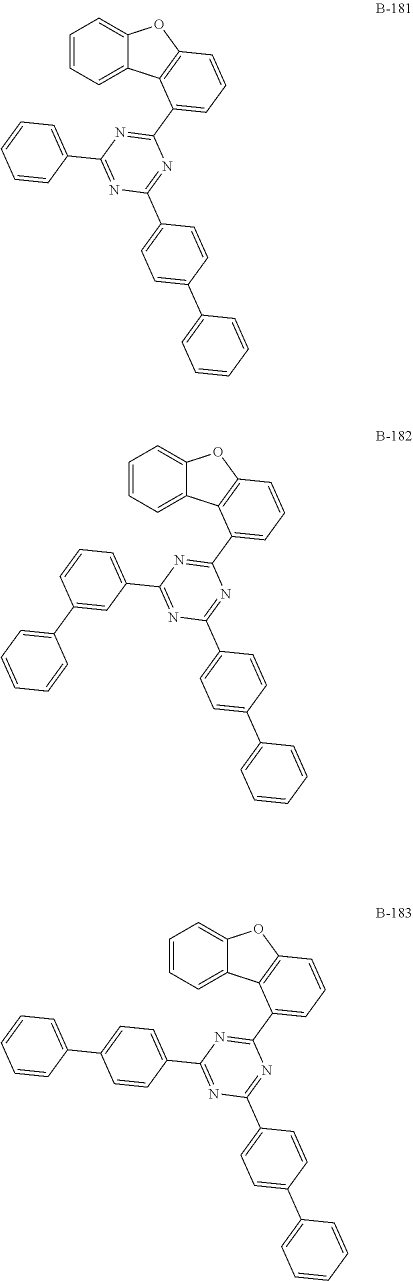 [SMILES: c1ccc(-c2ccc(-c3nc(-c4ccccc4)nc(-c4cccc5oc6ccccc6c45)n3)cc2)cc1 ; c1ccc(-c2ccc(-c3nc(-c4cccc(-c5ccccc5)c4)nc(-c4cccc5oc6ccccc6c45)n3)cc2)cc1 ; c1ccc(-c2ccc(-c3nc(-c4ccc(-c5ccccc5)cc4)nc(-c4cccc5oc6ccccc6c45)n3)cc2)cc1]

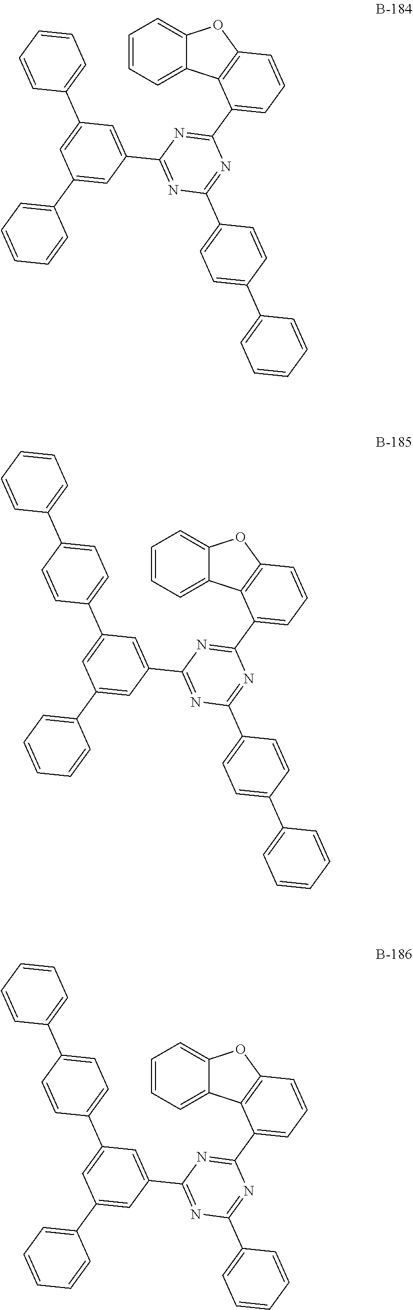 [SMILES: c1ccc(-c2ccc(-c3nc(-c4cc(-c5ccccc5)cc(-c5ccccc5)c4)nc(-c4cccc5oc6ccccc6c45)n3)cc2)cc1 ; c1ccc(-c2ccc(-c3cc(-c4ccccc4)cc(-c4nc(-c5ccc(-c6ccccc6)cc5)nc(-c5cccc6oc7ccccc7c56)n4)c3)cc2)cc1 ; c1ccc(-c2ccc(-c3cc(-c4ccccc4)cc(-c4nc(-c5ccccc5)nc(-c5cccc6oc7ccccc7c56)n4)c3)cc2)cc1]

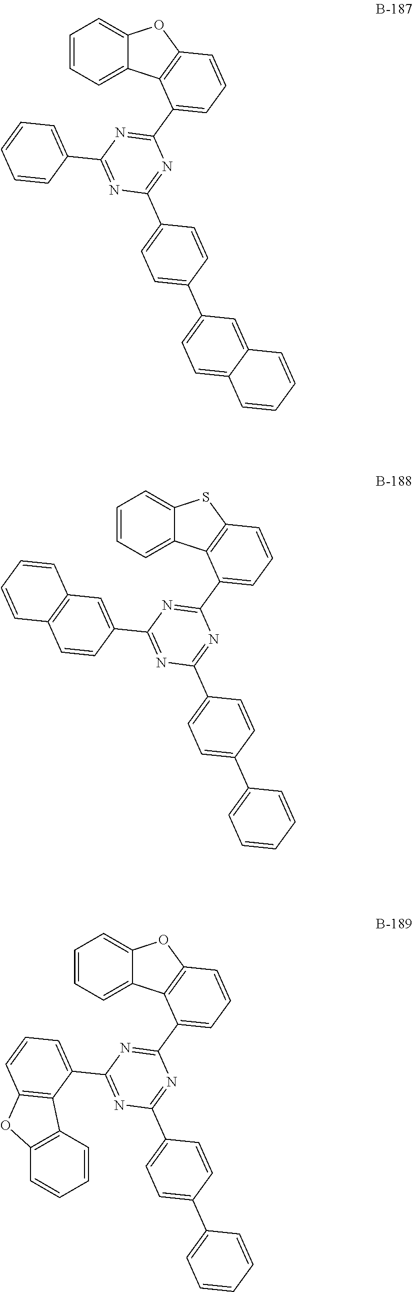 [SMILES: c1ccc(-c2nc(-c3ccc(-c4ccc5ccccc5c4)cc3)nc(-c3cccc4oc5ccccc5c34)n2)cc1 ; c1ccc(-c2ccc(-c3nc(-c4ccc5ccccc5c4)nc(-c4cccc5sc6ccccc6c45)n3)cc2)cc1 ; c1ccc(-c2ccc(-c3nc(-c4cccc5oc6ccccc6c45)nc(-c4cccc5oc6ccccc6c45)n3)cc2)cc1]

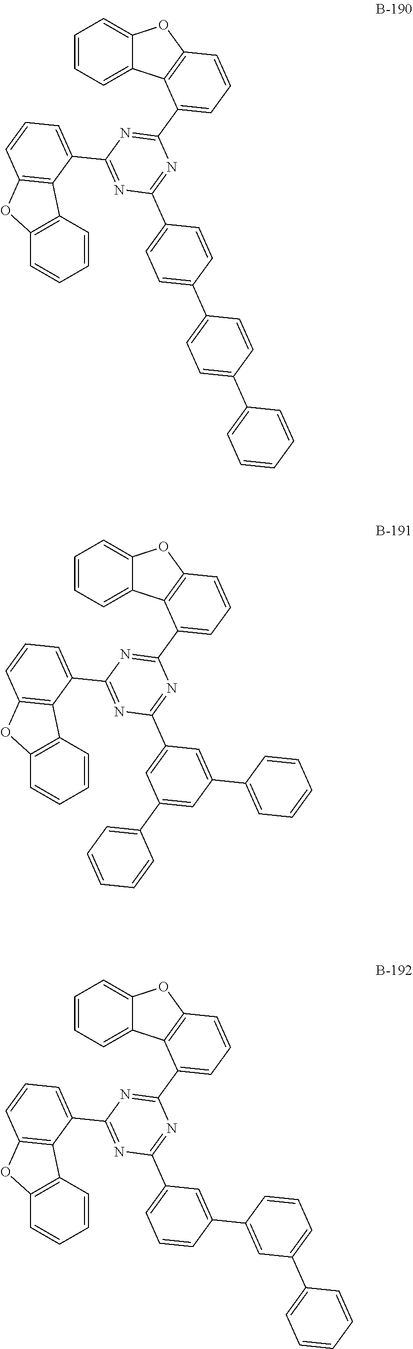 [SMILES: c1ccc(-c2ccc(-c3ccc(-c4nc(-c5cccc6oc7ccccc7c56)nc(-c5cccc6oc7ccccc7c56)n4)cc3)cc2)cc1 ; c1ccc(-c2cc(-c3ccccc3)cc(-c3nc(-c4cccc5oc6ccccc6c45)nc(-c4cccc5oc6ccccc6c45)n3)c2)cc1 ; c1ccc(-c2cccc(-c3cccc(-c4nc(-c5cccc6oc7ccccc7c56)nc(-c5cccc6oc7ccccc7c56)n4)c3)c2)cc1]

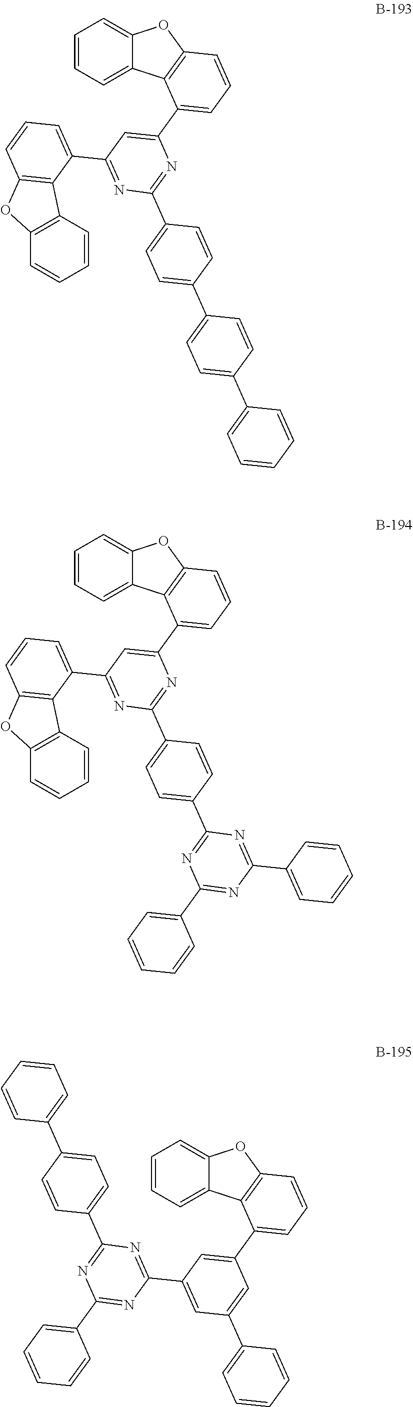 [SMILES: c1ccc(-c2ccc(-c3ccc(-c4nc(-c5cccc6oc7ccccc7c56)cc(-c5cccc6oc7ccccc7c56)n4)cc3)cc2)cc1 ; c1ccc(-c2nc(-c3ccccc3)nc(-c3ccc(-c4nc(-c5cccc6oc7ccccc7c56)cc(-c5cccc6oc7ccccc7c56)n4)cc3)n2)cc1 ; c1ccc(-c2ccc(-c3nc(-c4ccccc4)nc(-c4cc(-c5ccccc5)cc(-c5cccc6oc7ccccc7c56)c4)n3)cc2)cc1]

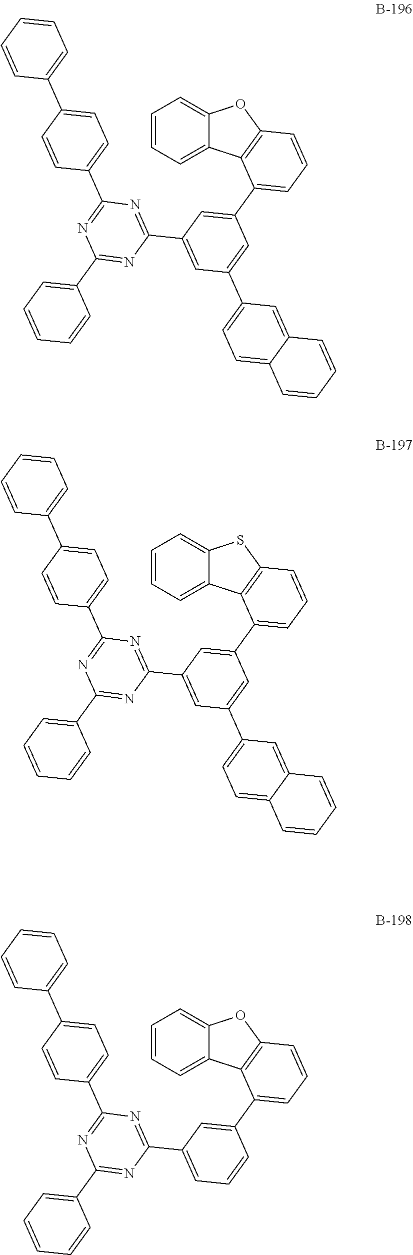 [SMILES: c1ccc(-c2ccc(-c3nc(-c4ccccc4)nc(-c4cc(-c5ccc6ccccc6c5)cc(-c5cccc6oc7ccccc7c56)c4)n3)cc2)cc1 ; c1ccc(-c2ccc(-c3nc(-c4ccccc4)nc(-c4cc(-c5ccc6ccccc6c5)cc(-c5cccc6sc7ccccc7c56)c4)n3)cc2)cc1 ; c1ccc(-c2ccc(-c3nc(-c4ccccc4)nc(-c4cccc(-c5cccc6oc7ccccc7c56)c4)n3)cc2)cc1]

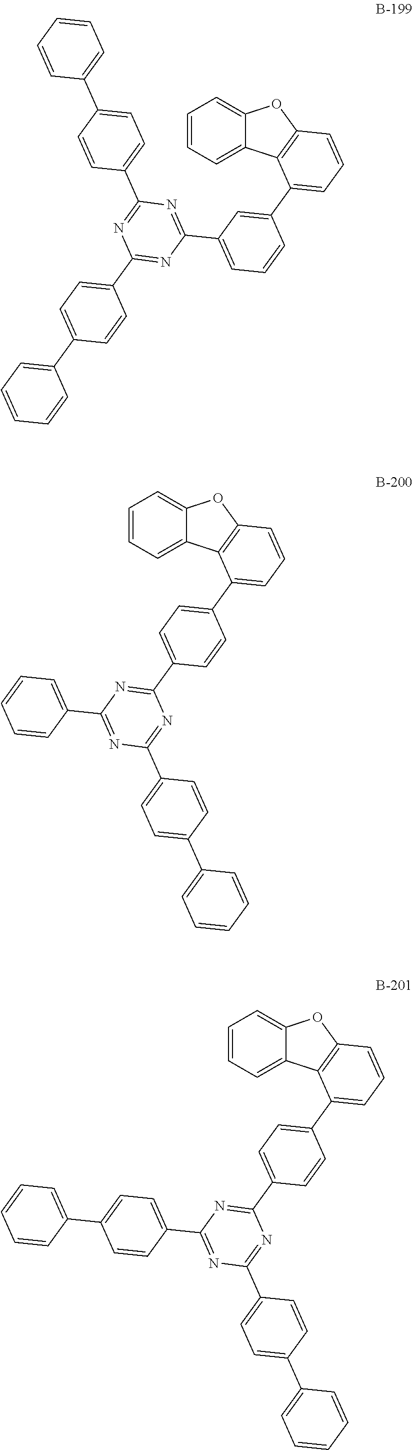 [SMILES: c1ccc(-c2ccc(-c3nc(-c4ccc(-c5ccccc5)cc4)nc(-c4cccc(-c5cccc6oc7ccccc7c56)c4)n3)cc2)cc1 ; c1ccc(-c2ccc(-c3nc(-c4ccccc4)nc(-c4ccc(-c5cccc6oc7ccccc7c56)cc4)n3)cc2)cc1 ; c1ccc(-c2ccc(-c3nc(-c4ccc(-c5ccccc5)cc4)nc(-c4ccc(-c5cccc6oc7ccccc7c56)cc4)n3)cc2)cc1]

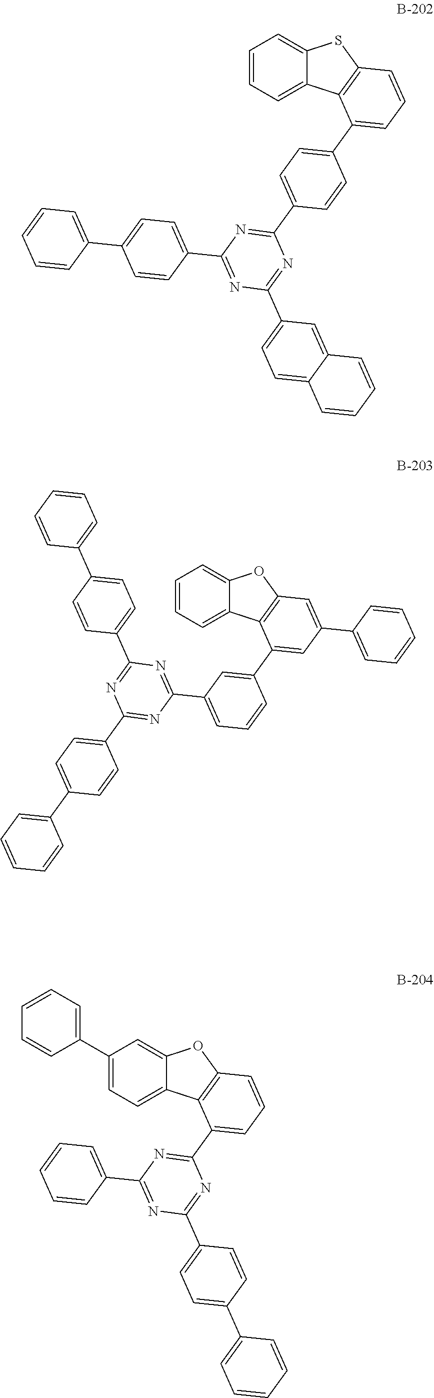 [SMILES: c1ccc(-c2ccc(-c3nc(-c4ccc(-c5cccc6sc7ccccc7c56)cc4)nc(-c4ccc5ccccc5c4)n3)cc2)cc1 ; c1ccc(-c2ccc(-c3nc(-c4ccc(-c5ccccc5)cc4)nc(-c4cccc(-c5cc(-c6ccccc6)cc6oc7ccccc7c56)c4)n3)cc2)cc1 ; c1ccc(-c2ccc(-c3nc(-c4ccccc4)nc(-c4cccc5oc6cc(-c7ccccc7)ccc6c45)n3)cc2)cc1]

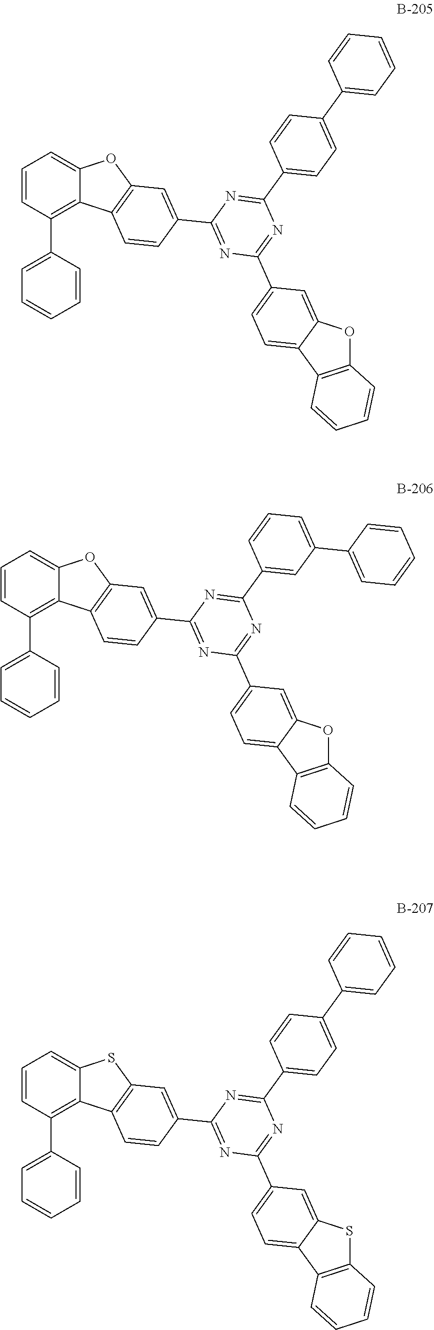 [SMILES: c1ccc(-c2ccc(-c3nc(-c4ccc5c(c4)oc4ccccc45)nc(-c4ccc5c(c4)oc4cccc(-c6ccccc6)c45)n3)cc2)cc1 ; c1ccc(-c2cccc(-c3nc(-c4ccc5c(c4)oc4ccccc45)nc(-c4ccc5c(c4)oc4cccc(-c6ccccc6)c45)n3)c2)cc1 ; c1ccc(-c2ccc(-c3nc(-c4ccc5c(c4)sc4ccccc45)nc(-c4ccc5c(c4)sc4cccc(-c6ccccc6)c45)n3)cc2)cc1]

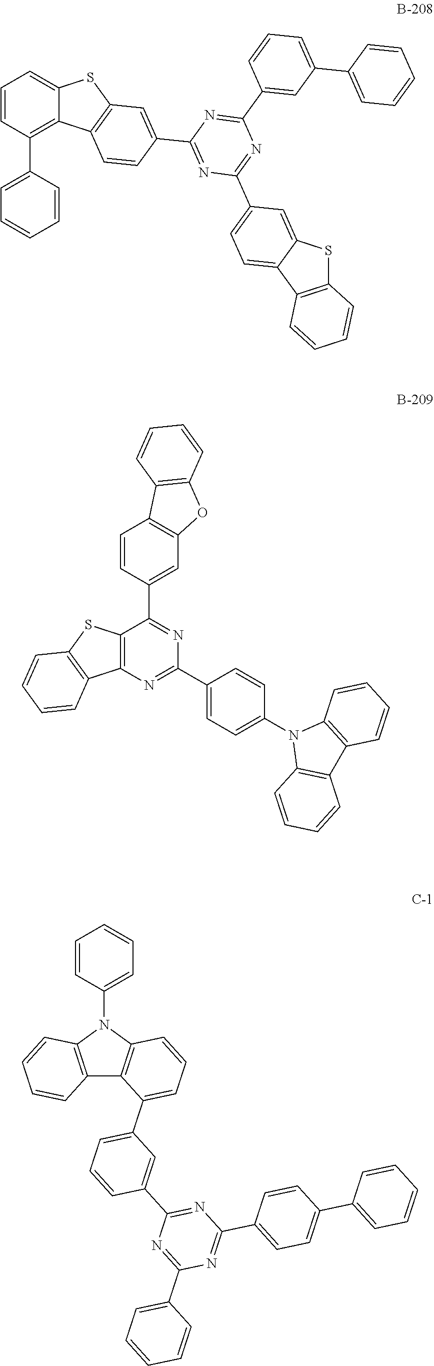 [SMILES: c1ccc(-c2cccc(-c3nc(-c4ccc5c(c4)sc4ccccc45)nc(-c4ccc5c(c4)sc4cccc(-c6ccccc6)c45)n3)c2)cc1 ; c1ccc2c(c1)oc1cc(-c3nc(-c4ccc(-n5c6ccccc6c6ccccc65)cc4)nc4c3sc3ccccc34)ccc12 ; c1ccc(-c2ccc(-c3nc(-c4ccccc4)nc(-c4cccc(-c5cccc6c5c5ccccc5n6-c5ccccc5)c4)n3)cc2)cc1]

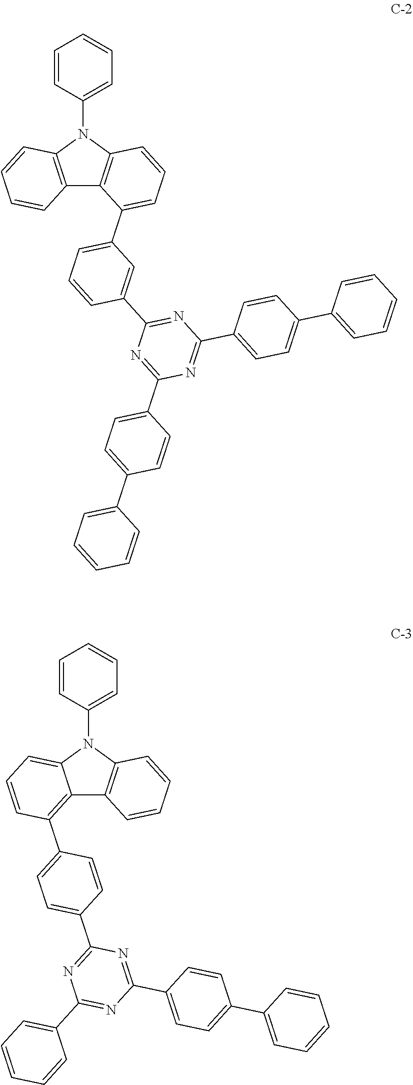 [SMILES: c1ccc(-c2ccc(-c3nc(-c4ccc(-c5ccccc5)cc4)nc(-c4cccc(-c5cccc6c5c5ccccc5n6-c5ccccc5)c4)n3)cc2)cc1 ; c1ccc(-c2ccc(-c3nc(-c4ccccc4)nc(-c4ccc(-c5cccc6c5c5ccccc5n6-c5ccccc5)cc4)n3)cc2)cc1]

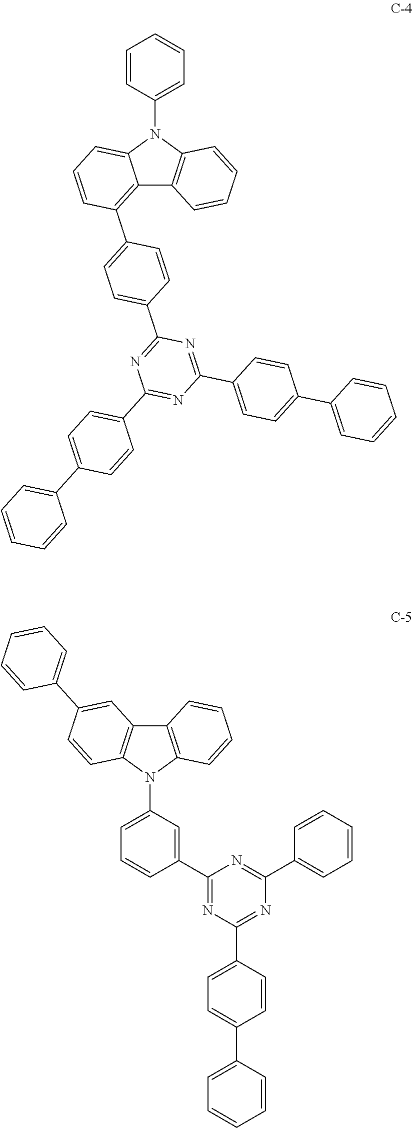 [SMILES: c1ccc(-c2ccc(-c3nc(-c4ccc(-c5ccccc5)cc4)nc(-c4ccc(-c5cccc6c5c5ccccc5n6-c5ccccc5)cc4)n3)cc2)cc1 ; c1ccc(-c2ccc(-c3nc(-c4ccccc4)nc(-c4cccc(-n5c6ccccc6c6cc(-c7ccccc7)ccc65)c4)n3)cc2)cc1]

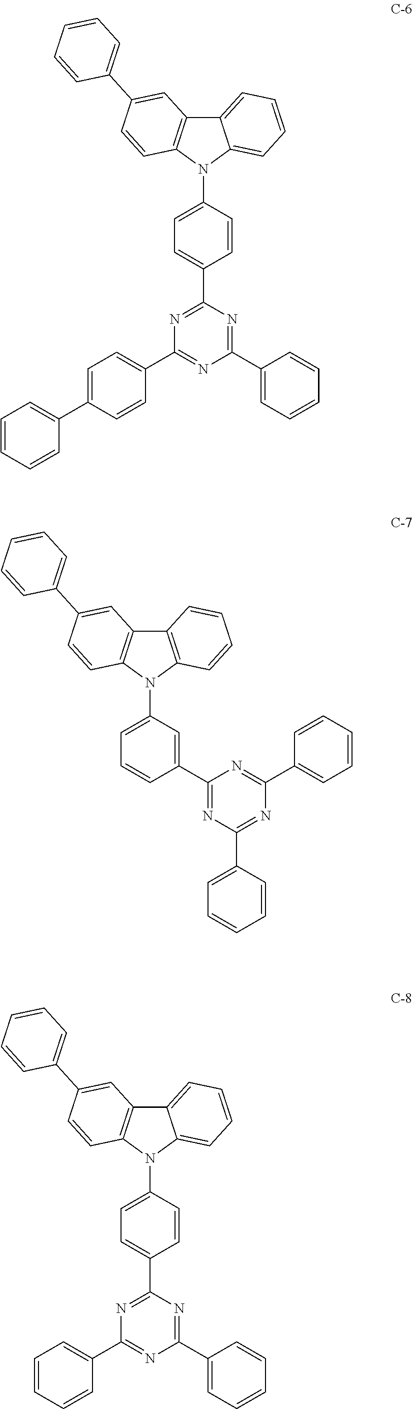 [SMILES: c1ccc(-c2ccc(-c3nc(-c4ccccc4)nc(-c4ccc(-n5c6ccccc6c6cc(-c7ccccc7)ccc65)cc4)n3)cc2)cc1 ; c1ccc(-c2ccc3c(c2)c2ccccc2n3-c2cccc(-c3nc(-c4ccccc4)nc(-c4ccccc4)n3)c2)cc1 ; c1ccc(-c2ccc3c(c2)c2ccccc2n3-c2ccc(-c3nc(-c4ccccc4)nc(-c4ccccc4)n3)cc2)cc1]

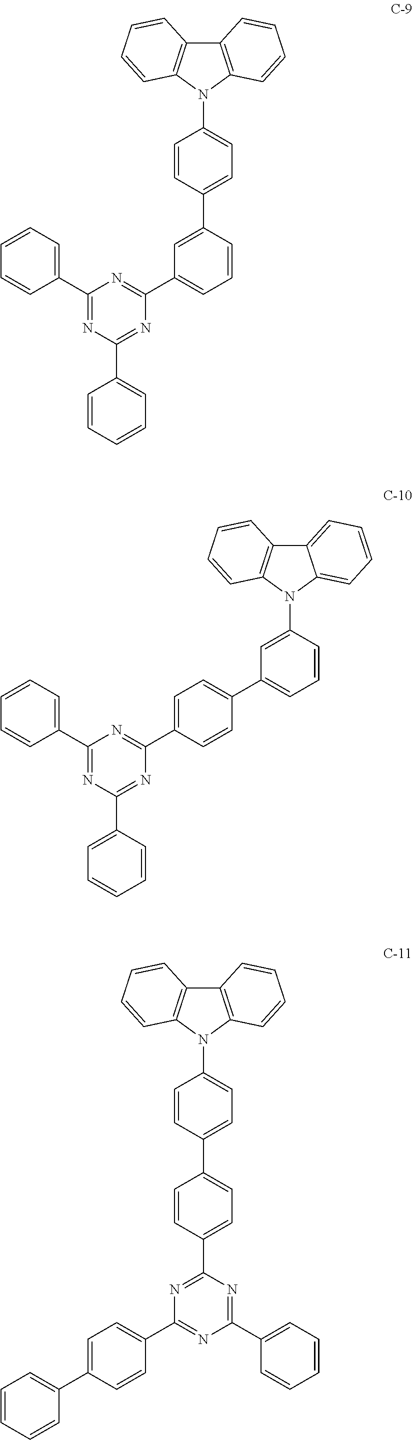 [SMILES: c1ccc(-c2nc(-c3ccccc3)nc(-c3cccc(-c4ccc(-n5c6ccccc6c6ccccc65)cc4)c3)n2)cc1 ; c1ccc(-c2nc(-c3ccccc3)nc(-c3ccc(-c4cccc(-n5c6ccccc6c6ccccc65)c4)cc3)n2)cc1 ; c1ccc(-c2ccc(-c3nc(-c4ccccc4)nc(-c4ccc(-c5ccc(-n6c7ccccc7c7ccccc76)cc5)cc4)n3)cc2)cc1]

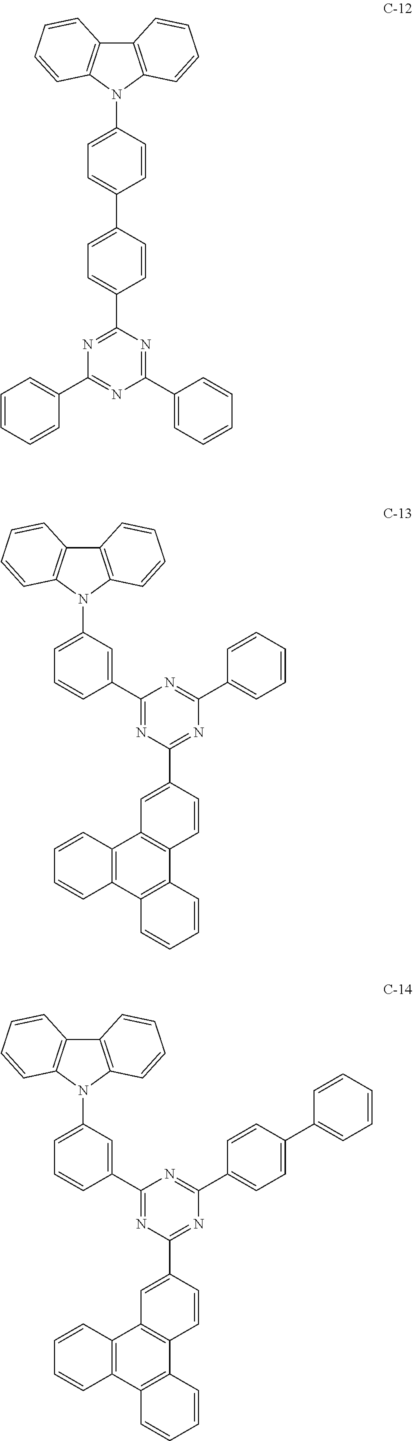 [SMILES: c1ccc(-c2nc(-c3ccccc3)nc(-c3ccc(-c4ccc(-n5c6ccccc6c6ccccc65)cc4)cc3)n2)cc1 ; c1ccc(-c2nc(-c3cccc(-n4c5ccccc5c5ccccc54)c3)nc(-c3ccc4c5ccccc5c5ccccc5c4c3)n2)cc1 ; c1ccc(-c2ccc(-c3nc(-c4cccc(-n5c6ccccc6c6ccccc65)c4)nc(-c4ccc5c6ccccc6c6ccccc6c5c4)n3)cc2)cc1]

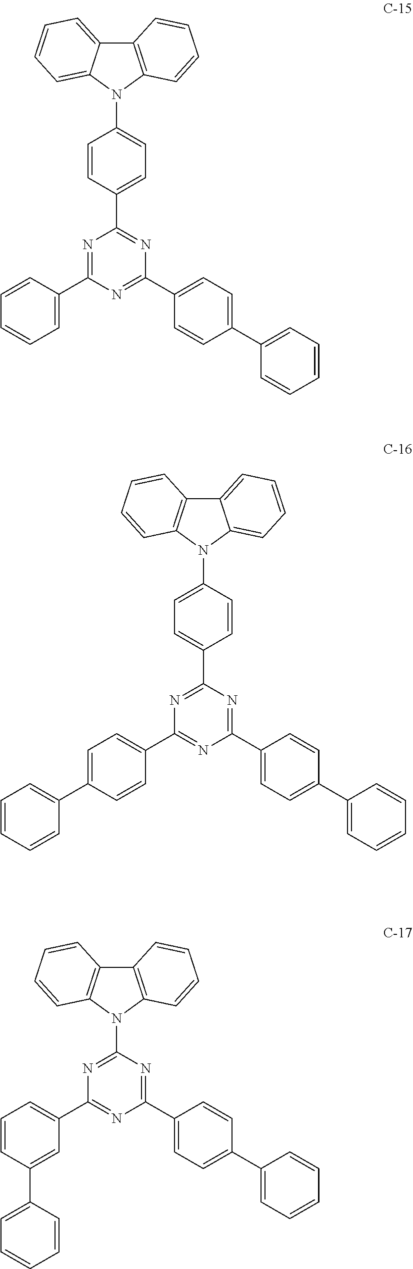 [SMILES: c1ccc(-c2ccc(-c3nc(-c4ccccc4)nc(-c4ccc(-n5c6ccccc6c6ccccc65)cc4)n3)cc2)cc1 ; c1ccc(-c2ccc(-c3nc(-c4ccc(-c5ccccc5)cc4)nc(-c4ccc(-n5c6ccccc6c6ccccc65)cc4)n3)cc2)cc1 ; c1ccc(-c2ccc(-c3nc(-c4cccc(-c5ccccc5)c4)nc(-n4c5ccccc5c5ccccc54)n3)cc2)cc1]

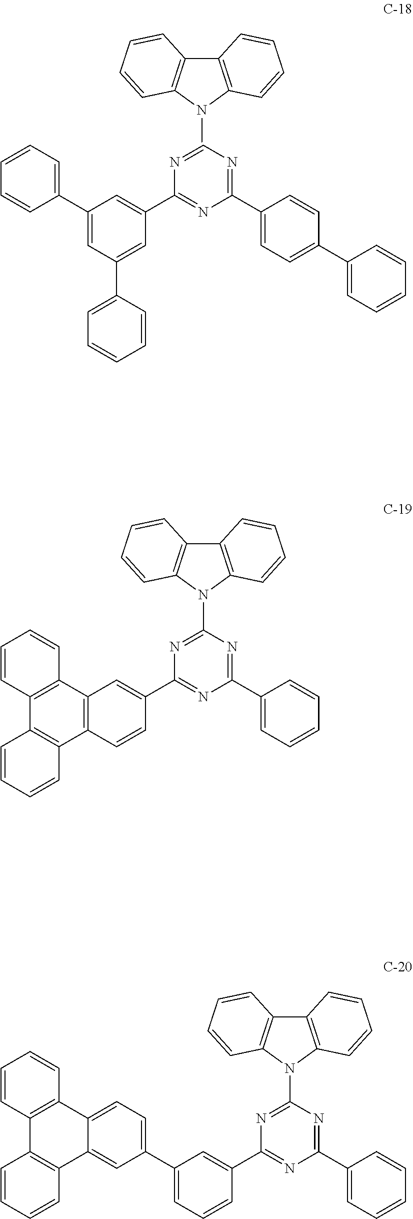

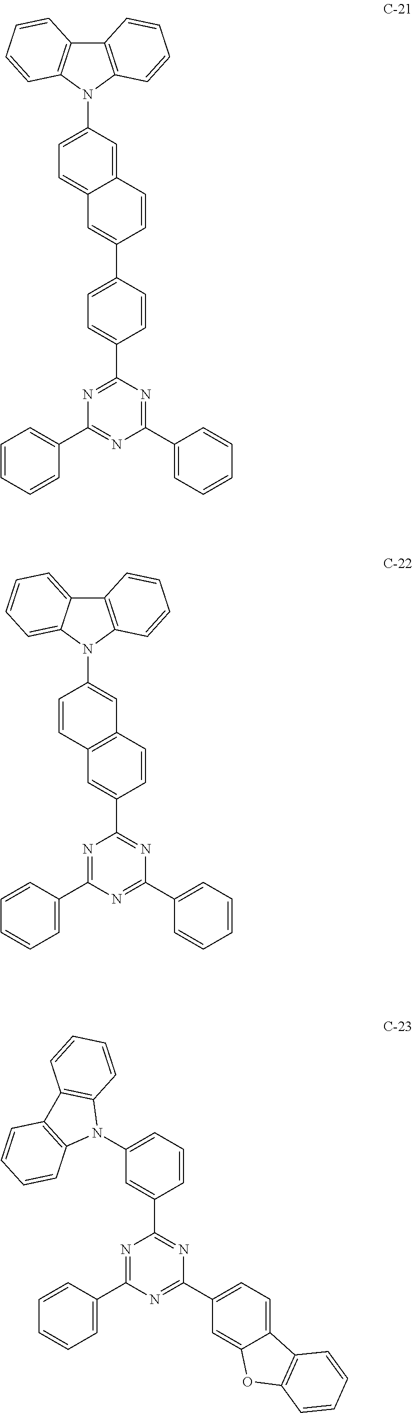 [SMILES: c1ccc(-c2nc(-c3ccccc3)nc(-c3ccc(-c4ccc5cc(-n6c7ccccc7c7ccccc76)ccc5c4)cc3)n2)cc1 ; c1ccc(-c2nc(-c3ccccc3)nc(-c3ccc4cc(-n5c6ccccc6c6ccccc65)ccc4c3)n2)cc1 ; c1ccc(-c2nc(-c3cccc(-n4c5ccccc5c5ccccc54)c3)nc(-c3ccc4c(c3)oc3ccccc34)n2)cc1]

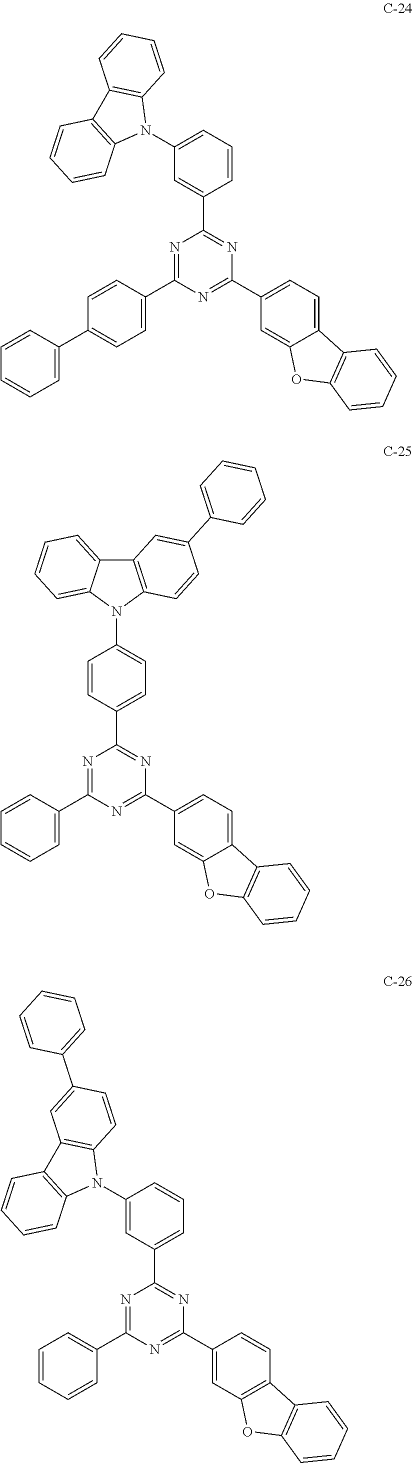 [SMILES: c1ccc(-c2ccc(-c3nc(-c4cccc(-n5c6ccccc6c6ccccc65)c4)nc(-c4ccc5c(c4)oc4ccccc45)n3)cc2)cc1 ; c1ccc(-c2ccc3c(c2)c2ccccc2n3-c2ccc(-c3nc(-c4ccccc4)nc(-c4ccc5c(c4)oc4ccccc45)n3)cc2)cc1 ; c1ccc(-c2ccc3c(c2)c2ccccc2n3-c2cccc(-c3nc(-c4ccccc4)nc(-c4ccc5c(c4)oc4ccccc45)n3)c2)cc1]

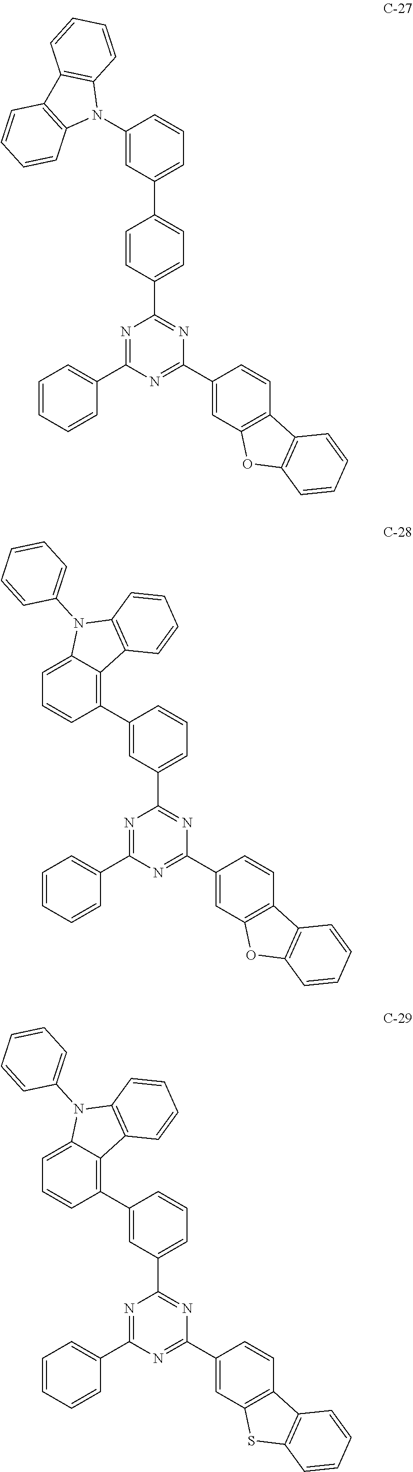 [SMILES: c1ccc(-c2nc(-c3ccc(-c4cccc(-n5c6ccccc6c6ccccc65)c4)cc3)nc(-c3ccc4c(c3)oc3ccccc34)n2)cc1 ; c1ccc(-c2nc(-c3cccc(-c4cccc5c4c4ccccc4n5-c4ccccc4)c3)nc(-c3ccc4c(c3)oc3ccccc34)n2)cc1 ; c1ccc(-c2nc(-c3cccc(-c4cccc5c4c4ccccc4n5-c4ccccc4)c3)nc(-c3ccc4c(c3)sc3ccccc34)n2)cc1]

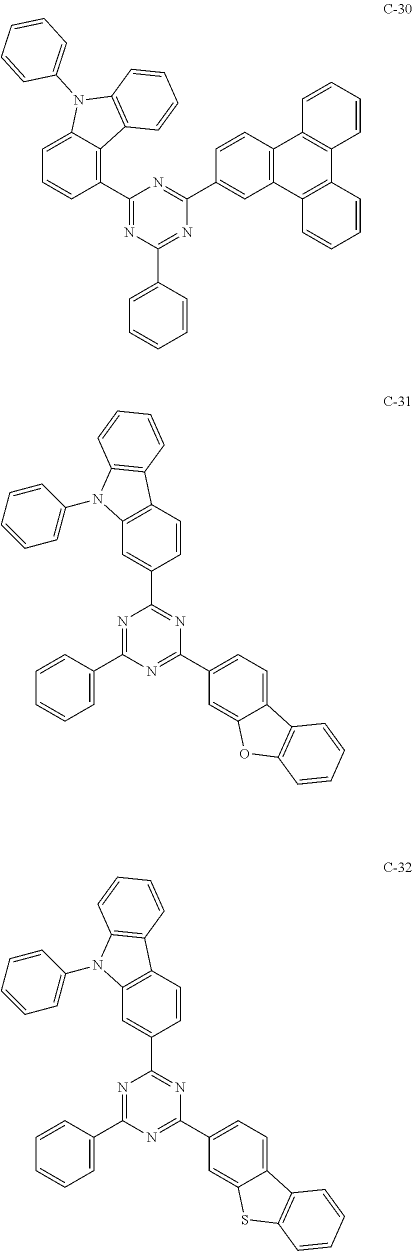 [SMILES: c1ccc(-c2nc(-c3ccc4c5ccccc5c5ccccc5c4c3)nc(-c3cccc4c3c3ccccc3n4-c3ccccc3)n2)cc1 ; c1ccc(-c2nc(-c3ccc4c(c3)oc3ccccc34)nc(-c3ccc4c5ccccc5n(-c5ccccc5)c4c3)n2)cc1 ; c1ccc(-c2nc(-c3ccc4c(c3)sc3ccccc34)nc(-c3ccc4c5ccccc5n(-c5ccccc5)c4c3)n2)cc1]

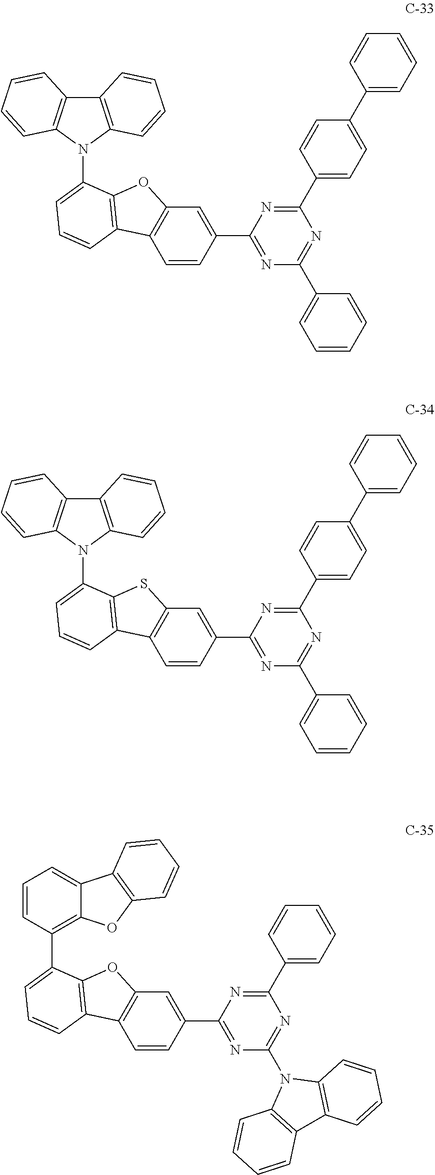 [SMILES: c1ccc(-c2ccc(-c3nc(-c4ccccc4)nc(-c4ccc5c(c4)oc4c(-n6c7ccccc7c7ccccc76)cccc45)n3)cc2)cc1 ; c1ccc(-c2ccc(-c3nc(-c4ccccc4)nc(-c4ccc5c(c4)sc4c(-n6c7ccccc7c7ccccc76)cccc45)n3)cc2)cc1 ; c1ccc(-c2nc(-c3ccc4c(c3)oc3c(-c5cccc6c5oc5ccccc56)cccc34)nc(-n3c4ccccc4c4ccccc43)n2)cc1]

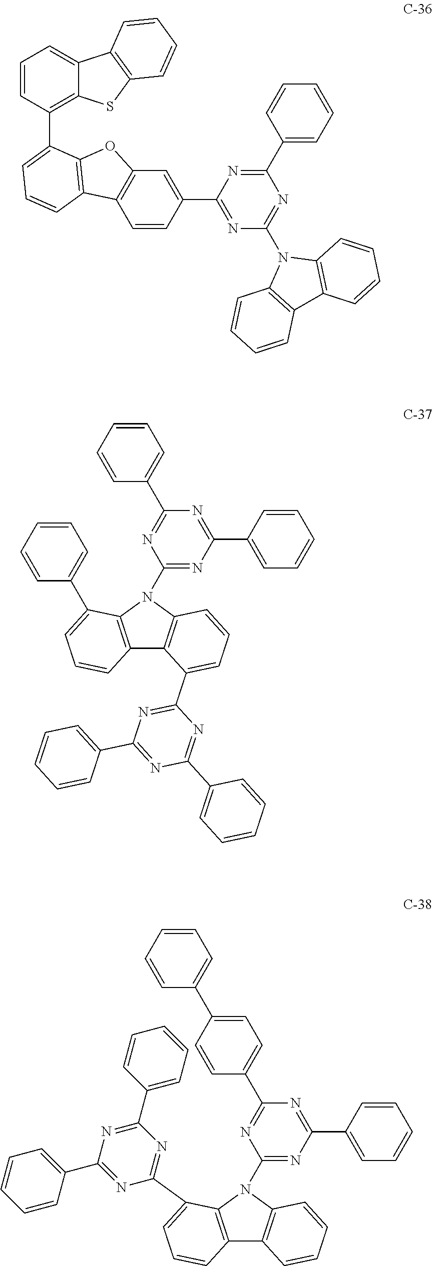 [SMILES: c1ccc(-c2nc(-c3ccc4c(c3)oc3c(-c5cccc6c5sc5ccccc56)cccc34)nc(-n3c4ccccc4c4ccccc43)n2)cc1 ; c1ccc(-c2nc(-c3ccccc3)nc(-c3cccc4c3c3cccc(-c5ccccc5)c3n4-c3nc(-c4ccccc4)nc(-c4ccccc4)n3)n2)cc1 ; c1ccc(-c2ccc(-c3nc(-c4ccccc4)nc(-n4c5ccccc5c5cccc(-c6nc(-c7ccccc7)nc(-c7ccccc7)n6)c54)n3)cc2)cc1]

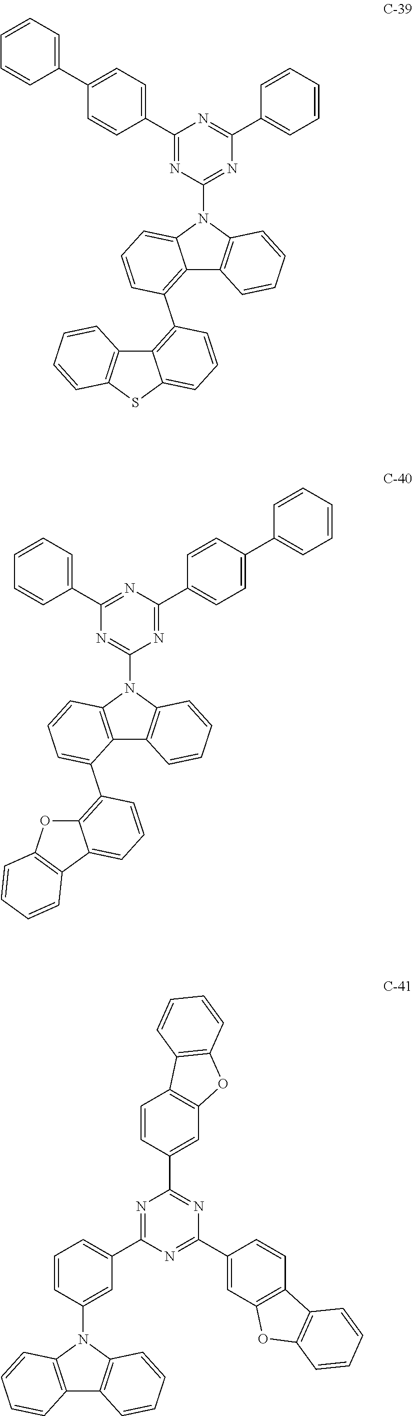 [SMILES: c1ccc(-c2ccc(-c3nc(-c4ccccc4)nc(-n4c5ccccc5c5c(-c6cccc7sc8ccccc8c67)cccc54)n3)cc2)cc1 ; c1ccc(-c2ccc(-c3nc(-c4ccccc4)nc(-n4c5ccccc5c5c(-c6cccc7c6oc6ccccc67)cccc54)n3)cc2)cc1 ; c1cc(-c2nc(-c3ccc4c(c3)oc3ccccc34)nc(-c3ccc4c(c3)oc3ccccc34)n2)cc(-n2c3ccccc3c3ccccc32)c1]

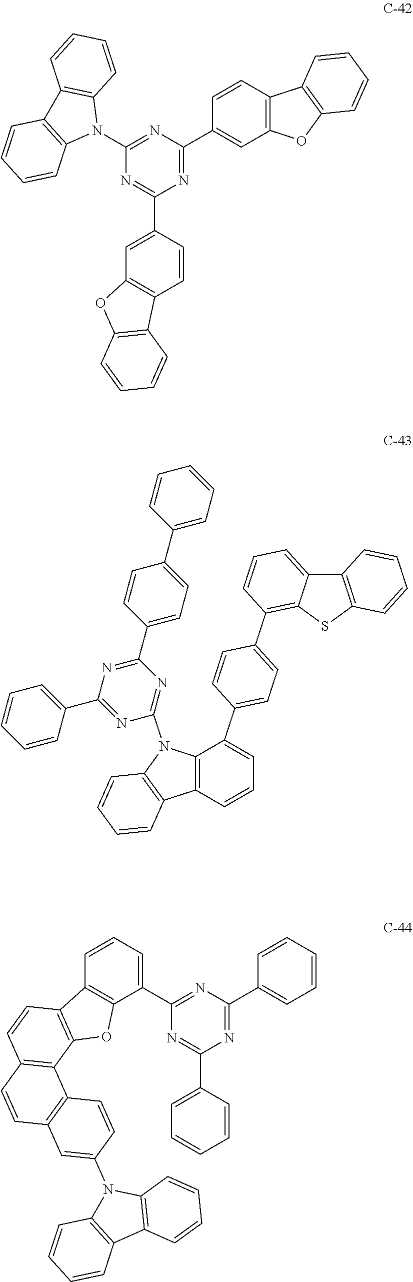 [SMILES: c1ccc2c(c1)oc1cc(-c3nc(-c4ccc5c(c4)oc4ccccc45)nc(-n4c5ccccc5c5ccccc54)n3)ccc12 ; c1ccc(-c2ccc(-c3nc(-c4ccccc4)nc(-n4c5ccccc5c5cccc(-c6ccc(-c7cccc8c7sc7ccccc78)cc6)c54)n3)cc2)cc1 ; c1ccc(-c2nc(-c3ccccc3)nc(-c3cccc4c3oc3c4ccc4ccc5cc(-n6c7ccccc7c7ccccc76)ccc5c43)n2)cc1]

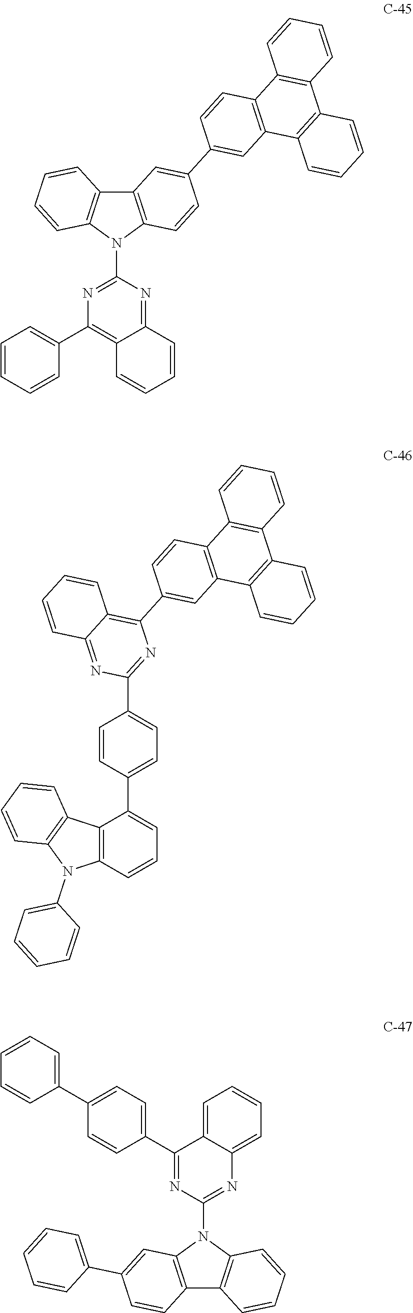 [SMILES: c1ccc(-c2nc(-n3c4ccccc4c4cc(-c5ccc6c7ccccc7c7ccccc7c6c5)ccc43)nc3ccccc23)cc1 ; c1ccc(-n2c3ccccc3c3c(-c4ccc(-c5nc(-c6ccc7c8ccccc8c8ccccc8c7c6)c6ccccc6n5)cc4)cccc32)cc1 ; c1ccc(-c2ccc(-c3nc(-n4c5ccccc5c5ccc(-c6ccccc6)cc54)nc4ccccc34)cc2)cc1]

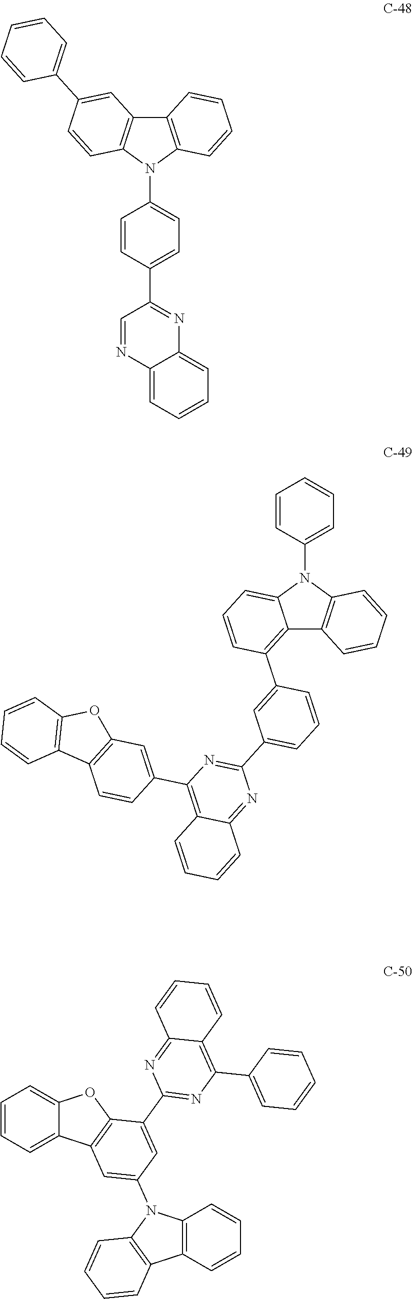 [SMILES: c1ccc(-c2ccc3c(c2)c2ccccc2n3-c2ccc(-c3cnc4ccccc4n3)cc2)cc1 ; c1ccc(-n2c3ccccc3c3c(-c4cccc(-c5nc(-c6ccc7c(c6)oc6ccccc67)c6ccccc6n5)c4)cccc32)cc1 ; c1ccc(-c2nc(-c3cc(-n4c5ccccc5c5ccccc54)cc4c3oc3ccccc34)nc3ccccc23)cc1]

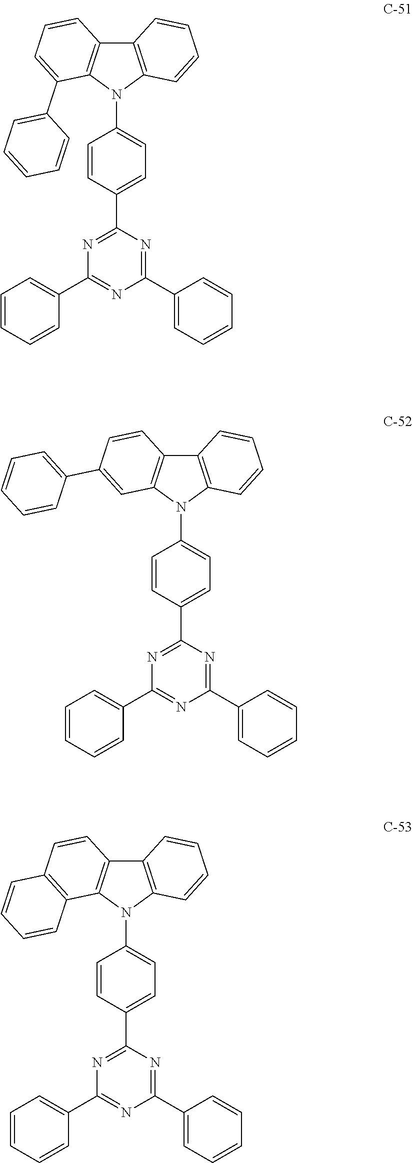 [SMILES: c1ccc(-c2nc(-c3ccccc3)nc(-c3ccc(-n4c5ccccc5c5cccc(-c6ccccc6)c54)cc3)n2)cc1 ; c1ccc(-c2ccc3c4ccccc4n(-c4ccc(-c5nc(-c6ccccc6)nc(-c6ccccc6)n5)cc4)c3c2)cc1 ; c1ccc(-c2nc(-c3ccccc3)nc(-c3ccc(-n4c5ccccc5c5ccc6ccccc6c54)cc3)n2)cc1]

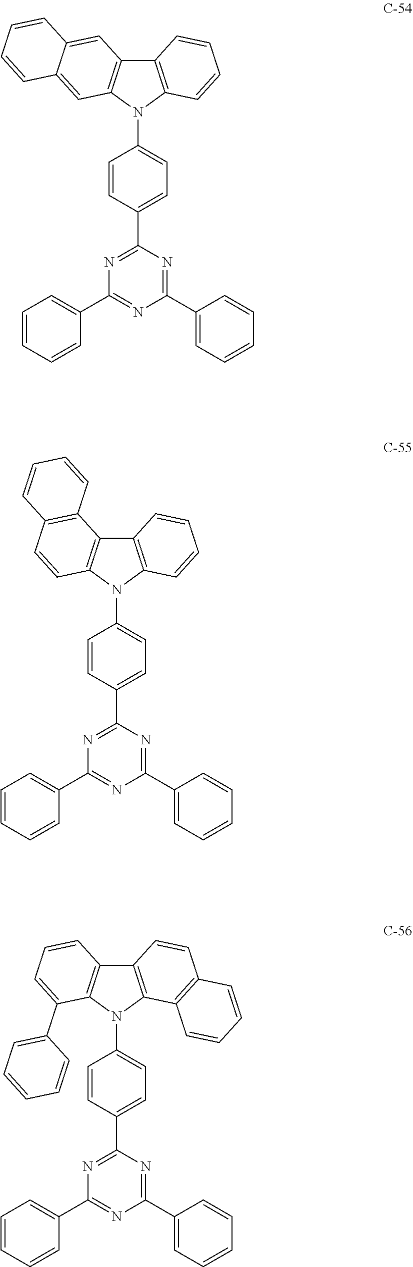 [SMILES: c1ccc(-c2nc(-c3ccccc3)nc(-c3ccc(-n4c5ccccc5c5cc6ccccc6cc54)cc3)n2)cc1 ; c1ccc(-c2nc(-c3ccccc3)nc(-c3ccc(-n4c5ccccc5c5c6ccccc6ccc54)cc3)n2)cc1 ; c1ccc(-c2nc(-c3ccccc3)nc(-c3ccc(-n4c5c(-c6ccccc6)cccc5c5ccc6ccccc6c54)cc3)n2)cc1]

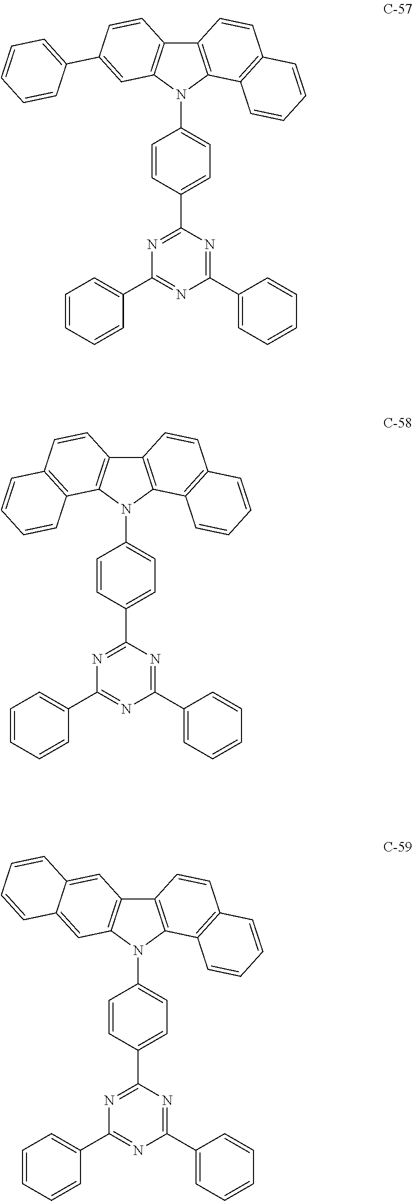 [SMILES: c1ccc(-c2ccc3c4ccc5ccccc5c4n(-c4ccc(-c5nc(-c6ccccc6)nc(-c6ccccc6)n5)cc4)c3c2)cc1 ; c1ccc(-c2nc(-c3ccccc3)nc(-c3ccc(-n4c5c6ccccc6ccc5c5ccc6ccccc6c54)cc3)n2)cc1 ; c1ccc(-c2nc(-c3ccccc3)nc(-c3ccc(-n4c5cc6ccccc6cc5c5ccc6ccccc6c54)cc3)n2)cc1]

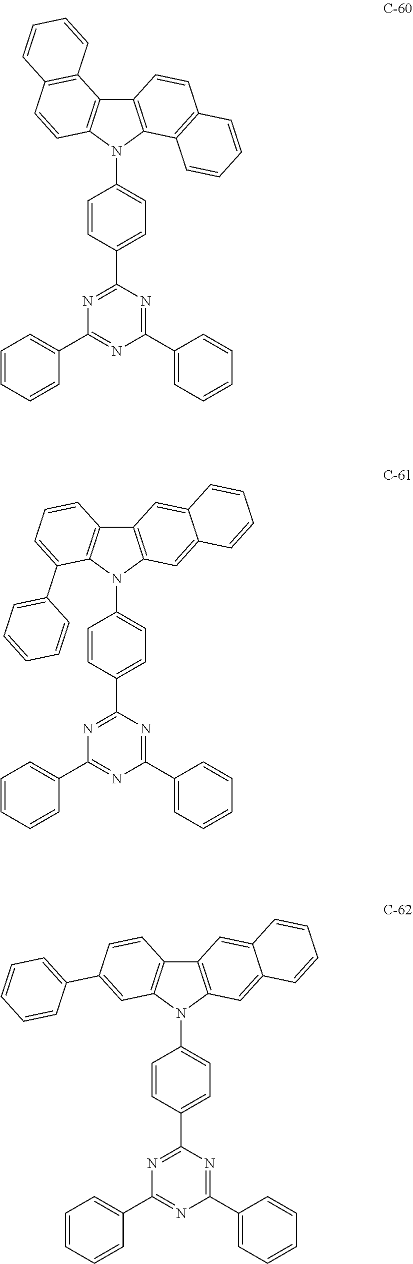 [SMILES: c1ccc(-c2nc(-c3ccccc3)nc(-c3ccc(-n4c5ccc6ccccc6c5c5ccc6ccccc6c54)cc3)n2)cc1 ; c1ccc(-c2nc(-c3ccccc3)nc(-c3ccc(-n4c5cc6ccccc6cc5c5cccc(-c6ccccc6)c54)cc3)n2)cc1 ; c1ccc(-c2ccc3c4cc5ccccc5cc4n(-c4ccc(-c5nc(-c6ccccc6)nc(-c6ccccc6)n5)cc4)c3c2)cc1]

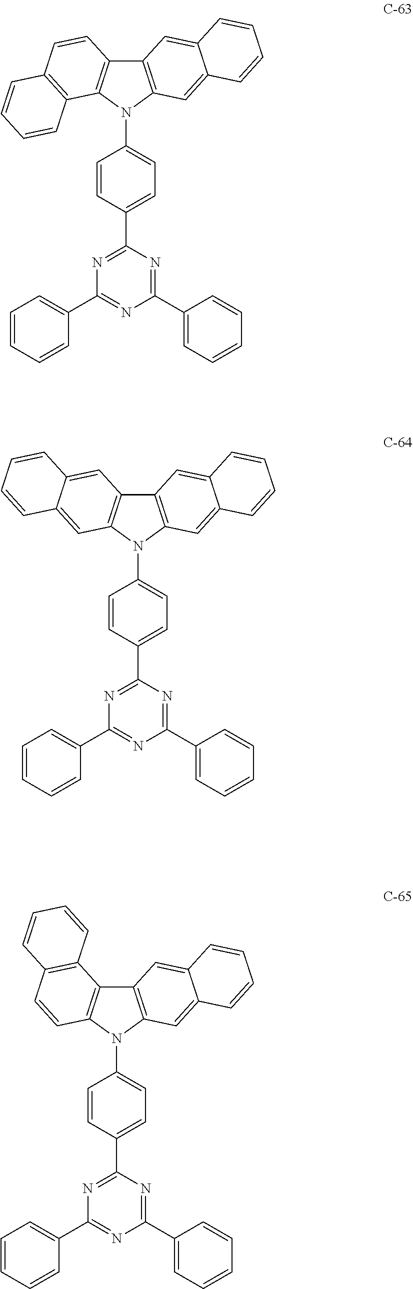 [SMILES: c1ccc(-c2nc(-c3ccccc3)nc(-c3ccc(-n4c5cc6ccccc6cc5c5ccc6ccccc6c54)cc3)n2)cc1 ; c1ccc(-c2nc(-c3ccccc3)nc(-c3ccc(-n4c5cc6ccccc6cc5c5cc6ccccc6cc54)cc3)n2)cc1 ; c1ccc(-c2nc(-c3ccccc3)nc(-c3ccc(-n4c5cc6ccccc6cc5c5c6ccccc6ccc54)cc3)n2)cc1]

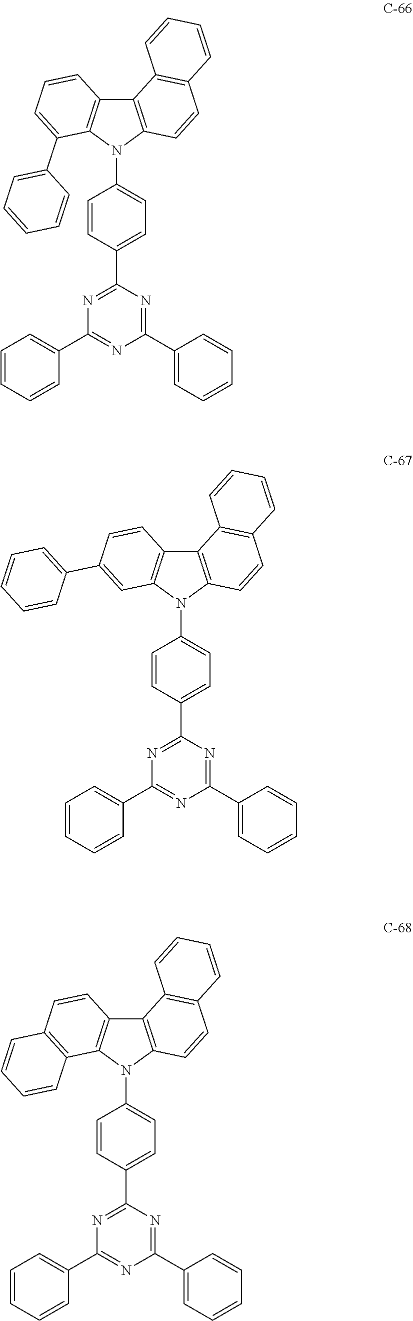 [SMILES: c1ccc(-c2nc(-c3ccccc3)nc(-c3ccc(-n4c5ccc6ccccc6c5c5cccc(-c6ccccc6)c54)cc3)n2)cc1 ; c1ccc(-c2ccc3c4c5ccccc5ccc4n(-c4ccc(-c5nc(-c6ccccc6)nc(-c6ccccc6)n5)cc4)c3c2)cc1 ; c1ccc(-c2nc(-c3ccccc3)nc(-c3ccc(-n4c5ccc6ccccc6c5c5ccc6ccccc6c54)cc3)n2)cc1]

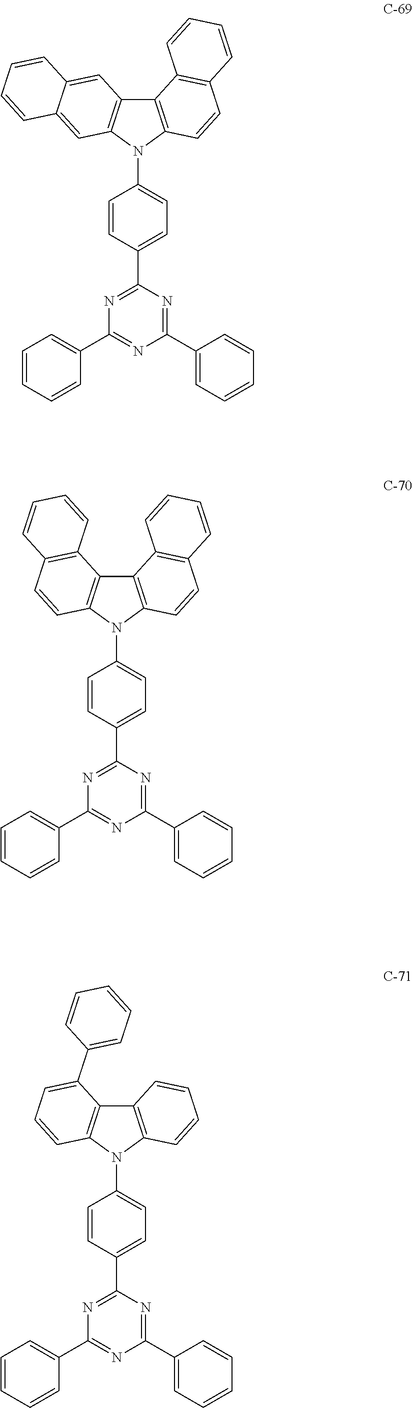 [SMILES: c1ccc(-c2nc(-c3ccccc3)nc(-c3ccc(-n4c5cc6ccccc6cc5c5c6ccccc6ccc54)cc3)n2)cc1 ; c1ccc(-c2nc(-c3ccccc3)nc(-c3ccc(-n4c5ccc6ccccc6c5c5c6ccccc6ccc54)cc3)n2)cc1 ; c1ccc(-c2nc(-c3ccccc3)nc(-c3ccc(-n4c5ccccc5c5c(-c6ccccc6)cccc54)cc3)n2)cc1]

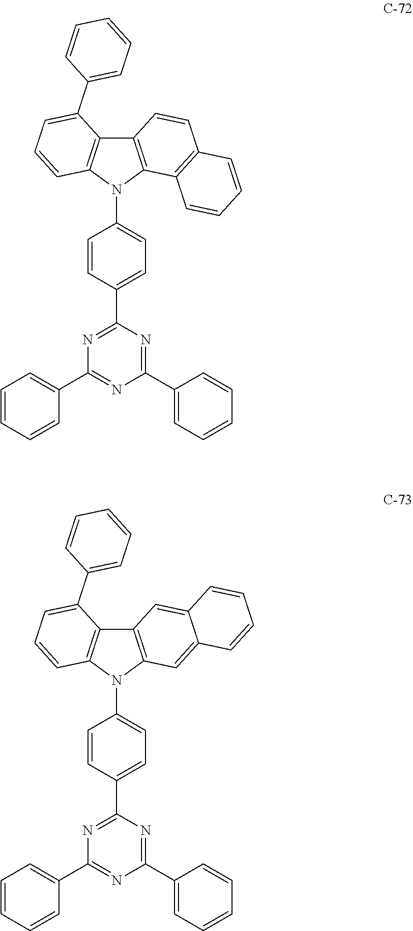 [SMILES: c1ccc(-c2nc(-c3ccccc3)nc(-c3ccc(-n4c5cccc(-c6ccccc6)c5c5ccc6ccccc6c54)cc3)n2)cc1 ; c1ccc(-c2nc(-c3ccccc3)nc(-c3ccc(-n4c5cc6ccccc6cc5c5c(-c6ccccc6)cccc54)cc3)n2)cc1]

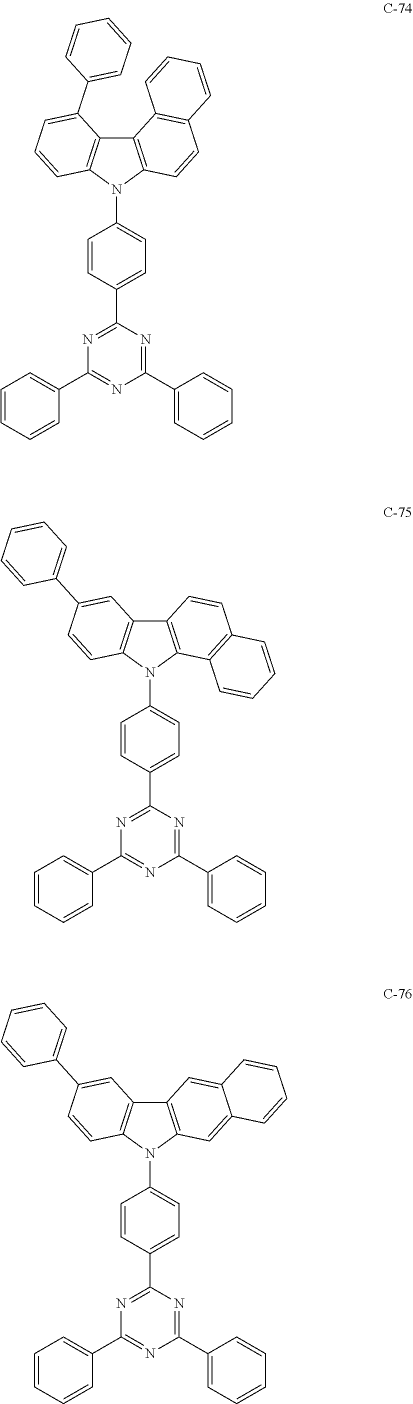 [SMILES: c1ccc(-c2nc(-c3ccccc3)nc(-c3ccc(-n4c5cccc(-c6ccccc6)c5c5c6ccccc6ccc54)cc3)n2)cc1 ; c1ccc(-c2ccc3c(c2)c2ccc4ccccc4c2n3-c2ccc(-c3nc(-c4ccccc4)nc(-c4ccccc4)n3)cc2)cc1 ; c1ccc(-c2ccc3c(c2)c2cc4ccccc4cc2n3-c2ccc(-c3nc(-c4ccccc4)nc(-c4ccccc4)n3)cc2)cc1]

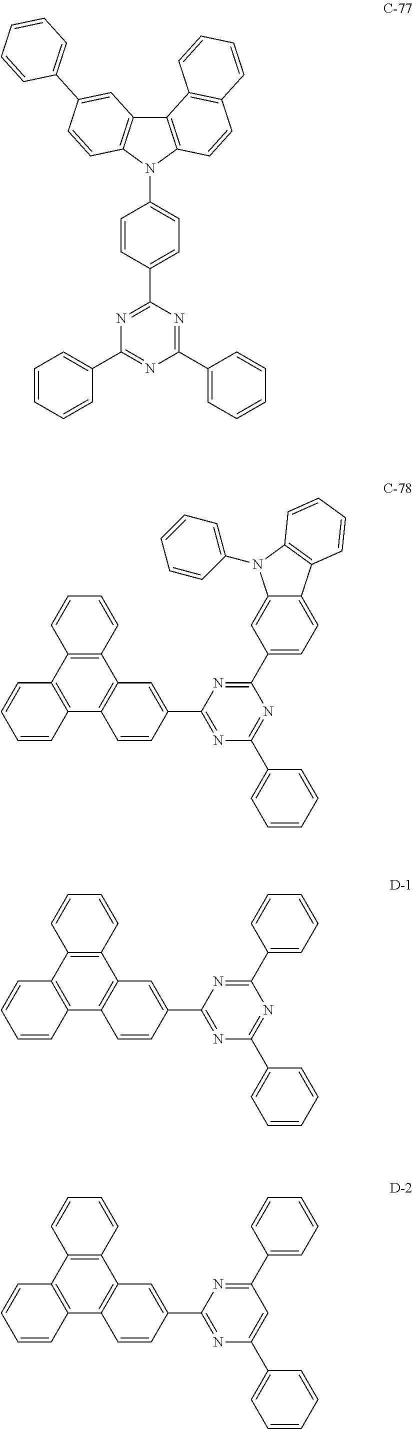 [SMILES: c1ccc(-c2ccc3c(c2)c2c4ccccc4ccc2n3-c2ccc(-c3nc(-c4ccccc4)nc(-c4ccccc4)n3)cc2)cc1 ; c1ccc(-c2nc(-c3ccc4c5ccccc5c5ccccc5c4c3)nc(-c3ccc4c5ccccc5n(-c5ccccc5)c4c3)n2)cc1 ; c1ccc(-c2nc(-c3ccccc3)nc(-c3ccc4c5ccccc5c5ccccc5c4c3)n2)cc1 ; c1ccc(-c2cc(-c3ccccc3)nc(-c3ccc4c5ccccc5c5ccccc5c4c3)n2)cc1]

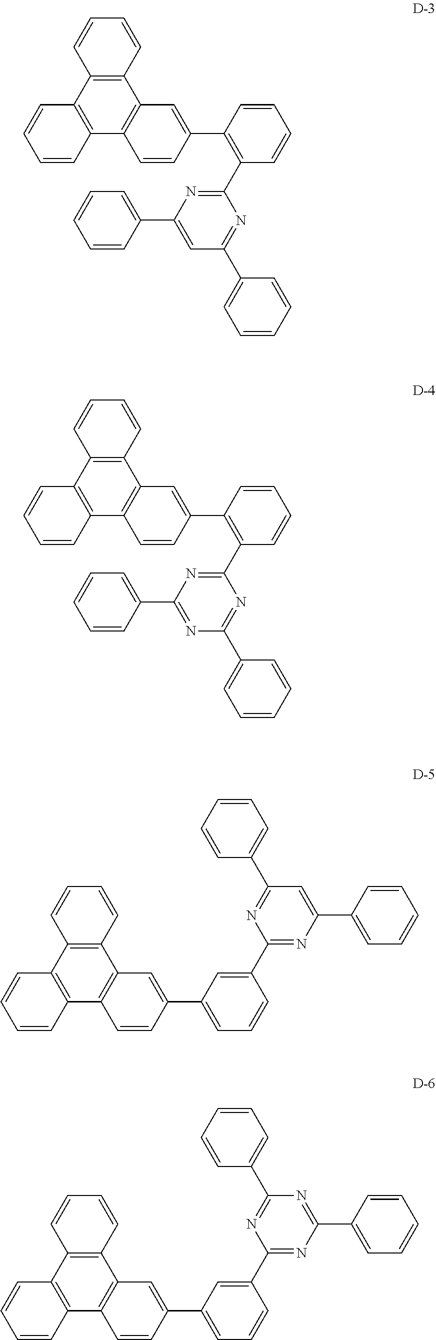 [SMILES: c1ccc(-c2cc(-c3ccccc3)nc(-c3ccccc3-c3ccc4c5ccccc5c5ccccc5c4c3)n2)cc1 ; c1ccc(-c2nc(-c3ccccc3)nc(-c3ccccc3-c3ccc4c5ccccc5c5ccccc5c4c3)n2)cc1 ; c1ccc(-c2cc(-c3ccccc3)nc(-c3cccc(-c4ccc5c6ccccc6c6ccccc6c5c4)c3)n2)cc1 ; c1ccc(-c2nc(-c3ccccc3)nc(-c3cccc(-c4ccc5c6ccccc6c6ccccc6c5c4)c3)n2)cc1]

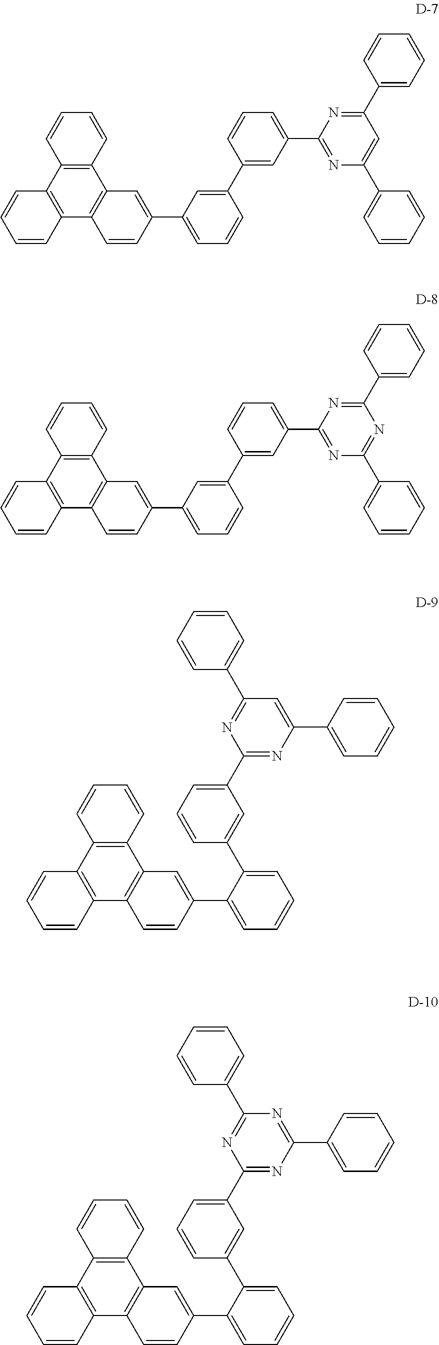 [SMILES: c1ccc(-c2cc(-c3ccccc3)nc(-c3cccc(-c4cccc(-c5ccc6c7ccccc7c7ccccc7c6c5)c4)c3)n2)cc1 ; c1ccc(-c2nc(-c3ccccc3)nc(-c3cccc(-c4cccc(-c5ccc6c7ccccc7c7ccccc7c6c5)c4)c3)n2)cc1 ; c1ccc(-c2cc(-c3ccccc3)nc(-c3cccc(-c4ccccc4-c4ccc5c6ccccc6c6ccccc6c5c4)c3)n2)cc1 ; c1ccc(-c2nc(-c3ccccc3)nc(-c3cccc(-c4ccccc4-c4ccc5c6ccccc6c6ccccc6c5c4)c3)n2)cc1]

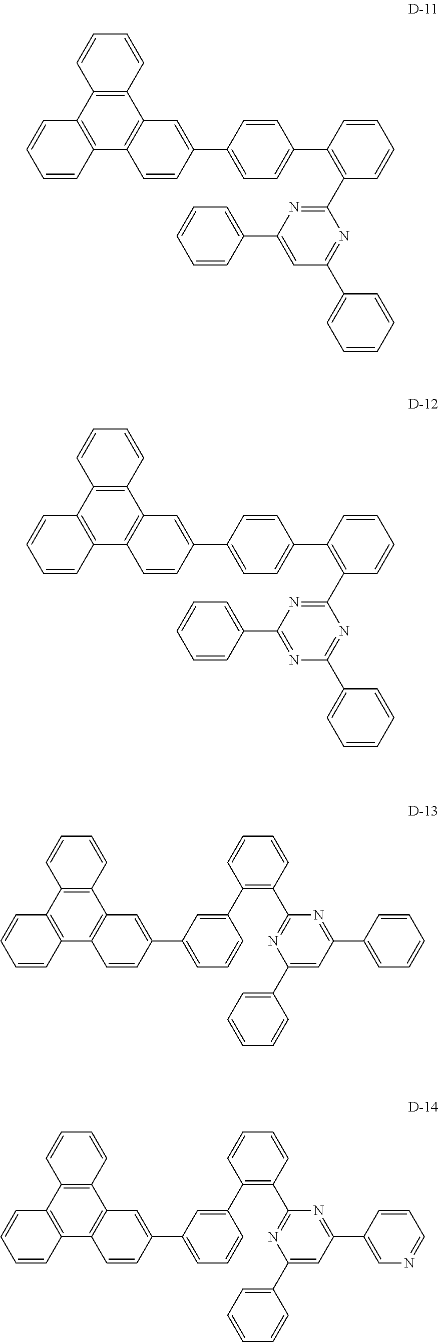 [SMILES: c1ccc(-c2cc(-c3ccccc3)nc(-c3ccccc3-c3ccc(-c4ccc5c6ccccc6c6ccccc6c5c4)cc3)n2)cc1 ; c1ccc(-c2nc(-c3ccccc3)nc(-c3ccccc3-c3ccc(-c4ccc5c6ccccc6c6ccccc6c5c4)cc3)n2)cc1 ; c1ccc(-c2cc(-c3ccccc3)nc(-c3ccccc3-c3cccc(-c4ccc5c6ccccc6c6ccccc6c5c4)c3)n2)cc1 ; c1ccc(-c2cc(-c3cccnc3)nc(-c3ccccc3-c3cccc(-c4ccc5c6ccccc6c6ccccc6c5c4)c3)n2)cc1]

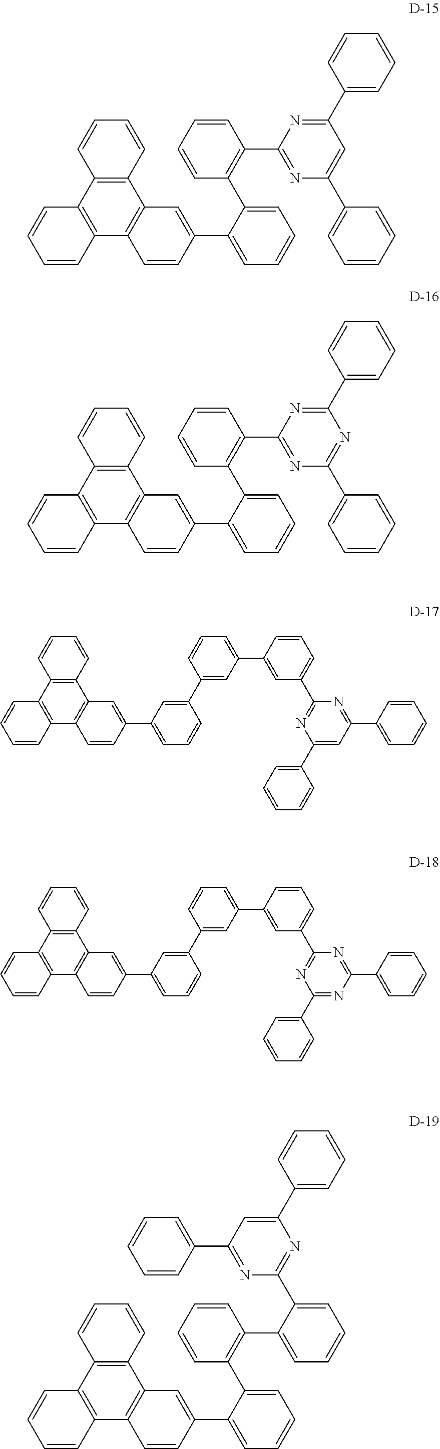 [SMILES: c1ccc(-c2cc(-c3ccccc3)nc(-c3ccccc3-c3ccccc3-c3ccc4c5ccccc5c5ccccc5c4c3)n2)cc1 ; c1ccc(-c2nc(-c3ccccc3)nc(-c3ccccc3-c3ccccc3-c3ccc4c5ccccc5c5ccccc5c4c3)n2)cc1 ; c1ccc(-c2cc(-c3ccccc3)nc(-c3cccc(-c4cccc(-c5cccc(-c6ccc7c8ccccc8c8ccccc8c7c6)c5)c4)c3)n2)cc1 ; c1ccc(-c2nc(-c3ccccc3)nc(-c3cccc(-c4cccc(-c5cccc(-c6ccc7c8ccccc8c8ccccc8c7c6)c5)c4)c3)n2)cc1 ; c1ccc(-c2cc(-c3ccccc3)nc(-c3ccccc3-c3ccccc3-c3ccccc3-c3ccc4c5ccccc5c5ccccc5c4c3)n2)cc1]

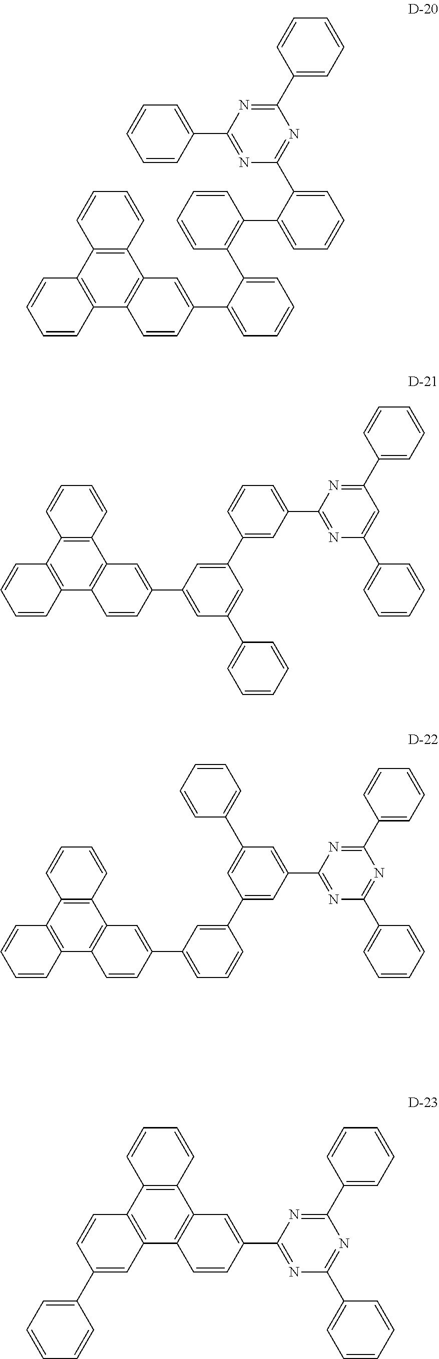 [SMILES: c1ccc(-c2nc(-c3ccccc3)nc(-c3ccccc3-c3ccccc3-c3ccccc3-c3ccc4c5ccccc5c5ccccc5c4c3)n2)cc1 ; c1ccc(-c2cc(-c3cccc(-c4nc(-c5ccccc5)cc(-c5ccccc5)n4)c3)cc(-c3ccc4c5ccccc5c5ccccc5c4c3)c2)cc1 ; c1ccc(-c2cc(-c3cccc(-c4ccc5c6ccccc6c6ccccc6c5c4)c3)cc(-c3nc(-c4ccccc4)nc(-c4ccccc4)n3)c2)cc1 ; c1ccc(-c2ccc3c4ccccc4c4cc(-c5nc(-c6ccccc6)nc(-c6ccccc6)n5)ccc4c3c2)cc1]

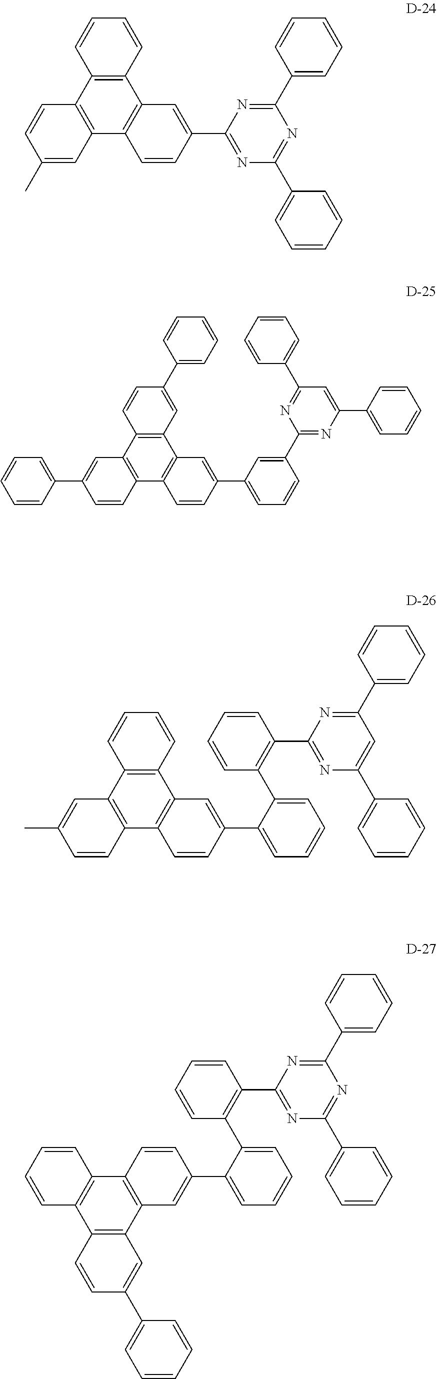 [SMILES: Cc1ccc2c3ccccc3c3cc(-c4nc(-c5ccccc5)nc(-c5ccccc5)n4)ccc3c2c1 ; c1ccc(-c2ccc3c4ccc(-c5cccc(-c6nc(-c7ccccc7)cc(-c7ccccc7)n6)c5)cc4c4cc(-c5ccccc5)ccc4c3c2)cc1 ; Cc1ccc2c3ccc(-c4ccccc4-c4ccccc4-c4nc(-c5ccccc5)cc(-c5ccccc5)n4)cc3c3ccccc3c2c1 ; c1ccc(-c2ccc3c4ccccc4c4ccc(-c5ccccc5-c5ccccc5-c5nc(-c6ccccc6)nc(-c6ccccc6)n5)cc4c3c2)cc1]

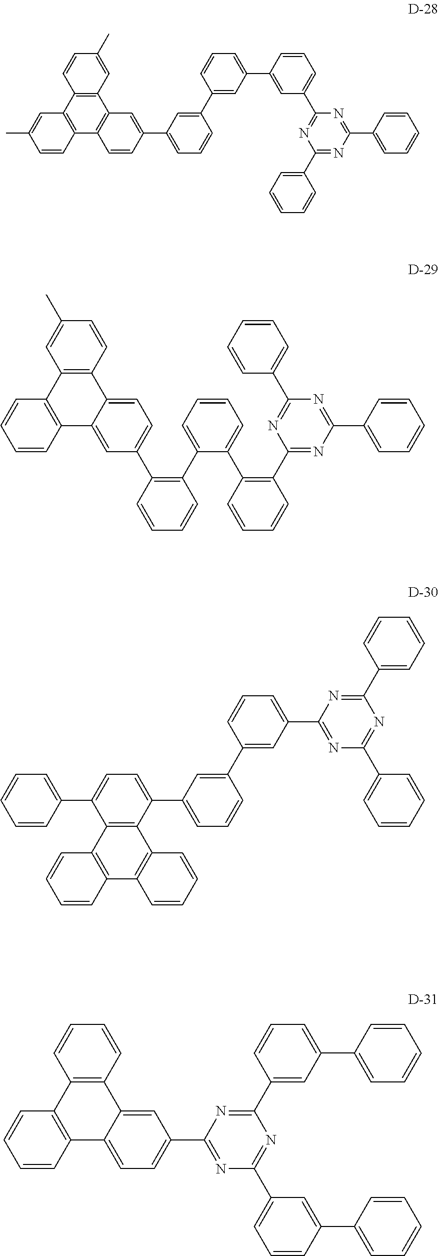 [SMILES: Cc1ccc2c3ccc(-c4cccc(-c5cccc(-c6cccc(-c7nc(-c8ccccc8)nc(-c8ccccc8)n7)c6)c5)c4)cc3c3cc(C)ccc3c2c1 ; Cc1ccc2c3ccc(-c4ccccc4-c4ccccc4-c4ccccc4-c4nc(-c5ccccc5)nc(-c5ccccc5)n4)cc3c3ccccc3c2c1 ; c1ccc(-c2nc(-c3ccccc3)nc(-c3cccc(-c4cccc(-c5ccc(-c6ccccc6)c6c7ccccc7c7ccccc7c56)c4)c3)n2)cc1 ; c1ccc(-c2cccc(-c3nc(-c4cccc(-c5ccccc5)c4)nc(-c4ccc5c6ccccc6c6ccccc6c5c4)n3)c2)cc1]

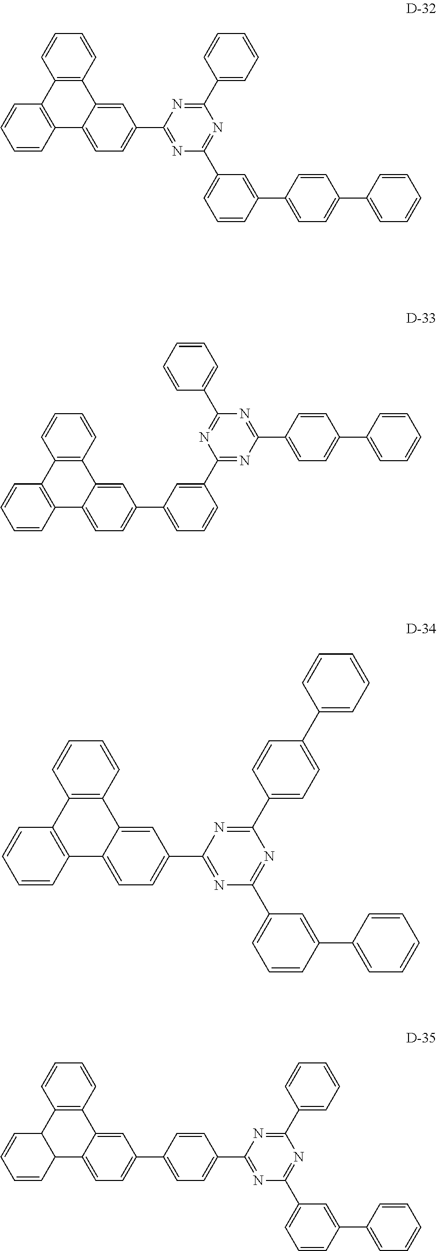 [SMILES: c1ccc(-c2ccc(-c3cccc(-c4nc(-c5ccccc5)nc(-c5ccc6c7ccccc7c7ccccc7c6c5)n4)c3)cc2)cc1 ; c1ccc(-c2ccc(-c3nc(-c4ccccc4)nc(-c4cccc(-c5ccc6c7ccccc7c7ccccc7c6c5)c4)n3)cc2)cc1 ; c1ccc(-c2ccc(-c3nc(-c4cccc(-c5ccccc5)c4)nc(-c4ccc5c6ccccc6c6ccccc6c5c4)n3)cc2)cc1 ; C1=CC2c3ccccc3-c3cc(-c4ccc(-c5nc(-c6ccccc6)nc(-c6cccc(-c7ccccc7)c6)n5)cc4)ccc3C2C=C1]

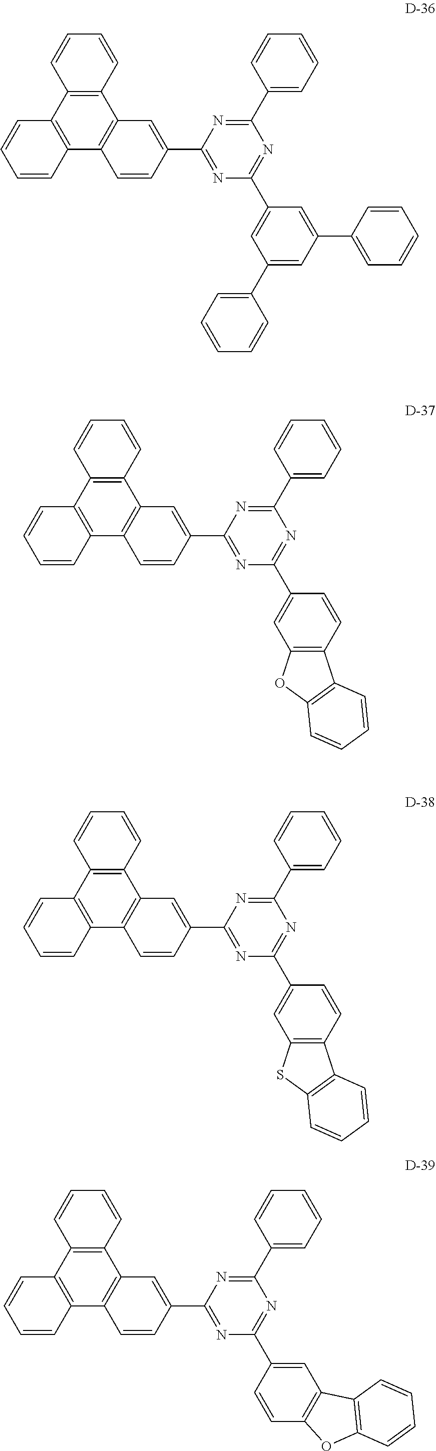 [SMILES: c1ccc(-c2cc(-c3ccccc3)cc(-c3nc(-c4ccccc4)nc(-c4ccc5c6ccccc6c6ccccc6c5c4)n3)c2)cc1 ; c1ccc(-c2nc(-c3ccc4c(c3)oc3ccccc34)nc(-c3ccc4c5ccccc5c5ccccc5c4c3)n2)cc1 ; c1ccc(-c2nc(-c3ccc4c(c3)sc3ccccc34)nc(-c3ccc4c5ccccc5c5ccccc5c4c3)n2)cc1 ; c1ccc(-c2nc(-c3ccc4oc5ccccc5c4c3)nc(-c3ccc4c5ccccc5c5ccccc5c4c3)n2)cc1]

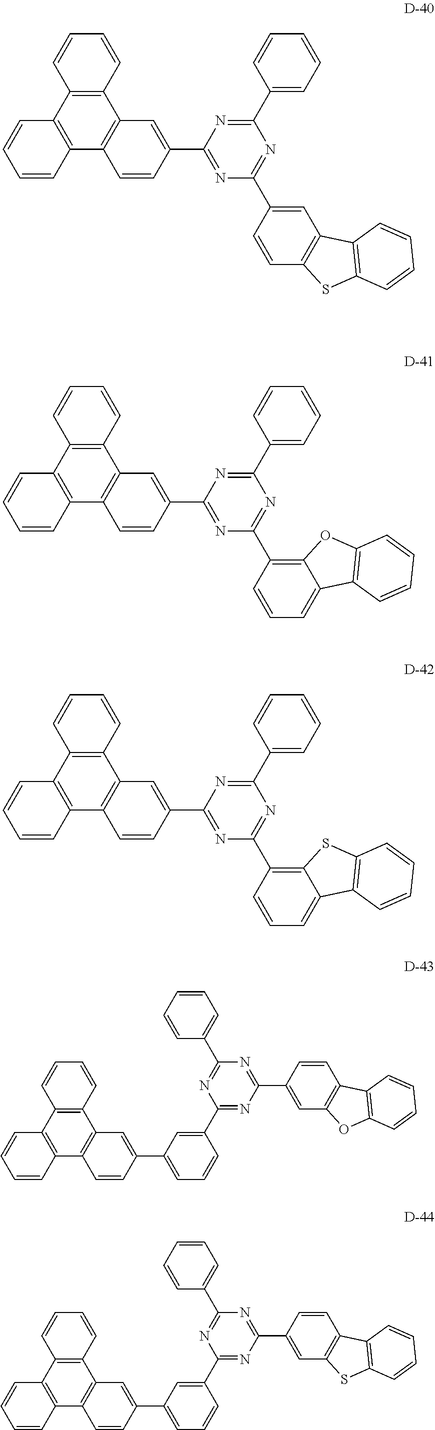 [SMILES: c1ccc(-c2nc(-c3ccc4sc5ccccc5c4c3)nc(-c3ccc4c5ccccc5c5ccccc5c4c3)n2)cc1 ; c1ccc(-c2nc(-c3ccc4c5ccccc5c5ccccc5c4c3)nc(-c3cccc4c3oc3ccccc34)n2)cc1 ; c1ccc(-c2nc(-c3ccc4c5ccccc5c5ccccc5c4c3)nc(-c3cccc4c3sc3ccccc34)n2)cc1 ; c1ccc(-c2nc(-c3cccc(-c4ccc5c6ccccc6c6ccccc6c5c4)c3)nc(-c3ccc4c(c3)oc3ccccc34)n2)cc1 ; c1ccc(-c2nc(-c3cccc(-c4ccc5c6ccccc6c6ccccc6c5c4)c3)nc(-c3ccc4c(c3)sc3ccccc34)n2)cc1]

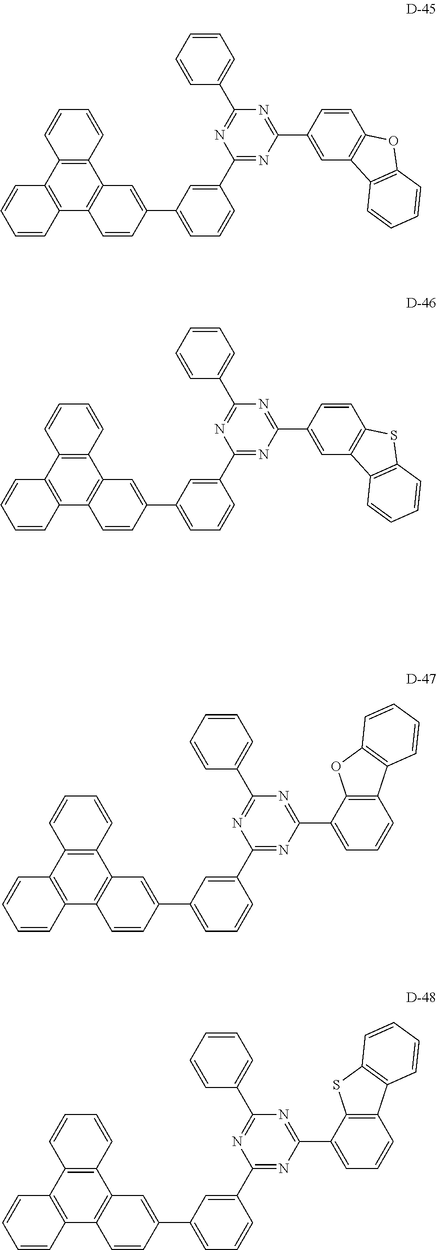 [SMILES: c1ccc(-c2nc(-c3cccc(-c4ccc5c6ccccc6c6ccccc6c5c4)c3)nc(-c3ccc4oc5ccccc5c4c3)n2)cc1 ; c1ccc(-c2nc(-c3cccc(-c4ccc5c6ccccc6c6ccccc6c5c4)c3)nc(-c3ccc4sc5ccccc5c4c3)n2)cc1 ; c1ccc(-c2nc(-c3cccc(-c4ccc5c6ccccc6c6ccccc6c5c4)c3)nc(-c3cccc4c3oc3ccccc34)n2)cc1 ; c1ccc(-c2nc(-c3cccc(-c4ccc5c6ccccc6c6ccccc6c5c4)c3)nc(-c3cccc4c3sc3ccccc34)n2)cc1]

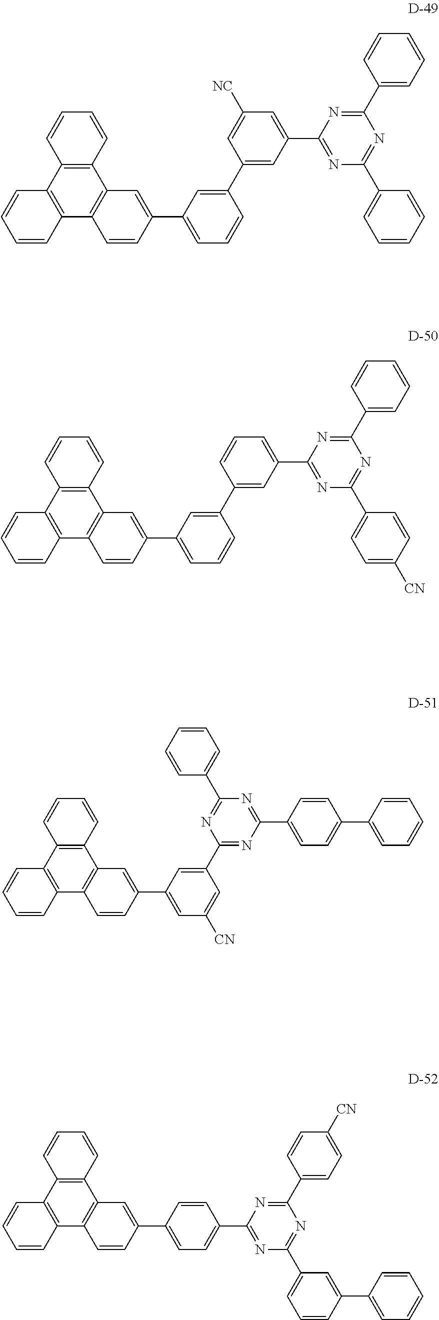 [SMILES: N#Cc1cc(-c2cccc(-c3ccc4c5ccccc5c5ccccc5c4c3)c2)cc(-c2nc(-c3ccccc3)nc(-c3ccccc3)n2)c1 ; N#Cc1ccc(-c2nc(-c3ccccc3)nc(-c3cccc(-c4cccc(-c5ccc6c7ccccc7c7ccccc7c6c5)c4)c3)n2)cc1 ; N#Cc1cc(-c2ccc3c4ccccc4c4ccccc4c3c2)cc(-c2nc(-c3ccccc3)nc(-c3ccc(-c4ccccc4)cc3)n2)c1 ; N#Cc1ccc(-c2nc(-c3ccc(-c4ccc5c6ccccc6c6ccccc6c5c4)cc3)nc(-c3cccc(-c4ccccc4)c3)n2)cc1]

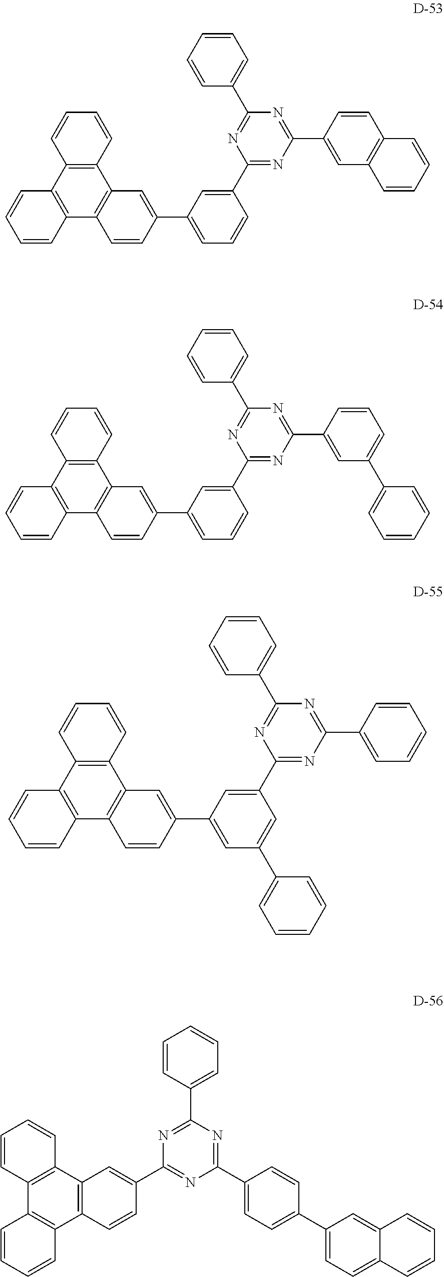 [SMILES: c1ccc(-c2nc(-c3cccc(-c4ccc5c6ccccc6c6ccccc6c5c4)c3)nc(-c3ccc4ccccc4c3)n2)cc1 ; c1ccc(-c2cccc(-c3nc(-c4ccccc4)nc(-c4cccc(-c5ccc6c7ccccc7c7ccccc7c6c5)c4)n3)c2)cc1 ; c1ccc(-c2cc(-c3ccc4c5ccccc5c5ccccc5c4c3)cc(-c3nc(-c4ccccc4)nc(-c4ccccc4)n3)c2)cc1 ; c1ccc(-c2nc(-c3ccc(-c4ccc5ccccc5c4)cc3)nc(-c3ccc4c5ccccc5c5ccccc5c4c3)n2)cc1]

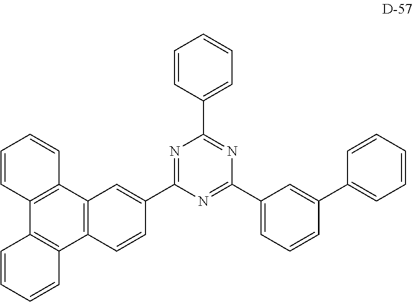 [SMILES: c1ccc(-c2cccc(-c3nc(-c4ccccc4)nc(-c4ccc5c6ccccc6c6ccccc6c5c4)n3)c2)cc1]

In an implementation, the first compound may be represented by Chemical Formula 1-2b and the second compound may be represented by Chemical Formula 2-1a or Chemical Formula 2-3a.

In an implementation, L¹ to L⁴ of Chemical Formula 1-2b may each independently be, e.g., a single bond or a substituted or unsubstituted phenylene group, and Ar¹ to Ar⁴ may each independently be, e.g., a substituted or unsubstituted phenyl group, a substituted or unsubstituted biphenyl group, or a substituted or unsubstituted naphthyl group,

In an implementation, L⁶, L⁸, and L¹⁰ of Chemical Formula 2-1a may each independently be, e.g., a single bond or a substituted or unsubstituted phenylene group.

In an implementation, R⁴, R⁶, and R⁸ may each independently be, e.g., a substituted or unsubstituted phenyl group, a substituted or unsubstituted biphenyl group, a substituted or unsubstituted terphenyl group, a substituted or unsubstituted naphthyl group, a substituted or unsubstituted triphenylene group, a substituted or unsubstituted dibenzofuranyl group, a substituted or unsubstituted dibenzothiophenyl group, or a substituted or unsubstituted carbazolyl group, and at least one of R⁴, R⁶, and R⁸ may be, e.g., a substituted or unsubstituted triphenylene group, a substituted or unsubstituted carbazolyl group, a substituted or unsubstituted dibenzofuranyl group, or a substituted or unsubstituted dibenzothiophenyl group,

In an implementation, X² of Chemical Formula 2-3a may be, e.g., O or S, L⁵ and L⁹ may each independently be, e.g., a single bond or a substituted or unsubstituted phenylene group, R³ and R⁷ may each independently be, e.g., a substituted or unsubstituted phenyl group, a substituted or unsubstituted biphenyl group, a substituted or unsubstituted terphenyl group, a substituted or unsubstituted naphthyl group, a substituted or unsubstituted triphenylene group, a substituted or unsubstituted dibenzofuranyl group, a substituted or unsubstituted dibenzothiophenyl group, or a substituted or unsubstituted carbazolyl group, and at least one of R³ and R⁷ may be, e.g., a substituted or unsubstituted carbazolyl group, a substituted or unsubstituted dibenzofuranyl group, or a substituted or unsubstituted dibenzothiophenyl group.

The first compound and the second compound may be, e.g., included in a weight ratio of about 1:99 to about 99:1. Within the range, a desirable weight ratio may be adjusted using a hole transport capability of the first compound and an electron hole transport capability of the second compound to realize bipolar characteristics and thus to improve efficiency and life-span. Within the range, they may be for example included in a weight ratio of about 10:90 to about 90:10, about 20:80 to about 80:20, about 30:70 to about 70:30, about 40:60 to about 60:40, or about 50:50. In an implementation, they may be included in a weight ratio of about 50:50 to about 60:40, e.g., about 60:40. For example, the first compound may be included in an greater amount than that of the second compound.

In an implementation, the first compound and the second compound may be included as hosts of the light emitting layer, e.g., a phosphorescent host.

In an implementation, the light emitting layer may further include at least one compound in addition to the aforementioned hosts.

The light emitting layer may further include a dopant. The dopant may be, e.g., a phosphorescent dopant, such as a red, green, or blue phosphorescent dopant, and may be e.g., a red phosphorescent dopant.

The dopant may be a material mixed with the aforementioned host in a small amount to facilitate light emission and may include a material such as a metal complex that emits light by multiple excitation into a triplet or more. The dopant may be, e.g., an inorganic, organic, or organic/inorganic compound, and one or more types thereof may be used.

Examples of the dopant may include a phosphorescent dopant and examples of the phosphorescent dopant may be an organometal compound including Ir, Pt, Os, Ti, Zr, Hf, Eu, Tb, Tm, Fe, Co, Ni, Ru, Rh, Pd, or a combination thereof. In an implementation, the phosphorescent dopant may be, e.g., a compound represented by Chemical Formula Z.

L¹⁵MX⁵ [Chemical Formula Z]

In Chemical Formula Z, M may be a metal, and L¹⁵ and X⁵ may each independently be a ligand to form a complex compound with M.

In an implementation, M may be, e.g., Ir, Pt, Os, Ti, Zr, Hf, Eu, Tb, Tm, Fe, Co, Ni, Ru, Rh, Pd, or a combination thereof and L¹⁵ and X⁵ may be, e.g., a bidendate ligand.

The hole transport auxiliary layer 142 may be between the light emitting layer 130 and the hole transport layer 141 that will be described be and may be in contact with the light emitting layer 130. The hole transport auxiliary layer 142 in contact with the light emitting layer 130 may help finely control hole mobility at the interface of the light emitting layer 130 and the hole transport layer (HTL) 141. The hole transport auxiliary layer 142 may include a plurality of layers.

The hole transport auxiliary layer 142 may include, e.g., a third compound represented by Chemical Formula 3.

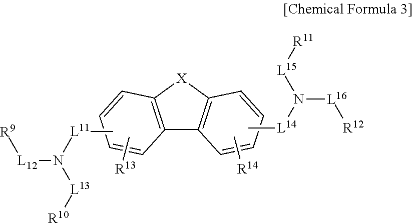

In Chemical Formula 3,

X may be, e.g., O or S,

L¹¹ to L¹⁶ may each independently be or include, e.g., a single bond, a substituted or unsubstituted C6 to C20 arylene group, a substituted or unsubstituted divalent C2 to C20 heterocyclic group, or a combination thereof,

R⁹ to R¹² may each independently be or include, e.g., a substituted or unsubstituted C6 to C30 aryl group, a substituted or unsubstituted C2 to C30 heterocyclic group, or a combination thereof, and

R¹³ and R¹⁴ may each independently be or include, e.g., hydrogen, deuterium, a cyano group, a substituted or unsubstituted C1 to C10 alkyl group.

The third compound may be a compound having a high HOMO energy level and may have good hole injection characteristics. For example, the third compound may be applied to the hole transport auxiliary layer 142 to help effectively lower a driving voltage of the organic optoelectronic device by effectively improving hole mobility at the interface of the light emitting layer 130 and the hole transport layer 141.

In an implementation, the third compound may be represented by one of Chemical Formula 3-1 to Chemical Formula 3-4 depending on a specific substitution position of the amine group.

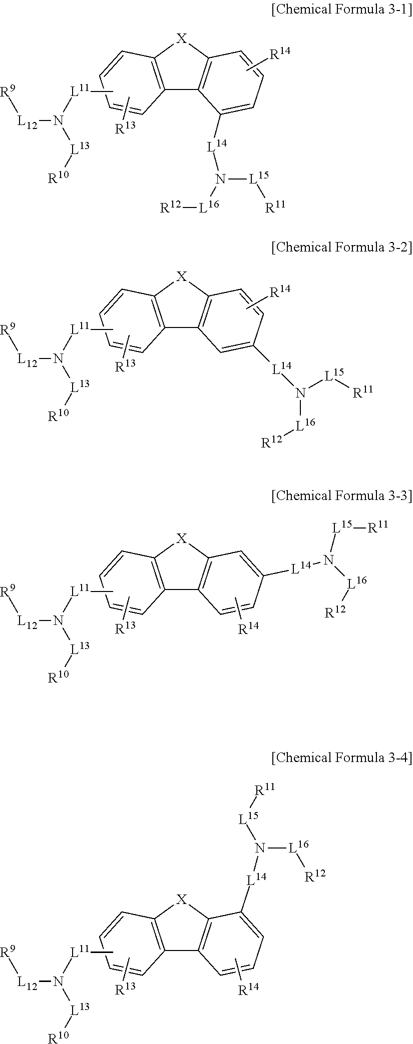

In Chemical Formula 3-1 to Chemical Formula 3-4, X, L¹¹ to L¹⁶, and R⁹ to R¹⁴ may be defined the same as described above.

In an implementation, the third compound may be, e.g., represented by one of Chemical Formula 3-1 to Chemical Formula 3-3.

In an implementation, the compound represented by Chemical Formula 3-1 may be, e.g., represented by one of Chemical Formula 3-1a, Chemical Formula 3-1b, Chemical Formula 3-1c, and Chemical Formula 3-1d.

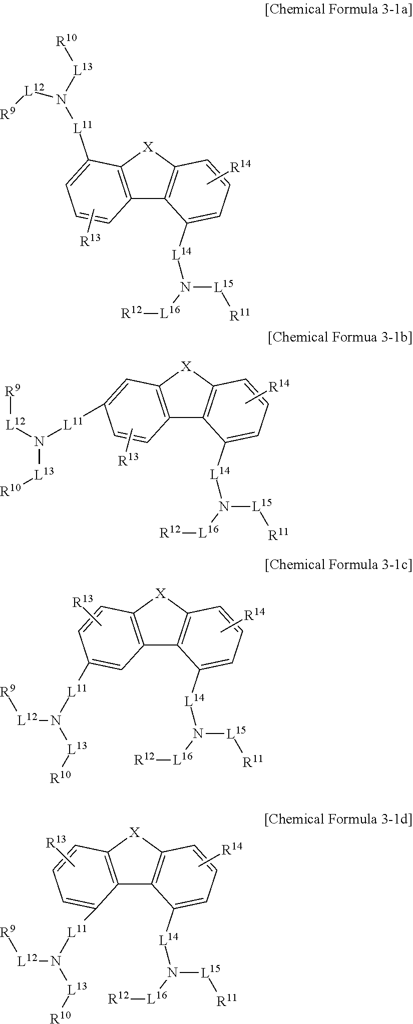

In Chemical Formula 3-1a to Chemical Formula 3-1d, X, L¹¹ to L¹⁶, R⁹ to R¹⁴ may be defined the same as described above.

In an implementation, the compound represented by Chemical Formula 3-2 may be, e.g., represented by one of Chemical Formula 3-2a, Chemical Formula 3-2b, Chemical Formula 3-2c, and Chemical Formula 3-2d.

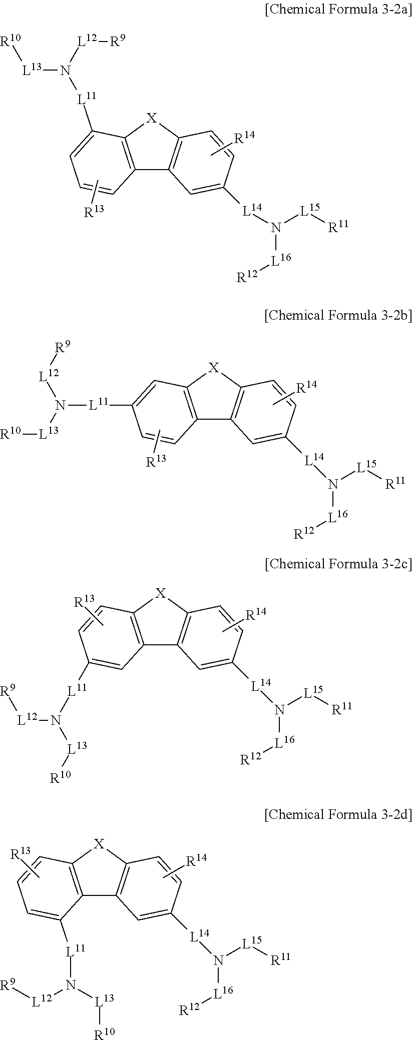

In Chemical Formula 3-2a to Chemical Formula 3-2d, X, L¹¹ to L¹⁶, and R⁹ to R¹⁴ may be the same as described above.

In an implementation, the compound represented by Chemical Formula 3-3 may be, e.g., represented by one of Chemical Formula 3-3a, Chemical Formula 3-3b, Chemical Formula 3-3c, and Chemical Formula 3-3d.

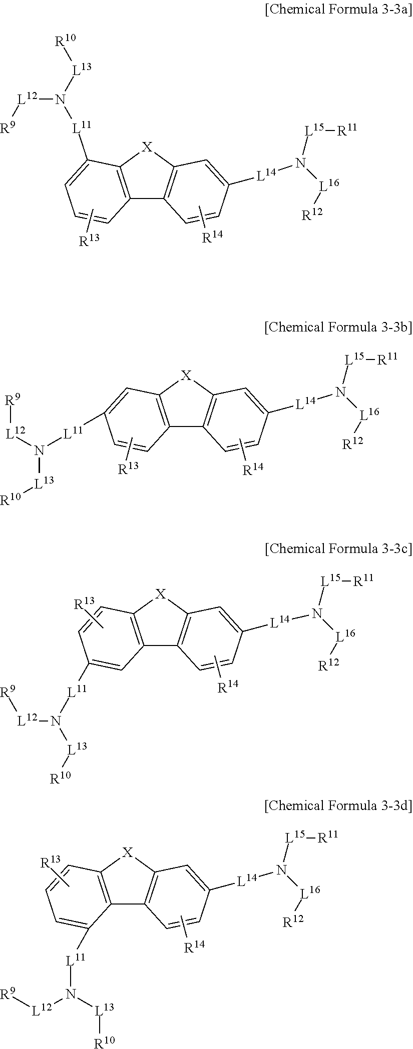

In Chemical Formula 3-3a to Chemical Formula 3-3d, X, L¹¹ to L¹⁶, and R⁹ to R¹⁴ may be defined the same as described above.

In an implementation, the third compound may be represented by one of Chemical Formula 3-1b, Chemical Formula 3-2b, Chemical Formula 3-2c, Chemical Formula 3-3b, Chemical Formula 3-3c, and Chemical Formula 3-3d.

In an implementation, L¹¹ and L¹⁴ may each independently be a single bond and L¹², L¹³, L¹⁵, and L¹⁶ may each independently be a single bond or a substituted or unsubstituted phenylene group.

In an implementation, R⁹ to R¹² may each independently be, e.g., a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, a substituted or unsubstituted triphenylene group, a substituted or unsubstituted fluorenyl group, a substituted or unsubstituted dibenzofuranyl group, a substituted or unsubstituted dibenzothiophenyl group, a substituted or unsubstituted fused dibenzofuranyl group, or a substituted or unsubstituted fused dibenzothiophenyl group.

In an implementation, R⁹ to R¹² may each independently be, e.g., a substituted or unsubstituted phenyl group, a substituted or unsubstituted naphthyl group, a substituted or unsubstituted dibenzofuranyl group, or a substituted or unsubstituted dibenzothiophenyl group.

In an implementation, at least one of R⁹ to R¹² may be, e.g., a substituted or unsubstituted dibenzofuranyl group or a substituted or unsubstituted dibenzothiophenyl group.

In an implementation, R¹³ and R¹⁴ may each independently be hydrogen or a C1 to C5 alkyl group.

In an implementation, R¹³ and R¹⁴ may each be hydrogen.

In an implementation, the first compound may be represented by Chemical Formula 1-2b, the second compound may be represented by Chemical Formula 2-1a or Chemical Formula 2-3a, and the third compound may be represented by Chemical Formula 3-1b, Chemical Formula 3-2b, Chemical Formula 3-2c, Chemical Formula 3-3b, Chemical Formula 3-3c, and Chemical Formula 3-3d.

In an implementation, the third compound may be, e.g., a compound of the following Group 3.

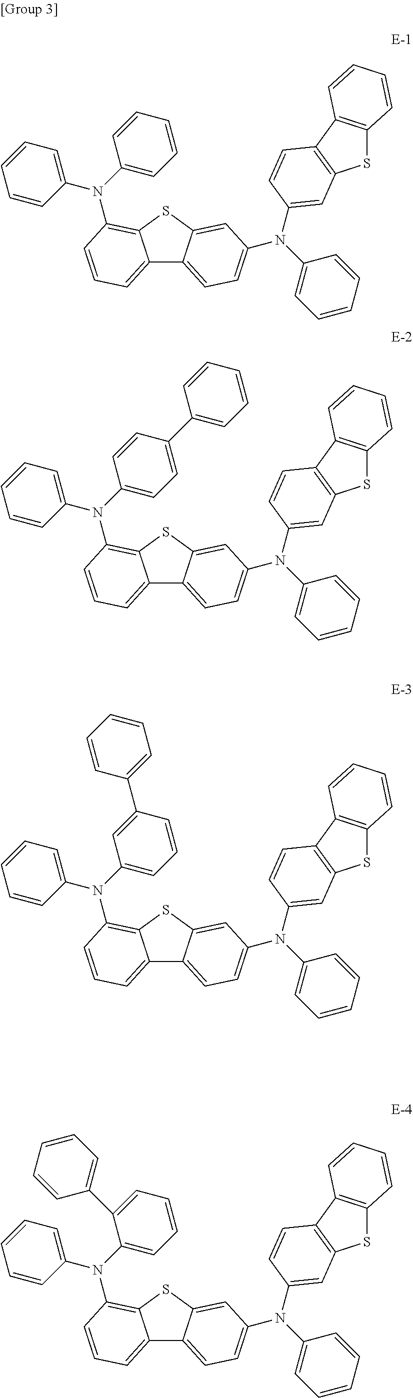 [SMILES: c1ccc(N(c2ccc3c(c2)sc2ccccc23)c2ccc3c(c2)sc2c(N(c4ccccc4)c4ccccc4)cccc23)cc1 ; c1ccc(-c2ccc(N(c3ccccc3)c3cccc4c3sc3cc(N(c5ccccc5)c5ccc6c(c5)sc5ccccc56)ccc34)cc2)cc1 ; c1ccc(-c2cccc(N(c3ccccc3)c3cccc4c3sc3cc(N(c5ccccc5)c5ccc6c(c5)sc5ccccc56)ccc34)c2)cc1 ; c1ccc(-c2ccccc2N(c2ccccc2)c2cccc3c2sc2cc(N(c4ccccc4)c4ccc5c(c4)sc4ccccc45)ccc23)cc1]

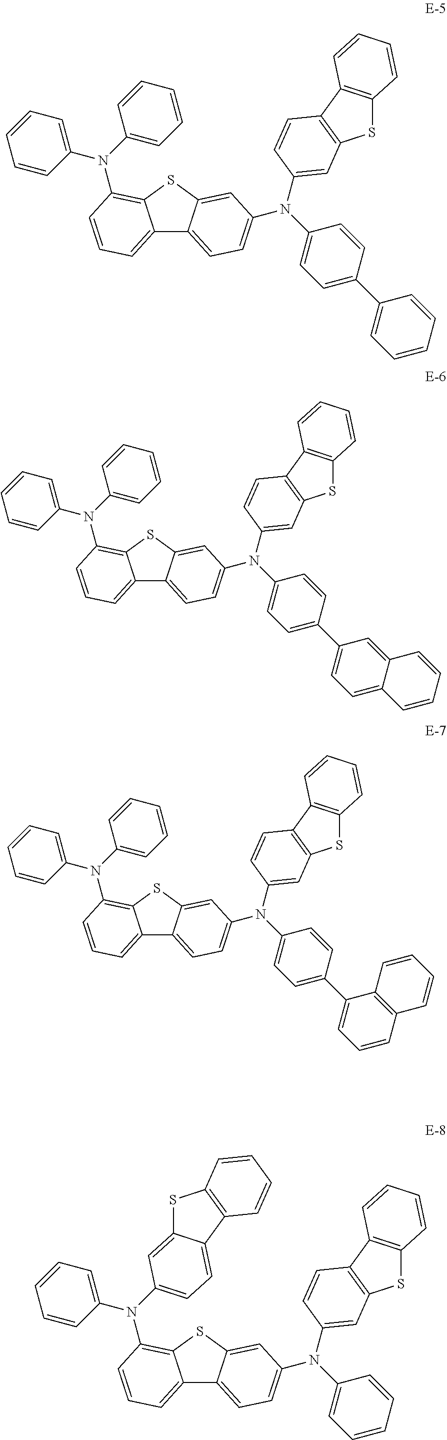 [SMILES: c1ccc(-c2ccc(N(c3ccc4c(c3)sc3ccccc34)c3ccc4c(c3)sc3c(N(c5ccccc5)c5ccccc5)cccc34)cc2)cc1 ; c1ccc(N(c2ccccc2)c2cccc3c2sc2cc(N(c4ccc(-c5ccc6ccccc6c5)cc4)c4ccc5c(c4)sc4ccccc45)ccc23)cc1 ; c1ccc(N(c2ccccc2)c2cccc3c2sc2cc(N(c4ccc(-c5cccc6ccccc56)cc4)c4ccc5c(c4)sc4ccccc45)ccc23)cc1 ; c1ccc(N(c2ccc3c(c2)sc2ccccc23)c2ccc3c(c2)sc2c(N(c4ccccc4)c4ccc5c(c4)sc4ccccc45)cccc23)cc1]

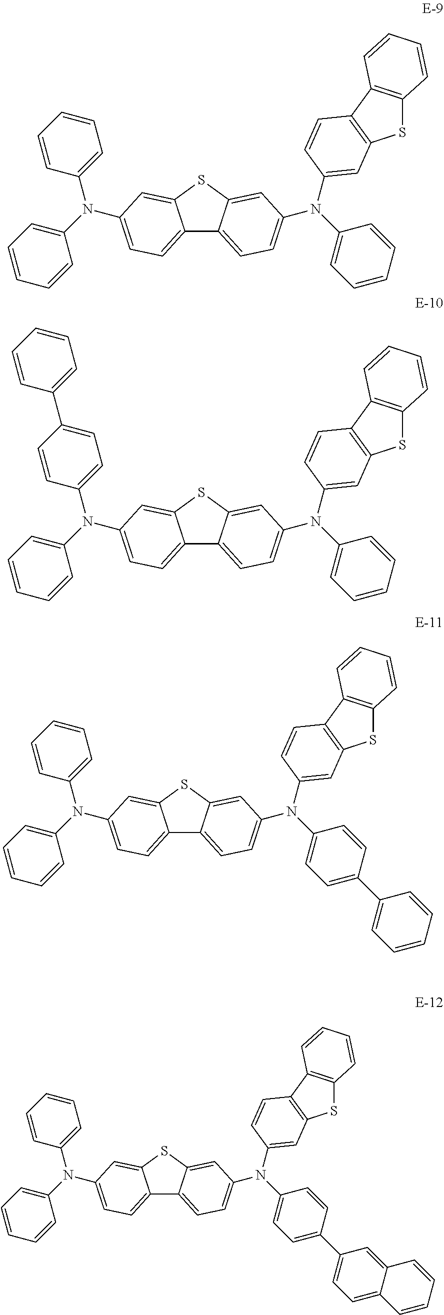

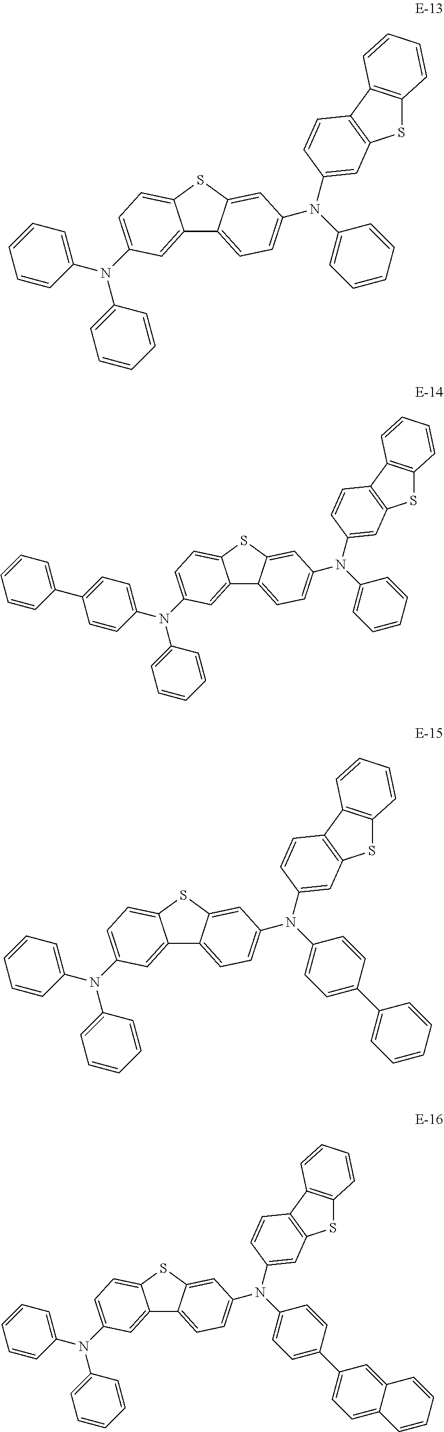 [SMILES: c1ccc(N(c2ccc3c(c2)sc2ccccc23)c2ccc3c(c2)sc2ccc(N(c4ccccc4)c4ccccc4)cc23)cc1 ; c1ccc(-c2ccc(N(c3ccccc3)c3ccc4sc5cc(N(c6ccccc6)c6ccc7c(c6)sc6ccccc67)ccc5c4c3)cc2)cc1 ; c1ccc(-c2ccc(N(c3ccc4c(c3)sc3ccccc34)c3ccc4c(c3)sc3ccc(N(c5ccccc5)c5ccccc5)cc34)cc2)cc1 ; c1ccc(N(c2ccccc2)c2ccc3sc4cc(N(c5ccc(-c6ccc7ccccc7c6)cc5)c5ccc6c(c5)sc5ccccc56)ccc4c3c2)cc1]

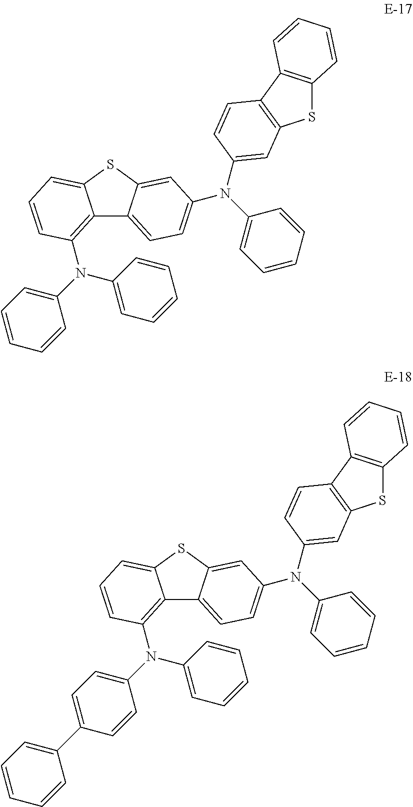 [SMILES: c1ccc(N(c2ccc3c(c2)sc2ccccc23)c2ccc3c(c2)sc2cccc(N(c4ccccc4)c4ccccc4)c23)cc1 ; c1ccc(-c2ccc(N(c3ccccc3)c3cccc4sc5cc(N(c6ccccc6)c6ccc7c(c6)sc6ccccc67)ccc5c34)cc2)cc1]

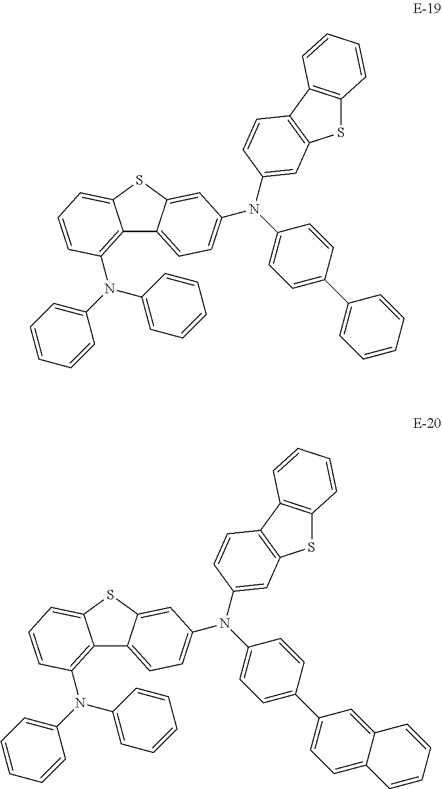 [SMILES: c1ccc(-c2ccc(N(c3ccc4c(c3)sc3ccccc34)c3ccc4c(c3)sc3cccc(N(c5ccccc5)c5ccccc5)c34)cc2)cc1 ; c1ccc(N(c2ccccc2)c2cccc3sc4cc(N(c5ccc(-c6ccc7ccccc7c6)cc5)c5ccc6c(c5)sc5ccccc56)ccc4c23)cc1]

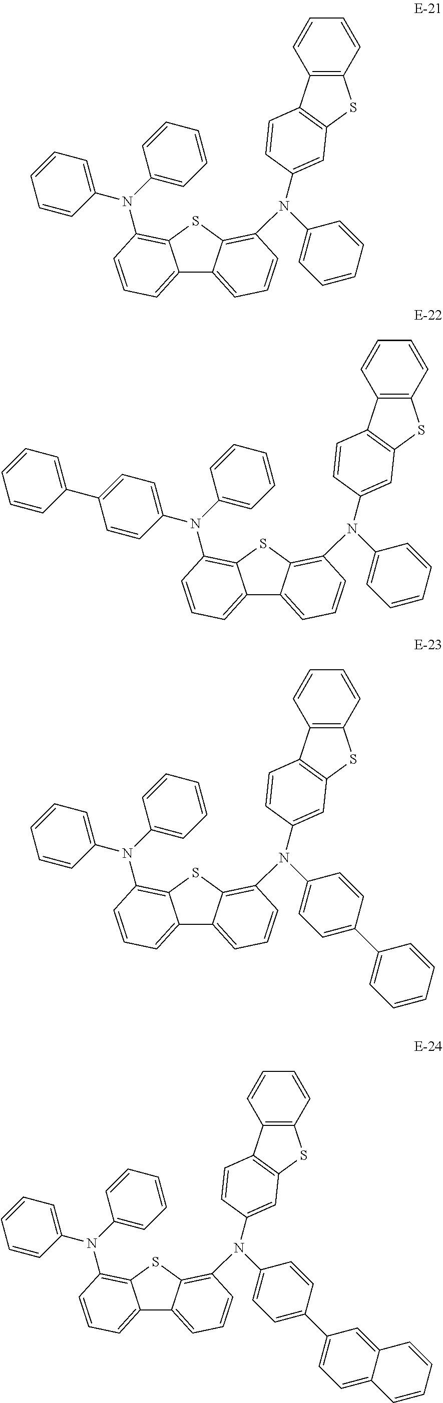 [SMILES: c1ccc(N(c2ccccc2)c2cccc3c2sc2c(N(c4ccccc4)c4ccc5c(c4)sc4ccccc45)cccc23)cc1 ; c1ccc(-c2ccc(N(c3ccccc3)c3cccc4c3sc3c(N(c5ccccc5)c5ccc6c(c5)sc5ccccc56)cccc34)cc2)cc1 ; c1ccc(-c2ccc(N(c3ccc4c(c3)sc3ccccc34)c3cccc4c3sc3c(N(c5ccccc5)c5ccccc5)cccc34)cc2)cc1 ; c1ccc(N(c2ccccc2)c2cccc3c2sc2c(N(c4ccc(-c5ccc6ccccc6c5)cc4)c4ccc5c(c4)sc4ccccc45)cccc23)cc1]

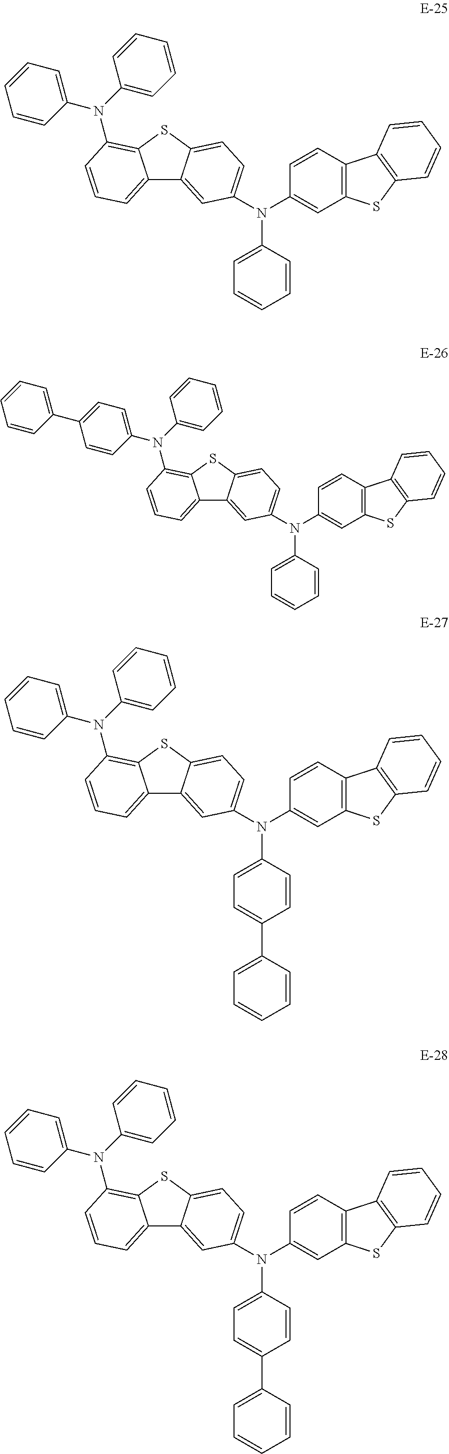 [SMILES: c1ccc(N(c2ccc3c(c2)sc2ccccc23)c2ccc3sc4c(N(c5ccccc5)c5ccccc5)cccc4c3c2)cc1 ; c1ccc(-c2ccc(N(c3ccccc3)c3cccc4c3sc3ccc(N(c5ccccc5)c5ccc6c(c5)sc5ccccc56)cc34)cc2)cc1 ; c1ccc(-c2ccc(N(c3ccc4c(c3)sc3ccccc34)c3ccc4sc5c(N(c6ccccc6)c6ccccc6)cccc5c4c3)cc2)cc1 ; c1ccc(-c2ccc(N(c3ccc4c(c3)sc3ccccc34)c3ccc4sc5c(N(c6ccccc6)c6ccccc6)cccc5c4c3)cc2)cc1]

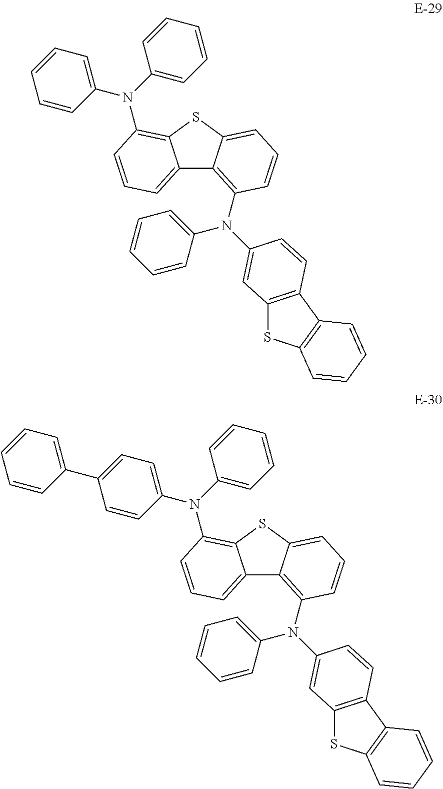 [SMILES: c1ccc(N(c2ccccc2)c2cccc3c2sc2cccc(N(c4ccccc4)c4ccc5c(c4)sc4ccccc45)c23)cc1 ; c1ccc(-c2ccc(N(c3ccccc3)c3cccc4c3sc3cccc(N(c5ccccc5)c5ccc6c(c5)sc5ccccc56)c34)cc2)cc1]

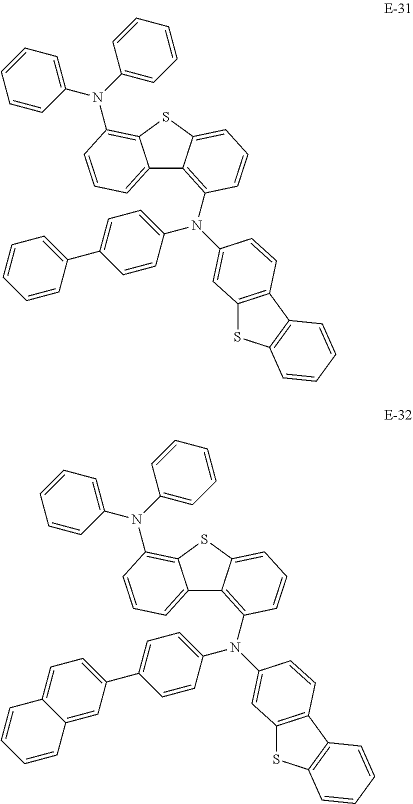 [SMILES: c1ccc(-c2ccc(N(c3ccc4c(c3)sc3ccccc34)c3cccc4sc5c(N(c6ccccc6)c6ccccc6)cccc5c34)cc2)cc1 ; c1ccc(N(c2ccccc2)c2cccc3c2sc2cccc(N(c4ccc(-c5ccc6ccccc6c5)cc4)c4ccc5c(c4)sc4ccccc45)c23)cc1]

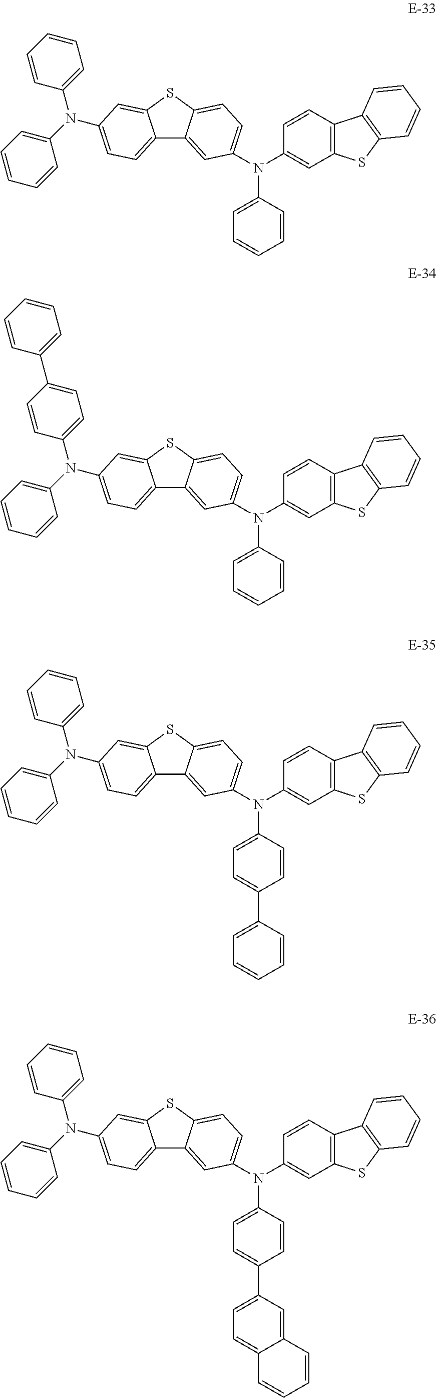 [SMILES: c1ccc(N(c2ccccc2)c2ccc3c(c2)sc2ccc(N(c4ccccc4)c4ccc5c(c4)sc4ccccc45)cc23)cc1 ; c1ccc(-c2ccc(N(c3ccccc3)c3ccc4c(c3)sc3ccc(N(c5ccccc5)c5ccc6c(c5)sc5ccccc56)cc34)cc2)cc1 ; c1ccc(-c2ccc(N(c3ccc4c(c3)sc3ccccc34)c3ccc4sc5cc(N(c6ccccc6)c6ccccc6)ccc5c4c3)cc2)cc1 ; c1ccc(N(c2ccccc2)c2ccc3c(c2)sc2ccc(N(c4ccc(-c5ccc6ccccc6c5)cc4)c4ccc5c(c4)sc4ccccc45)cc23)cc1]

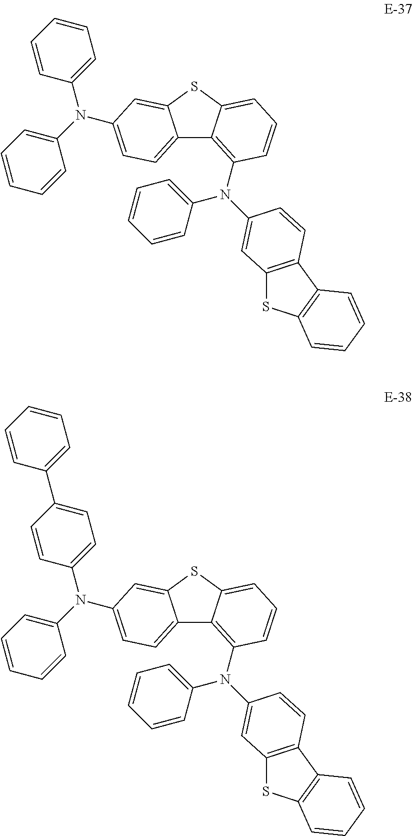 [SMILES: c1ccc(N(c2ccccc2)c2ccc3c(c2)sc2cccc(N(c4ccccc4)c4ccc5c(c4)sc4ccccc45)c23)cc1 ; c1ccc(-c2ccc(N(c3ccccc3)c3ccc4c(c3)sc3cccc(N(c5ccccc5)c5ccc6c(c5)sc5ccccc56)c34)cc2)cc1]

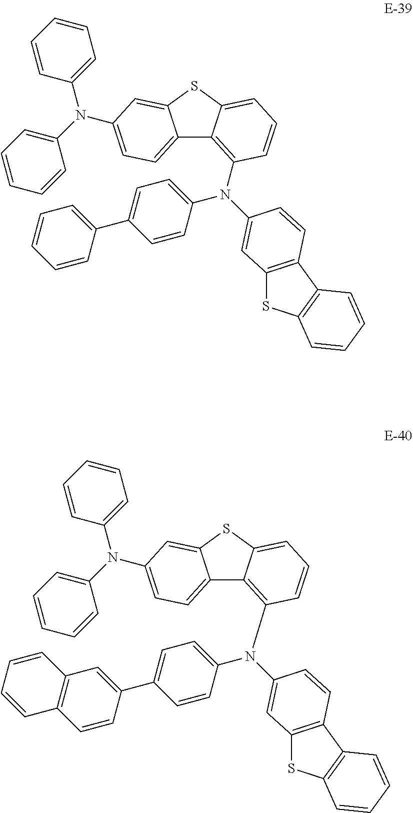 [SMILES: c1ccc(-c2ccc(N(c3ccc4c(c3)sc3ccccc34)c3cccc4sc5cc(N(c6ccccc6)c6ccccc6)ccc5c34)cc2)cc1 ; c1ccc(N(c2ccccc2)c2ccc3c(c2)sc2cccc(N(c4ccc(-c5ccc6ccccc6c5)cc4)c4ccc5c(c4)sc4ccccc45)c23)cc1]

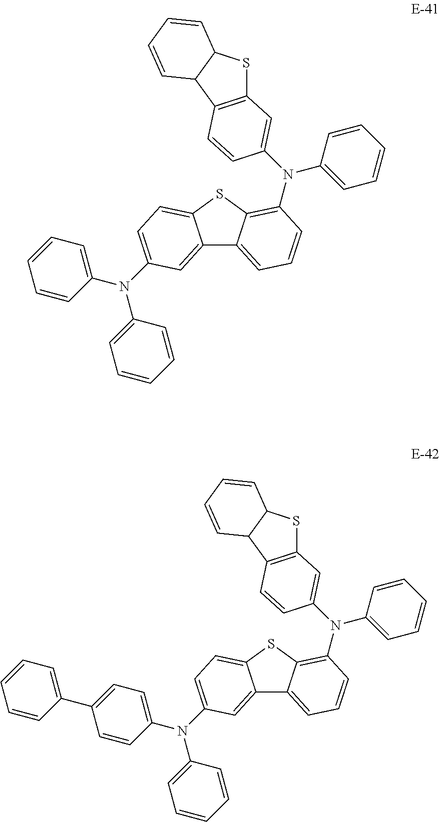 [SMILES: C1=CC2Sc3cc(N(c4ccccc4)c4cccc5c4sc4ccc(N(c6ccccc6)c6ccccc6)cc45)ccc3C2C=C1 ; C1=CC2Sc3cc(N(c4ccccc4)c4cccc5c4sc4ccc(N(c6ccccc6)c6ccc(-c7ccccc7)cc6)cc45)ccc3C2C=C1]

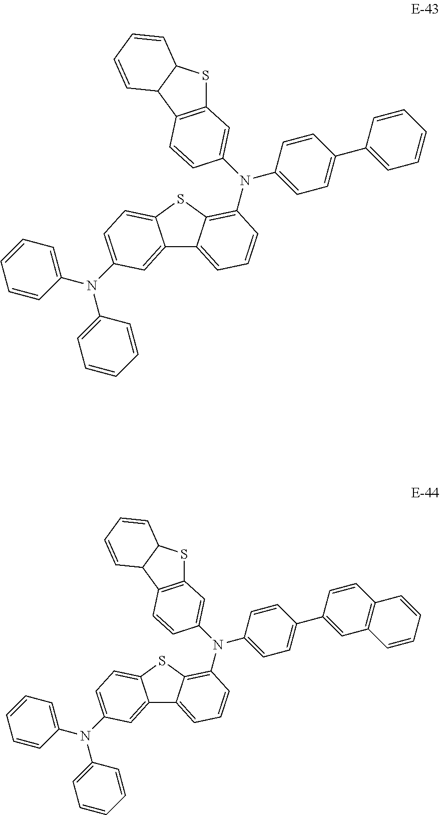 [SMILES: C1=CC2Sc3cc(N(c4ccc(-c5ccccc5)cc4)c4cccc5c4sc4ccc(N(c6ccccc6)c6ccccc6)cc45)ccc3C2C=C1 ; C1=CC2Sc3cc(N(c4ccc(-c5ccc6ccccc6c5)cc4)c4cccc5c4sc4ccc(N(c6ccccc6)c6ccccc6)cc45)ccc3C2C=C1]

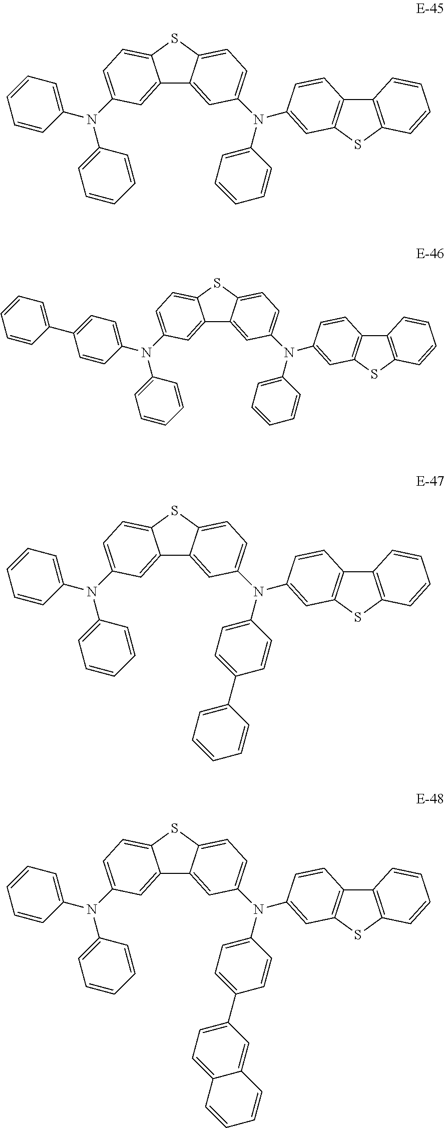 [SMILES: c1ccc(N(c2ccccc2)c2ccc3sc4ccc(N(c5ccccc5)c5ccc6c(c5)sc5ccccc56)cc4c3c2)cc1 ; c1ccc(-c2ccc(N(c3ccccc3)c3ccc4sc5ccc(N(c6ccccc6)c6ccc7c(c6)sc6ccccc67)cc5c4c3)cc2)cc1 ; c1ccc(-c2ccc(N(c3ccc4c(c3)sc3ccccc34)c3ccc4sc5ccc(N(c6ccccc6)c6ccccc6)cc5c4c3)cc2)cc1 ; c1ccc(N(c2ccccc2)c2ccc3sc4ccc(N(c5ccc(-c6ccc7ccccc7c6)cc5)c5ccc6c(c5)sc5ccccc56)cc4c3c2)cc1]

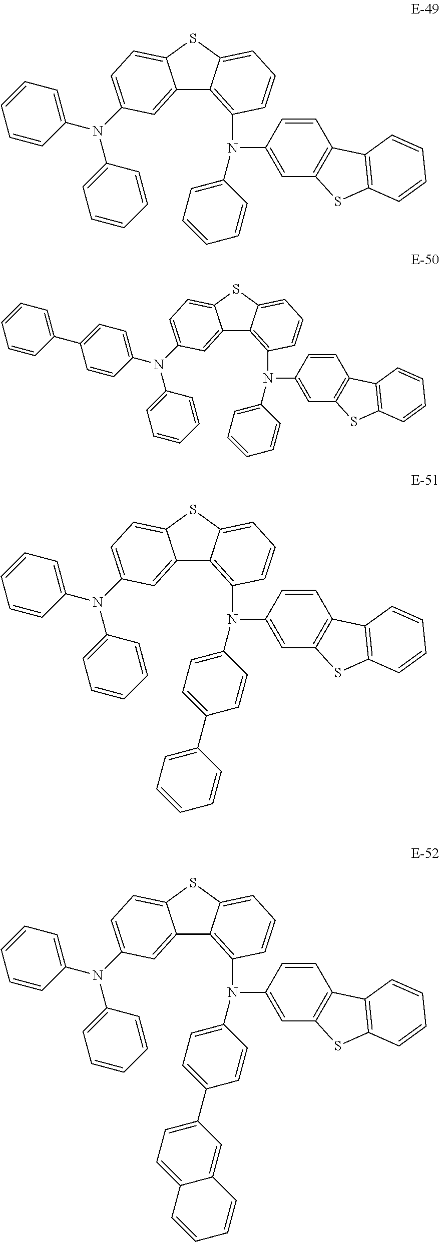 [SMILES: c1ccc(N(c2ccccc2)c2ccc3sc4cccc(N(c5ccccc5)c5ccc6c(c5)sc5ccccc56)c4c3c2)cc1 ; c1ccc(-c2ccc(N(c3ccccc3)c3ccc4sc5cccc(N(c6ccccc6)c6ccc7c(c6)sc6ccccc67)c5c4c3)cc2)cc1 ; c1ccc(-c2ccc(N(c3ccc4c(c3)sc3ccccc34)c3cccc4sc5ccc(N(c6ccccc6)c6ccccc6)cc5c34)cc2)cc1 ; c1ccc(N(c2ccccc2)c2ccc3sc4cccc(N(c5ccc(-c6ccc7ccccc7c6)cc5)c5ccc6c(c5)sc5ccccc56)c4c3c2)cc1]

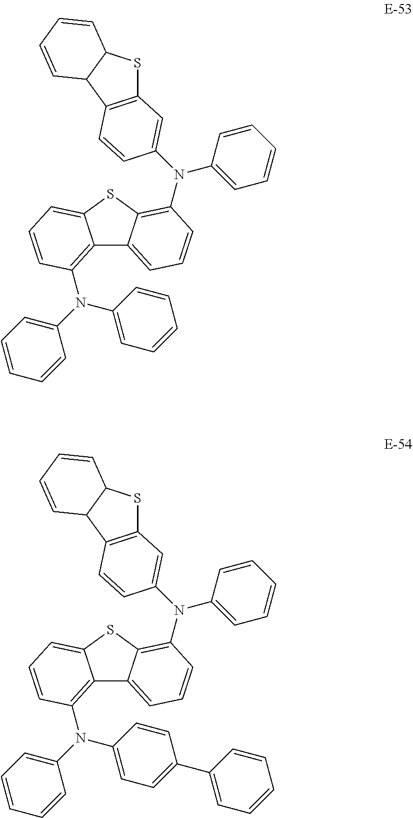 [SMILES: C1=CC2Sc3cc(N(c4ccccc4)c4cccc5c4sc4cccc(N(c6ccccc6)c6ccccc6)c45)ccc3C2C=C1 ; C1=CC2Sc3cc(N(c4ccccc4)c4cccc5c4sc4cccc(N(c6ccccc6)c6ccc(-c7ccccc7)cc6)c45)ccc3C2C=C1]

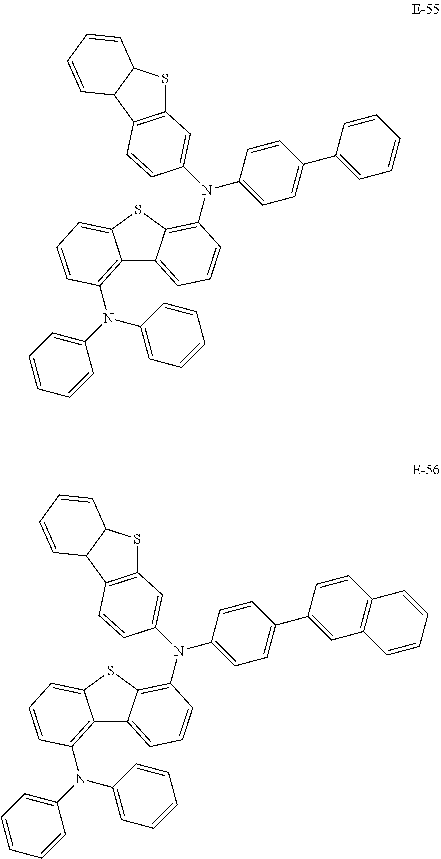 [SMILES: C1=CC2Sc3cc(N(c4ccc(-c5ccccc5)cc4)c4cccc5c4sc4cccc(N(c6ccccc6)c6ccccc6)c45)ccc3C2C=C1 ; C1=CC2Sc3cc(N(c4ccc(-c5ccc6ccccc6c5)cc4)c4cccc5c4sc4cccc(N(c6ccccc6)c6ccccc6)c45)ccc3C2C=C1]

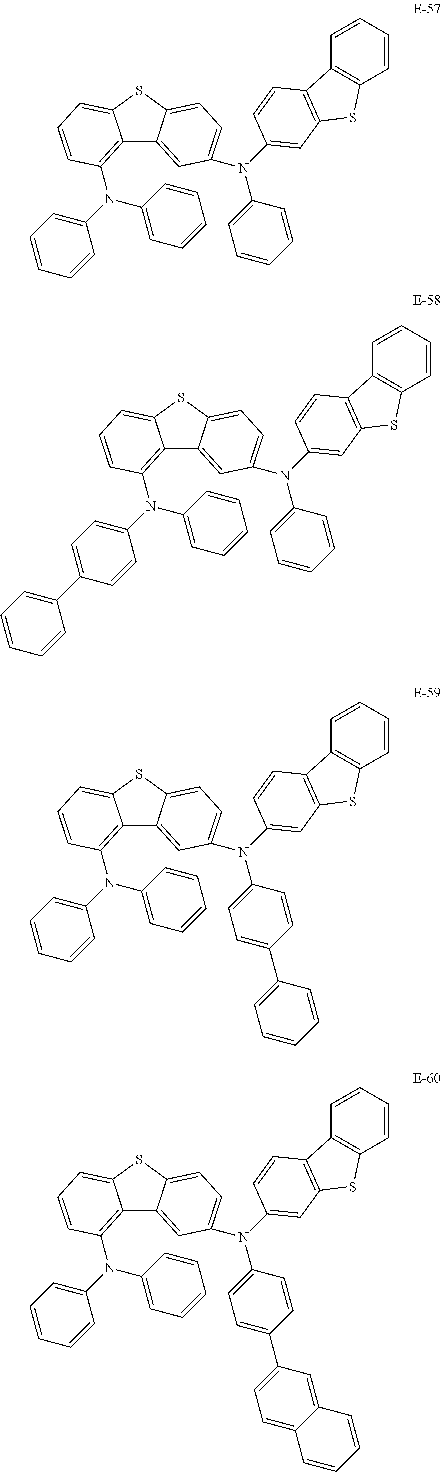 [SMILES: c1ccc(N(c2ccc3c(c2)sc2ccccc23)c2ccc3sc4cccc(N(c5ccccc5)c5ccccc5)c4c3c2)cc1 ; c1ccc(-c2ccc(N(c3ccccc3)c3cccc4sc5ccc(N(c6ccccc6)c6ccc7c(c6)sc6ccccc67)cc5c34)cc2)cc1 ; c1ccc(-c2ccc(N(c3ccc4c(c3)sc3ccccc34)c3ccc4sc5cccc(N(c6ccccc6)c6ccccc6)c5c4c3)cc2)cc1 ; c1ccc(N(c2ccccc2)c2cccc3sc4ccc(N(c5ccc(-c6ccc7ccccc7c6)cc5)c5ccc6c(c5)sc5ccccc56)cc4c23)cc1]

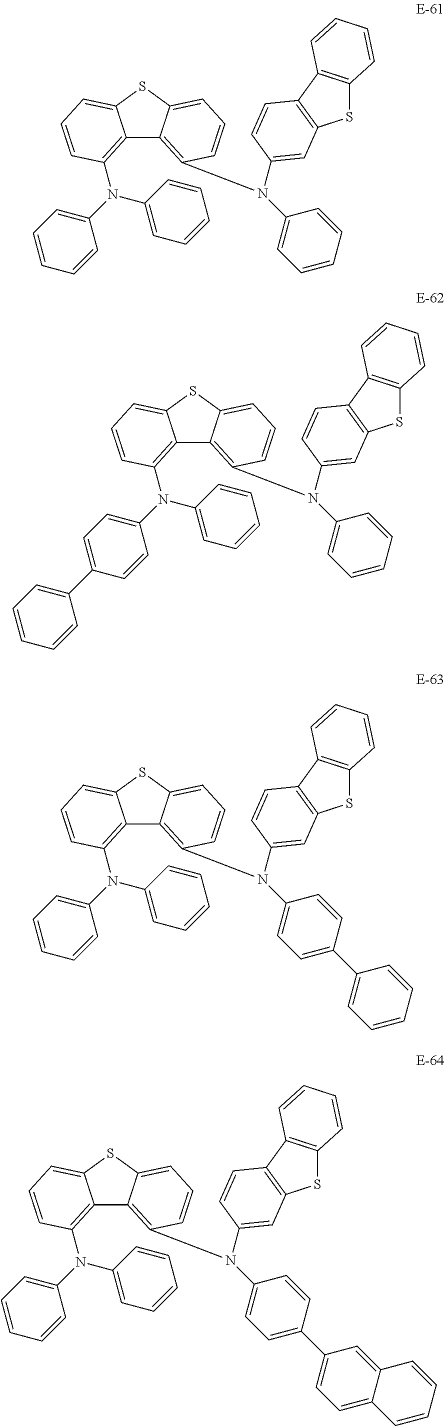 [SMILES: c1ccc(N(c2ccccc2)c2cccc3sc4cccc(N(c5ccccc5)c5ccc6c(c5)sc5ccccc56)c4c23)cc1 ; c1ccc(-c2ccc(N(c3ccccc3)c3cccc4sc5cccc(N(c6ccccc6)c6ccc7c(c6)sc6ccccc67)c5c34)cc2)cc1 ; c1ccc(-c2ccc(N(c3ccc4c(c3)sc3ccccc34)c3cccc4sc5cccc(N(c6ccccc6)c6ccccc6)c5c34)cc2)cc1 ; c1ccc(N(c2ccccc2)c2cccc3sc4cccc(N(c5ccc(-c6ccc7ccccc7c6)cc5)c5ccc6c(c5)sc5ccccc56)c4c23)cc1]

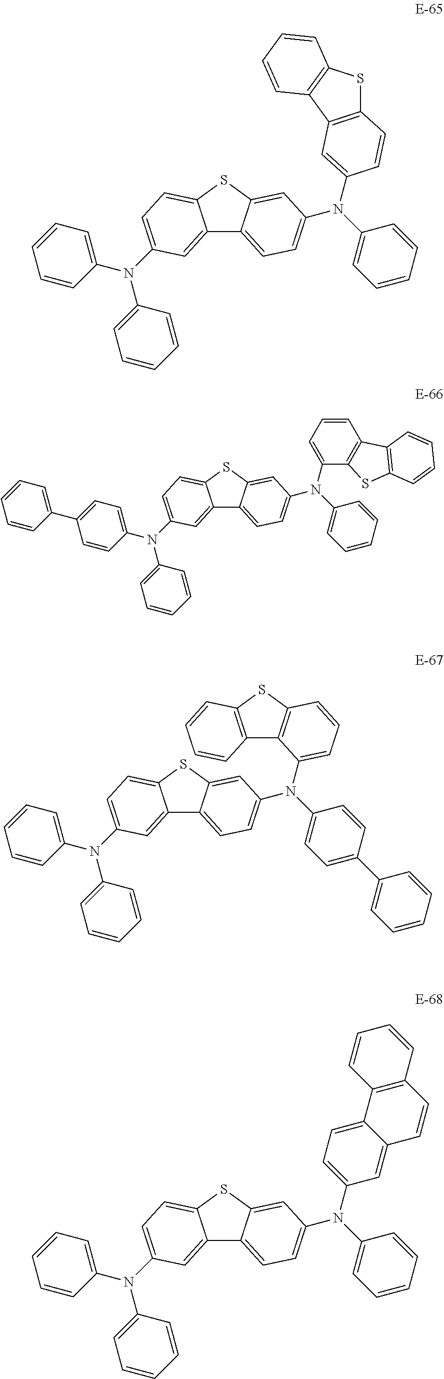 [SMILES: c1ccc(N(c2ccc3c(c2)sc2ccc(N(c4ccccc4)c4ccccc4)cc23)c2ccc3sc4ccccc4c3c2)cc1 ; c1ccc(-c2ccc(N(c3ccccc3)c3ccc4sc5cc(N(c6ccccc6)c6cccc7c6sc6ccccc67)ccc5c4c3)cc2)cc1 ; c1ccc(-c2ccc(N(c3ccc4c(c3)sc3ccc(N(c5ccccc5)c5ccccc5)cc34)c3cccc4sc5ccccc5c34)cc2)cc1 ; c1ccc(N(c2ccc3c(ccc4ccccc43)c2)c2ccc3c(c2)sc2ccc(N(c4ccccc4)c4ccccc4)cc23)cc1]

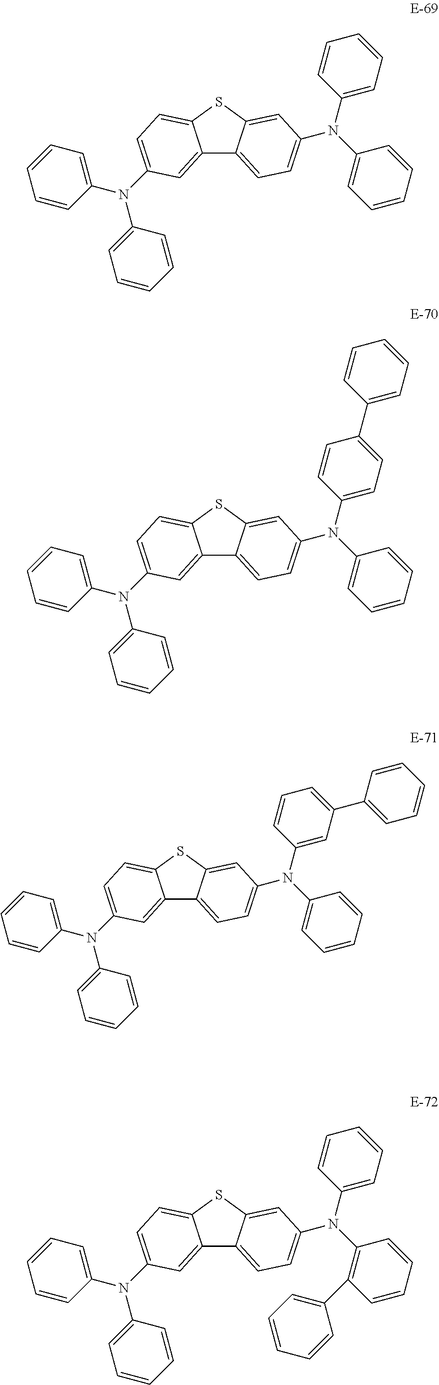 [SMILES: c1ccc(N(c2ccccc2)c2ccc3c(c2)sc2ccc(N(c4ccccc4)c4ccccc4)cc23)cc1 ; c1ccc(-c2ccc(N(c3ccccc3)c3ccc4c(c3)sc3ccc(N(c5ccccc5)c5ccccc5)cc34)cc2)cc1 ; c1ccc(-c2cccc(N(c3ccccc3)c3ccc4c(c3)sc3ccc(N(c5ccccc5)c5ccccc5)cc34)c2)cc1 ; c1ccc(-c2ccccc2N(c2ccccc2)c2ccc3c(c2)sc2ccc(N(c4ccccc4)c4ccccc4)cc23)cc1]

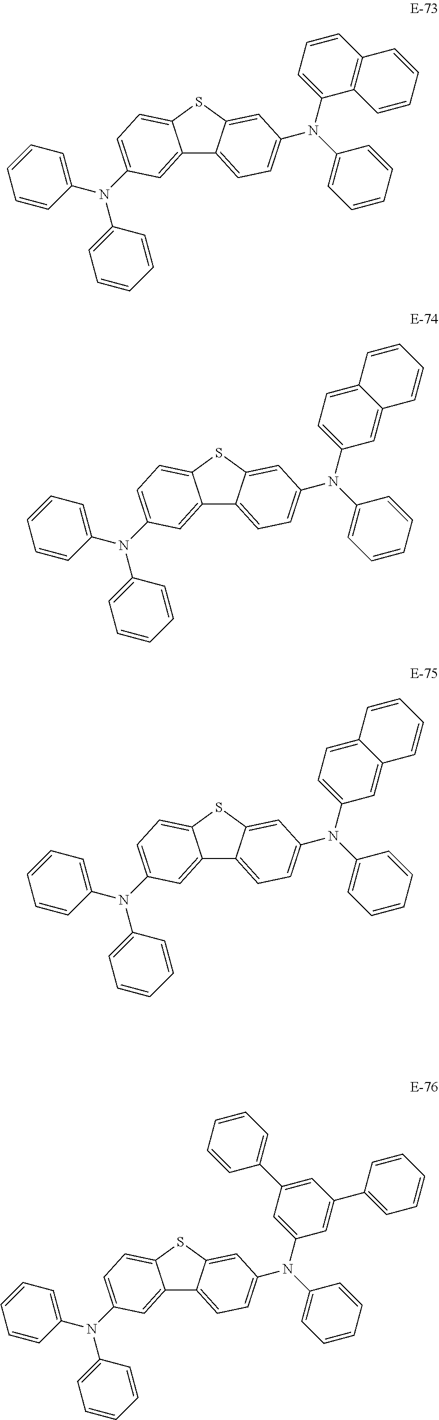 [SMILES: c1ccc(N(c2ccccc2)c2ccc3sc4cc(N(c5ccccc5)c5cccc6ccccc56)ccc4c3c2)cc1 ; c1ccc(N(c2ccc3ccccc3c2)c2ccc3c(c2)sc2ccc(N(c4ccccc4)c4ccccc4)cc23)cc1 ; c1ccc(N(c2ccc3ccccc3c2)c2ccc3c(c2)sc2ccc(N(c4ccccc4)c4ccccc4)cc23)cc1 ; c1ccc(-c2cc(-c3ccccc3)cc(N(c3ccccc3)c3ccc4c(c3)sc3ccc(N(c5ccccc5)c5ccccc5)cc34)c2)cc1]

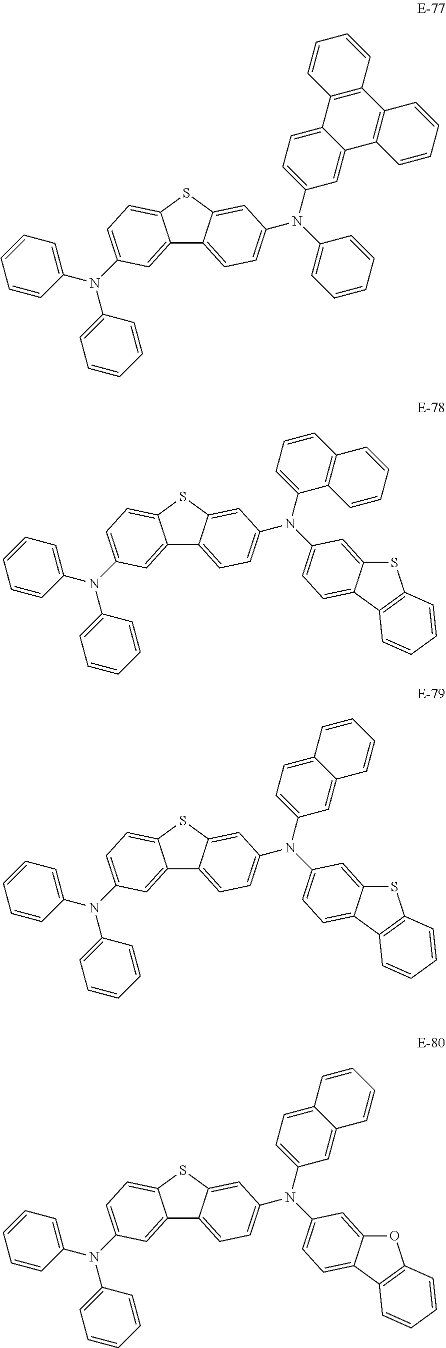 [SMILES: c1ccc(N(c2ccccc2)c2ccc3sc4cc(N(c5ccccc5)c5ccc6c7ccccc7c7ccccc7c6c5)ccc4c3c2)cc1 ; c1ccc(N(c2ccccc2)c2ccc3sc4cc(N(c5ccc6c(c5)sc5ccccc56)c5cccc6ccccc56)ccc4c3c2)cc1 ; c1ccc(N(c2ccccc2)c2ccc3sc4cc(N(c5ccc6ccccc6c5)c5ccc6c(c5)sc5ccccc56)ccc4c3c2)cc1 ; c1ccc(N(c2ccccc2)c2ccc3sc4cc(N(c5ccc6ccccc6c5)c5ccc6c(c5)oc5ccccc56)ccc4c3c2)cc1]

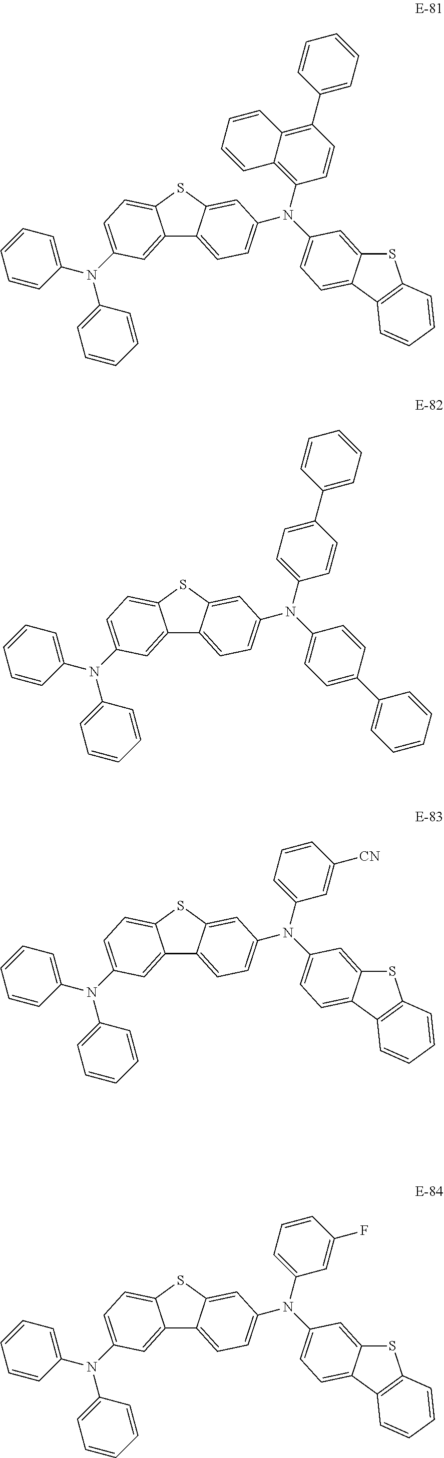 [SMILES: c1ccc(-c2ccc(N(c3ccc4c(c3)sc3ccccc34)c3ccc4c(c3)sc3ccc(N(c5ccccc5)c5ccccc5)cc34)c3ccccc23)cc1 ; c1ccc(-c2ccc(N(c3ccc(-c4ccccc4)cc3)c3ccc4c(c3)sc3ccc(N(c5ccccc5)c5ccccc5)cc34)cc2)cc1 ; N#Cc1cccc(N(c2ccc3c(c2)sc2ccccc23)c2ccc3c(c2)sc2ccc(N(c4ccccc4)c4ccccc4)cc23)c1 ; Fc1cccc(N(c2ccc3c(c2)sc2ccccc23)c2ccc3c(c2)sc2ccc(N(c4ccccc4)c4ccccc4)cc23)c1]

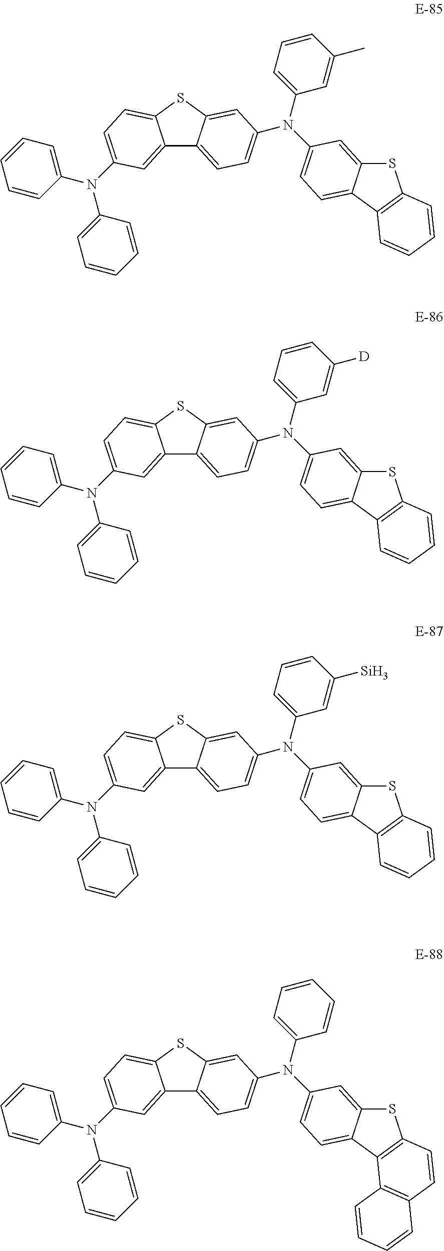

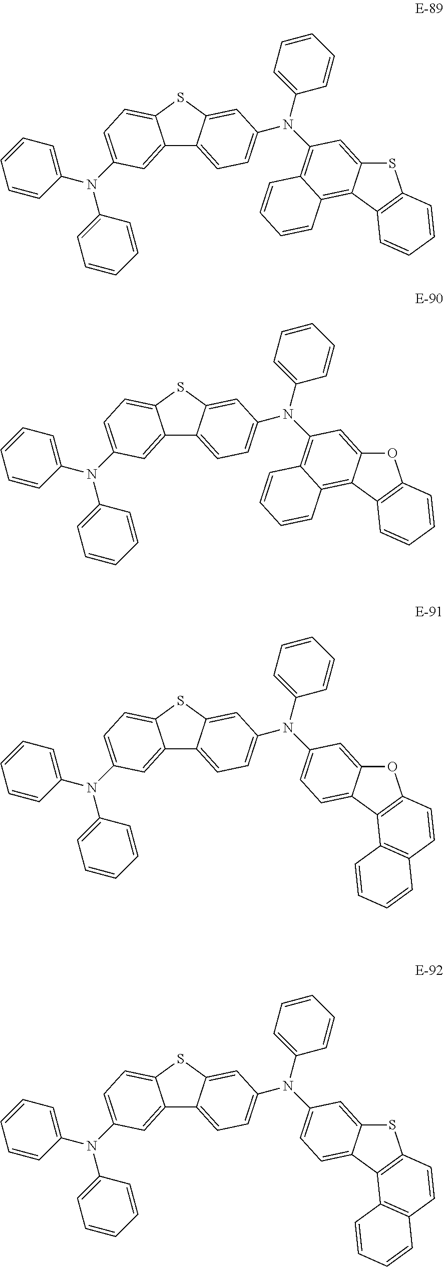 [SMILES: c1ccc(N(c2ccccc2)c2ccc3sc4cc(N(c5ccccc5)c5cc6sc7ccccc7c6c6ccccc56)ccc4c3c2)cc1 ; c1ccc(N(c2ccccc2)c2ccc3sc4cc(N(c5ccccc5)c5cc6oc7ccccc7c6c6ccccc56)ccc4c3c2)cc1 ; c1ccc(N(c2ccc3c(c2)oc2ccc4ccccc4c23)c2ccc3c(c2)sc2ccc(N(c4ccccc4)c4ccccc4)cc23)cc1 ; c1ccc(N(c2ccc3c(c2)sc2ccc(N(c4ccccc4)c4ccccc4)cc23)c2ccc3c(c2)sc2ccc4ccccc4c23)cc1]

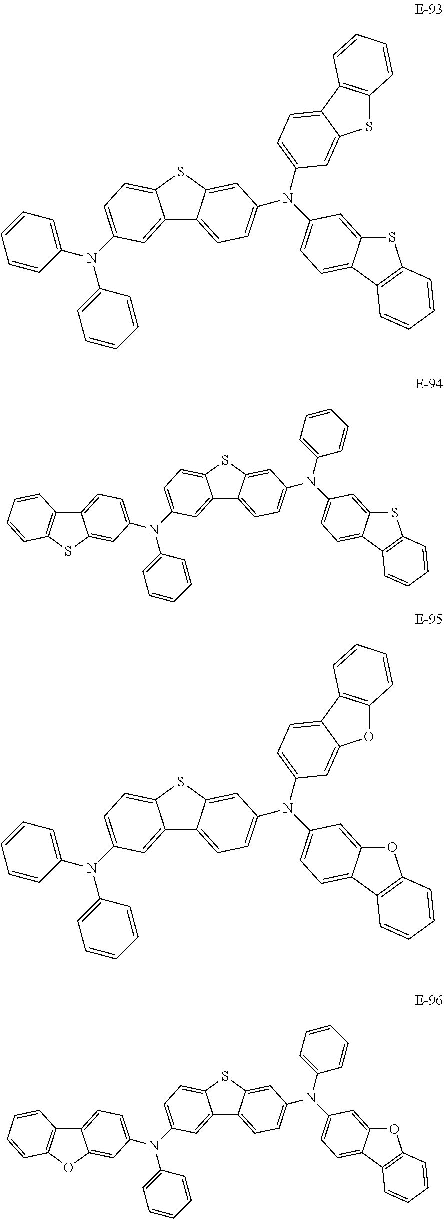 [SMILES: c1ccc(N(c2ccccc2)c2ccc3sc4cc(N(c5ccc6c(c5)sc5ccccc56)c5ccc6c(c5)sc5ccccc56)ccc4c3c2)cc1 ; c1ccc(N(c2ccc3c(c2)sc2ccccc23)c2ccc3c(c2)sc2ccc(N(c4ccccc4)c4ccc5c(c4)sc4ccccc45)cc23)cc1 ; c1ccc(N(c2ccccc2)c2ccc3sc4cc(N(c5ccc6c(c5)oc5ccccc56)c5ccc6c(c5)oc5ccccc56)ccc4c3c2)cc1 ; c1ccc(N(c2ccc3c(c2)oc2ccccc23)c2ccc3c(c2)sc2ccc(N(c4ccccc4)c4ccc5c(c4)oc4ccccc45)cc23)cc1]

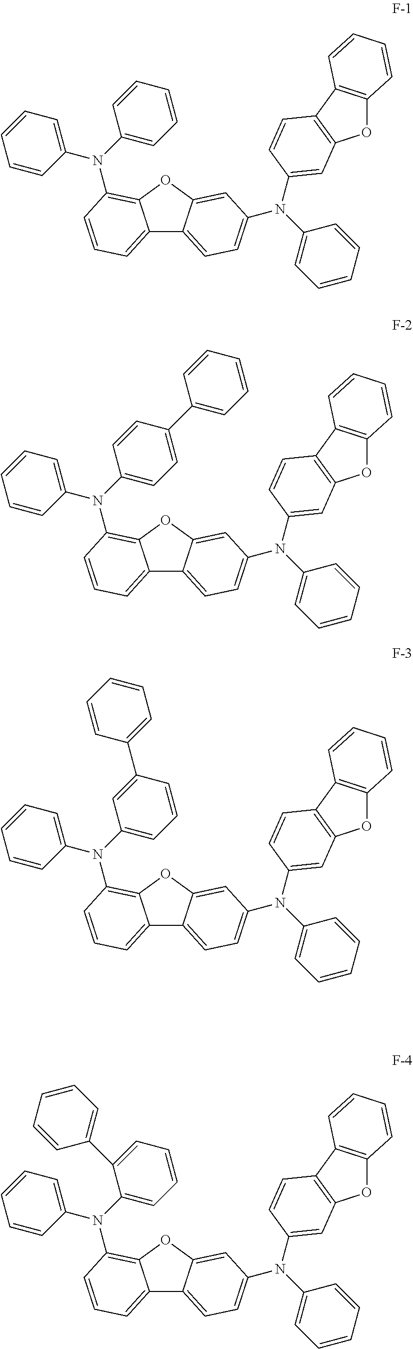 [SMILES: c1ccc(N(c2ccc3c(c2)oc2ccccc23)c2ccc3c(c2)oc2c(N(c4ccccc4)c4ccccc4)cccc23)cc1 ; c1ccc(-c2ccc(N(c3ccccc3)c3cccc4c3oc3cc(N(c5ccccc5)c5ccc6c(c5)oc5ccccc56)ccc34)cc2)cc1 ; c1ccc(-c2cccc(N(c3ccccc3)c3cccc4c3oc3cc(N(c5ccccc5)c5ccc6c(c5)oc5ccccc56)ccc34)c2)cc1 ; c1ccc(-c2ccccc2N(c2ccccc2)c2cccc3c2oc2cc(N(c4ccccc4)c4ccc5c(c4)oc4ccccc45)ccc23)cc1]

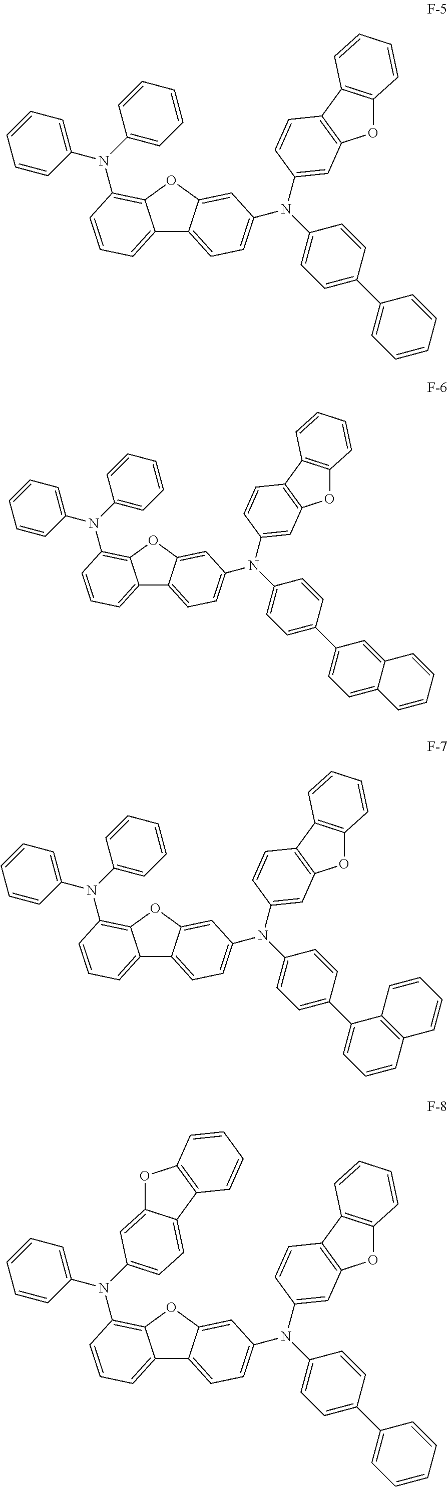 [SMILES: c1ccc(-c2ccc(N(c3ccc4c(c3)oc3ccccc34)c3ccc4c(c3)oc3c(N(c5ccccc5)c5ccccc5)cccc34)cc2)cc1 ; c1ccc(N(c2ccccc2)c2cccc3c2oc2cc(N(c4ccc(-c5ccc6ccccc6c5)cc4)c4ccc5c(c4)oc4ccccc45)ccc23)cc1 ; c1ccc(N(c2ccccc2)c2cccc3c2oc2cc(N(c4ccc(-c5cccc6ccccc56)cc4)c4ccc5c(c4)oc4ccccc45)ccc23)cc1 ; c1ccc(-c2ccc(N(c3ccc4c(c3)oc3ccccc34)c3ccc4c(c3)oc3c(N(c5ccccc5)c5ccc6c(c5)oc5ccccc56)cccc34)cc2)cc1]

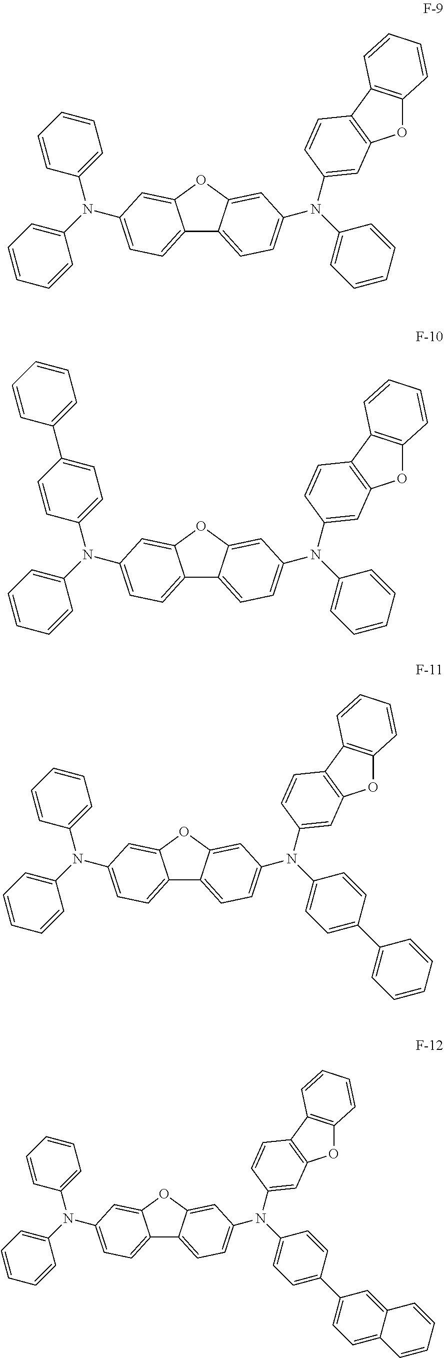 [SMILES: c1ccc(N(c2ccccc2)c2ccc3c(c2)oc2cc(N(c4ccccc4)c4ccc5c(c4)oc4ccccc45)ccc23)cc1 ; c1ccc(-c2ccc(N(c3ccccc3)c3ccc4c(c3)oc3cc(N(c5ccccc5)c5ccc6c(c5)oc5ccccc56)ccc34)cc2)cc1 ; c1ccc(-c2ccc(N(c3ccc4c(c3)oc3ccccc34)c3ccc4c(c3)oc3cc(N(c5ccccc5)c5ccccc5)ccc34)cc2)cc1 ; c1ccc(N(c2ccccc2)c2ccc3c(c2)oc2cc(N(c4ccc(-c5ccc6ccccc6c5)cc4)c4ccc5c(c4)oc4ccccc45)ccc23)cc1]

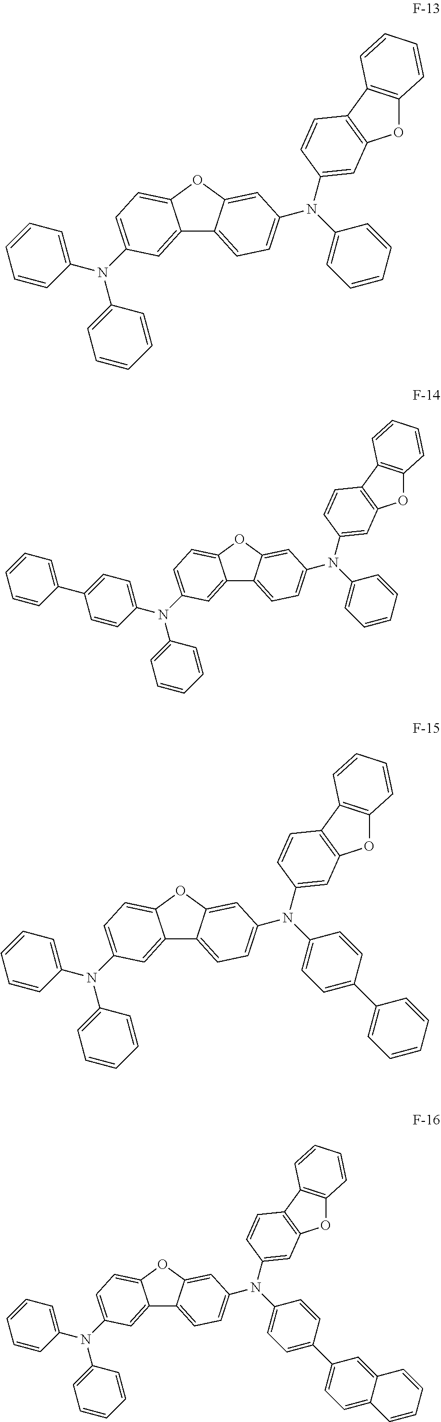 [SMILES: c1ccc(N(c2ccc3c(c2)oc2ccccc23)c2ccc3c(c2)oc2ccc(N(c4ccccc4)c4ccccc4)cc23)cc1 ; c1ccc(-c2ccc(N(c3ccccc3)c3ccc4oc5cc(N(c6ccccc6)c6ccc7c(c6)oc6ccccc67)ccc5c4c3)cc2)cc1 ; c1ccc(-c2ccc(N(c3ccc4c(c3)oc3ccccc34)c3ccc4c(c3)oc3ccc(N(c5ccccc5)c5ccccc5)cc34)cc2)cc1 ; c1ccc(N(c2ccccc2)c2ccc3oc4cc(N(c5ccc(-c6ccc7ccccc7c6)cc5)c5ccc6c(c5)oc5ccccc56)ccc4c3c2)cc1]

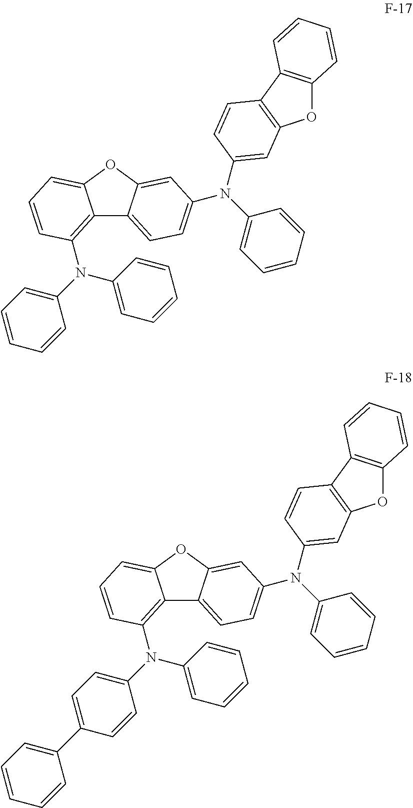 [SMILES: c1ccc(N(c2ccc3c(c2)oc2ccccc23)c2ccc3c(c2)oc2cccc(N(c4ccccc4)c4ccccc4)c23)cc1 ; c1ccc(-c2ccc(N(c3ccccc3)c3cccc4oc5cc(N(c6ccccc6)c6ccc7c(c6)oc6ccccc67)ccc5c34)cc2)cc1]

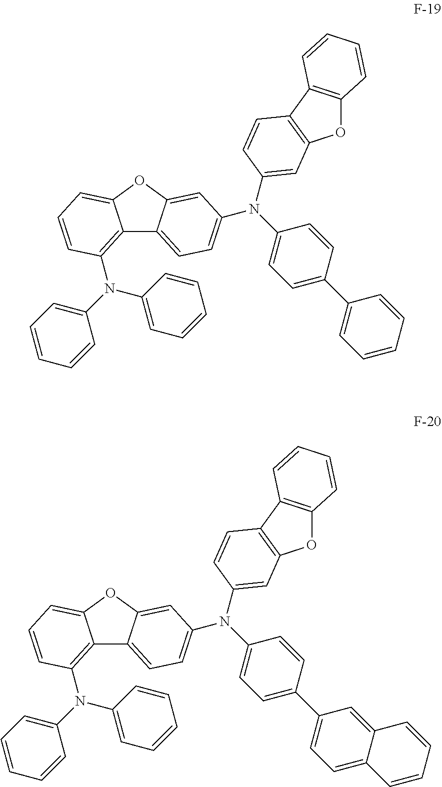 [SMILES: c1ccc(-c2ccc(N(c3ccc4c(c3)oc3ccccc34)c3ccc4c(c3)oc3cccc(N(c5ccccc5)c5ccccc5)c34)cc2)cc1 ; c1ccc(N(c2ccccc2)c2cccc3oc4cc(N(c5ccc(-c6ccc7ccccc7c6)cc5)c5ccc6c(c5)oc5ccccc56)ccc4c23)cc1]

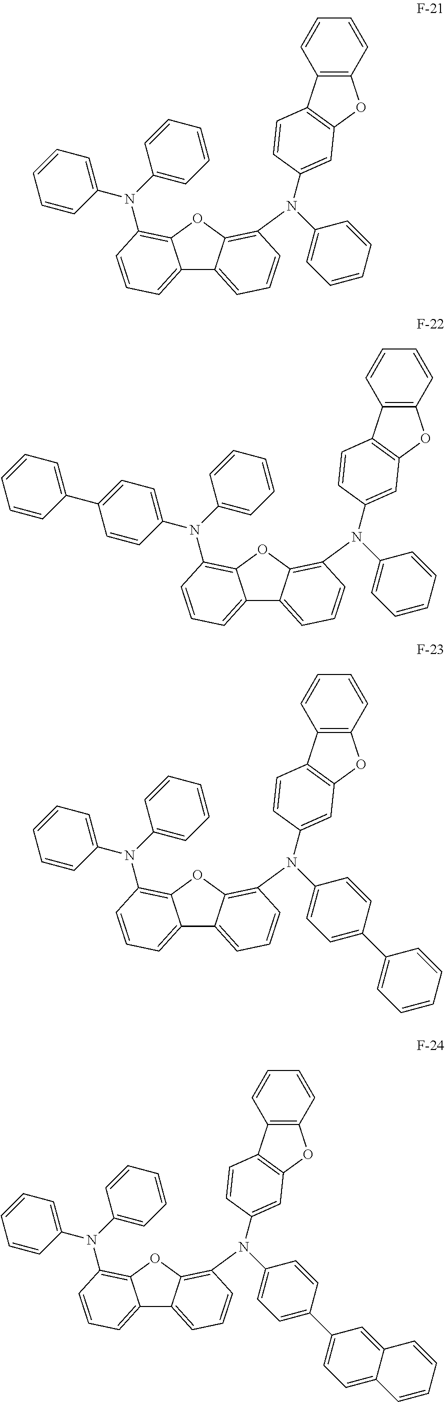 [SMILES: c1ccc(N(c2ccccc2)c2cccc3c2oc2c(N(c4ccccc4)c4ccc5c(c4)oc4ccccc45)cccc23)cc1 ; c1ccc(-c2ccc(N(c3ccccc3)c3cccc4c3oc3c(N(c5ccccc5)c5ccc6c(c5)oc5ccccc56)cccc34)cc2)cc1 ; c1ccc(-c2ccc(N(c3ccc4c(c3)oc3ccccc34)c3cccc4c3oc3c(N(c5ccccc5)c5ccccc5)cccc34)cc2)cc1 ; c1ccc(N(c2ccccc2)c2cccc3c2oc2c(N(c4ccc(-c5ccc6ccccc6c5)cc4)c4ccc5c(c4)oc4ccccc45)cccc23)cc1]

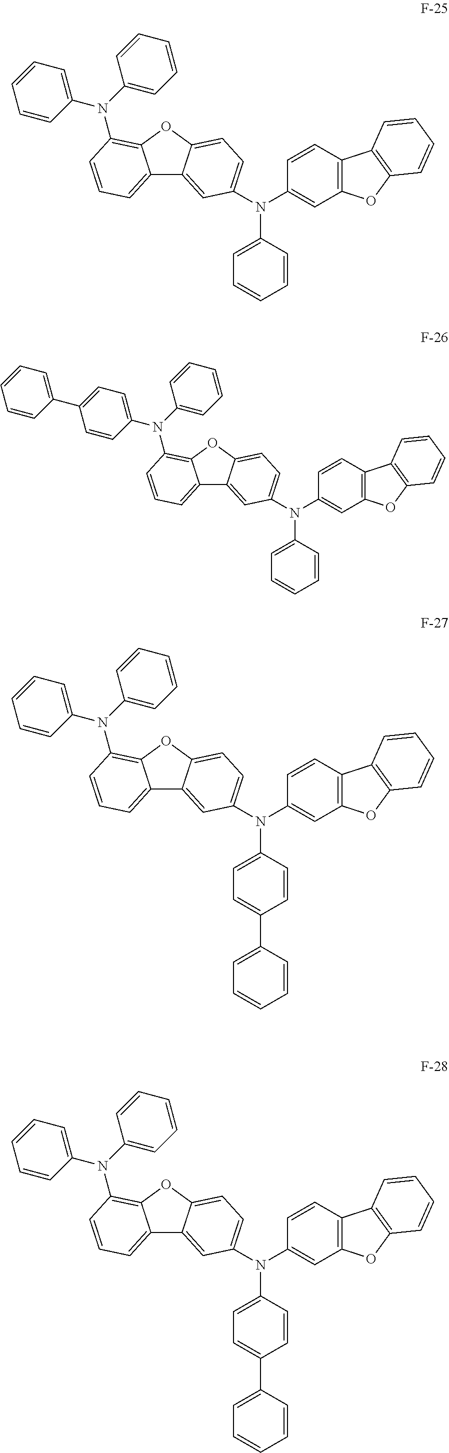 [SMILES: c1ccc(N(c2ccc3c(c2)oc2ccccc23)c2ccc3oc4c(N(c5ccccc5)c5ccccc5)cccc4c3c2)cc1 ; c1ccc(-c2ccc(N(c3ccccc3)c3cccc4c3oc3ccc(N(c5ccccc5)c5ccc6c(c5)oc5ccccc56)cc34)cc2)cc1 ; c1ccc(-c2ccc(N(c3ccc4c(c3)oc3ccccc34)c3ccc4oc5c(N(c6ccccc6)c6ccccc6)cccc5c4c3)cc2)cc1 ; c1ccc(-c2ccc(N(c3ccc4c(c3)oc3ccccc34)c3ccc4oc5c(N(c6ccccc6)c6ccccc6)cccc5c4c3)cc2)cc1]

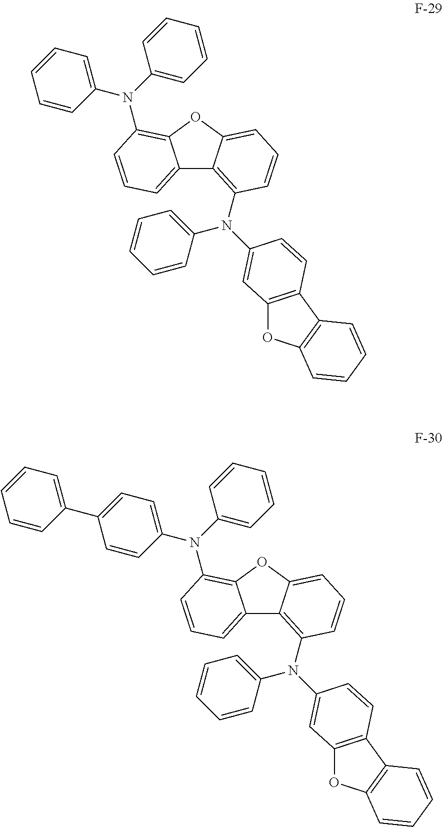 [SMILES: c1ccc(N(c2ccccc2)c2cccc3c2oc2cccc(N(c4ccccc4)c4ccc5c(c4)oc4ccccc45)c23)cc1 ; c1ccc(-c2ccc(N(c3ccccc3)c3cccc4c3oc3cccc(N(c5ccccc5)c5ccc6c(c5)oc5ccccc56)c34)cc2)cc1]

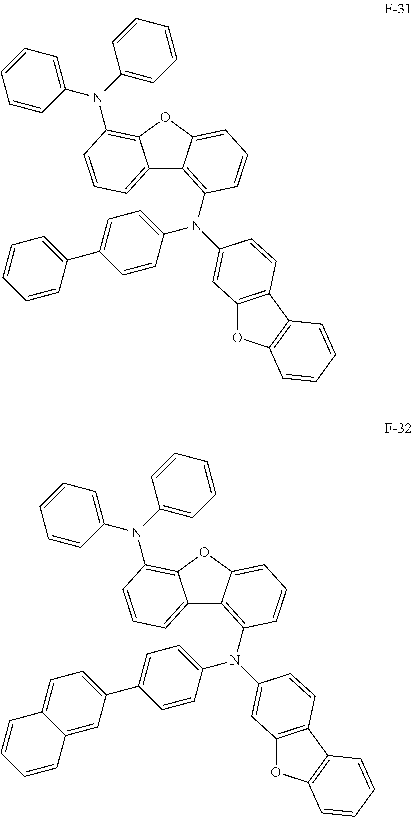 [SMILES: c1ccc(-c2ccc(N(c3ccc4c(c3)oc3ccccc34)c3cccc4oc5c(N(c6ccccc6)c6ccccc6)cccc5c34)cc2)cc1 ; c1ccc(N(c2ccccc2)c2cccc3c2oc2cccc(N(c4ccc(-c5ccc6ccccc6c5)cc4)c4ccc5c(c4)oc4ccccc45)c23)cc1]

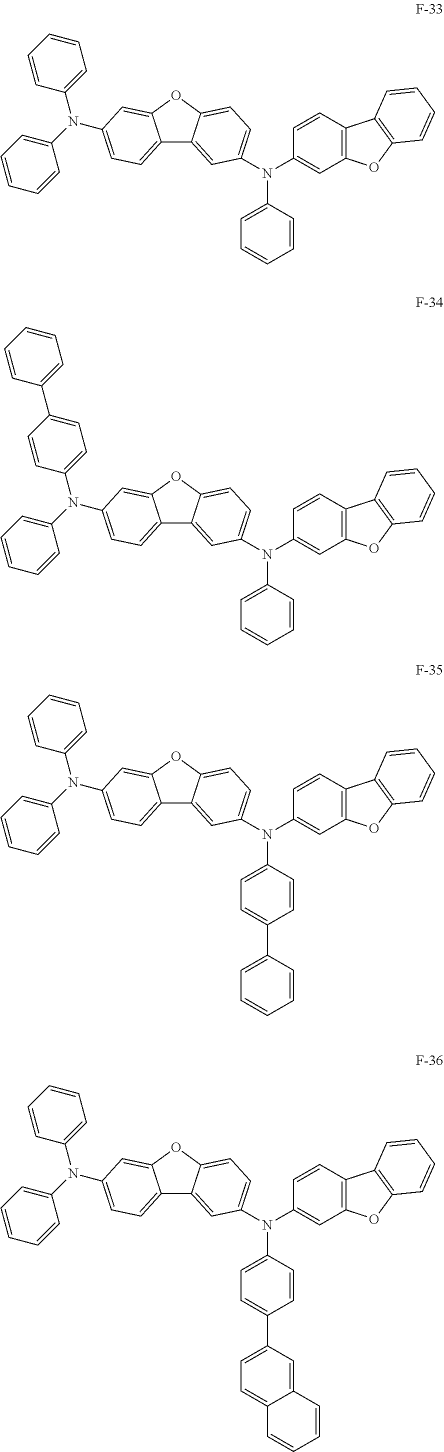 [SMILES: c1ccc(N(c2ccccc2)c2ccc3c(c2)oc2ccc(N(c4ccccc4)c4ccc5c(c4)oc4ccccc45)cc23)cc1 ; c1ccc(-c2ccc(N(c3ccccc3)c3ccc4c(c3)oc3ccc(N(c5ccccc5)c5ccc6c(c5)oc5ccccc56)cc34)cc2)cc1 ; c1ccc(-c2ccc(N(c3ccc4c(c3)oc3ccccc34)c3ccc4oc5cc(N(c6ccccc6)c6ccccc6)ccc5c4c3)cc2)cc1 ; c1ccc(N(c2ccccc2)c2ccc3c(c2)oc2ccc(N(c4ccc(-c5ccc6ccccc6c5)cc4)c4ccc5c(c4)oc4ccccc45)cc23)cc1]

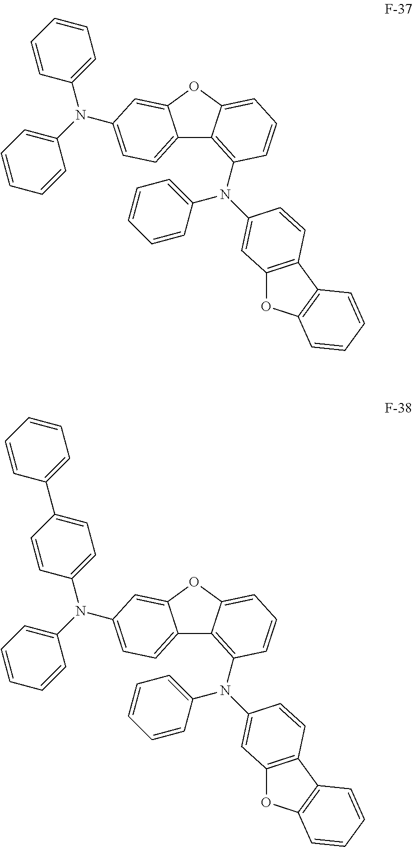 [SMILES: c1ccc(N(c2ccccc2)c2ccc3c(c2)oc2cccc(N(c4ccccc4)c4ccc5c(c4)oc4ccccc45)c23)cc1 ; c1ccc(-c2ccc(N(c3ccccc3)c3ccc4c(c3)oc3cccc(N(c5ccccc5)c5ccc6c(c5)oc5ccccc56)c34)cc2)cc1]

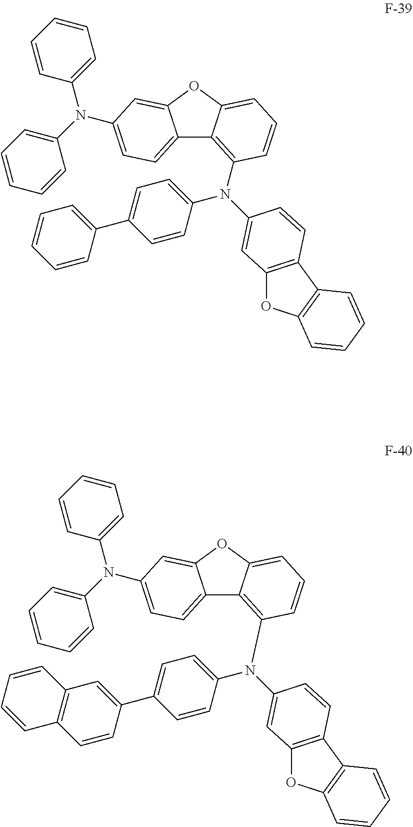 [SMILES: c1ccc(-c2ccc(N(c3ccc4c(c3)oc3ccccc34)c3cccc4oc5cc(N(c6ccccc6)c6ccccc6)ccc5c34)cc2)cc1 ; c1ccc(N(c2ccccc2)c2ccc3c(c2)oc2cccc(N(c4ccc(-c5ccc6ccccc6c5)cc4)c4ccc5c(c4)oc4ccccc45)c23)cc1]

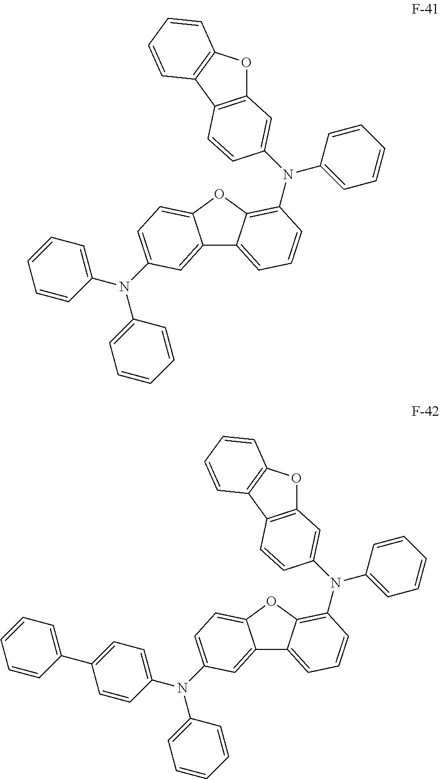 [SMILES: c1ccc(N(c2ccccc2)c2ccc3oc4c(N(c5ccccc5)c5ccc6c(c5)oc5ccccc56)cccc4c3c2)cc1 ; c1ccc(-c2ccc(N(c3ccccc3)c3ccc4oc5c(N(c6ccccc6)c6ccc7c(c6)oc6ccccc67)cccc5c4c3)cc2)cc1]

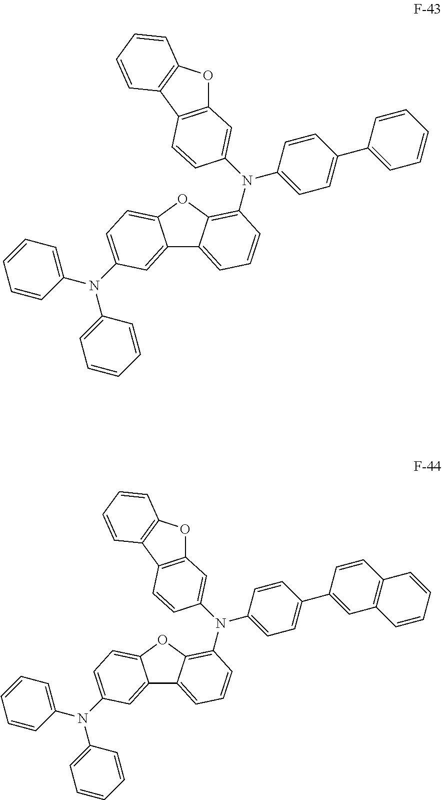 [SMILES: c1ccc(-c2ccc(N(c3ccc4c(c3)oc3ccccc34)c3cccc4c3oc3ccc(N(c5ccccc5)c5ccccc5)cc34)cc2)cc1 ; c1ccc(N(c2ccccc2)c2ccc3oc4c(N(c5ccc(-c6ccc7ccccc7c6)cc5)c5ccc6c(c5)oc5ccccc56)cccc4c3c2)cc1]

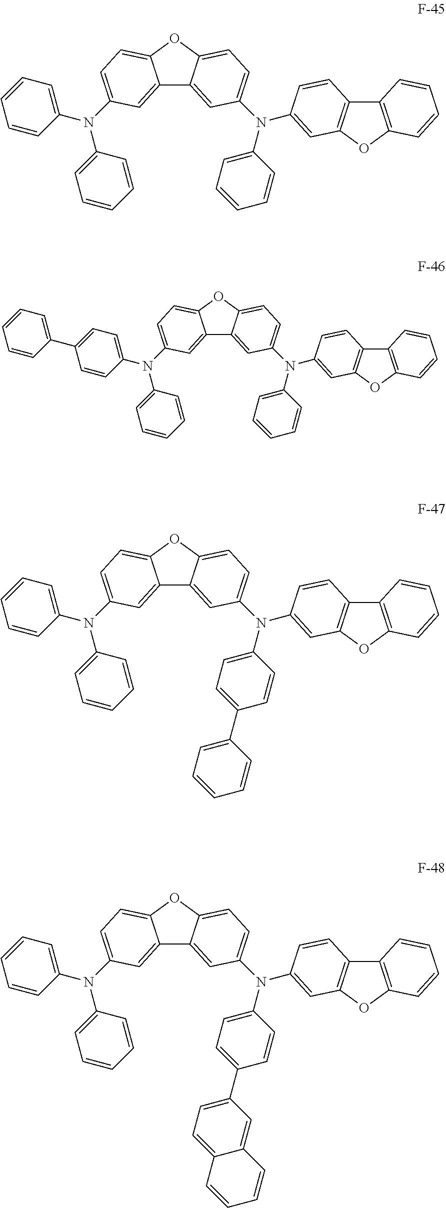 [SMILES: c1ccc(N(c2ccccc2)c2ccc3oc4ccc(N(c5ccccc5)c5ccc6c(c5)oc5ccccc56)cc4c3c2)cc1 ; c1ccc(-c2ccc(N(c3ccccc3)c3ccc4oc5ccc(N(c6ccccc6)c6ccc7c(c6)oc6ccccc67)cc5c4c3)cc2)cc1 ; c1ccc(-c2ccc(N(c3ccc4c(c3)oc3ccccc34)c3ccc4oc5ccc(N(c6ccccc6)c6ccccc6)cc5c4c3)cc2)cc1 ; c1ccc(N(c2ccccc2)c2ccc3oc4ccc(N(c5ccc(-c6ccc7ccccc7c6)cc5)c5ccc6c(c5)oc5ccccc56)cc4c3c2)cc1]

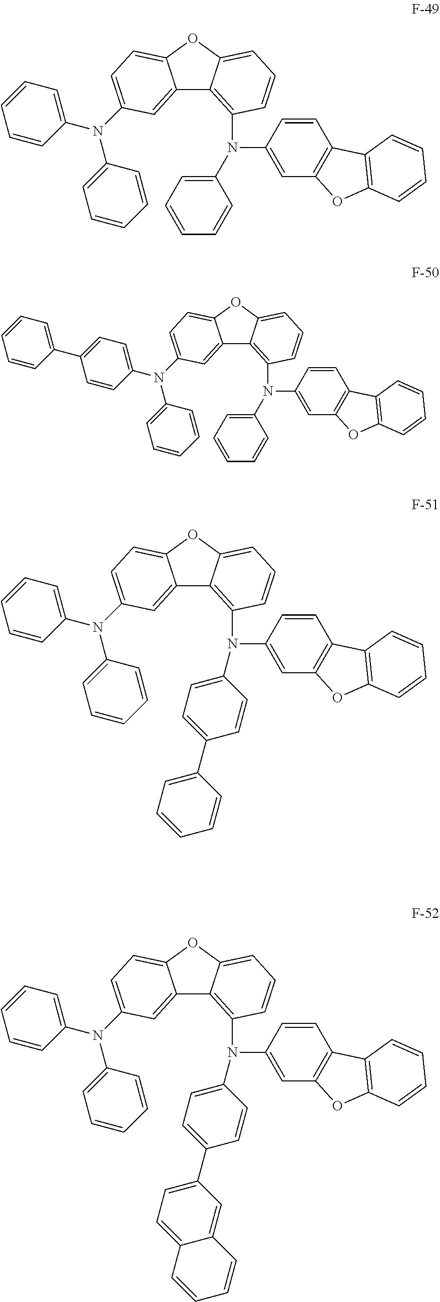 [SMILES: c1ccc(N(c2ccccc2)c2ccc3oc4cccc(N(c5ccccc5)c5ccc6c(c5)oc5ccccc56)c4c3c2)cc1 ; c1ccc(-c2ccc(N(c3ccccc3)c3ccc4oc5cccc(N(c6ccccc6)c6ccc7c(c6)oc6ccccc67)c5c4c3)cc2)cc1 ; c1ccc(-c2ccc(N(c3ccc4c(c3)oc3ccccc34)c3cccc4oc5ccc(N(c6ccccc6)c6ccccc6)cc5c34)cc2)cc1 ; c1ccc(N(c2ccccc2)c2ccc3oc4cccc(N(c5ccc(-c6ccc7ccccc7c6)cc5)c5ccc6c(c5)oc5ccccc56)c4c3c2)cc1]

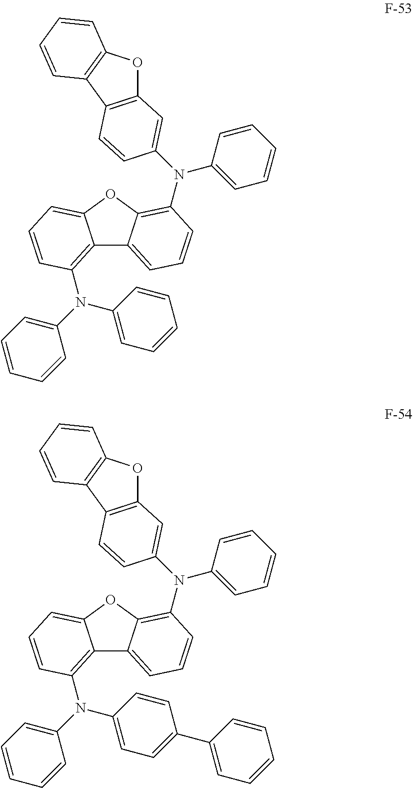

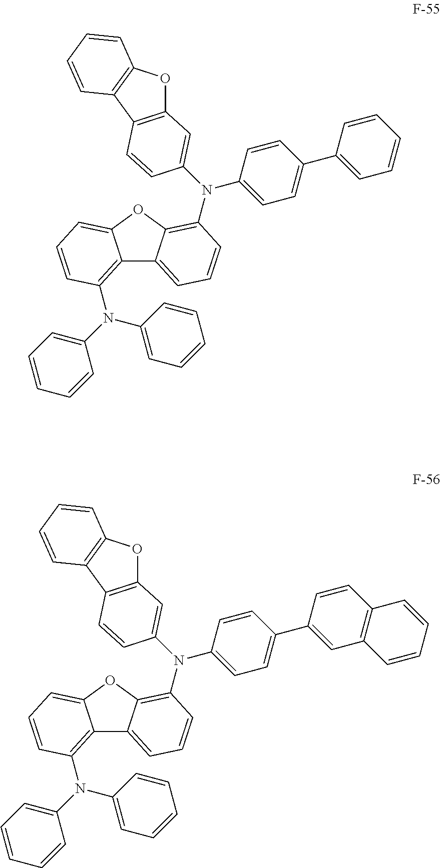 [SMILES: c1ccc(-c2ccc(N(c3ccc4c(c3)oc3ccccc34)c3cccc4c3oc3cccc(N(c5ccccc5)c5ccccc5)c34)cc2)cc1 ; c1ccc(N(c2ccccc2)c2cccc3oc4c(N(c5ccc(-c6ccc7ccccc7c6)cc5)c5ccc6c(c5)oc5ccccc56)cccc4c23)cc1]

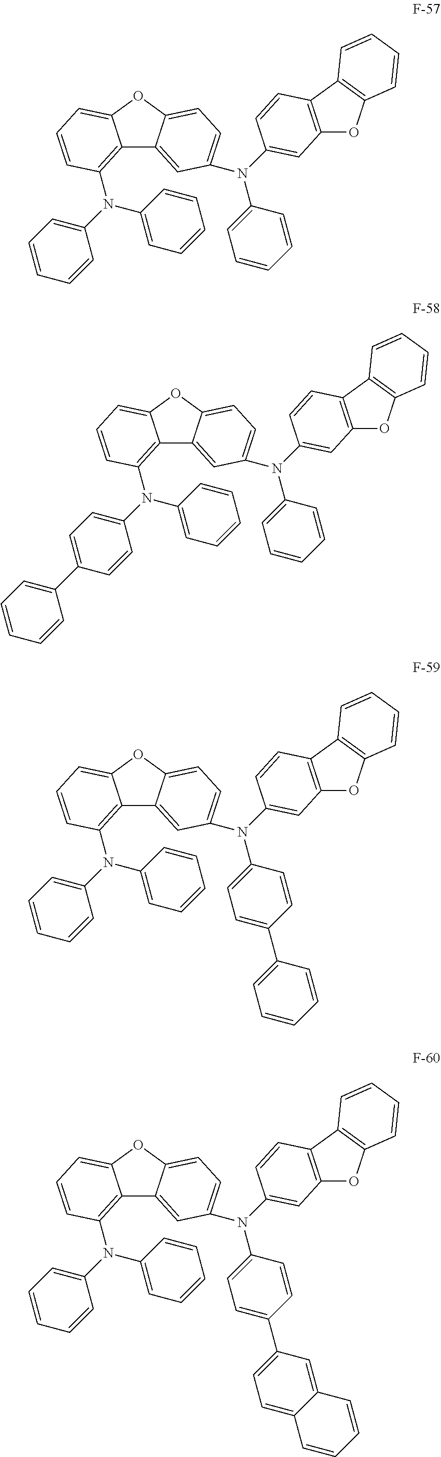 [SMILES: c1ccc(N(c2ccc3c(c2)oc2ccccc23)c2ccc3oc4cccc(N(c5ccccc5)c5ccccc5)c4c3c2)cc1 ; c1ccc(-c2ccc(N(c3ccccc3)c3cccc4oc5ccc(N(c6ccccc6)c6ccc7c(c6)oc6ccccc67)cc5c34)cc2)cc1 ; c1ccc(-c2ccc(N(c3ccc4c(c3)oc3ccccc34)c3ccc4oc5cccc(N(c6ccccc6)c6ccccc6)c5c4c3)cc2)cc1 ; c1ccc(N(c2ccccc2)c2cccc3oc4ccc(N(c5ccc(-c6ccc7ccccc7c6)cc5)c5ccc6c(c5)oc5ccccc56)cc4c23)cc1]

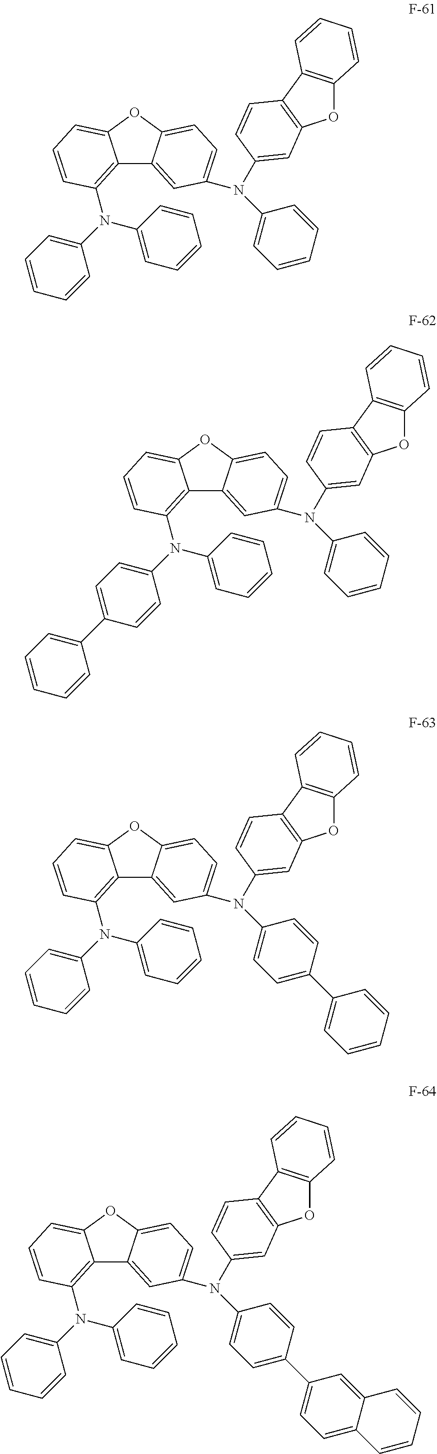 [SMILES: c1ccc(N(c2ccc3c(c2)oc2ccccc23)c2ccc3oc4cccc(N(c5ccccc5)c5ccccc5)c4c3c2)cc1 ; c1ccc(-c2ccc(N(c3ccccc3)c3cccc4oc5ccc(N(c6ccccc6)c6ccc7c(c6)oc6ccccc67)cc5c34)cc2)cc1 ; c1ccc(-c2ccc(N(c3ccc4c(c3)oc3ccccc34)c3ccc4oc5cccc(N(c6ccccc6)c6ccccc6)c5c4c3)cc2)cc1 ; c1ccc(N(c2ccccc2)c2cccc3oc4ccc(N(c5ccc(-c6ccc7ccccc7c6)cc5)c5ccc6c(c5)oc5ccccc56)cc4c23)cc1]

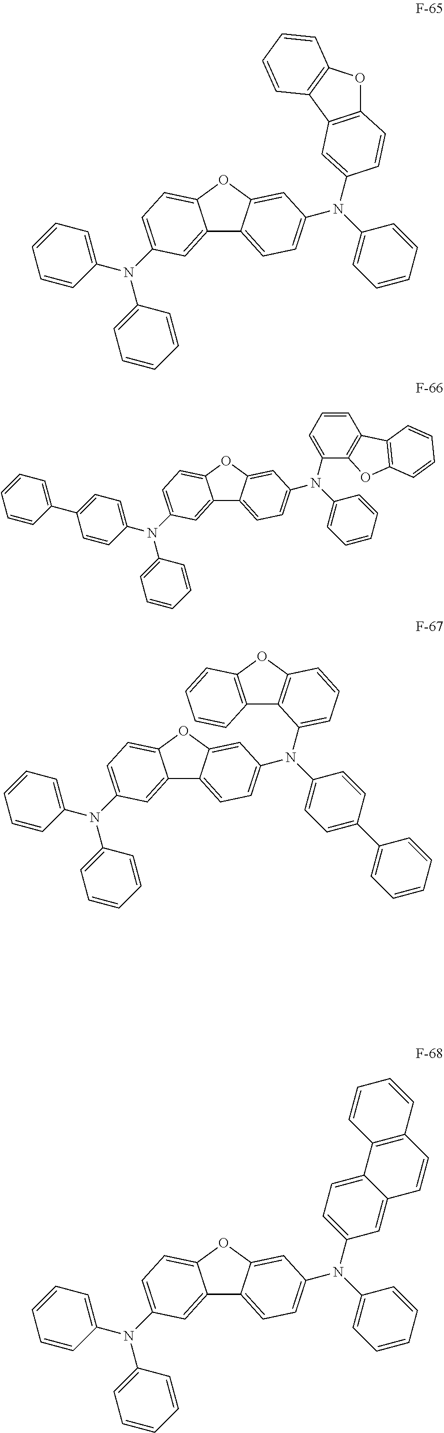 [SMILES: c1ccc(N(c2ccc3c(c2)oc2ccc(N(c4ccccc4)c4ccccc4)cc23)c2ccc3oc4ccccc4c3c2)cc1 ; c1ccc(-c2ccc(N(c3ccccc3)c3ccc4oc5cc(N(c6ccccc6)c6cccc7c6oc6ccccc67)ccc5c4c3)cc2)cc1 ; c1ccc(-c2ccc(N(c3ccc4c(c3)oc3ccc(N(c5ccccc5)c5ccccc5)cc34)c3cccc4oc5ccccc5c34)cc2)cc1 ; c1ccc(N(c2ccc3c(ccc4ccccc43)c2)c2ccc3c(c2)oc2ccc(N(c4ccccc4)c4ccccc4)cc23)cc1]

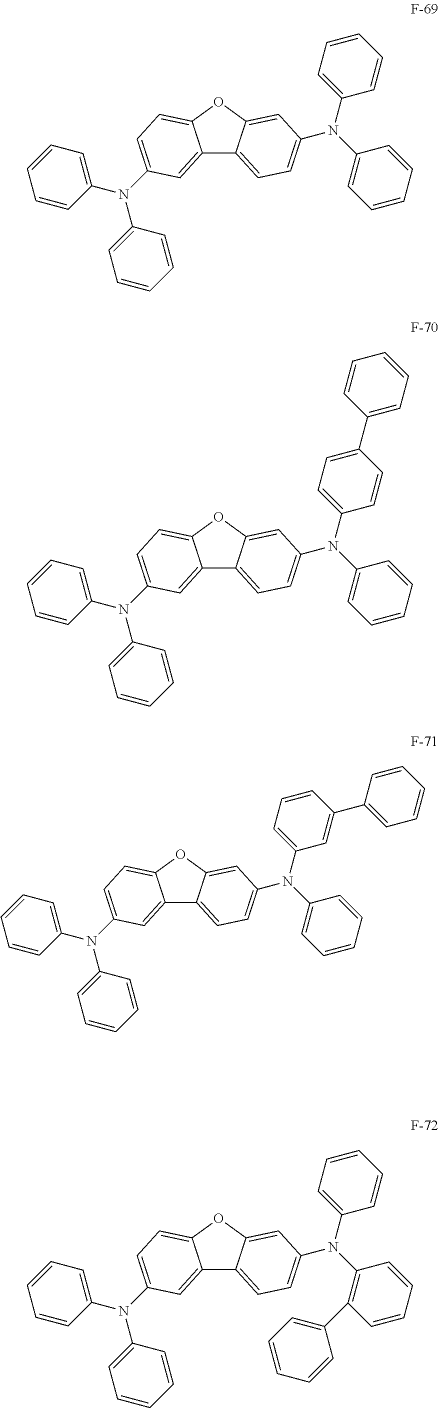 [SMILES: c1ccc(N(c2ccccc2)c2ccc3c(c2)oc2ccc(N(c4ccccc4)c4ccccc4)cc23)cc1 ; c1ccc(-c2ccc(N(c3ccccc3)c3ccc4c(c3)oc3ccc(N(c5ccccc5)c5ccccc5)cc34)cc2)cc1 ; c1ccc(-c2cccc(N(c3ccccc3)c3ccc4c(c3)oc3ccc(N(c5ccccc5)c5ccccc5)cc34)c2)cc1 ; c1ccc(-c2ccccc2N(c2ccccc2)c2ccc3c(c2)oc2ccc(N(c4ccccc4)c4ccccc4)cc23)cc1]

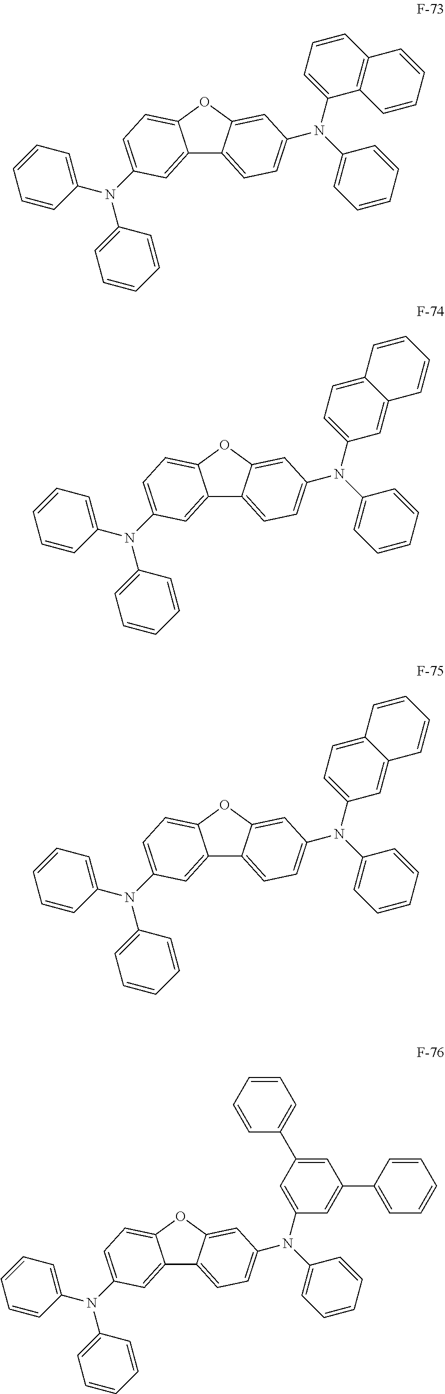 [SMILES: c1ccc(N(c2ccccc2)c2ccc3oc4cc(N(c5ccccc5)c5cccc6ccccc56)ccc4c3c2)cc1 ; c1ccc(N(c2ccc3ccccc3c2)c2ccc3c(c2)oc2ccc(N(c4ccccc4)c4ccccc4)cc23)cc1 ; c1ccc(N(c2ccc3ccccc3c2)c2ccc3c(c2)oc2ccc(N(c4ccccc4)c4ccccc4)cc23)cc1 ; c1ccc(-c2cc(-c3ccccc3)cc(N(c3ccccc3)c3ccc4c(c3)oc3ccc(N(c5ccccc5)c5ccccc5)cc34)c2)cc1]

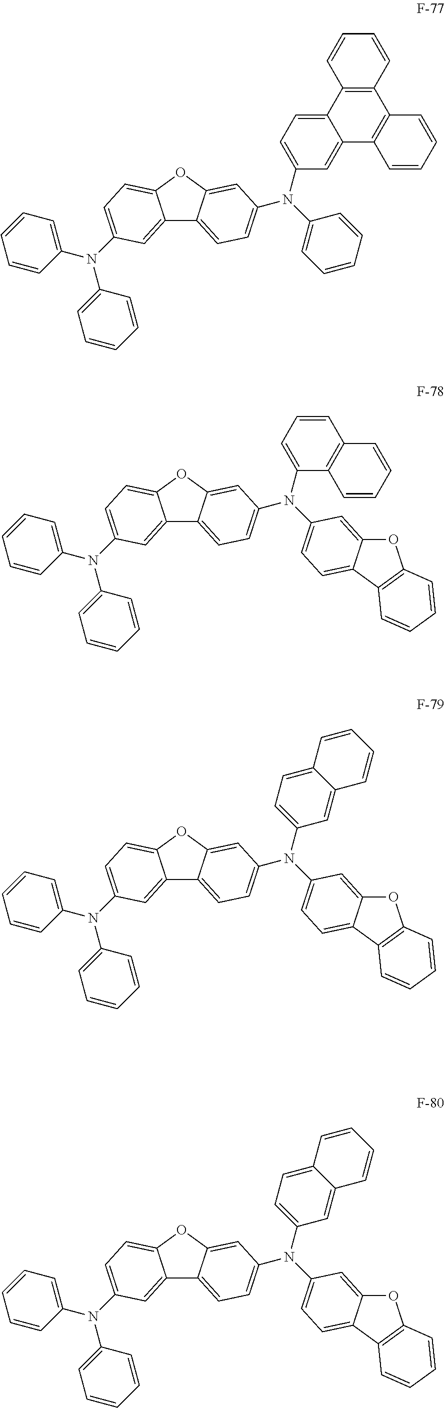 [SMILES: c1ccc(N(c2ccccc2)c2ccc3oc4cc(N(c5ccccc5)c5ccc6c7ccccc7c7ccccc7c6c5)ccc4c3c2)cc1 ; c1ccc(N(c2ccccc2)c2ccc3oc4cc(N(c5ccc6c(c5)oc5ccccc56)c5cccc6ccccc56)ccc4c3c2)cc1 ; c1ccc(N(c2ccccc2)c2ccc3oc4cc(N(c5ccc6ccccc6c5)c5ccc6c(c5)oc5ccccc56)ccc4c3c2)cc1 ; c1ccc(N(c2ccccc2)c2ccc3oc4cc(N(c5ccc6ccccc6c5)c5ccc6c(c5)oc5ccccc56)ccc4c3c2)cc1]

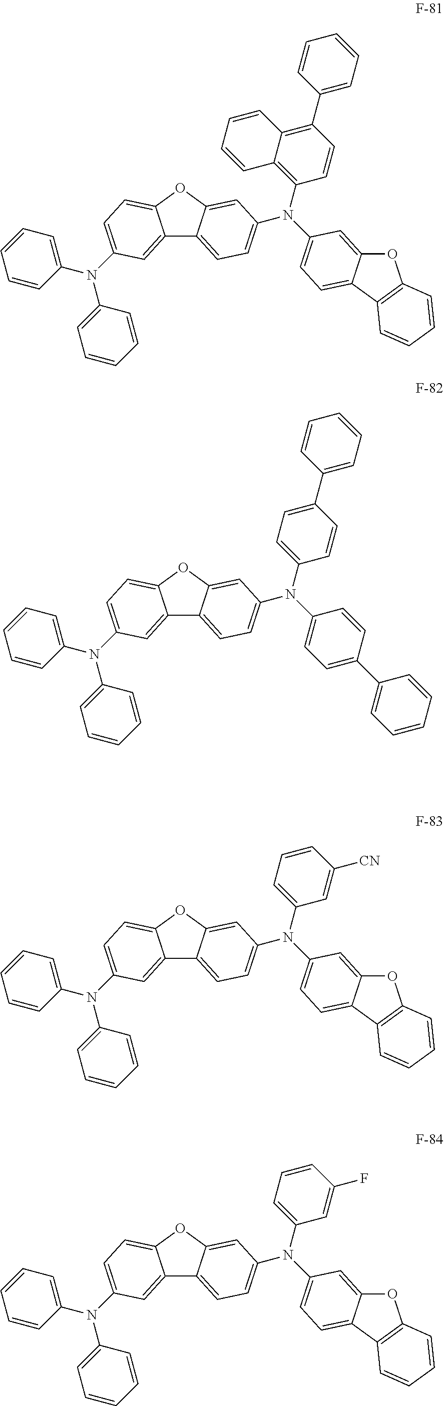 [SMILES: c1ccc(-c2ccc(N(c3ccc4c(c3)oc3ccccc34)c3ccc4c(c3)oc3ccc(N(c5ccccc5)c5ccccc5)cc34)c3ccccc23)cc1 ; c1ccc(-c2ccc(N(c3ccc(-c4ccccc4)cc3)c3ccc4c(c3)oc3ccc(N(c5ccccc5)c5ccccc5)cc34)cc2)cc1 ; N#Cc1cccc(N(c2ccc3c(c2)oc2ccccc23)c2ccc3c(c2)oc2ccc(N(c4ccccc4)c4ccccc4)cc23)c1 ; Fc1cccc(N(c2ccc3c(c2)oc2ccccc23)c2ccc3c(c2)oc2ccc(N(c4ccccc4)c4ccccc4)cc23)c1]

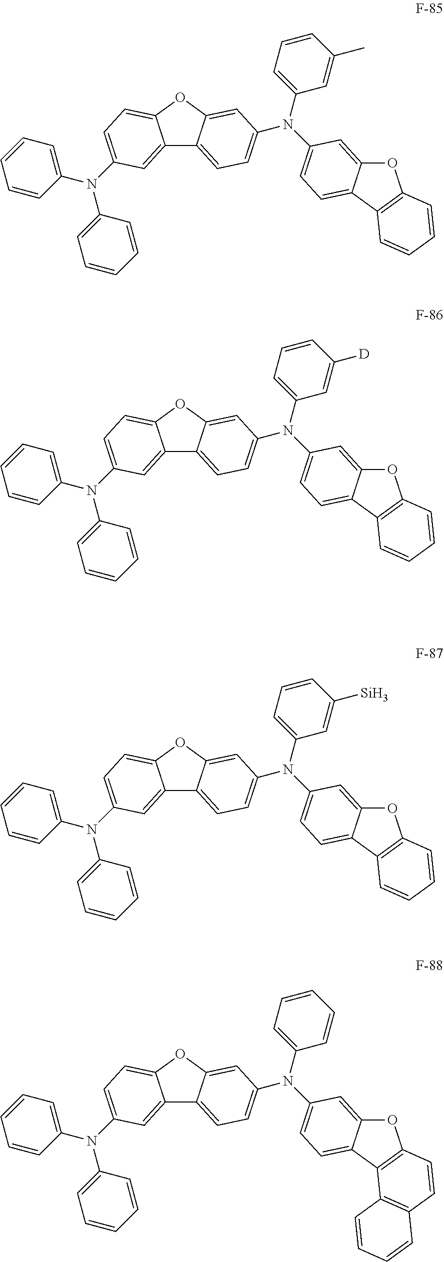

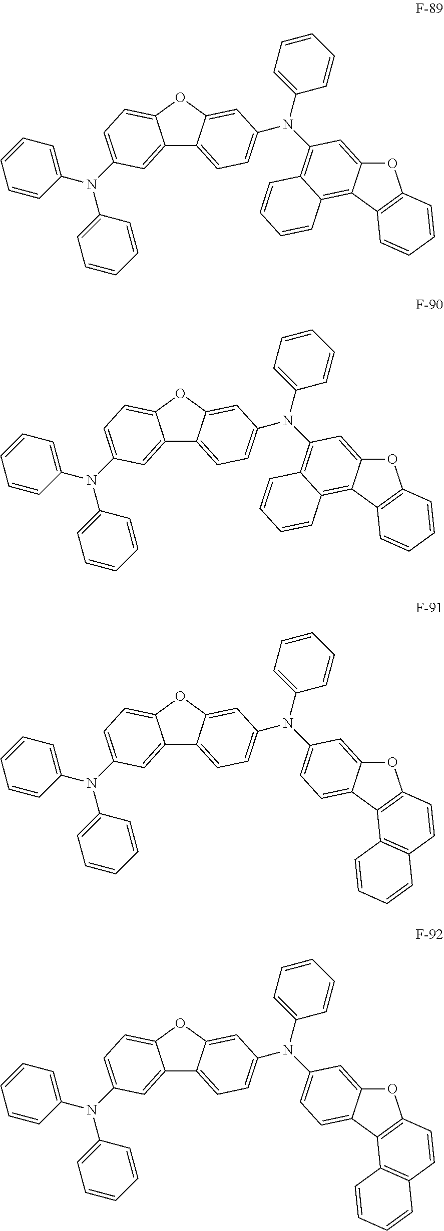 [SMILES: c1ccc(N(c2ccccc2)c2ccc3oc4cc(N(c5ccccc5)c5cc6oc7ccccc7c6c6ccccc56)ccc4c3c2)cc1 ; c1ccc(N(c2ccccc2)c2ccc3oc4cc(N(c5ccccc5)c5cc6oc7ccccc7c6c6ccccc56)ccc4c3c2)cc1 ; c1ccc(N(c2ccc3c(c2)oc2ccc(N(c4ccccc4)c4ccccc4)cc23)c2ccc3c(c2)oc2ccc4ccccc4c23)cc1 ; c1ccc(N(c2ccc3c(c2)oc2ccc(N(c4ccccc4)c4ccccc4)cc23)c2ccc3c(c2)oc2ccc4ccccc4c23)cc1]

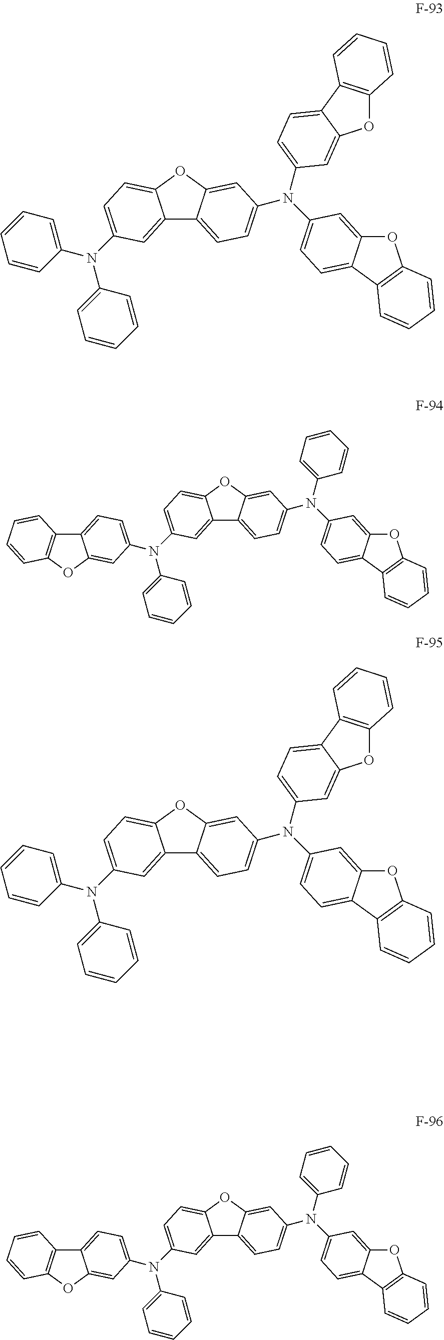 [SMILES: c1ccc(N(c2ccccc2)c2ccc3oc4cc(N(c5ccc6c(c5)oc5ccccc56)c5ccc6c(c5)oc5ccccc56)ccc4c3c2)cc1 ; c1ccc(N(c2ccc3c(c2)oc2ccccc23)c2ccc3c(c2)oc2ccc(N(c4ccccc4)c4ccc5c(c4)oc4ccccc45)cc23)cc1 ; c1ccc(N(c2ccccc2)c2ccc3oc4cc(N(c5ccc6c(c5)oc5ccccc56)c5ccc6c(c5)oc5ccccc56)ccc4c3c2)cc1 ; c1ccc(N(c2ccc3c(c2)oc2ccccc23)c2ccc3c(c2)oc2ccc(N(c4ccccc4)c4ccc5c(c4)oc4ccccc45)cc23)cc1]

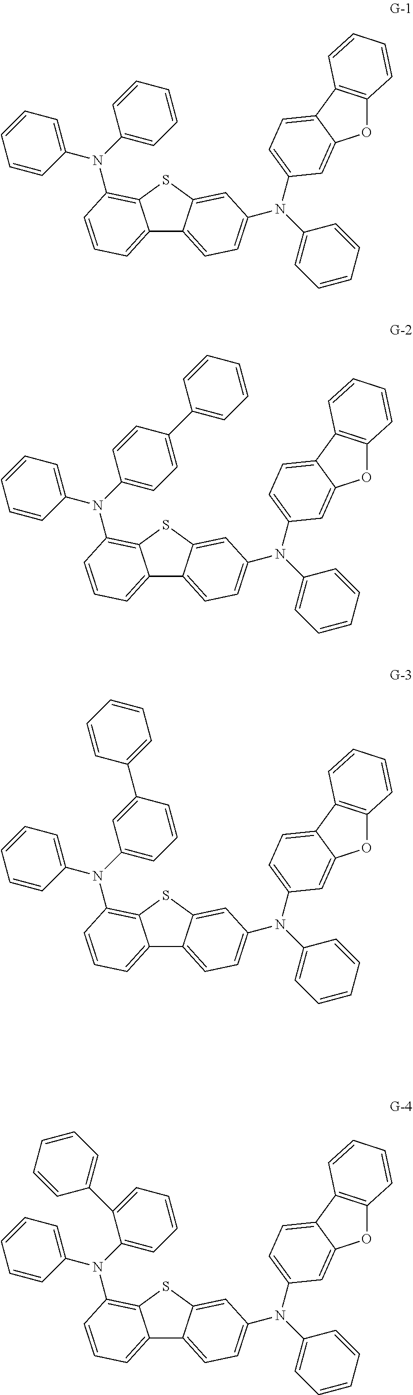 [SMILES: c1ccc(N(c2ccc3c(c2)oc2ccccc23)c2ccc3c(c2)sc2c(N(c4ccccc4)c4ccccc4)cccc23)cc1 ; c1ccc(-c2ccc(N(c3ccccc3)c3cccc4c3sc3cc(N(c5ccccc5)c5ccc6c(c5)oc5ccccc56)ccc34)cc2)cc1 ; c1ccc(-c2cccc(N(c3ccccc3)c3cccc4c3sc3cc(N(c5ccccc5)c5ccc6c(c5)oc5ccccc56)ccc34)c2)cc1 ; c1ccc(-c2ccccc2N(c2ccccc2)c2cccc3c2sc2cc(N(c4ccccc4)c4ccc5c(c4)oc4ccccc45)ccc23)cc1]

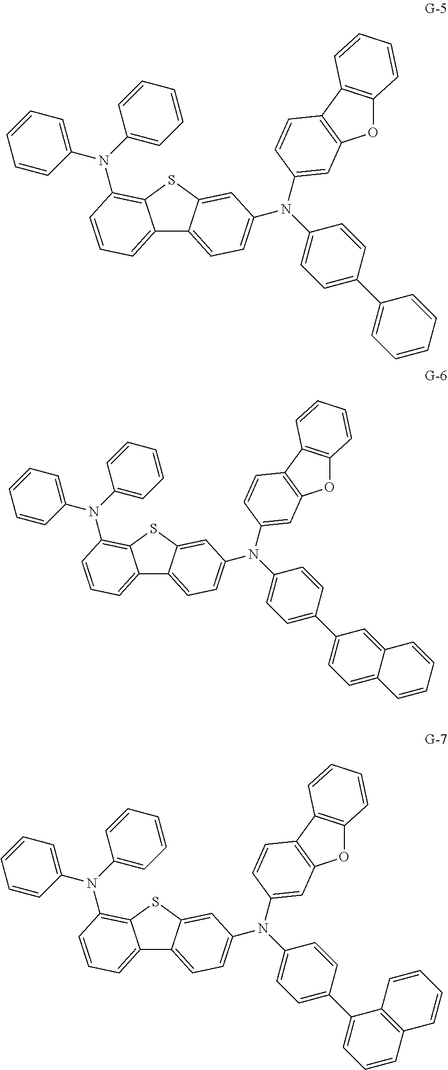 [SMILES: c1ccc(-c2ccc(N(c3ccc4c(c3)oc3ccccc34)c3ccc4c(c3)sc3c(N(c5ccccc5)c5ccccc5)cccc34)cc2)cc1 ; c1ccc(N(c2ccccc2)c2cccc3c2sc2cc(N(c4ccc(-c5ccc6ccccc6c5)cc4)c4ccc5c(c4)oc4ccccc45)ccc23)cc1 ; c1ccc(N(c2ccccc2)c2cccc3c2sc2cc(N(c4ccc(-c5cccc6ccccc56)cc4)c4ccc5c(c4)oc4ccccc45)ccc23)cc1]

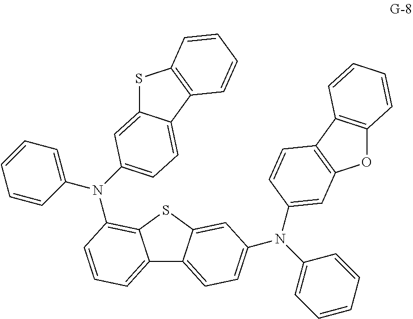 [SMILES: c1ccc(N(c2ccc3c(c2)oc2ccccc23)c2ccc3c(c2)sc2c(N(c4ccccc4)c4ccc5c(c4)sc4ccccc45)cccc23)cc1]

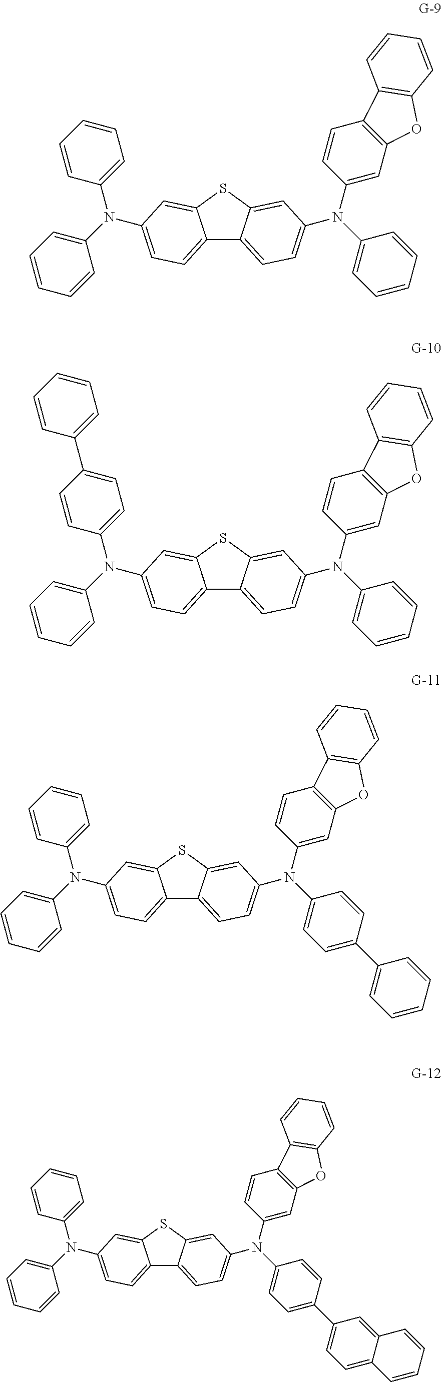 [SMILES: c1ccc(N(c2ccccc2)c2ccc3c(c2)sc2cc(N(c4ccccc4)c4ccc5c(c4)oc4ccccc45)ccc23)cc1 ; c1ccc(-c2ccc(N(c3ccccc3)c3ccc4c(c3)sc3cc(N(c5ccccc5)c5ccc6c(c5)oc5ccccc56)ccc34)cc2)cc1 ; c1ccc(-c2ccc(N(c3ccc4c(c3)oc3ccccc34)c3ccc4c(c3)sc3cc(N(c5ccccc5)c5ccccc5)ccc34)cc2)cc1 ; c1ccc(N(c2ccccc2)c2ccc3c(c2)sc2cc(N(c4ccc(-c5ccc6ccccc6c5)cc4)c4ccc5c(c4)oc4ccccc45)ccc23)cc1]

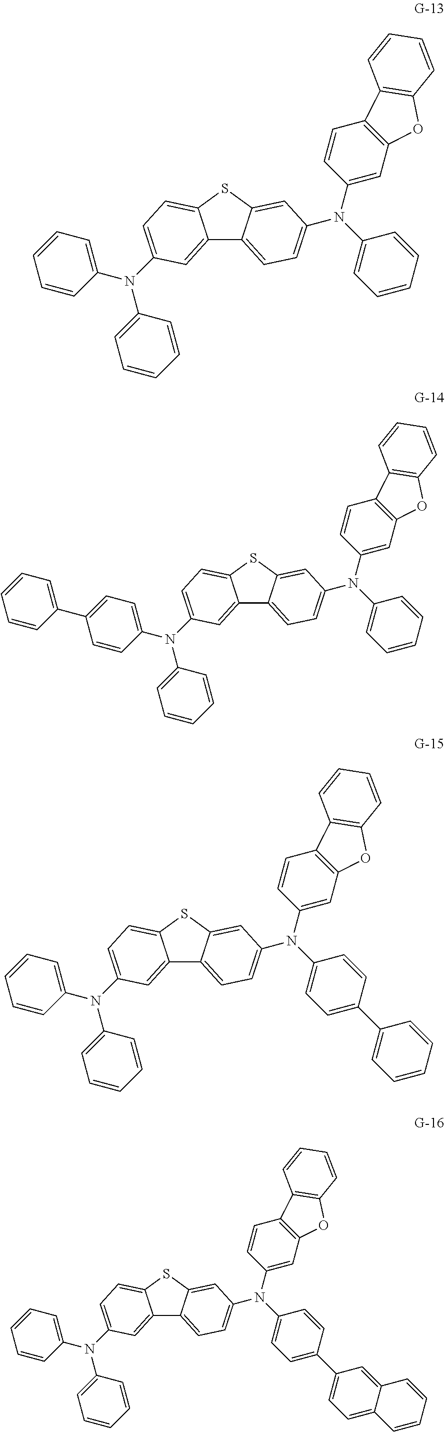 [SMILES: c1ccc(N(c2ccc3c(c2)oc2ccccc23)c2ccc3c(c2)sc2ccc(N(c4ccccc4)c4ccccc4)cc23)cc1 ; c1ccc(-c2ccc(N(c3ccccc3)c3ccc4sc5cc(N(c6ccccc6)c6ccc7c(c6)oc6ccccc67)ccc5c4c3)cc2)cc1 ; c1ccc(-c2ccc(N(c3ccc4c(c3)oc3ccccc34)c3ccc4c(c3)sc3ccc(N(c5ccccc5)c5ccccc5)cc34)cc2)cc1 ; c1ccc(N(c2ccccc2)c2ccc3sc4cc(N(c5ccc(-c6ccc7ccccc7c6)cc5)c5ccc6c(c5)oc5ccccc56)ccc4c3c2)cc1]

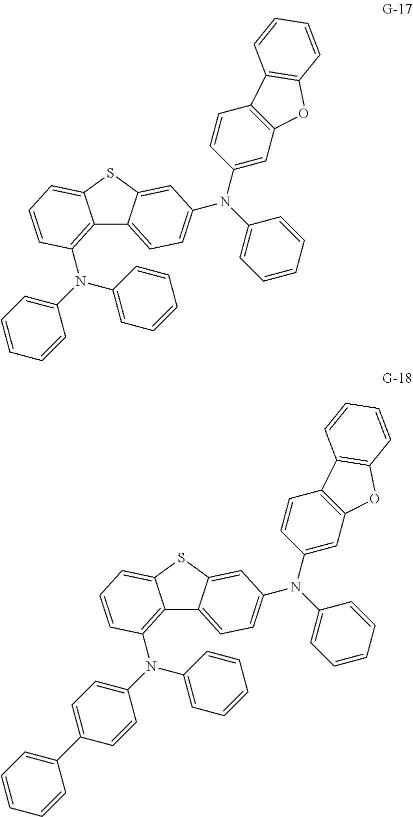 [SMILES: c1ccc(N(c2ccc3c(c2)oc2ccccc23)c2ccc3c(c2)sc2cccc(N(c4ccccc4)c4ccccc4)c23)cc1 ; c1ccc(-c2ccc(N(c3ccccc3)c3cccc4sc5cc(N(c6ccccc6)c6ccc7c(c6)oc6ccccc67)ccc5c34)cc2)cc1]

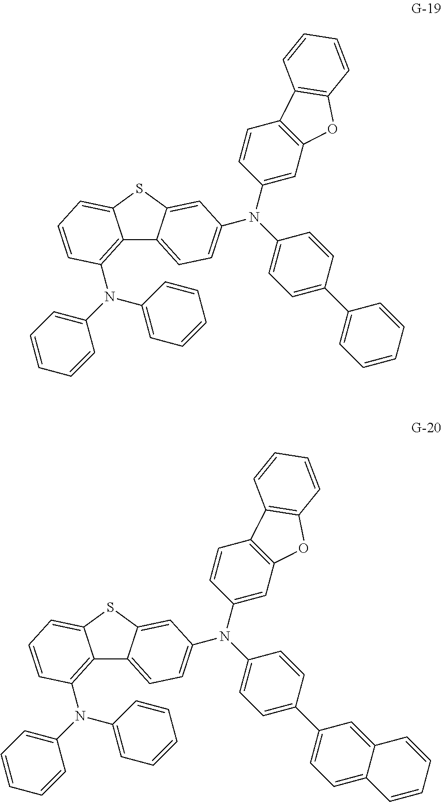 [SMILES: c1ccc(-c2ccc(N(c3ccc4c(c3)oc3ccccc34)c3ccc4c(c3)sc3cccc(N(c5ccccc5)c5ccccc5)c34)cc2)cc1 ; c1ccc(N(c2ccccc2)c2cccc3sc4cc(N(c5ccc(-c6ccc7ccccc7c6)cc5)c5ccc6c(c5)oc5ccccc56)ccc4c23)cc1]

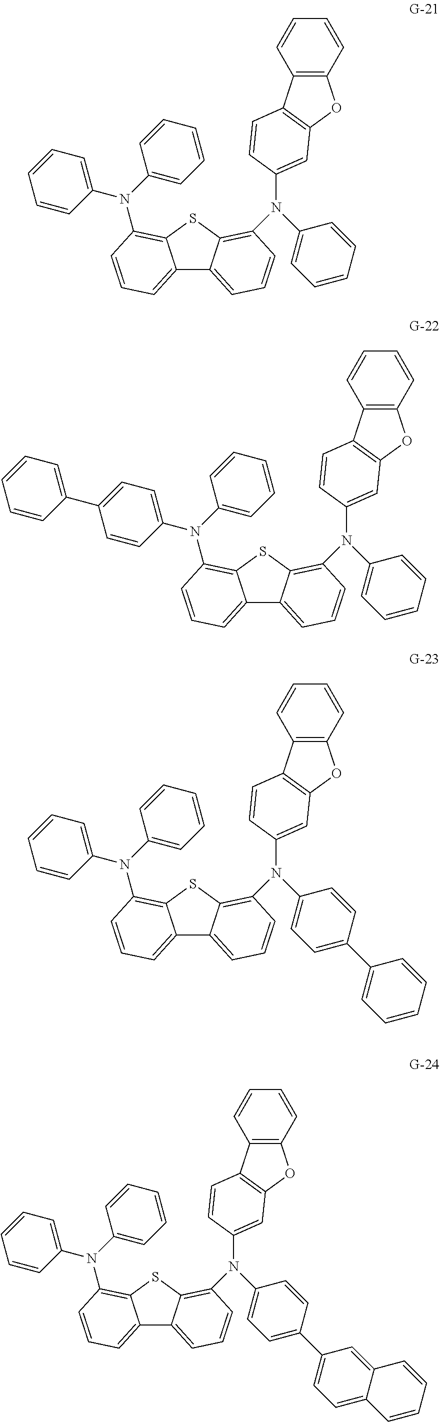 [SMILES: c1ccc(N(c2ccccc2)c2cccc3c2sc2c(N(c4ccccc4)c4ccc5c(c4)oc4ccccc45)cccc23)cc1 ; c1ccc(-c2ccc(N(c3ccccc3)c3cccc4c3sc3c(N(c5ccccc5)c5ccc6c(c5)oc5ccccc56)cccc34)cc2)cc1 ; c1ccc(-c2ccc(N(c3ccc4c(c3)oc3ccccc34)c3cccc4c3sc3c(N(c5ccccc5)c5ccccc5)cccc34)cc2)cc1 ; c1ccc(N(c2ccccc2)c2cccc3c2sc2c(N(c4ccc(-c5ccc6ccccc6c5)cc4)c4ccc5c(c4)oc4ccccc45)cccc23)cc1]

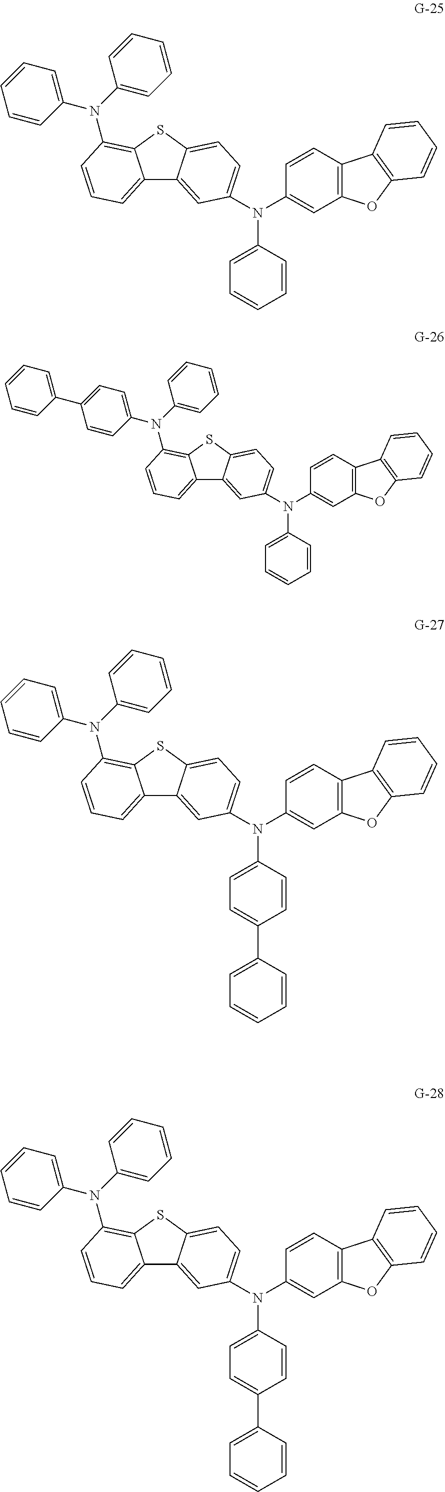 [SMILES: c1ccc(N(c2ccc3c(c2)oc2ccccc23)c2ccc3sc4c(N(c5ccccc5)c5ccccc5)cccc4c3c2)cc1 ; c1ccc(-c2ccc(N(c3ccccc3)c3cccc4c3sc3ccc(N(c5ccccc5)c5ccc6c(c5)oc5ccccc56)cc34)cc2)cc1 ; c1ccc(-c2ccc(N(c3ccc4c(c3)oc3ccccc34)c3ccc4sc5c(N(c6ccccc6)c6ccccc6)cccc5c4c3)cc2)cc1 ; c1ccc(-c2ccc(N(c3ccc4c(c3)oc3ccccc34)c3ccc4sc5c(N(c6ccccc6)c6ccccc6)cccc5c4c3)cc2)cc1]

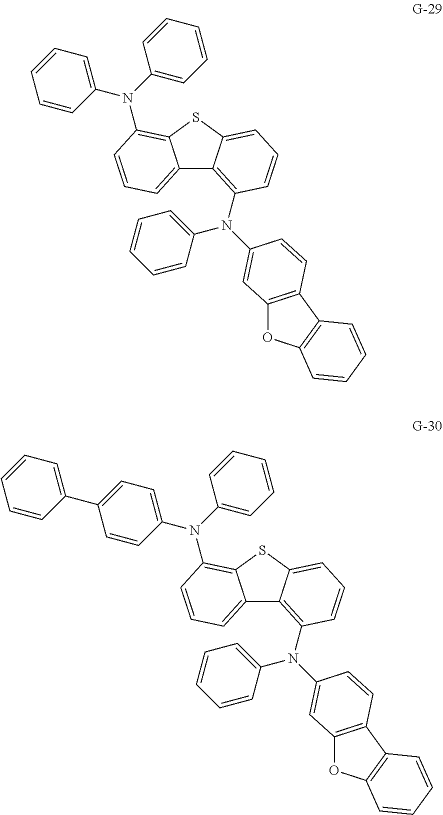 [SMILES: c1ccc(N(c2ccccc2)c2cccc3c2sc2cccc(N(c4ccccc4)c4ccc5c(c4)oc4ccccc45)c23)cc1 ; c1ccc(-c2ccc(N(c3ccccc3)c3cccc4c3sc3cccc(N(c5ccccc5)c5ccc6c(c5)oc5ccccc56)c34)cc2)cc1]

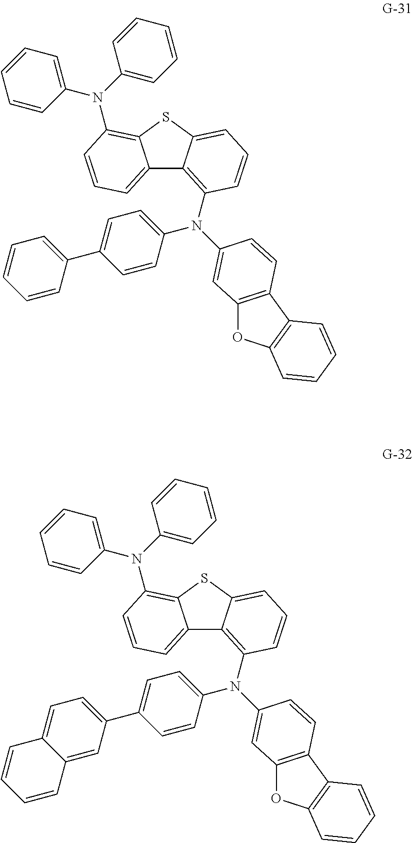 [SMILES: c1ccc(-c2ccc(N(c3ccc4c(c3)oc3ccccc34)c3cccc4sc5c(N(c6ccccc6)c6ccccc6)cccc5c34)cc2)cc1 ; c1ccc(N(c2ccccc2)c2cccc3c2sc2cccc(N(c4ccc(-c5ccc6ccccc6c5)cc4)c4ccc5c(c4)oc4ccccc45)c23)cc1]

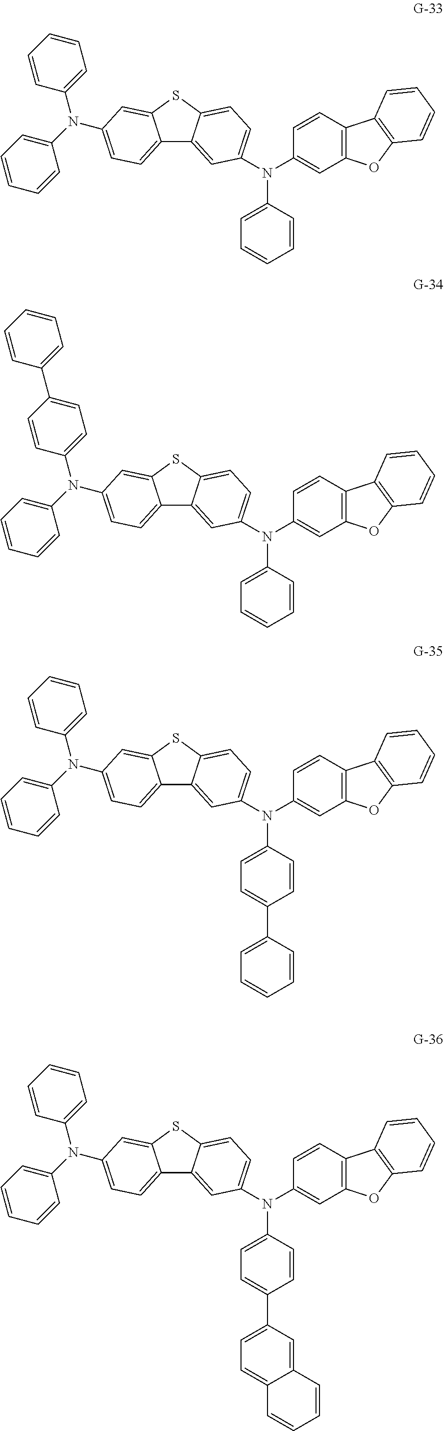 [SMILES: c1ccc(N(c2ccccc2)c2ccc3c(c2)sc2ccc(N(c4ccccc4)c4ccc5c(c4)oc4ccccc45)cc23)cc1 ; c1ccc(-c2ccc(N(c3ccccc3)c3ccc4c(c3)sc3ccc(N(c5ccccc5)c5ccc6c(c5)oc5ccccc56)cc34)cc2)cc1 ; c1ccc(-c2ccc(N(c3ccc4c(c3)oc3ccccc34)c3ccc4sc5cc(N(c6ccccc6)c6ccccc6)ccc5c4c3)cc2)cc1 ; c1ccc(N(c2ccccc2)c2ccc3c(c2)sc2ccc(N(c4ccc(-c5ccc6ccccc6c5)cc4)c4ccc5c(c4)oc4ccccc45)cc23)cc1]

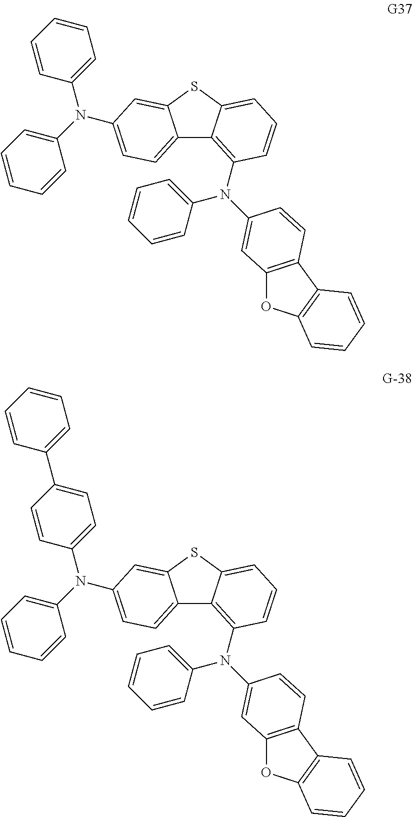 [SMILES: c1ccc(N(c2ccccc2)c2ccc3c(c2)sc2cccc(N(c4ccccc4)c4ccc5c(c4)oc4ccccc45)c23)cc1 ; c1ccc(-c2ccc(N(c3ccccc3)c3ccc4c(c3)sc3cccc(N(c5ccccc5)c5ccc6c(c5)oc5ccccc56)c34)cc2)cc1]

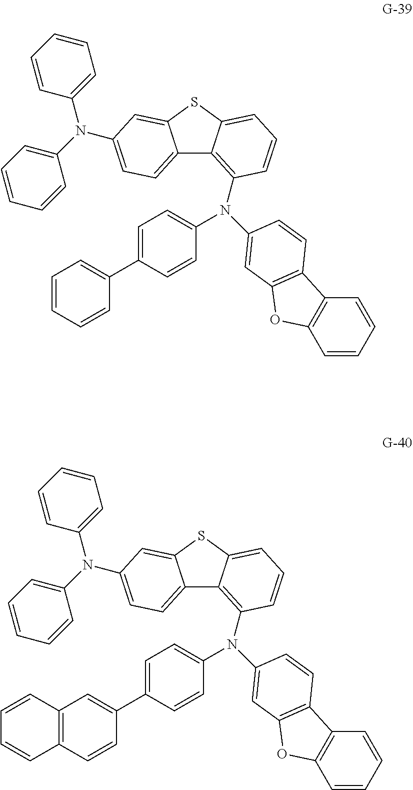 [SMILES: c1ccc(-c2ccc(N(c3ccc4c(c3)oc3ccccc34)c3cccc4sc5cc(N(c6ccccc6)c6ccccc6)ccc5c34)cc2)cc1 ; c1ccc(N(c2ccccc2)c2ccc3c(c2)sc2cccc(N(c4ccc(-c5ccc6ccccc6c5)cc4)c4ccc5c(c4)oc4ccccc45)c23)cc1]

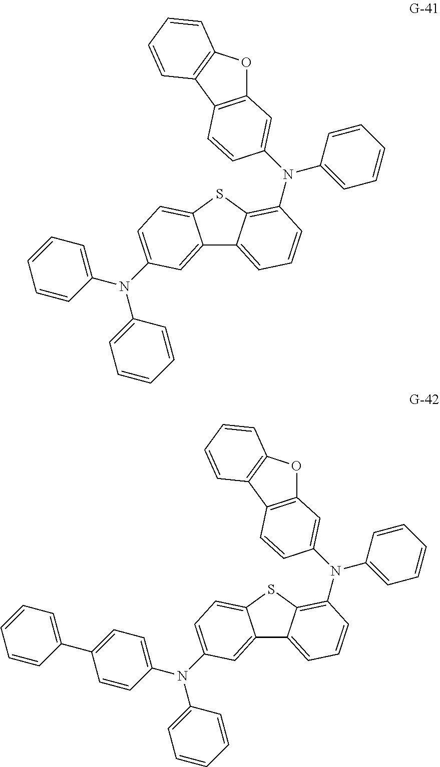 [SMILES: c1ccc(N(c2ccccc2)c2ccc3sc4c(N(c5ccccc5)c5ccc6c(c5)oc5ccccc56)cccc4c3c2)cc1 ; c1ccc(-c2ccc(N(c3ccccc3)c3ccc4sc5c(N(c6ccccc6)c6ccc7c(c6)oc6ccccc67)cccc5c4c3)cc2)cc1]

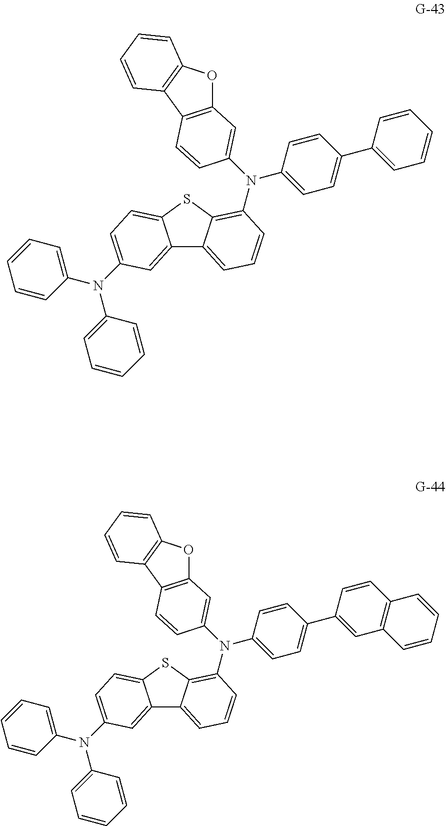 [SMILES: c1ccc(-c2ccc(N(c3ccc4c(c3)oc3ccccc34)c3cccc4c3sc3ccc(N(c5ccccc5)c5ccccc5)cc34)cc2)cc1 ; c1ccc(N(c2ccccc2)c2ccc3sc4c(N(c5ccc(-c6ccc7ccccc7c6)cc5)c5ccc6c(c5)oc5ccccc56)cccc4c3c2)cc1]

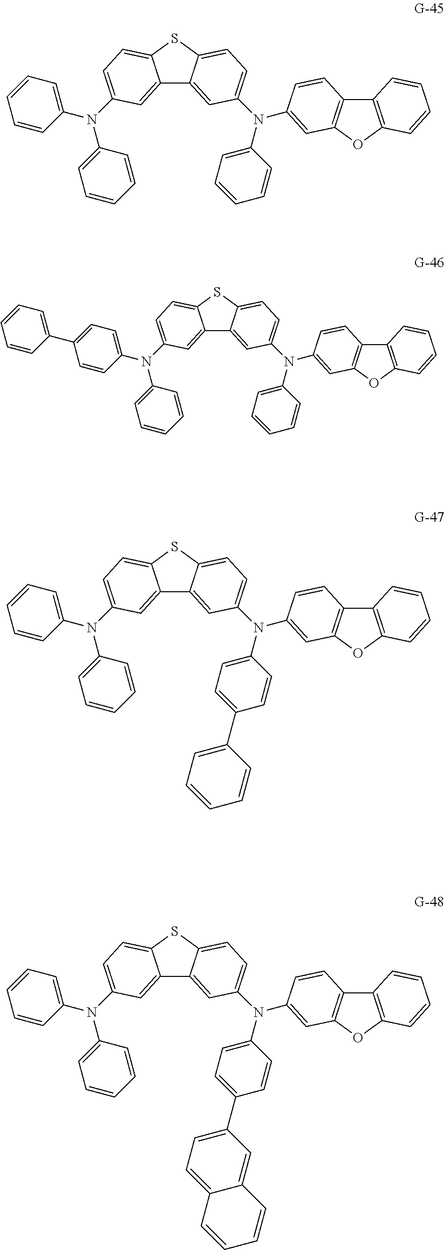

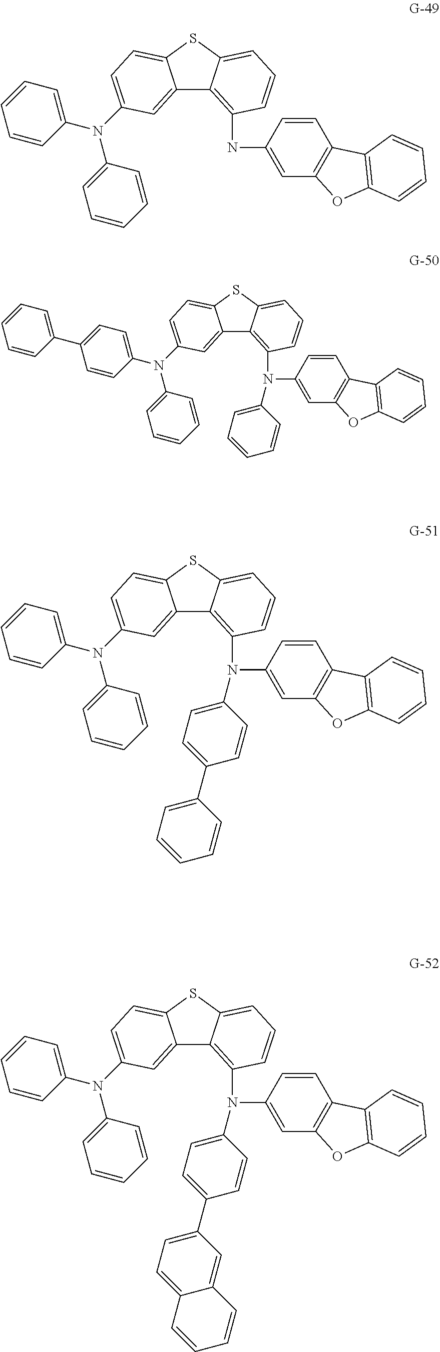 [SMILES: c1ccc(N(c2ccccc2)c2ccc3sc4cccc(Nc5ccc6c(c5)oc5ccccc56)c4c3c2)cc1 ; c1ccc(-c2ccc(N(c3ccccc3)c3ccc4sc5cccc(N(c6ccccc6)c6ccc7c(c6)oc6ccccc67)c5c4c3)cc2)cc1 ; c1ccc(-c2ccc(N(c3ccc4c(c3)oc3ccccc34)c3cccc4sc5ccc(N(c6ccccc6)c6ccccc6)cc5c34)cc2)cc1 ; c1ccc(N(c2ccccc2)c2ccc3sc4cccc(N(c5ccc(-c6ccc7ccccc7c6)cc5)c5ccc6c(c5)oc5ccccc56)c4c3c2)cc1]

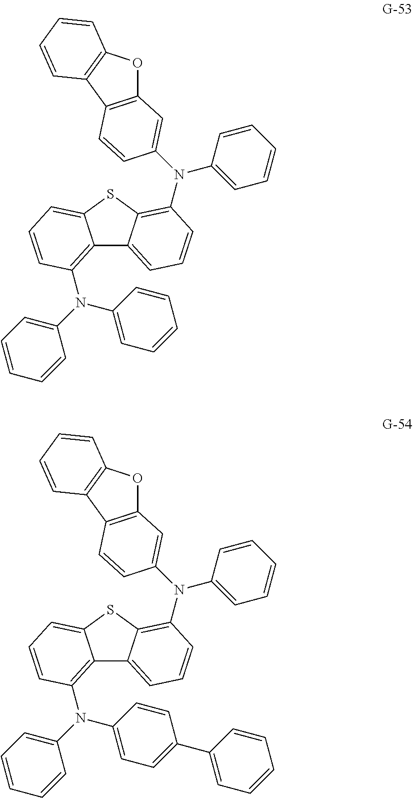 [SMILES: c1ccc(N(c2ccc3c(c2)oc2ccccc23)c2cccc3c2sc2cccc(N(c4ccccc4)c4ccccc4)c23)cc1 ; c1ccc(-c2ccc(N(c3ccccc3)c3cccc4sc5c(N(c6ccccc6)c6ccc7c(c6)oc6ccccc67)cccc5c34)cc2)cc1]

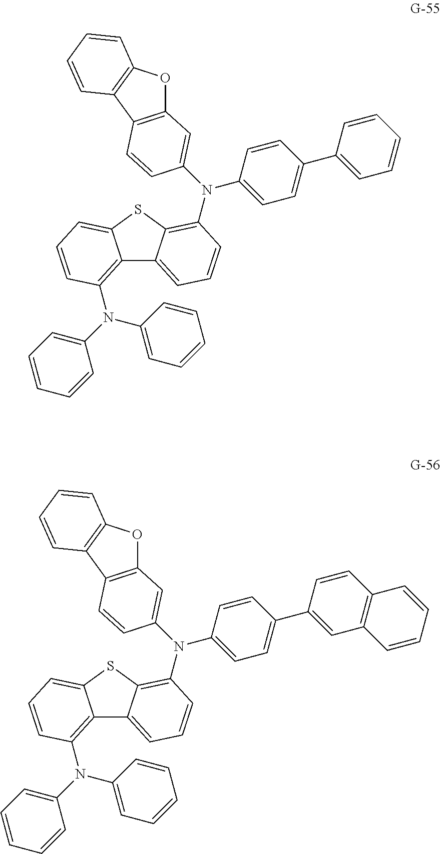 [SMILES: c1ccc(-c2ccc(N(c3ccc4c(c3)oc3ccccc34)c3cccc4c3sc3cccc(N(c5ccccc5)c5ccccc5)c34)cc2)cc1 ; c1ccc(N(c2ccccc2)c2cccc3sc4c(N(c5ccc(-c6ccc7ccccc7c6)cc5)c5ccc6c(c5)oc5ccccc56)cccc4c23)cc1]

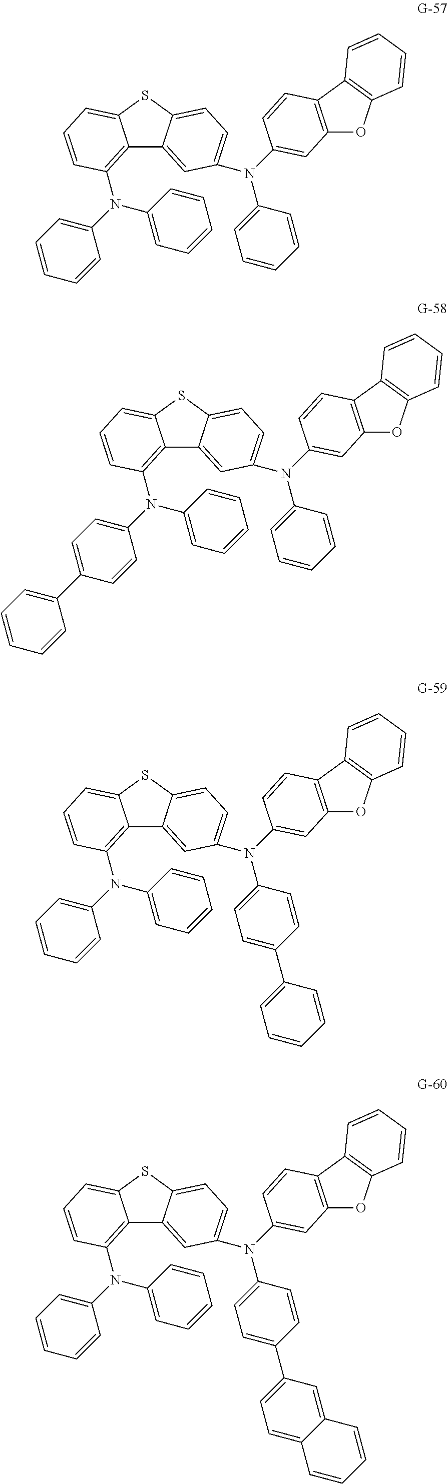 [SMILES: c1ccc(N(c2ccc3c(c2)oc2ccccc23)c2ccc3sc4cccc(N(c5ccccc5)c5ccccc5)c4c3c2)cc1 ; c1ccc(-c2ccc(N(c3ccccc3)c3cccc4sc5ccc(N(c6ccccc6)c6ccc7c(c6)oc6ccccc67)cc5c34)cc2)cc1 ; c1ccc(-c2ccc(N(c3ccc4c(c3)oc3ccccc34)c3ccc4sc5cccc(N(c6ccccc6)c6ccccc6)c5c4c3)cc2)cc1 ; c1ccc(N(c2ccccc2)c2cccc3sc4ccc(N(c5ccc(-c6ccc7ccccc7c6)cc5)c5ccc6c(c5)oc5ccccc56)cc4c23)cc1]

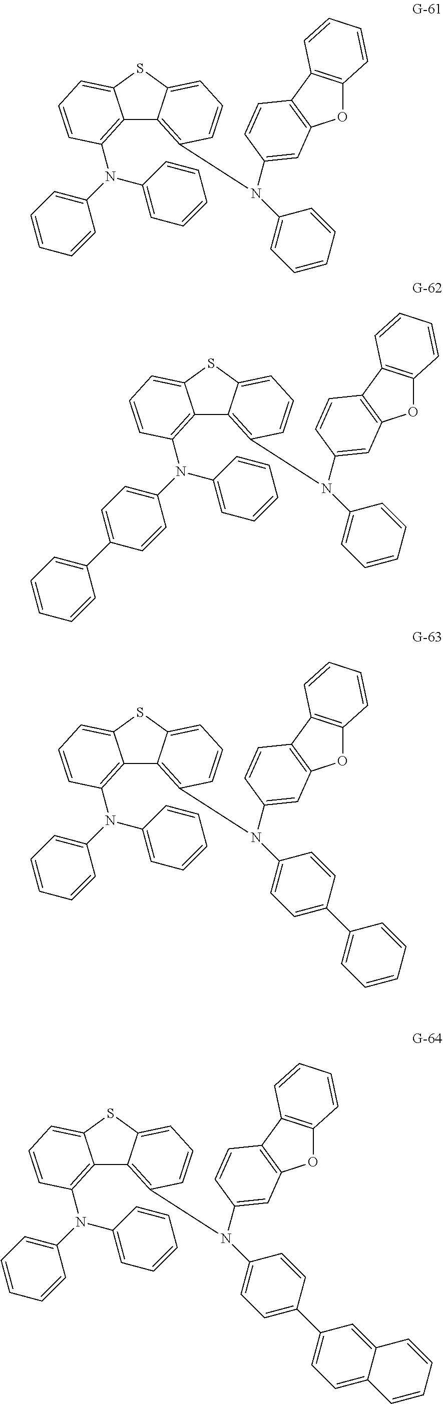 [SMILES: c1ccc(N(c2ccccc2)c2cccc3sc4cccc(N(c5ccccc5)c5ccc6c(c5)oc5ccccc56)c4c23)cc1 ; c1ccc(-c2ccc(N(c3ccccc3)c3cccc4sc5cccc(N(c6ccccc6)c6ccc7c(c6)oc6ccccc67)c5c34)cc2)cc1 ; c1ccc(-c2ccc(N(c3ccc4c(c3)oc3ccccc34)c3cccc4sc5cccc(N(c6ccccc6)c6ccccc6)c5c34)cc2)cc1 ; c1ccc(N(c2ccccc2)c2cccc3sc4cccc(N(c5ccc(-c6ccc7ccccc7c6)cc5)c5ccc6c(c5)oc5ccccc56)c4c23)cc1]

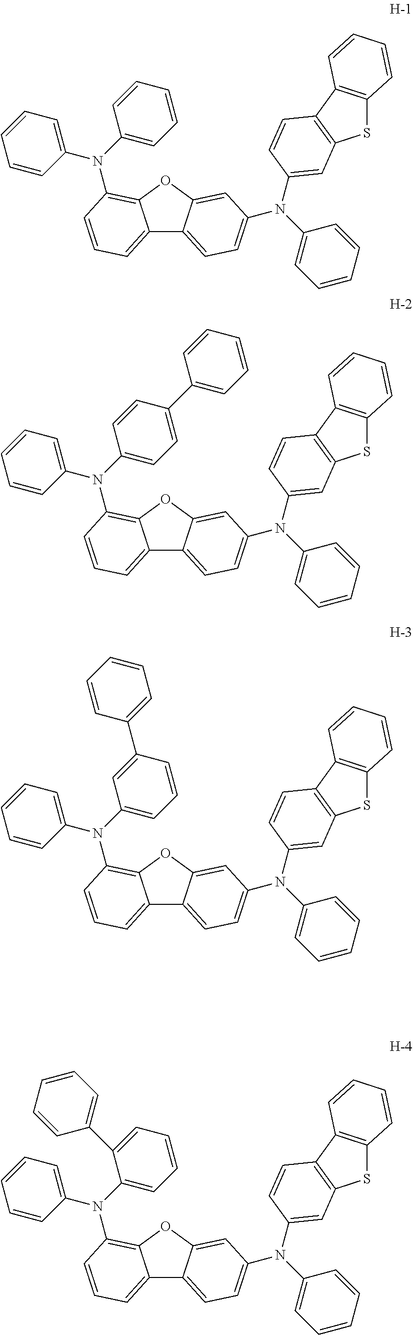 [SMILES: c1ccc(N(c2ccc3c(c2)oc2c(N(c4ccccc4)c4ccccc4)cccc23)c2ccc3c(c2)sc2ccccc23)cc1 ; c1ccc(-c2ccc(N(c3ccccc3)c3cccc4c3oc3cc(N(c5ccccc5)c5ccc6c(c5)sc5ccccc56)ccc34)cc2)cc1 ; c1ccc(-c2cccc(N(c3ccccc3)c3cccc4c3oc3cc(N(c5ccccc5)c5ccc6c(c5)sc5ccccc56)ccc34)c2)cc1 ; c1ccc(-c2ccccc2N(c2ccccc2)c2cccc3c2oc2cc(N(c4ccccc4)c4ccc5c(c4)sc4ccccc45)ccc23)cc1]

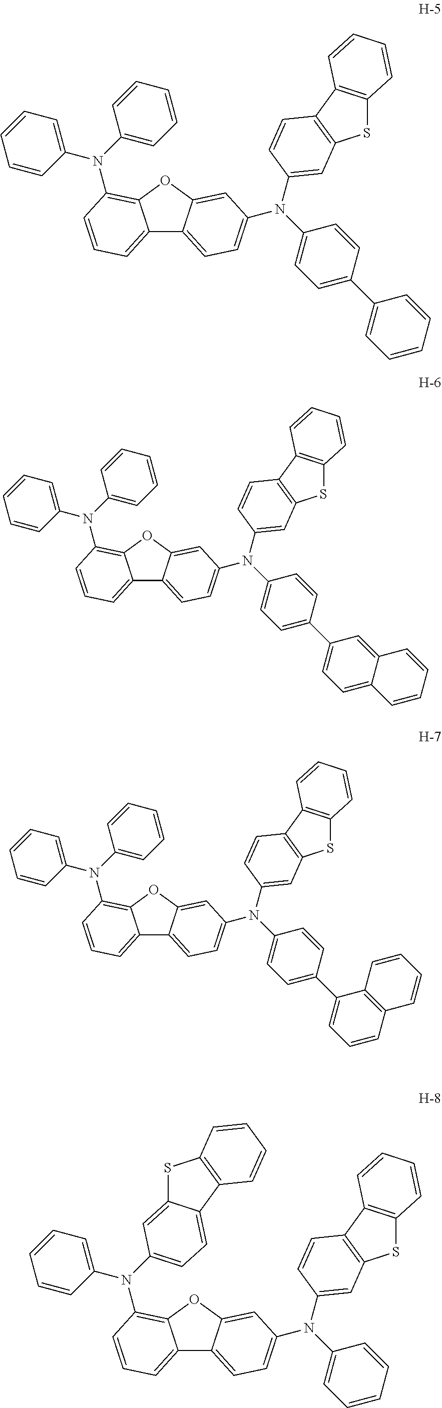 [SMILES: c1ccc(-c2ccc(N(c3ccc4c(c3)oc3c(N(c5ccccc5)c5ccccc5)cccc34)c3ccc4c(c3)sc3ccccc34)cc2)cc1 ; c1ccc(N(c2ccccc2)c2cccc3c2oc2cc(N(c4ccc(-c5ccc6ccccc6c5)cc4)c4ccc5c(c4)sc4ccccc45)ccc23)cc1 ; c1ccc(N(c2ccccc2)c2cccc3c2oc2cc(N(c4ccc(-c5cccc6ccccc56)cc4)c4ccc5c(c4)sc4ccccc45)ccc23)cc1 ; c1ccc(N(c2ccc3c(c2)oc2c(N(c4ccccc4)c4ccc5c(c4)sc4ccccc45)cccc23)c2ccc3c(c2)sc2ccccc23)cc1]

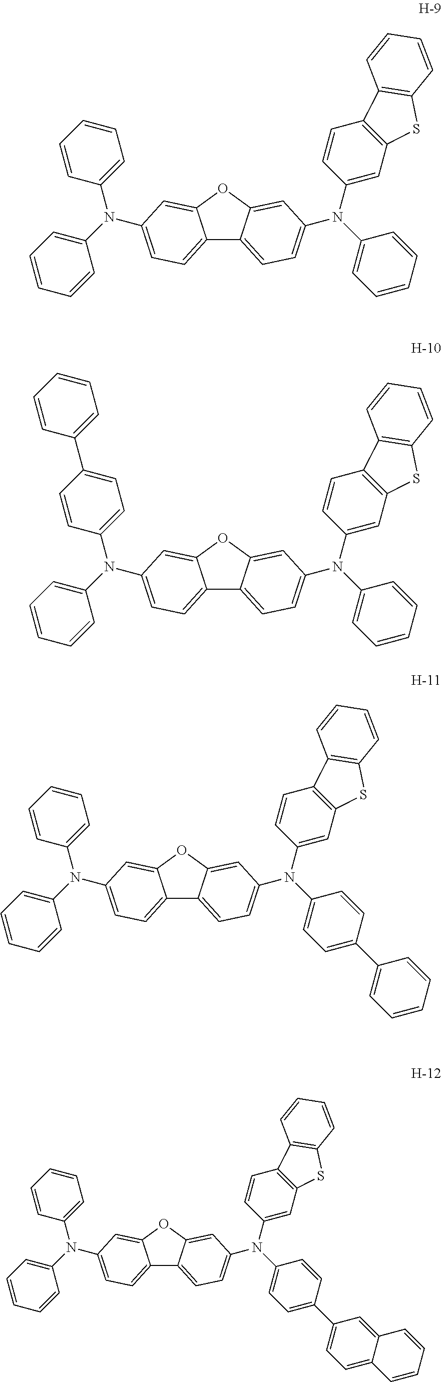 [SMILES: c1ccc(N(c2ccccc2)c2ccc3c(c2)oc2cc(N(c4ccccc4)c4ccc5c(c4)sc4ccccc45)ccc23)cc1 ; c1ccc(-c2ccc(N(c3ccccc3)c3ccc4c(c3)oc3cc(N(c5ccccc5)c5ccc6c(c5)sc5ccccc56)ccc34)cc2)cc1 ; c1ccc(-c2ccc(N(c3ccc4c(c3)oc3cc(N(c5ccccc5)c5ccccc5)ccc34)c3ccc4c(c3)sc3ccccc34)cc2)cc1 ; c1ccc(N(c2ccccc2)c2ccc3c(c2)oc2cc(N(c4ccc(-c5ccc6ccccc6c5)cc4)c4ccc5c(c4)sc4ccccc45)ccc23)cc1]

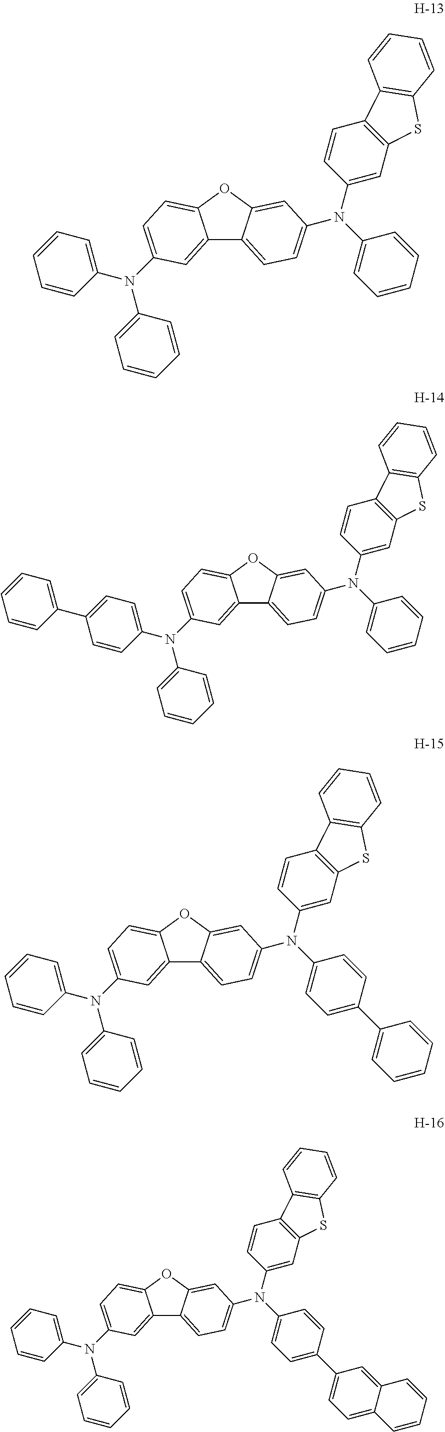 [SMILES: c1ccc(N(c2ccc3c(c2)oc2ccc(N(c4ccccc4)c4ccccc4)cc23)c2ccc3c(c2)sc2ccccc23)cc1 ; c1ccc(-c2ccc(N(c3ccccc3)c3ccc4oc5cc(N(c6ccccc6)c6ccc7c(c6)sc6ccccc67)ccc5c4c3)cc2)cc1 ; c1ccc(-c2ccc(N(c3ccc4c(c3)oc3ccc(N(c5ccccc5)c5ccccc5)cc34)c3ccc4c(c3)sc3ccccc34)cc2)cc1 ; c1ccc(N(c2ccccc2)c2ccc3oc4cc(N(c5ccc(-c6ccc7ccccc7c6)cc5)c5ccc6c(c5)sc5ccccc56)ccc4c3c2)cc1]

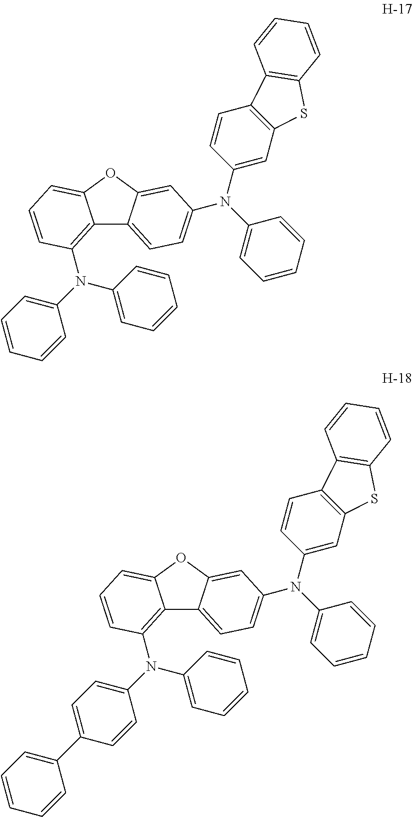 [SMILES: c1ccc(N(c2ccc3c(c2)oc2cccc(N(c4ccccc4)c4ccccc4)c23)c2ccc3c(c2)sc2ccccc23)cc1 ; c1ccc(-c2ccc(N(c3ccccc3)c3cccc4oc5cc(N(c6ccccc6)c6ccc7c(c6)sc6ccccc67)ccc5c34)cc2)cc1]

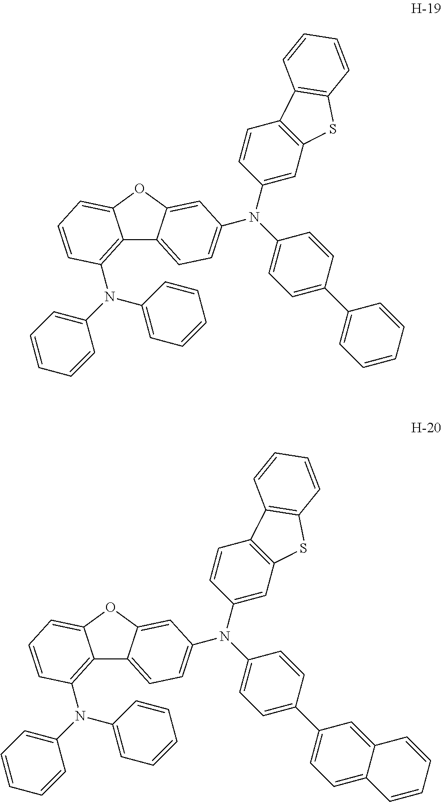 [SMILES: c1ccc(-c2ccc(N(c3ccc4c(c3)oc3cccc(N(c5ccccc5)c5ccccc5)c34)c3ccc4c(c3)sc3ccccc34)cc2)cc1 ; c1ccc(N(c2ccccc2)c2cccc3oc4cc(N(c5ccc(-c6ccc7ccccc7c6)cc5)c5ccc6c(c5)sc5ccccc56)ccc4c23)cc1]

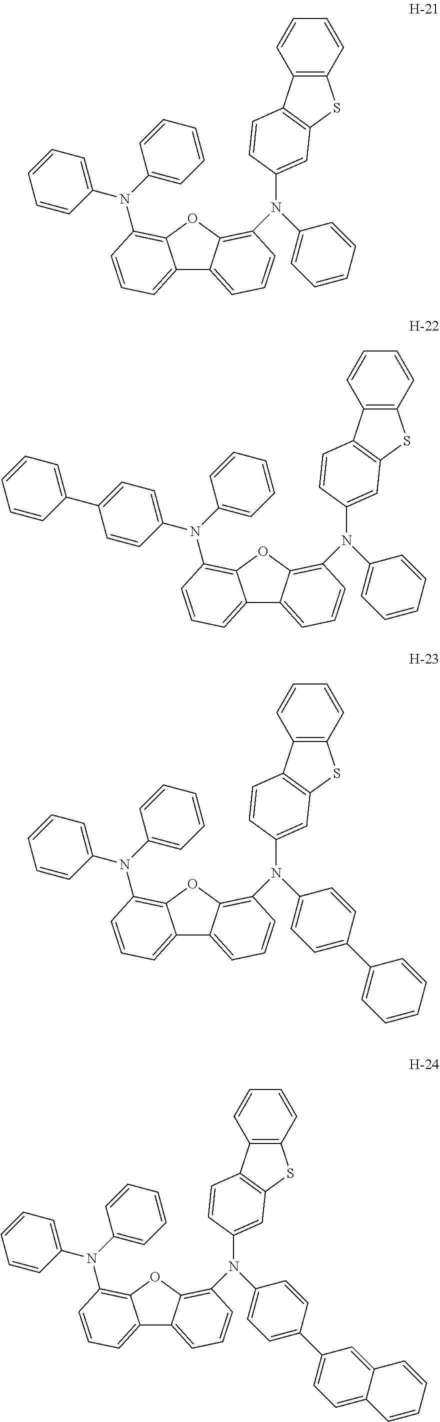 [SMILES: c1ccc(N(c2ccccc2)c2cccc3c2oc2c(N(c4ccccc4)c4ccc5c(c4)sc4ccccc45)cccc23)cc1 ; c1ccc(-c2ccc(N(c3ccccc3)c3cccc4c3oc3c(N(c5ccccc5)c5ccc6c(c5)sc5ccccc56)cccc34)cc2)cc1 ; c1ccc(-c2ccc(N(c3ccc4c(c3)sc3ccccc34)c3cccc4c3oc3c(N(c5ccccc5)c5ccccc5)cccc34)cc2)cc1 ; c1ccc(N(c2ccccc2)c2cccc3c2oc2c(N(c4ccc(-c5ccc6ccccc6c5)cc4)c4ccc5c(c4)sc4ccccc45)cccc23)cc1]

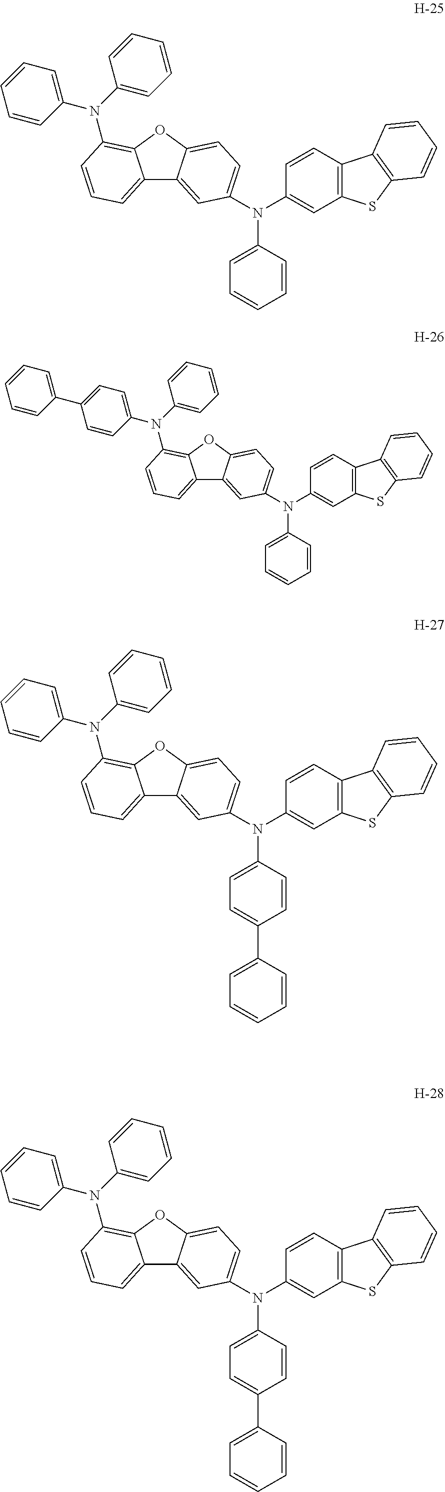 [SMILES: c1ccc(N(c2ccc3c(c2)sc2ccccc23)c2ccc3oc4c(N(c5ccccc5)c5ccccc5)cccc4c3c2)cc1 ; c1ccc(-c2ccc(N(c3ccccc3)c3cccc4c3oc3ccc(N(c5ccccc5)c5ccc6c(c5)sc5ccccc56)cc34)cc2)cc1 ; c1ccc(-c2ccc(N(c3ccc4c(c3)sc3ccccc34)c3ccc4oc5c(N(c6ccccc6)c6ccccc6)cccc5c4c3)cc2)cc1 ; c1ccc(-c2ccc(N(c3ccc4c(c3)sc3ccccc34)c3ccc4oc5c(N(c6ccccc6)c6ccccc6)cccc5c4c3)cc2)cc1]

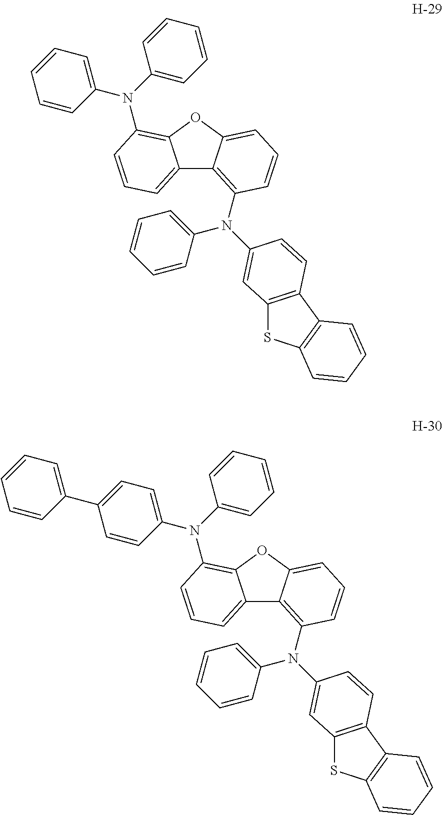 [SMILES: c1ccc(N(c2ccccc2)c2cccc3c2oc2cccc(N(c4ccccc4)c4ccc5c(c4)sc4ccccc45)c23)cc1 ; c1ccc(-c2ccc(N(c3ccccc3)c3cccc4c3oc3cccc(N(c5ccccc5)c5ccc6c(c5)sc5ccccc56)c34)cc2)cc1]

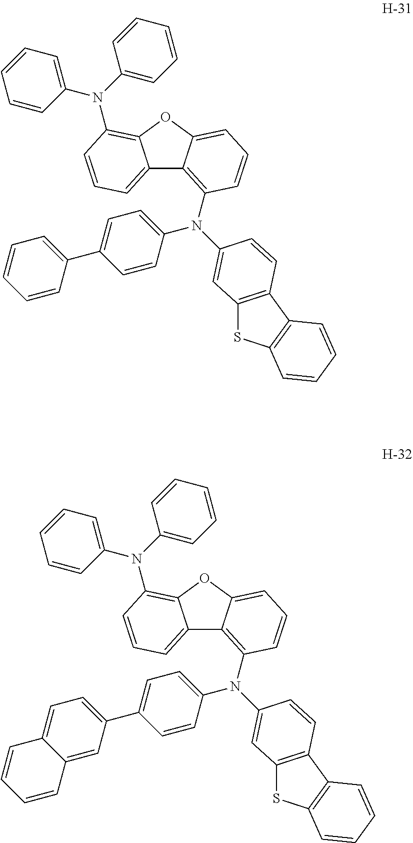 [SMILES: c1ccc(-c2ccc(N(c3ccc4c(c3)sc3ccccc34)c3cccc4oc5c(N(c6ccccc6)c6ccccc6)cccc5c34)cc2)cc1 ; c1ccc(N(c2ccccc2)c2cccc3c2oc2cccc(N(c4ccc(-c5ccc6ccccc6c5)cc4)c4ccc5c(c4)sc4ccccc45)c23)cc1]

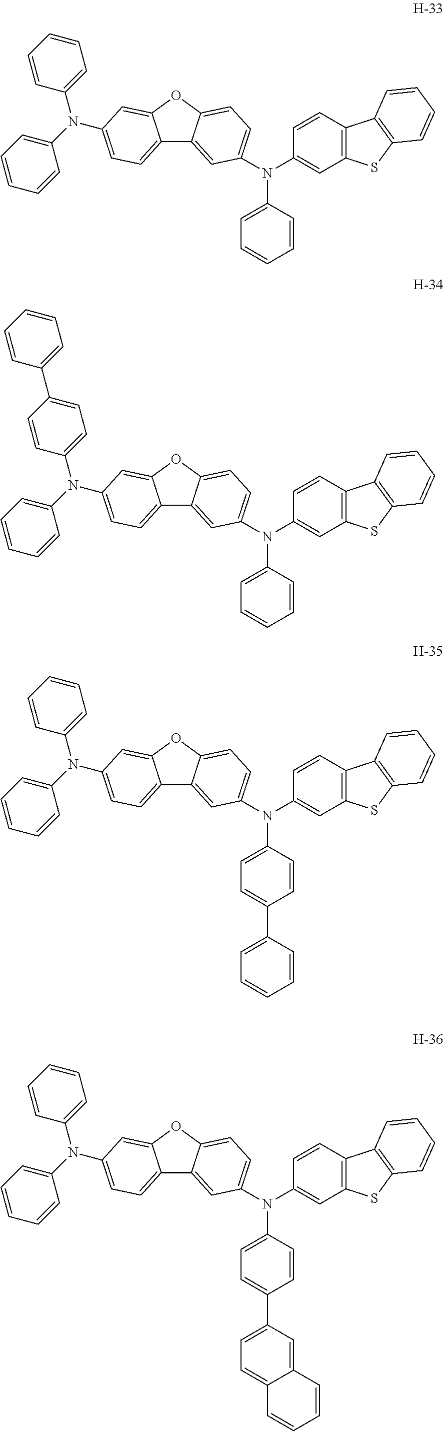 [SMILES: c1ccc(N(c2ccccc2)c2ccc3c(c2)oc2ccc(N(c4ccccc4)c4ccc5c(c4)sc4ccccc45)cc23)cc1 ; c1ccc(-c2ccc(N(c3ccccc3)c3ccc4c(c3)oc3ccc(N(c5ccccc5)c5ccc6c(c5)sc5ccccc56)cc34)cc2)cc1 ; c1ccc(-c2ccc(N(c3ccc4c(c3)sc3ccccc34)c3ccc4oc5cc(N(c6ccccc6)c6ccccc6)ccc5c4c3)cc2)cc1 ; c1ccc(N(c2ccccc2)c2ccc3c(c2)oc2ccc(N(c4ccc(-c5ccc6ccccc6c5)cc4)c4ccc5c(c4)sc4ccccc45)cc23)cc1]

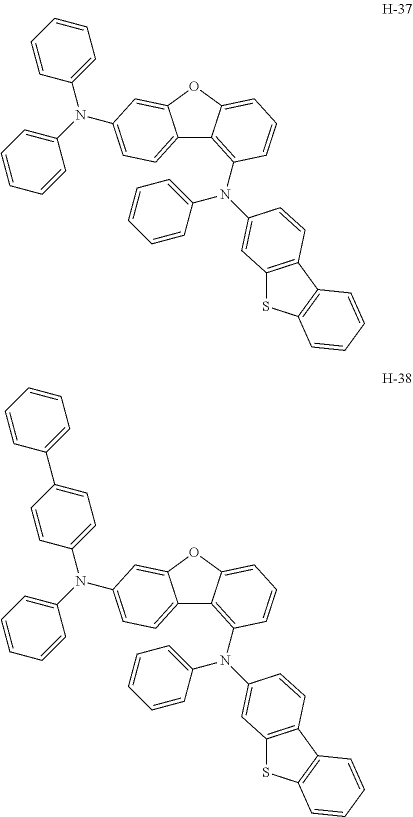 [SMILES: c1ccc(N(c2ccccc2)c2ccc3c(c2)oc2cccc(N(c4ccccc4)c4ccc5c(c4)sc4ccccc45)c23)cc1 ; c1ccc(-c2ccc(N(c3ccccc3)c3ccc4c(c3)oc3cccc(N(c5ccccc5)c5ccc6c(c5)sc5ccccc56)c34)cc2)cc1]

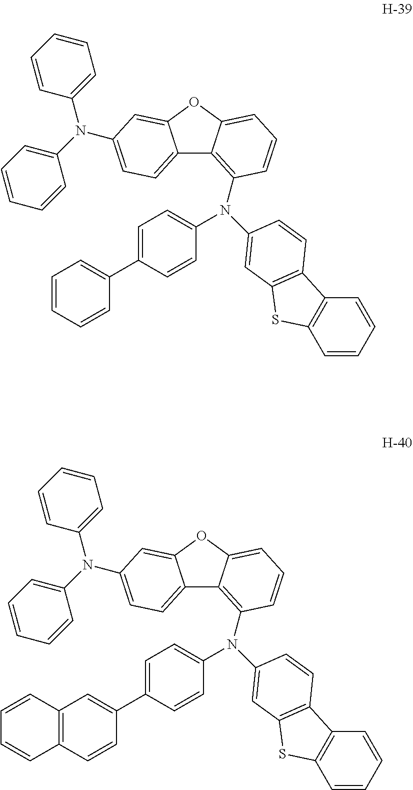 [SMILES: c1ccc(-c2ccc(N(c3ccc4c(c3)sc3ccccc34)c3cccc4oc5cc(N(c6ccccc6)c6ccccc6)ccc5c34)cc2)cc1 ; c1ccc(N(c2ccccc2)c2ccc3c(c2)oc2cccc(N(c4ccc(-c5ccc6ccccc6c5)cc4)c4ccc5c(c4)sc4ccccc45)c23)cc1]

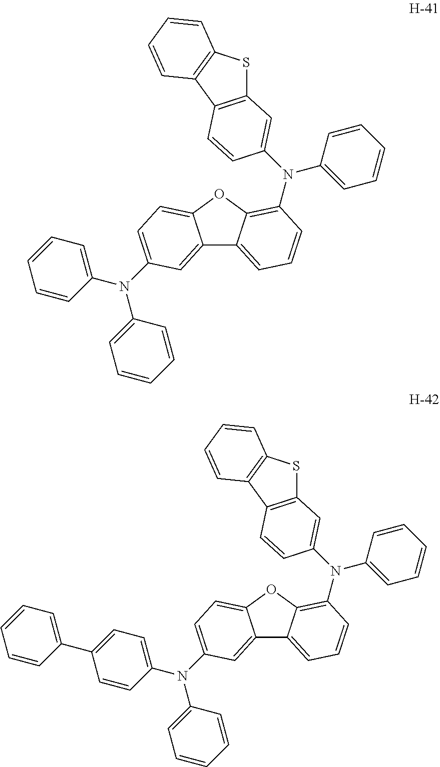 [SMILES: c1ccc(N(c2ccccc2)c2ccc3oc4c(N(c5ccccc5)c5ccc6c(c5)sc5ccccc56)cccc4c3c2)cc1 ; c1ccc(-c2ccc(N(c3ccccc3)c3ccc4oc5c(N(c6ccccc6)c6ccc7c(c6)sc6ccccc67)cccc5c4c3)cc2)cc1]

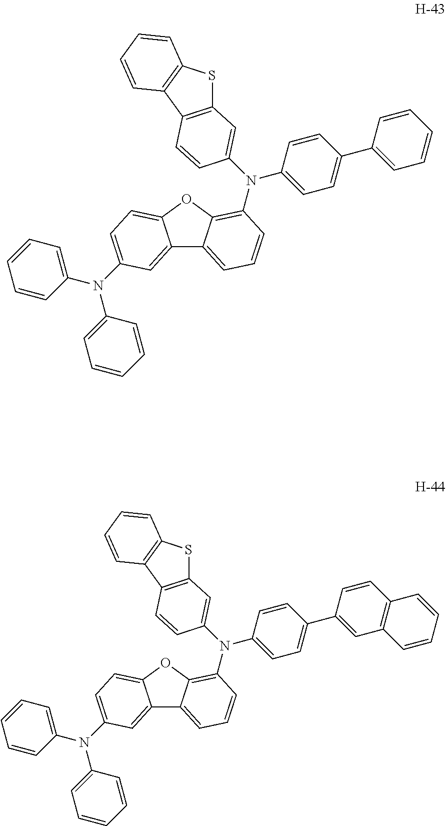 [SMILES: c1ccc(-c2ccc(N(c3ccc4c(c3)sc3ccccc34)c3cccc4c3oc3ccc(N(c5ccccc5)c5ccccc5)cc34)cc2)cc1 ; c1ccc(N(c2ccccc2)c2ccc3oc4c(N(c5ccc(-c6ccc7ccccc7c6)cc5)c5ccc6c(c5)sc5ccccc56)cccc4c3c2)cc1]

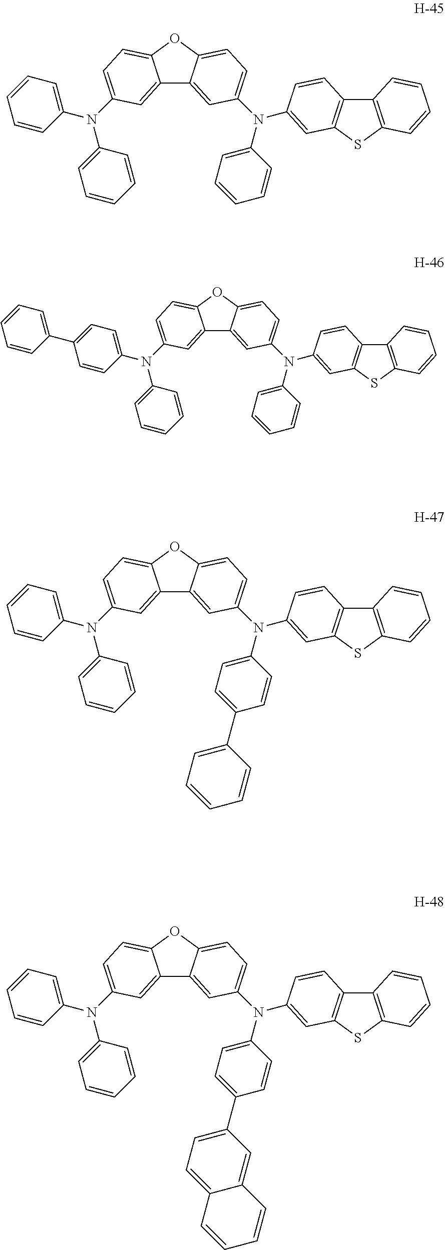

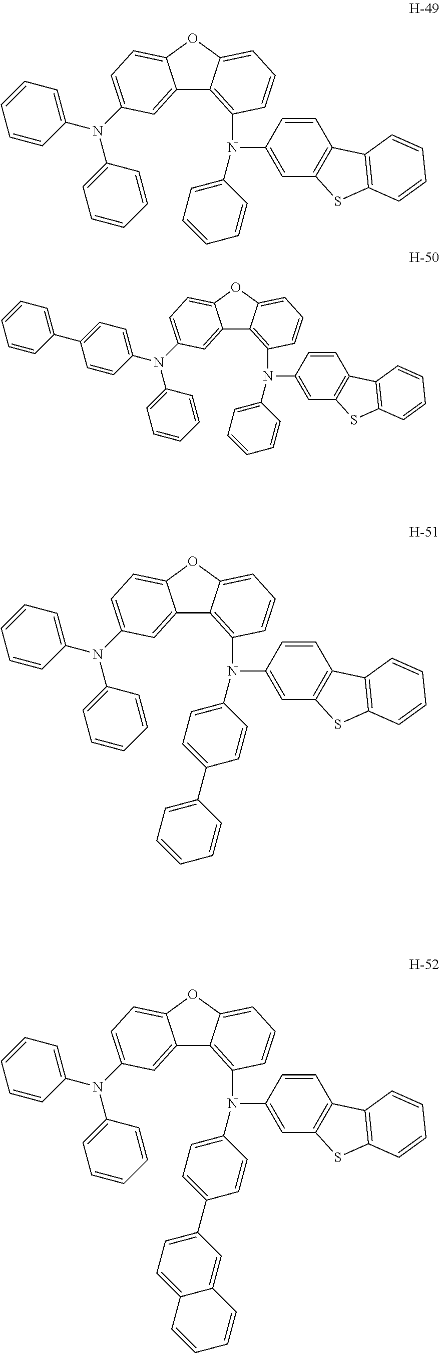 [SMILES: c1ccc(N(c2ccccc2)c2ccc3oc4cccc(N(c5ccccc5)c5ccc6c(c5)sc5ccccc56)c4c3c2)cc1 ; c1ccc(-c2ccc(N(c3ccccc3)c3ccc4oc5cccc(N(c6ccccc6)c6ccc7c(c6)sc6ccccc67)c5c4c3)cc2)cc1 ; c1ccc(-c2ccc(N(c3ccc4c(c3)sc3ccccc34)c3cccc4oc5ccc(N(c6ccccc6)c6ccccc6)cc5c34)cc2)cc1 ; c1ccc(N(c2ccccc2)c2ccc3oc4cccc(N(c5ccc(-c6ccc7ccccc7c6)cc5)c5ccc6c(c5)sc5ccccc56)c4c3c2)cc1]

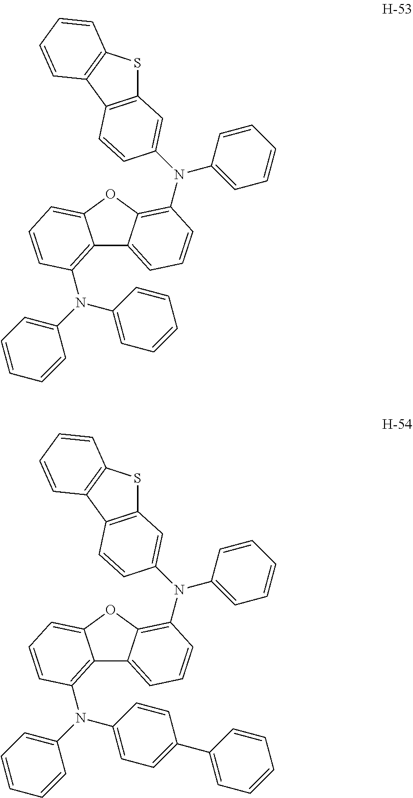 [SMILES: c1ccc(N(c2ccc3c(c2)sc2ccccc23)c2cccc3c2oc2cccc(N(c4ccccc4)c4ccccc4)c23)cc1 ; c1ccc(-c2ccc(N(c3ccccc3)c3cccc4oc5c(N(c6ccccc6)c6ccc7c(c6)sc6ccccc67)cccc5c34)cc2)cc1]

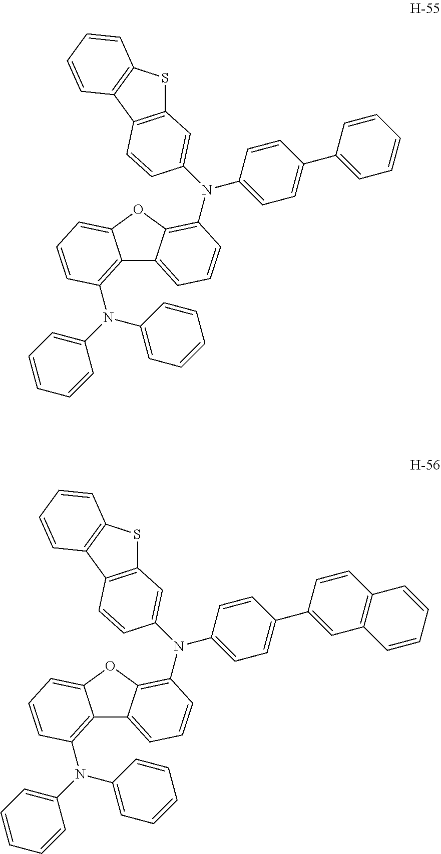 [SMILES: c1ccc(-c2ccc(N(c3ccc4c(c3)sc3ccccc34)c3cccc4c3oc3cccc(N(c5ccccc5)c5ccccc5)c34)cc2)cc1 ; c1ccc(N(c2ccccc2)c2cccc3oc4c(N(c5ccc(-c6ccc7ccccc7c6)cc5)c5ccc6c(c5)sc5ccccc56)cccc4c23)cc1]

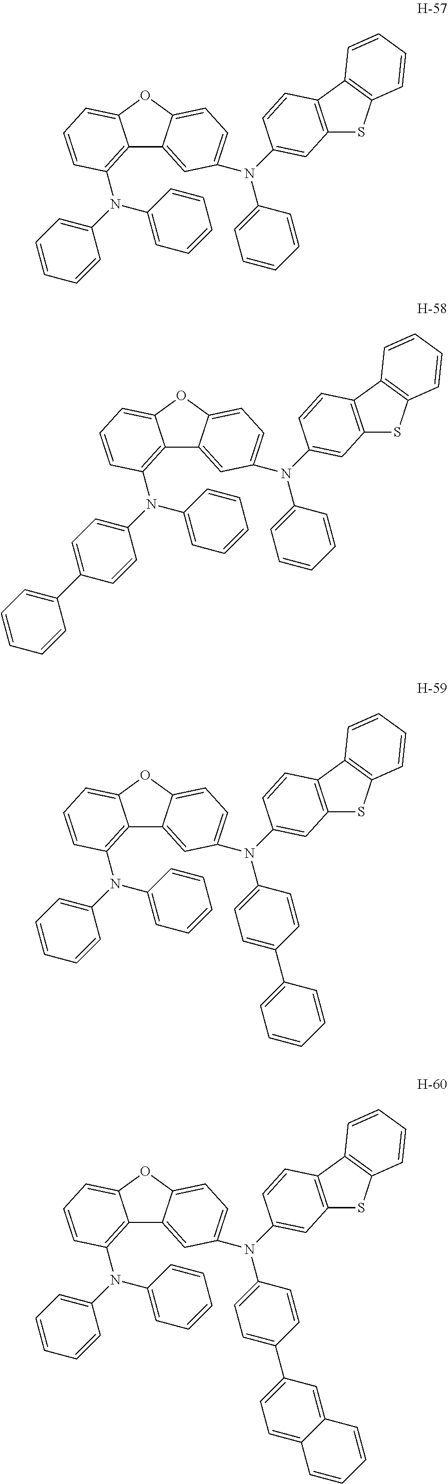 [SMILES: c1ccc(N(c2ccc3c(c2)sc2ccccc23)c2ccc3oc4cccc(N(c5ccccc5)c5ccccc5)c4c3c2)cc1 ; c1ccc(-c2ccc(N(c3ccccc3)c3cccc4oc5ccc(N(c6ccccc6)c6ccc7c(c6)sc6ccccc67)cc5c34)cc2)cc1 ; c1ccc(-c2ccc(N(c3ccc4c(c3)sc3ccccc34)c3ccc4oc5cccc(N(c6ccccc6)c6ccccc6)c5c4c3)cc2)cc1 ; c1ccc(N(c2ccccc2)c2cccc3oc4ccc(N(c5ccc(-c6ccc7ccccc7c6)cc5)c5ccc6c(c5)sc5ccccc56)cc4c23)cc1]

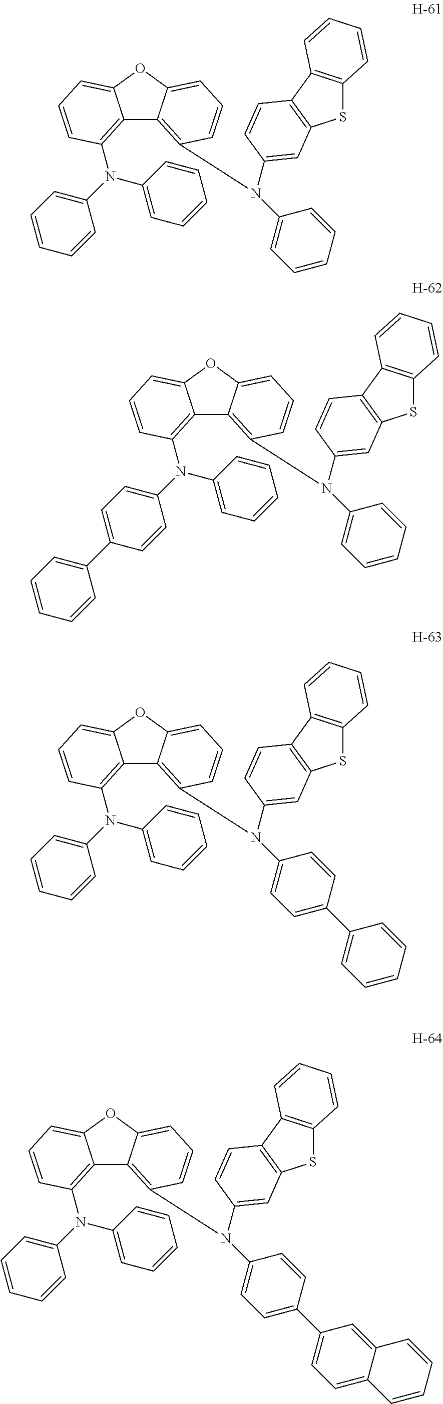 [SMILES: c1ccc(N(c2ccccc2)c2cccc3oc4cccc(N(c5ccccc5)c5ccc6c(c5)sc5ccccc56)c4c23)cc1 ; c1ccc(-c2ccc(N(c3ccccc3)c3cccc4oc5cccc(N(c6ccccc6)c6ccc7c(c6)sc6ccccc67)c5c34)cc2)cc1 ; c1ccc(-c2ccc(N(c3ccc4c(c3)sc3ccccc34)c3cccc4oc5cccc(N(c6ccccc6)c6ccccc6)c5c34)cc2)cc1 ; c1ccc(N(c2ccccc2)c2cccc3oc4cccc(N(c5ccc(-c6ccc7ccccc7c6)cc5)c5ccc6c(c5)sc5ccccc56)c4c23)cc1]

The hole transport layer 141 may be between the anode 110 and the light emitting layer 130, and may facilitate hole transport from the anode 110 to the light emitting layer 130. In an implementation, the hole transport layer 141 may include a material having a HOMO energy level between the work function of the conductor constituting the anode 110 and the HOMO energy level of the material constituting the light emitting layer 130.

The hole transport layer 141 may include, e.g., an amine derivative (e.g., an amine-containing compound).

In an implementation, the hole transport layer 141 may include, e.g., a compound represented by Chemical Formula 4.

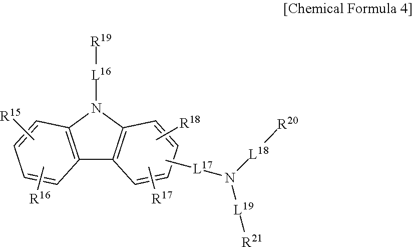

In Chemical Formula 4, R¹⁵ to R¹⁸ may each independently be or include, e.g., hydrogen, deuterium, a substituted or unsubstituted C1 to C10 alkyl group, a substituted or unsubstituted C6 to C30 aryl group, a substituted or unsubstituted C2 to C30 heterocyclic group, or a combination thereof.

In an implementation, R¹⁵ and R¹⁶ may be separate or may be linked to form a ring.

In an implementation, R¹⁷ and R¹⁸ may be separate or may be linked to form a ring.

R¹⁹ to R²¹ may each independently be or include, e.g., a substituted or unsubstituted C6 to C30 aryl group, or a substituted or unsubstituted C2 to C30 heterocyclic group.

L¹⁶ to L¹⁹ may each independently be or include, e.g., a single bond, a substituted or unsubstituted C6 to C30 arylene group, a substituted or unsubstituted divalent C2 to C30 heterocyclic group, or a combination thereof.

In an implementation, R¹⁹ may be, e.g., a substituted or unsubstituted C6 to C30 aryl group. In an implementation, R¹⁹ may be, e.g., a substituted or unsubstituted phenyl group, or a substituted or unsubstituted biphenyl group.

In an implementation, R²⁰ and R²¹ may each independently be, e.g., a substituted or unsubstituted phenyl group, a substituted or unsubstituted biphenyl group, a substituted or unsubstituted fluorenyl group, a substituted or unsubstituted bisfluorene group, a substituted or unsubstituted triphenylenyl group, a substituted or unsubstituted anthracenyl group, a substituted or unsubstituted terphenyl group, a substituted or unsubstituted dibenzofuranyl group, a substituted or unsubstituted dibenzothiophenyl group, or a combination thereof.

In an implementation, the compound represented by Chemical Formula 4 may be, e.g., a compound of the following Group 4.

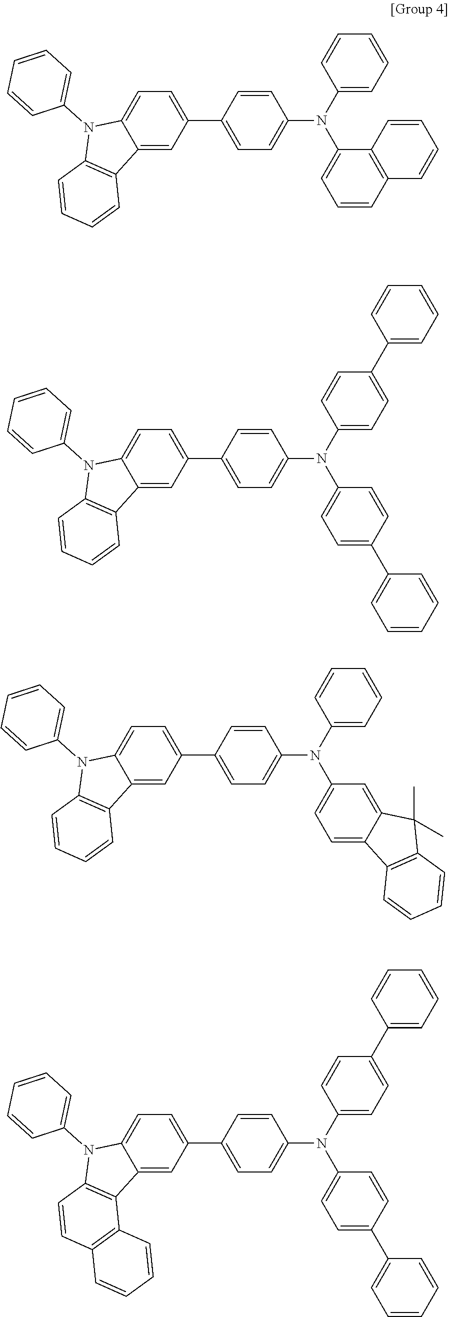

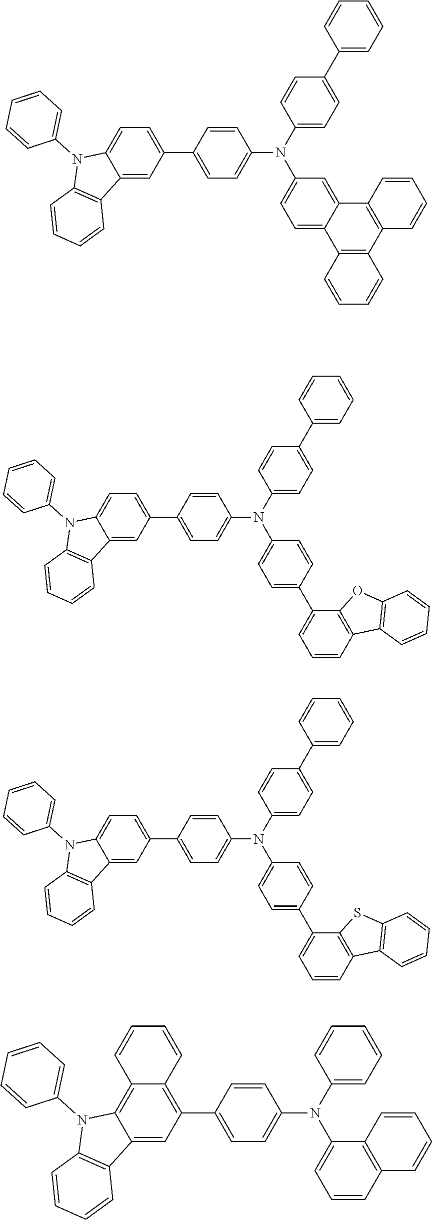 [SMILES: c1ccc(-c2ccc(N(c3ccc(-c4ccc5c(c4)c4ccccc4n5-c4ccccc4)cc3)c3ccc4c5ccccc5c5ccccc5c4c3)cc2)cc1 ; c1ccc(-c2ccc(N(c3ccc(-c4ccc5c(c4)c4ccccc4n5-c4ccccc4)cc3)c3ccc(-c4cccc5c4oc4ccccc45)cc3)cc2)cc1 ; c1ccc(-c2ccc(N(c3ccc(-c4ccc5c(c4)c4ccccc4n5-c4ccccc4)cc3)c3ccc(-c4cccc5c4sc4ccccc45)cc3)cc2)cc1 ; c1ccc(N(c2ccc(-c3cc4c5ccccc5n(-c5ccccc5)c4c4ccccc34)cc2)c2cccc3ccccc23)cc1]

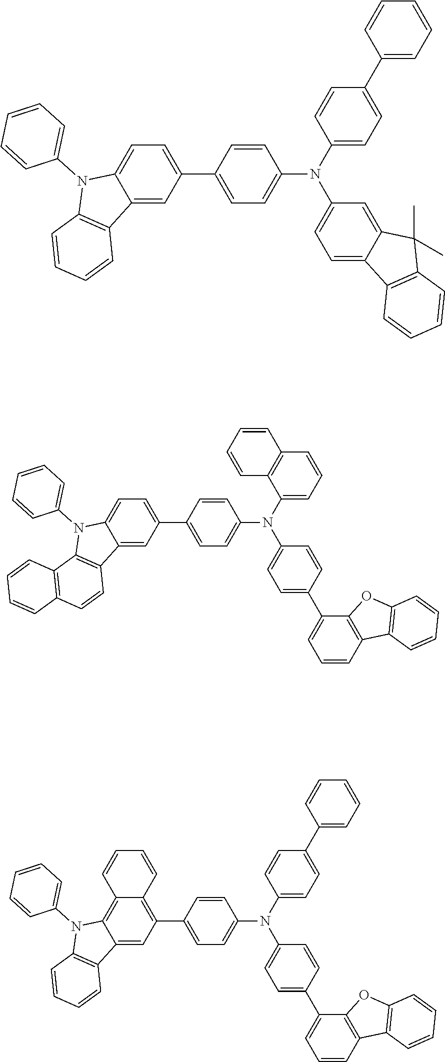 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(-n2c3ccc(-c4ccc(N(c5ccc(-c6cccc7c6oc6ccccc67)cc5)c5cccc6ccccc56)cc4)cc3c3ccc4ccccc4c32)cc1 ; c1ccc(-c2ccc(N(c3ccc(-c4cc5c6ccccc6n(-c6ccccc6)c5c5ccccc45)cc3)c3ccc(-c4cccc5c4oc4ccccc45)cc3)cc2)cc1]

In an implementation, the organic layer 105 may further include a hole injection layer, an electron blocking layer, an electron transport layer, an electron injection layer, and/or a hole blocking layer in addition to the light emitting layer 130, the hole transport auxiliary layer 142, and the hole transport layer 141.

The organic light emitting diode 300 may be manufactured by forming an anode or a cathode on a substrate, forming an organic layer using a dry film formation method such as a vacuum deposition method (evaporation), sputtering, plasma plating, and ion plating, or a solution process, and forming a cathode or an anode thereon.

The aforementioned organic optoelectronic device may be applied to a display device. For example, the organic light emitting diode may be applied to an organic light emitting diode (OLED) display.

The following Examples and Comparative Examples are provided in order to highlight characteristics of one or more embodiments, but it will be understood that the Examples and Comparative Examples are not to be construed as limiting the scope of the embodiments, nor are the Comparative Examples to be construed as being outside the scope of the embodiments. Further, it will be understood that the embodiments are not limited to the particular details described in the Examples and Comparative Examples.

(Preparation of First Compound)

### Synthesis Example 1: Synthesis of Compound A-2

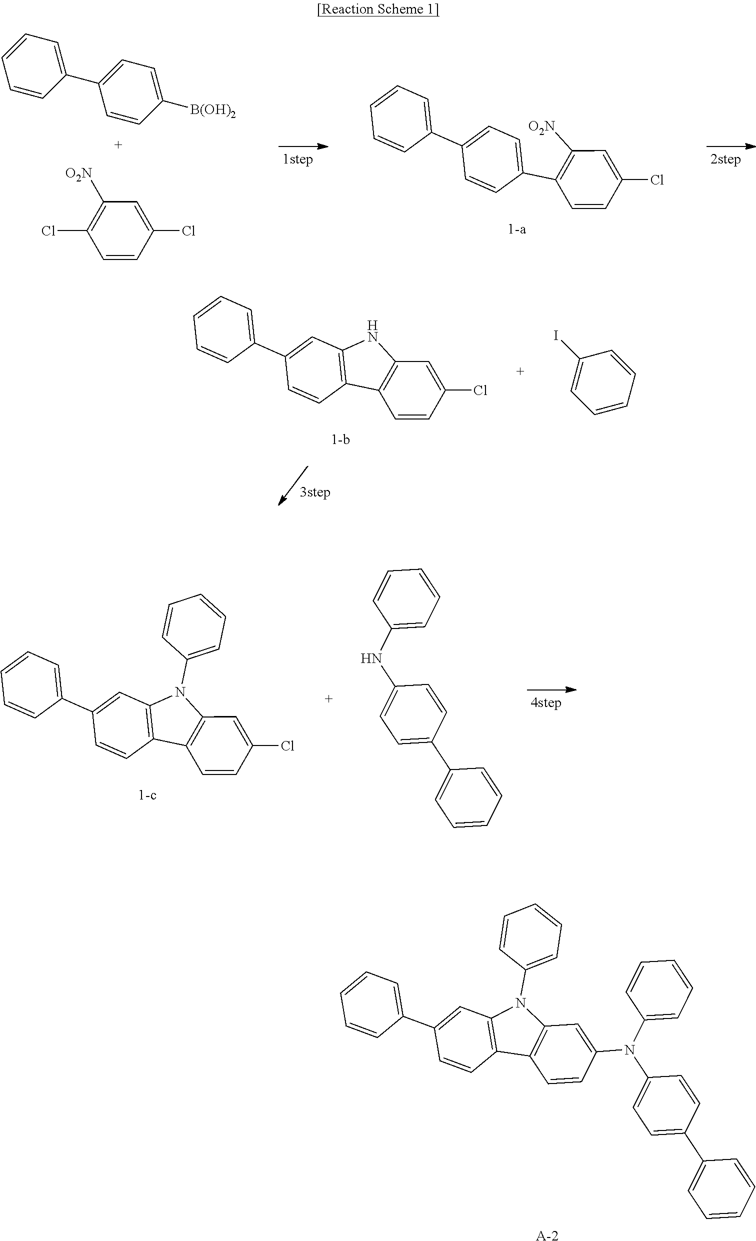

1st Step: Synthesis of Intermediate 1-a

1 eq (30.3 g) of 1,4-dichloro-2-nitrobenzene, 1 eq (31.3 g) of 4-biphenyl boronic acid, 5 mol % (9.1 g) of Pd(PP₃)₄, and 2 eq (43.6 g) of K₂CO₃ were suspended in 310 ml of tetrahydrofuran and 220 ml of distilled water and then, refluxed and stirred under a nitrogen flow for 12 hours. When a reaction was complete, tetrahydrofuran and distilled water were used for an extraction, and then, an organic layer therefrom was dried with magnesium sulfate (MgSO₄) and filtered, and the filtrate was concentrated under a reduced pressure. A solid product was recrystallized with dichloromethane and hexane to obtain 37 g (Y=74%) of Intermediate 1-a.

2nd Step: Synthesis of Intermediate 1-b

37 g of Intermediate 1-a and 100 g of triphenylphosphine were suspended in 400 ml of 1,2-dichlorobenzene and then, refluxed and stirred under a nitrogen flow for 18 hours. When a reaction was complete, after extracting a solvent, an organic layer therein was recrystallized with 200 ml of acetone to obtain 20 g (Y=61%) of Intermediate 1-b.

3rd Step: Synthesis of Intermediate 1-c

20 g of Intermediate 1-b, 45 g of iodobenzene, 2.7 g of 1,10-phenathroline, 2.8 g of CuI, and 15.2 g of K₂CO₃ were suspended in 250 ml of dimethyl formamide and then, reflexed and stirred under a nitrogen flow. When a reaction was complete, the resultant was precipitated in methanol and filtered, and then, a solid therefrom was dissolved in monochlorobenzene, silica-filtered, and recrystallized to obtain 18 g (Y=69%) of Intermediate 1-c.

4th Step: Synthesis of Compound A-2

1 eq (18 g) of Intermediate 1-c, 1 eq (12.5 g) of phenyl-(4-biphenyl)-amine, 2 eq (9.7 g) of sodium-t-butoxide, and 0.03 eq (1.4 g) of Pd₂(dba)₃ were suspended in 170 ml of toluene, and 0.09 eq of tri-t-butylphosphine was added thereto and then, refluxed and stirred for 12 hours. When a reaction was complete, toluene and distilled water were used for an extraction, and an organic layer therefrom was dried with magnesium sulfate and filtered, and the filtrate was concentrated under a reduced pressure. After removing an organic solution, the residue was silica gel columned with hexane:dichloromethane=8:2 (v/v), and a solid product therefrom was recrystallized with dichloromethane and acetone to obtain 21.2 g (Y=74%) of Compound A-2.

LC-Mass measurement (theoretical value: 562.70 g/mol, measured value: M=562.92 g/mol)

### Synthesis Example 2: Synthesis of Compound A-3

1 eq (16.6 g) of Intermediate 1-c, 1 eq (15.1 g) of bis(4-biphenyl)-amine, 2 eq (9.0 g) of sodium-t-butoxide, and 0.03 eq (1.3 g) of Pd₂(dba)₃ were suspended in 200 ml of toluene, and 0.09 eq of tri-t-butylphosphine was added thereto and then, refluxed and stirred for 12 hours. When a reaction was complete, toluene and distilled water were used for an extraction, and then, an organic layer therefrom was dried with magnesium sulfate and filtered and then, concentrated under a reduced pressure. After removing an organic solution, a solid product was recrystallized with dichloromethane and acetone to obtain 24.3 g (Y=84%) of Compound A-3.

LC-Mass measurement (theoretical value: 638.80 g/mol, measured value: M=639.15 g/mol)

### Synthesis Example 3: Synthesis of Compound A-7

23.5 g (Y=78%) of Compound A-7 was obtained according to the same method as Synthesis Example 2 except that 1 eq of Intermediate 1-c and 1 eq of phenyl-(4-terphenyl)-amine were used.

LC-Mass measurement (theoretical value: 638.80 g/mol, measured value: M=639.40 g/mol)

### Synthesis Example 4: Synthesis of Compound A-17

22.6 g (Y=75%) of Compound A-17 was obtained according to the same method as Synthesis Example 2 except that 17.3 g of Intermediate 1-c and 14.5 g of 4-(2-naphthalenyl)-N-phenylbenzeneamine were reacted.

LC-Mass measurement (theoretical value: 612.76 g/mol, measured value: M=613.76 g/mol)

### Synthesis Example 5: Synthesis of Compound A-23

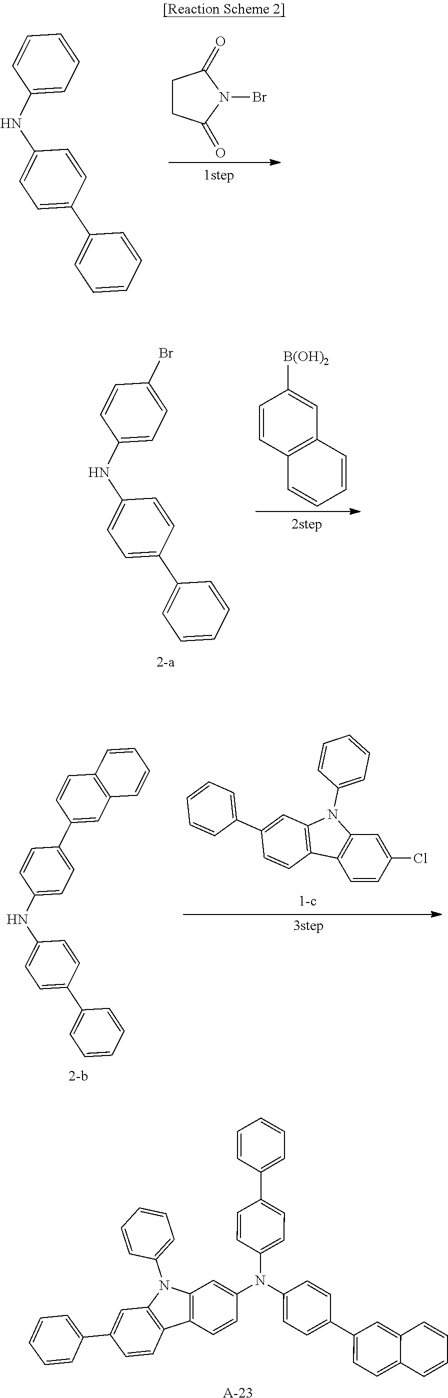

1st Step: Synthesis of Intermediate 2-a

1 eq (22.7 g) of phenyl-(4-biphenyl)-amine and 0.95 eq (15.7 g) of N-bromo succinimide (NBS) were dissolved in 300 ml of dichloromethane and then stirred at 0° C. for 8 hours. When a reaction was complete, distilled water was used for an extraction, and then, an organic layer therefrom was dried with magnesium sulfate and filtered, and the filtrate was concentrated under a reduced pressure and recrystallized with acetone to obtain 28.5 g (Y=95%) of Intermediate 2-a.

2nd Step: Synthesis of Intermediate 2-b

1 eq (26.2 g) of Intermediate 2-a and 1 eq (13.9 g) of naphthalene-2-boronic acid along with 5 mol % (9.1 g) of Pd(PP₃)₄ and 2 eq (43.6 g) of K₂CO₃ were suspended in 150 ml of tetrahydrofuran and 80 ml of distilled water and then, refluxed and stirred under a nitrogen stream for 12 hours. When a reaction was complete, tetrahydrofuran and distilled water were used for an extraction, and an organic layer therefrom was dried with magnesium sulfate (MgSO₄) and filtered, and the filtrate was concentrated under a reduced pressure. A solid product therefrom was recrystallized with dichloromethane and hexane to obtain 21 g (Y=70%) of Intermediate 2-b.

3rd Step: Synthesis of Compound A-23

23.0 g (Y=77%) of Compound A-23 was obtained according to the same method as Synthesis Example 2 except that 1 eq (15.4 g) of Intermediate 1-c and 1 eq (16.2 g) of Intermediate 2-b were reacted.

LC-Mass measurement (theoretical value: 688.86 g/mol, measured value: M=689.86 g/mol)

### Synthesis Example 6: Synthesis of Compound A-27

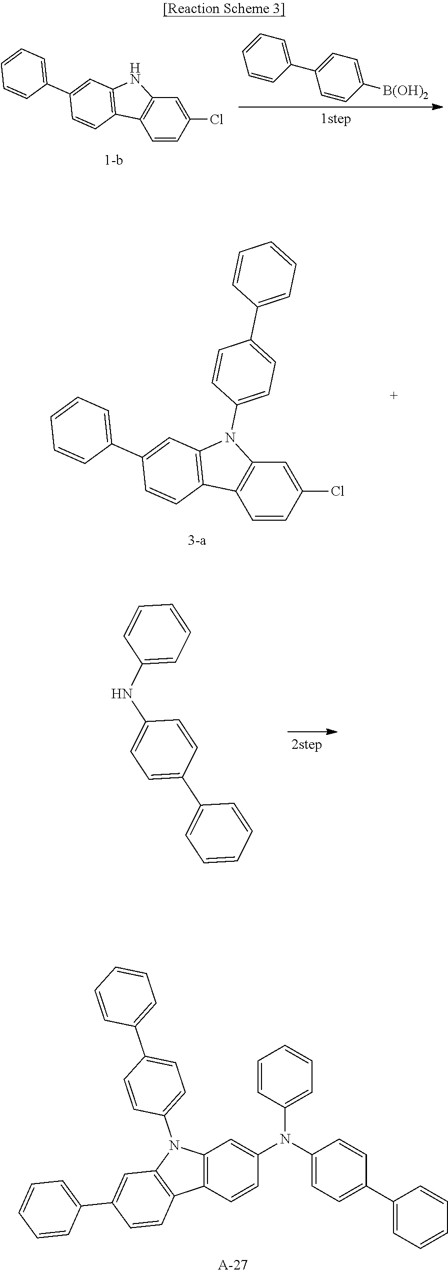

1st Step: Synthesis of Intermediate 3-a

16.1 g of Intermediate 1-b, 41 g of 4-bromobiphenyl, 2.1 g of 1,10-phenathroline, 2.2 g of CuI, and 12.0 g of K₂CO₃ were suspended in 200 ml of dimethyl formamide and then, refluxed and stirred under a nitrogen flow. When a reaction was complete, the resultant was precipitated in methanol, and a solid therefrom was filtered, dissolved in monochlorobenzene, silica-filtered, and recrystallized to obtain 15.7 g (Y=63%) of Intermediate 3-a.

2nd Step: Synthesis of Compound A-27

1 eq (15.5 g) of Intermediate 3-a, 1 eq (8.8 g) of phenyl-(4-biphenyl)-amine, 2 eq of sodium-t-butoxide, and 0.03 eq of Pd₂(dba)₃ were suspended in 200 ml of xylene, and 0.09 eq of tri-t-butylphosphine was added thereto and then, refluxed and stirred for 12 hours. When a reaction was complete, toluene and distilled water were used for an extraction, an organic layer therefrom was dried with magnesium sulfate and filtered, and the filtrate was concentrated under a reduced pressure. After removing an organic solution, the residue was silica gel columned with hexane:dichloromethane=8:2 (v/v), and a solid product therefrom was recrystallized by using dichloromethane and acetone to obtain 17.5 g (Y=76%) of Compound A-27.

LC-Mass measurement (theoretical value: 638.80 g/mol, measured value: M=639.60 g/mol)

### Synthesis Example 7: Synthesis of Compound A-33

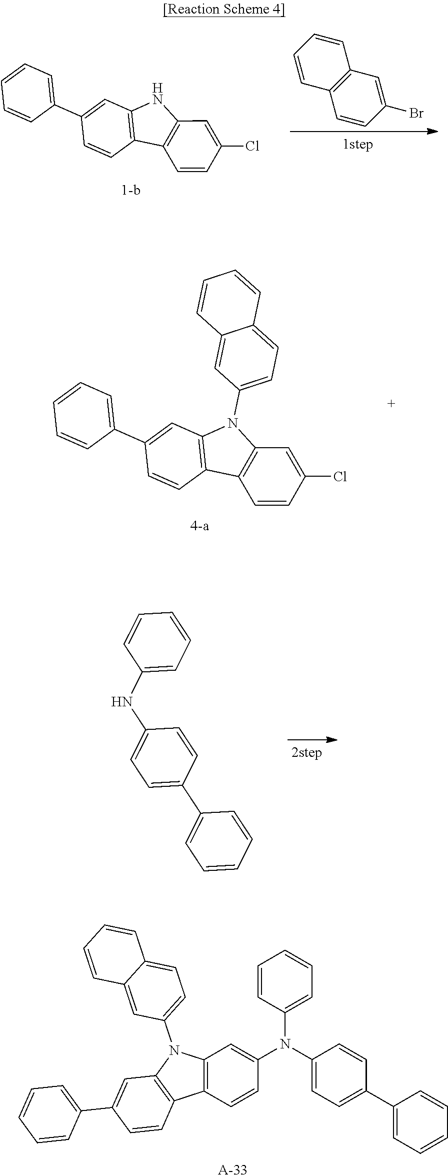

1st Step: Synthesis of Intermediate 4-a

17.2 g of Intermediate 1-b, 38.5 g of 2-bromonaphthalene, 2.2 g of 1,10-phenathroline, 2.4 g of CuI, and 12.8 g of K₂CO₃ were suspended in 210 ml of dimethyl formamide and then, refluxed and stirred under a nitrogen flow. When a reaction was complete, the resultant was precipitated in methanol and filtered, and a solid therefrom was dissolved in monochlorobenzene, silica-filtered, and then, recrystallized to obtain 18.5 g (Y=74%) of Intermediate 4-a.

2nd Step: Synthesis of Compound A-33

1 eq (16.5 g) of Intermediate 4-a, 1 eq (10.0 g) of phenyl-(4-biphenyl)-amine, 2 eq of sodium t-butoxide, and 0.03 eq of Pd₂(dba)₃ were suspended in 180 ml of xylene, and 0.09 eq of tri-t-butylphosphine was added thereto and then, refluxed and stirred for 12 hours. When a reaction was complete, toluene and distilled water were used for an extraction, an organic layer therefrom was dried with magnesium sulfate and filtered, and the filtrate was concentrated under a reduced pressure. After removing an organic solution, a solid product therefrom was obtained through silica gel column by using hexane:dichloromethane=8:2 (v/v) and recrystallized with dichloromethane and acetone to obtain 20.1 g (Y=80%) of Compound A-33.

LC-Mass measurement (theoretical value: 612.76 g/mol, measured value: M=613.56 g/mol)

### Synthesis Example 8: Synthesis of Compound A-47

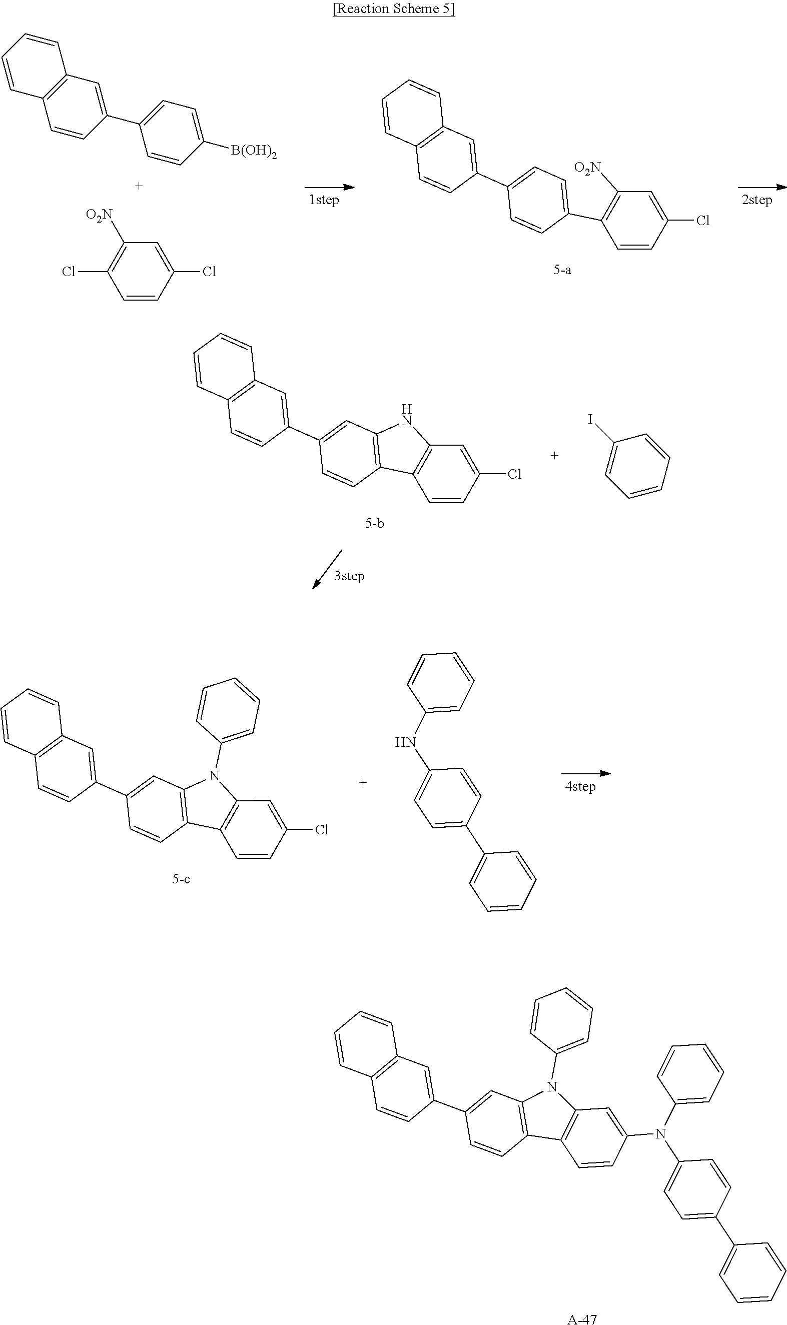

1st Step: Synthesis of Intermediate 5-a

1 eq (26.7 g) of 1,4-dichloro-2-nitrobenzene, 1 eq (34.5 g) of 4-(2-naphthalenyl)phenyl boronic acid, 5 mol % (8.03 g) of Pd(PP₃)₄, and 2 eq (38.4 g) of K₂CO₃ were suspended in 340 ml of tetrahydrofuran and 200 ml of distilled water and then, refluxed and stirred under a nitrogen flow for 12 hours. When a reaction was complete, tetrahydrofuran and distilled water were used for an extraction, an organic layer therefrom was dried with magnesium sulfate (MgSO₄) and filtered, and the filtrate was concentrated under a reduced pressure. A solid product therefrom was recrystallized with dichloromethane and hexane to obtain 34 g (Y=68%) of Intermediate 5-a.

2nd Step: Synthesis of Intermediate 5-b

34 g of Intermediate 5-a and 100 g of triphenylphosphine were suspended in 300 ml of 1,2-dichlorobenzene and then, refluxed and stirred under a nitrogen flow for 18 hours. When a reaction was complete, after extracting a solvent, an organic layer therefrom was recrystallized with 150 ml of acetone to obtain 17 g (Y=55%) of Intermediate 5-b.

3rd Step: Synthesis of Intermediate 5-c

17 g of Intermediate 5-b, 36 g of iodobenzene, 1.9 g of 1,10-phenathroline, 2.0 g of CuI, and 10.7 g of K₂CO₃ were suspended in 180 ml of dimethylformamide and then, refluxed and stirred under a nitrogen flow. When a reaction was complete, the resultant was precipitated in methanol, and a solid therefrom was filtered and then, dissolved in monochlorobenzene, silica-filtered, and recrystallized to obtain 16.4 g (Y 78%) of Intermediate 5-c.

4th Step: Synthesis of Compound A-47

1 eq (16.4 g) of Intermediate 5-c, 1 eq (10.0 g) of phenyl-(4-biphenyl)-amine, 2 eq (7.8 g) of sodium-t-butoxide, and 0.03 eq (1.12 g) of Pd₂(dba)₃ were suspended in 150 ml of xylene, and 0.09 eq of tri-t-butylphosphine was added thereto and then, refluxed and stirred for 12 hours. When a reaction was complete, toluene and distilled water were used for an extraction, an organic layer therefrom was dried with magnesium sulfate and filtered, and the filtrate was concentrated under a reduced pressure. After removing an organic solution, the residue was silica gel-columned with hexane:dichloromethane=8:2 (v/v), and a solid product therefrom was recrystallized with dichloromethane and acetone to obtain 19.6 g (Y=78%) of Compound A-47.

LC-Mass measurement (theoretical value: 612.76 g/mol, measured value: M=613.77 g/mol)

### Comparative Synthesis Example 1: Synthesis of Compound V-1

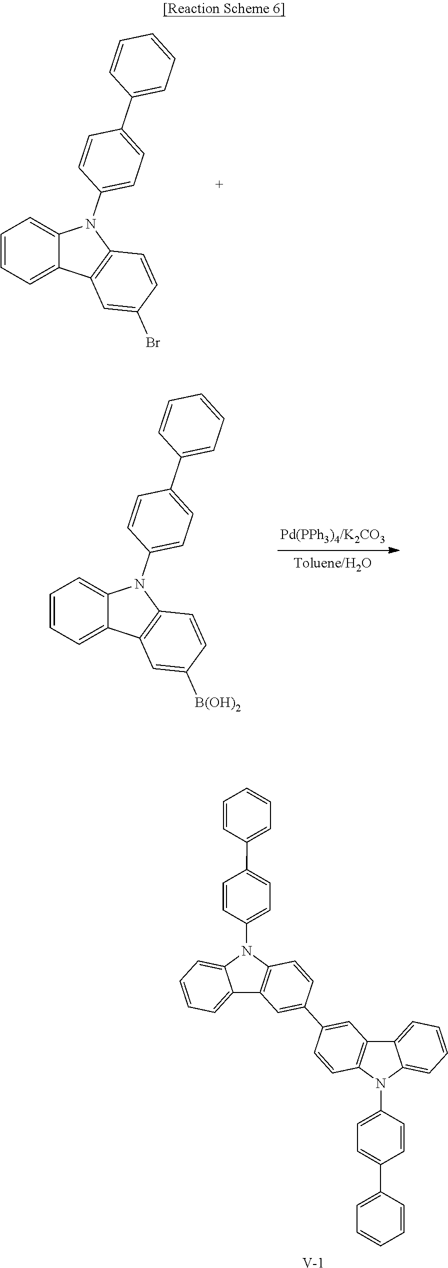

Biphenylcarbazolyl bromide (12.33 g, 30.95 mmol) was dissolved in 200 mL of toluene in a nitrogen environment, and biphenylcarbazolylboronic acid (12.37 g, 34.05 mmol) and tetrakis(triphenylphosphine)palladium (1.07 g, 0.93 mmol) were added thereto and then, stirred. Subsequently, potassium carbonate saturated in water (12.83 g, 92.86 mmol) was added thereto and then, refluxed and stirred at 90° C. for 12 hours. When a reaction was complete, water was added to the reaction solution, dichloromethane (DCM) was used for an extraction, moisture was removed with anhydrous MgSO₄, and the residue was filtered and concentrated under a reduced pressure. The obtained residue was separated and purified through flash column chromatography to obtain Compound V-1 (18.7 g, 92%).

LC/MS calculated for: C48H32N2 Exact Mass: 636.26 found for 636.30 [M+H].

(Preparation of Second Compound)

### Synthesis Example 9: Synthesis of Compound B-17

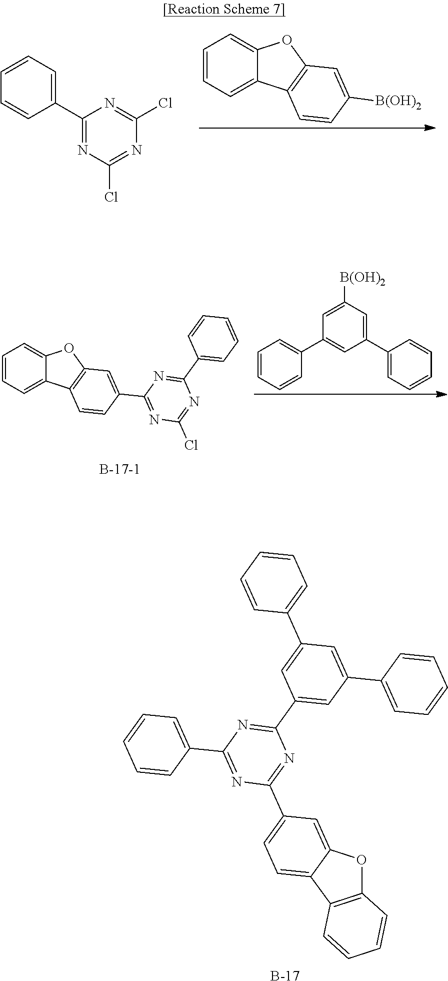

1st Step: Synthesis of Intermediate B-17-1

22.6 g (100 mmol) of 2,4-dichloro-6-phenyltriazine was added to 100 mL of tetrahydrofuran, 100 mL of toluene, and 100 mL of distilled water in a 500 mL round-bottomed flask, and 0.9 equivalent of dibenzofuran-3-boronic acid (CAS No.: 395087-89-5), 0.03 equivalent of tetrakis(triphenylphosphine) palladium, and 2 equivalents of potassium carbonate were added thereto and then, heated and refluxed under a nitrogen atmosphere. After 6 hours, the reaction solution was cooled down, and after removing an aqueous layer, an organic layer therein was dried under a reduced pressure. The obtained solid was washed with water and hexane and recrystallized with 200 mL of toluene to obtain 21.4 g (Yield of 60%) of Intermediate B-17-1.

2nd Step: Synthesis of Compound B-17

Intermediate B-17-1 (56.9 mmol) was added to 200 mL of tetrahydrofuran and 100 mL of distilled water in a 500 mL round-bottomed flask, and 1.1 equivalent of 3,5-diphenylbenzeneboronic acid (CAS No.: 128388-54-5), 0.03 equivalent of tetrakis(triphenylphosphine) palladium, and 2 equivalent of potassium carbonate were added thereto and then, heated and refluxed under a nitrogen atmosphere. After 18 hours, the reaction solution was cooled down, and a solid precipitated therein was filtered and washed with 500 mL of water. The solid was recrystallized with 500 mL of monochlorobenzene to obtain Compound B-17.

LC/MS measurement (C39H25N3O, theoretical value: 555.1998 g/mol, measured value: 556.21 g/mol)

### Synthesis Example 10: Synthesis of Compound B-135

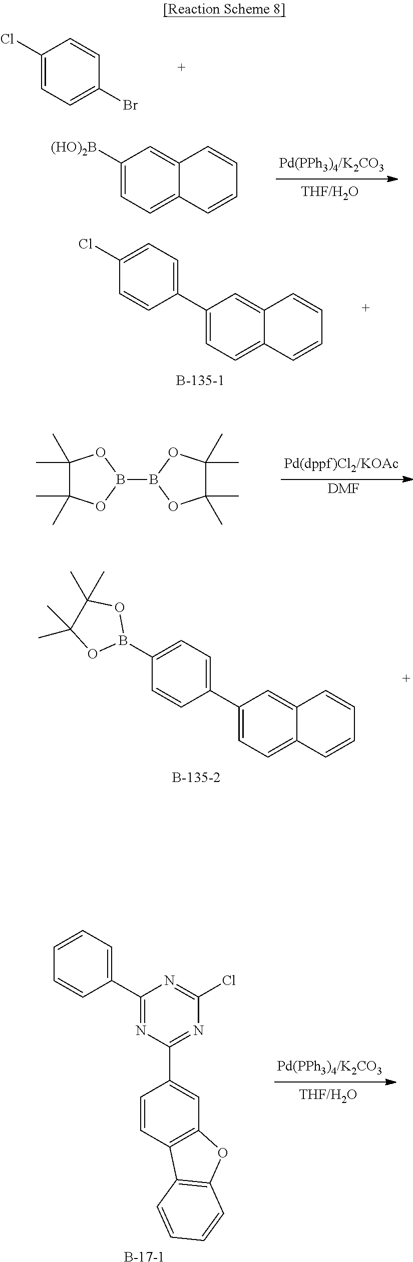

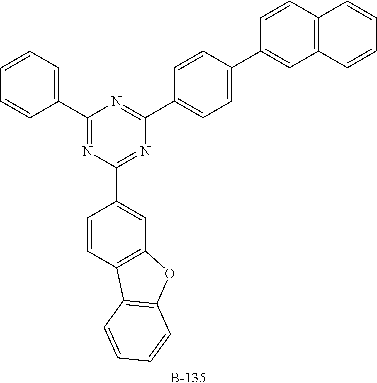

1st Step: Synthesis of Intermediate B-135-1

Intermediate B-135-1 was synthesized according to the same method as the 1st step of Synthesis Example 9 except that 1-bromo-4-chloro-benzene and 2-naphthalene boronic acid were used in each amount of 1.0 equivalent.

2nd Step: Synthesis of Intermediate B-135-2

1 equivalent of Intermediate B-135-1 was put in 250 mL of DMF in a 500 mL round-bottomed flask, and 0.05 equivalent of dichlorodiphenylphosphinoferrocene palladium, 1.2 equivalent of bis(pinacolato) diboron, and 2 equivalent of potassium acetate were added thereto and then, heated and refluxed under a nitrogen atmosphere for 18 hours. The reaction solution was cooled down and added to 1 L of water in a dropwise fashion to obtain a solid. The obtained solid was dissolved in boiling toluene and treated with activated carbon and then, filtered with silica gel, and the filtrate was concentrated. The concentrated solid was stirred with a small amount of hexane, and then, a solid was filtered therefrom to synthesize Intermediate B-135-2.

3rd Step: Synthesis of Compound B-135

Compound B-135 was synthesized according to the same method as the 2nd step of Synthesis Example 9 except that Intermediate B-135-2 and Intermediate B-17-1 were used in each amount of 1.0 equivalent.

LC/MS measurement (C37H23N3O, theoretical value: 525.18 g/mol, measured value: M=525.22 g/mol)

### Synthesis Example 11: Synthesis of Compound B-205

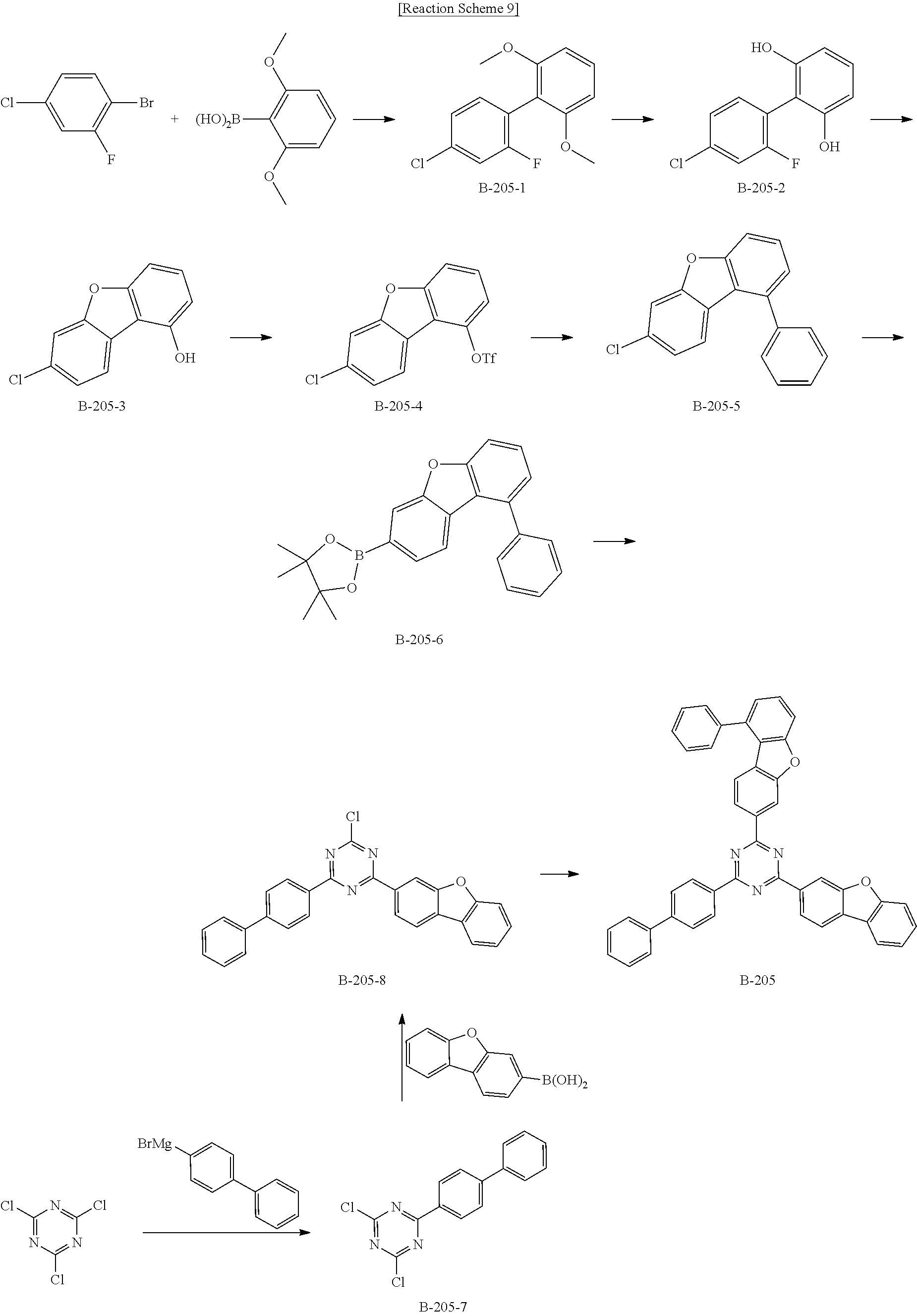

1st Step: Synthesis of Intermediate B-205-1

1-bromo-4-chloro-2-fluorobenzene (61 g, 291 mmol), 2,6-dimethoxyphenylboronic acid (50.4 g, 277 mmol), K₂CO₃ (60.4 g, 437 mmol), and Pd(PPh₃)₄ (10.1 g, 8.7 mmol) were put in a round-bottomed flask and dissolved in THE (500 ml) and distilled water (200 ml) and then, refluxed and stirred at 60° C. for 12 hours. When the reaction was completed, an aqueous layer was removed therefrom to obtain 38 g (51%) of Intermediate B-205-1 by using column chromatography (hexane: DCM 20%).

2nd Step: Synthesis of Intermediate B-205-2

Intermediate B-205-1 (38 g, 142 mmol) and pyridine hydrochloride (165 g, 1425 mmol) were put in a round-bottomed flask and then, refluxed and stirred at 200° C. for 24 hours. When a reaction was complete, the resultant was cooled down to ambient temperature and then, slowly poured into distilled water and then, stirred for one hour. A solid therefrom was filtered to obtain 23 g (68%) of Intermediate B-205-2.

3rd Step: Synthesis of Intermediate B-205-3

Intermediate B-205-2 (23 g, 96 mmol) and K₂CO₃ (20 g, 144 mmol) were put in a round-bottomed flask and dissolved in 100 ml of NMP and then, refluxed and stirred at 180° C. for 12 hours. When a reaction was complete, the mixture was poured into an excess of distilled water. A solid therefrom was filtered, dissolved in ethyl acetate, and dried with MgSO₄, and an organic layer therefrom was removed under a reduced pressure. Column chromatography (hexane: EA 30%) was used to obtain 16 g (76%) of Intermediate B-205-3.

4th Step: Synthesis of Intermediate B-205-4

Intermediate B-205-3 (16 g, 73 mmol) and pyridine (12 ml, 146 mmol) were put in a round-bottomed flask and dissolved in 200 ml of DCM. A temperature was decreased to 0° C., and then, trifluoromethanesulfonic anhydride (14.7 ml, 88 mmol) was slowly added thereto in a dropwise fashion. After stirring the mixture for 6 hours, when a reaction was complete, an excess of distilled water was added thereto and then, stirred for 30 minutes and extracted with DCM. An organic solvent was removed therefrom under a reduced pressure, and the residue was vacuum-verified to obtain 22.5 g (88%) of Intermediate B-205-4.

5th Step: Synthesis of Intermediate B-205-5

14.4 g (81%) of intermediate B-205-5 was obtained according to the same method as Synthesis Example 1 except that Intermediate B-205-4 (22.5 g, 64 mmol), phenylboronic acid (7.8 g, 64 mmol), K₂CO₃ (13.3 g, 96 mmol), and Pd(PPh₃)₄ (3.7 g, 3.2 mmol) were used.

6th Step: Synthesis of Intermediate B-205-6

Intermediate B-205-5 (22.5 g, 80 mmol), bis(pinacolato)diboron (24.6 g, 97 mmol), Pd(dppf)Cl₂ (2 g, 2.4 mmol), tricyclohexylphosphine (3.9 g, 16 mmol), and potassium acetate (16 g, 161 mmol) were put in a round-bottomed flask and dissolved in 320 ml of DMF. The obtained mixture was refluxed and stirred at 120° C. for 10 hours. When a reaction was complete, the mixture was poured into an excess of distilled water and then, stirred for 1 hour. A solid therefrom was filtered and dissolved in DCM. After removing moisture with MgSO₄, an organic solvent was filtered with a silica gel pad and then, removed under a reduced pressure. A solid therefrom was recrystallized with EA and hexane to obtain 26.9 g (90%) of Intermediate B-205-6.

7th Step: Synthesis of Intermediate B-205-7

15 g (81.34 mmol) of cyanuricchloride was put in a 500 mL round-bottomed flask and dissolved in 200 mL of anhydrous tetrahydrofuran, 1 equivalent of a 4-biphenyl magnesium bromide solution (0.5 M tetrahydrofuran) was added thereto in a dropwise fashion at 0° C. under a nitrogen atmosphere and then, slowly heated to ambient temperature. The reaction solution was stirred at ambient temperature for 1 hour and then, put in 500 mL of ice water to separate layers. An organic layer was separated therefrom, treated with magnesium sulfate anhydrous, and then, concentrated. The concentrated residue was recrystallized with tetrahydrofuran and methanol to obtain 17.2 g of Intermediate B-205-7.

8th Step: Synthesis of Intermediate B-205-8

Intermediate B-205-8 was synthesized according to the same method as the 1st step of Synthesis Example 9 except that Intermediate B-205-7 was used.

9th Step: Synthesis of Compound B-205

15.5 g (70%) of Compound B-205 was synthesized according to the same method as the 2nd step of Synthesis Example 9 except that Intermediate B-205-6 (12.8 g, 35 mmol), Intermediate B-205-8 (15 g, 35 mmol), K₂CO₃ (7.2 g, 52 mmol), and Pd(PPh₃)₄ (2 g, 1.7 mmol) were used in a round-bottomed flask under a nitrogen condition.

LC/MS measurement (C45H27N3O2, theoretical value: 641.21 g/mol, measured value: M=641.25 g/mol)

### Synthesis Example 12: Synthesis of Compound B-183

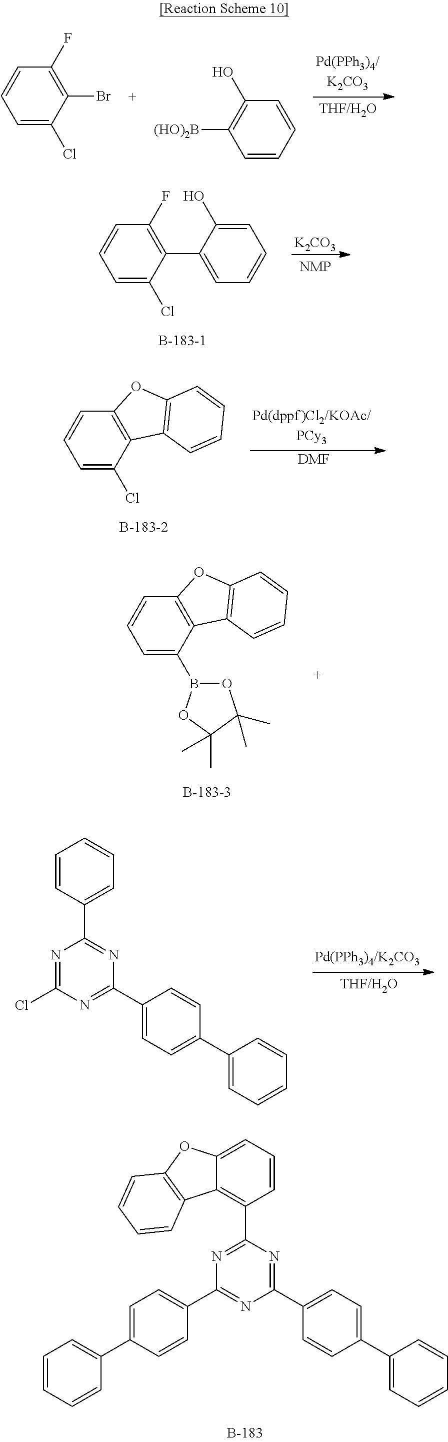

1st Step: Synthesis of Intermediate B-183-1

Intermediate B-183-1 was synthesized according to the same method as the 1st step of Synthesis Example 11 except that 2-bromo-1-chloro-3-fluoro-benzene and 2-hydroxyphenylboronic acid were used in each amount of 1.0 equivalent.

2nd Step: Synthesis of Intermediate B-183-2

Intermediate B-183-2 was synthesized according to the same method as the 3rd step of Synthesis Example 11 except that Intermediate B-183-1 and K₂CO₃ were used in an equivalent ratio of 1:1.5.

3rd Step: Synthesis of Intermediate B-183-3

Intermediate B-183-3 was synthesized according to the same method as the 6th step of Synthesis Example 11 except that Intermediate B-183-2 and bis(pinacolato)diboron were used in an equivalent ratio of 1:1.2.

4th Step: Synthesis of Compound B-183

Compound B-183 was synthesized according to the same method as the 2nd step of Synthesis Example 9 except that Intermediate B-183-3 and 2,4-bis([1,1′-biphenyl]-4-yl)-6-chloro-1,3,5-triazine were respectively used in an amount of 1.0 equivalent.

LC/MS measurement (C39H25N3O theoretical value: 551.20 g/mol, measured value: M=551.24 g/mol)

### Synthesis Example 13: Synthesis of Compound B-209

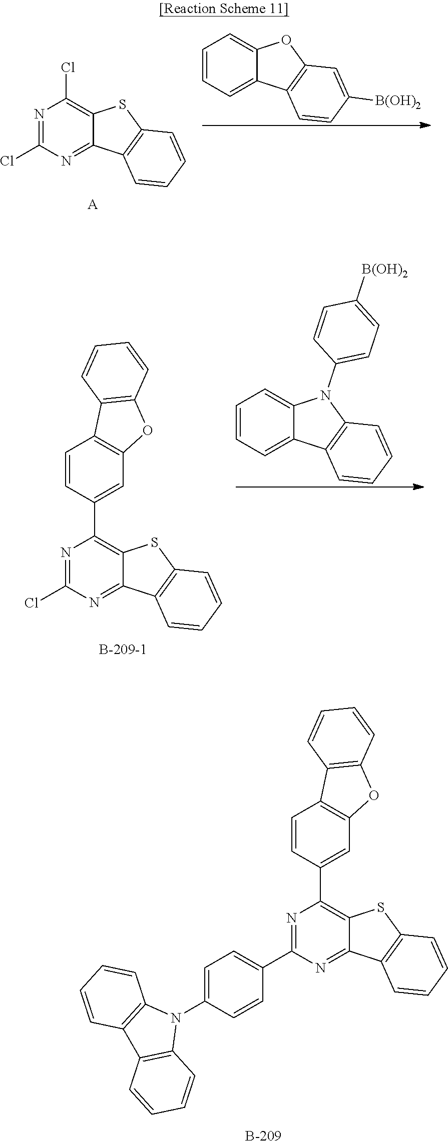

1st Step: Synthesis of Intermediate B-209-1

10.5 g of Intermediate A, 8.8 g of 3-dibenzofuran boronic acid, 11.4 g of potassium carbonate, and 2.4 g of tetrakis(triphenylphosphine) palladium (0) were added to 140 mL of 1,4-dioxane and 70 mL of water in a 500 mL flask and then, heated under a nitrogen flow for 12 hours at 60° C. The obtained mixture was added to 500 mL of methanol to filter a solid crystallized there, and the solid was dissolved in monochlorobenzene, filtered through silica gel/Celite, and after removing an appropriate amount of an organic solvent, recrystallized with methanol to obtain Intermediate B-209-1 (10.7 g, Yield=67%).

2nd Step: Synthesis of Compound B-209

10.4 g of Intermediate B-209-1, 7.8 g of 4-(9-carbazolyl)phenylboronic acid, 7.5 g of potassium carbonate, and 1.6 g of tetrakis(triphenylphosphine) palladium (0) were added to 90 mL of 1,4-dioxane and 45 mL of water in a 250 mL flask and then, heated under a nitrogen flow for 12 hours at 70° C. The obtained mixture was added to 250 mL of methanol, and a solid crystallized therein was filtered, dissolved in 1,2-dichlorobenzene, filtered through silica gel/Celite, and after removing an appropriate amount of an organic solvent, recrystallized with methanol to obtain Compound B-209 (13.0 g, Yield of 74%).

LC/MS measurement (C40H23N3OS), theoretical value: 593.16 g/mol, measured value: M=593.23 g/mol)

### Synthesis Example 14: Synthesis of Compound C-25

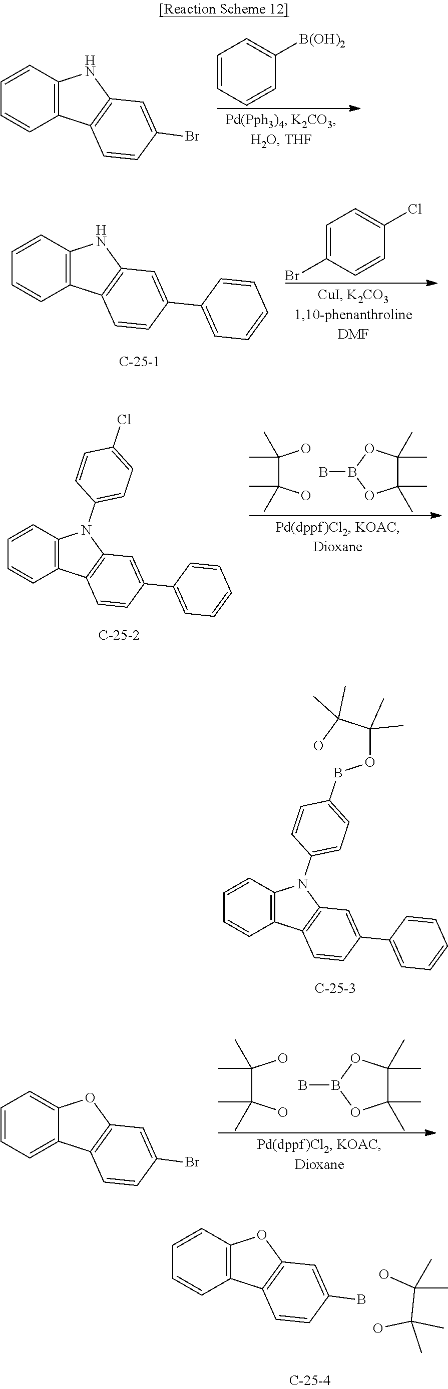

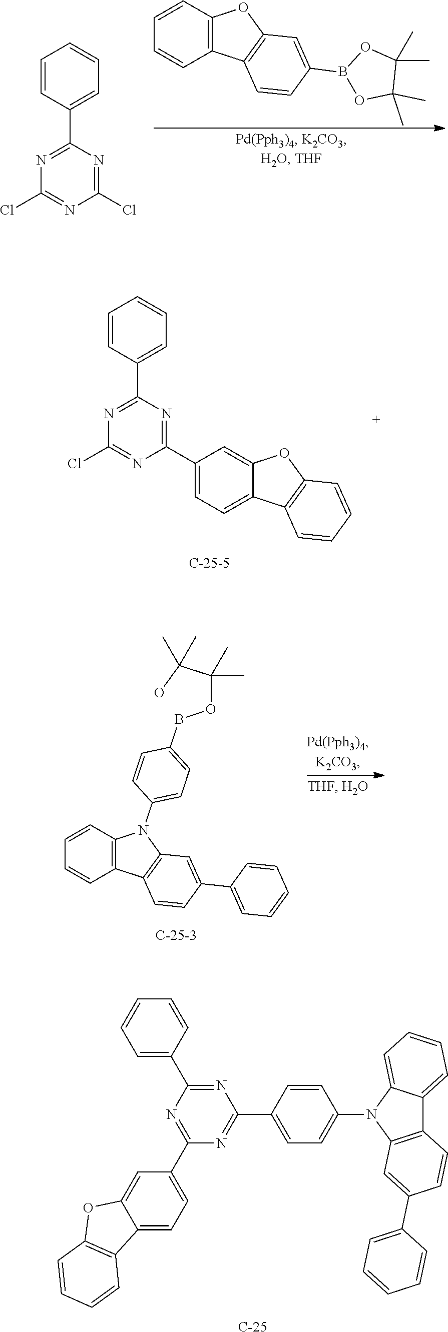

1st Step: Synthesis of Intermediate C-25-1

2-bromocarbazole (35 g, 142 mmol) was dissolved in 0.5 L of tetrahydrofuran (THF), and phenyl boronic acid (17.3 g, 142 mmol) and tetrakis(triphenylphosphine)palladium (8.2 g, 7.1 mmol) were added thereto and then, stirred. Subsequently, potassium carbonate saturated in water (49.1 g, 356 mmol) was added thereto and then, heated and refluxed at 80° C. for 12 hours. When a reaction was complete, water was added to the reaction solution, dichloromethane (DCM) was used for an extraction, magnesium sulfate anhydrous was used to remove moisture therefrom, and the residue was filtered and concentrated under a reduced pressure. The obtained residue was separated and purified through flash column chromatography to obtain 22 g (63.6%) of Intermediate C-25-1.

2nd Step: Synthesis of Intermediate C-25-2

Intermediate C-25-1 (22 g, 90.4 mmol), 1-bromo-4-chloro-benzene (25.96 g 135.61 mmol), CuI (1.71 g, 9 mmol), K₂CO₃ (18.74 g, 135.61 mmol), and 1,10-phenanthroline (1.62 g, 9 mmol) were put in a round-bottomed flask and dissolved in 700 ml of DMF. The obtained mixture was stirred at 180° C. for 18 hours. When a reaction was complete, after removing a reaction solvent under a reduced pressure, the residue was dissolved in dichloromethane and silica gel-filtered. After concentrating the dichloromethane, hexane was used for a recrystallization to obtain 18 g (56.3%) of Intermediate C-25-2.

3rd Step: Synthesis of Intermediate C-25-3

Intermediate C-25-2 (18 g, 51 mmol), bis(pinacolato)diboron (19.43 g, 76.5 mmol), Pd(dppf)Cl₂ (2.24 g, 8.64 mmol), tricyclohexylphosphine (2.86 g, 10.2 mmol), and potassium acetate (15.02 g, 153.01 mmol) were put in a round-bottomed flask and dissolved in 720 ml of DMF. The mixture was refluxed and stirred at 120° C. for 12 hours. When a reaction was complete, the mixture was poured into an excessive amount of distilled water and then, stirred for 1 hour. A solid therefrom was filtered and then, dissolved in DCM. MgSO₄ was used to remove moisture therefrom, an organic solvent was filtered with a silica gel pad and removed under a reduced pressure. A solid therefrom was recrystallized with EA and hexane to obtain 14.8 g (65.3%) of Intermediate C-25-3.

4th Step: Synthesis of Intermediate C-25-4

31 g (65.1%) of Intermediate C-25-4 was synthesized according to the same method as the 3rd step of Synthesis Example 12 except that 3-bromo-dibenzofuran (40 g, 162 mmol) was used instead of Intermediate B-183-2.

5th Step: Synthesis of Intermediate C-25-5

Intermediate C-25-4 was dissolved in 0.3 L of tetrahydrofuran (THF), and 2,4-chloro-6-phenyl-1,3,5-triazine (21 g, 93 mmol) and tetrakis(triphenylphosphine)palladium (5.38 g, 4.65 mmol) were added thereto and then, stirred. Subsequently, potassium carbonate saturated in water (32.14 g, 232 mmol) was added thereto and then, heated and refluxed at 80° C. for 12 hours. When a reaction was complete, water was added to the reaction solution and then, stirred for 30 minutes and filtered, and a solid therefrom was dissolved in monochlorobenzene at 133° C., and after removing moisture with magnesium sulfate anhydrous, filtered by using silica gel, and the filtrate was cooled down to ambient temperature and filtered. The obtained solid was repetitively purified by using monochlorobenzene to obtain 15 g (64.8%) of Intermediate C-25-5.

6th Step: Synthesis of Compound C-25

12.7 g (67.5%) of Compound C-25 was synthesized according to the same method as the 4th step of Synthesis Example 12 except that Intermediate C-25-5 (10.5 g 29.3 mmol) and Intermediate C-25-3 (14.38 g, 32.28 mmol) were used.

LC/MS measurement (C45H28NO), theoretical value: 640.23 g/mol, measured value: M=641.38 g/mol)

### Synthesis Example 15: Synthesis of Compound C-23

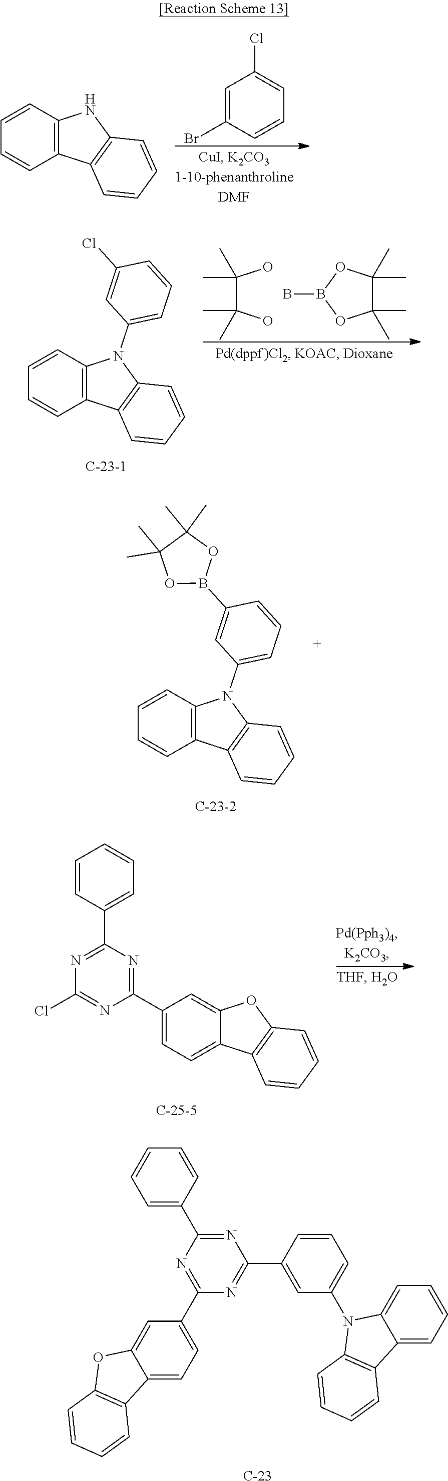

1st Step: Synthesis of Intermediate C-23-1

31.5 g (79%) of Intermediate C-23-1 was obtained according to the same method as the 2nd step of Synthesis Example 14 except that 9H-carbazole (24.1 g, 144 mmol) and 1-bromo-3-chloro-benzene (27.6 g 144 mmol) were used.

2nd Step: Synthesis of Intermediate C-23-2

16.8 g (70%) of Intermediate C-23-2 was obtained according to the same method as the 3rd step of Synthesis Example 14 except that Intermediate C-23-1 (18 g, 65 mmol) was used instead of Intermediate C-25-2.

3rd Step: Synthesis of Compound C-23

16.4 g (66%) of Intermediate C-23 was obtained according to the same method as the 6th step of Synthesis Example 14 except that Intermediate C-23-2 (16.3 g, 44.3 mmol) and Intermediate C-25-5 (15.8 g 44.3 mmol) were used.

LC/MS measurement (C39H24N4O), theoretical value: 564.20 g/mol, measured value: M=565.36 g/mol)

### Synthesis Example 16: Synthesis of Compound D-57

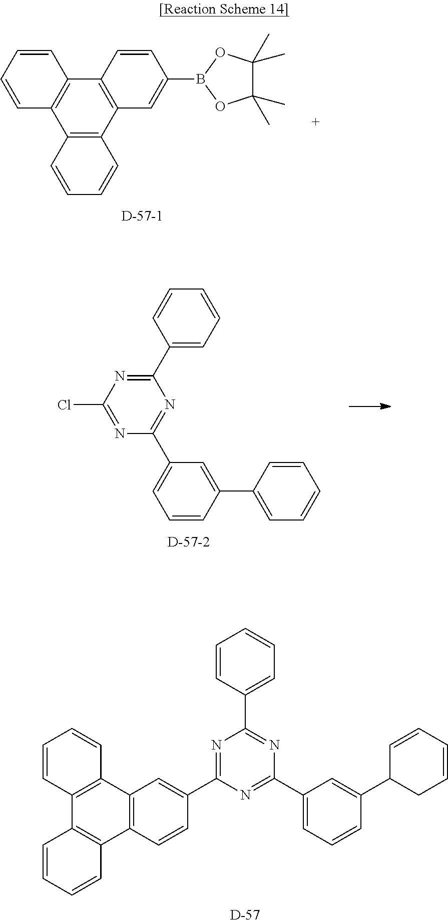

Compound D-57 (Yield: 88%) was synthesized by using Intermediate D-57-1 and Intermediate D-57-2 according to a suitable method.

LC/MS measurement (C39H25N3), theoretical value: 535.20 g/mol, measured value: M=535.83 g/mol)

### Comparative Synthesis Example 2: Synthesis of Compound V-2

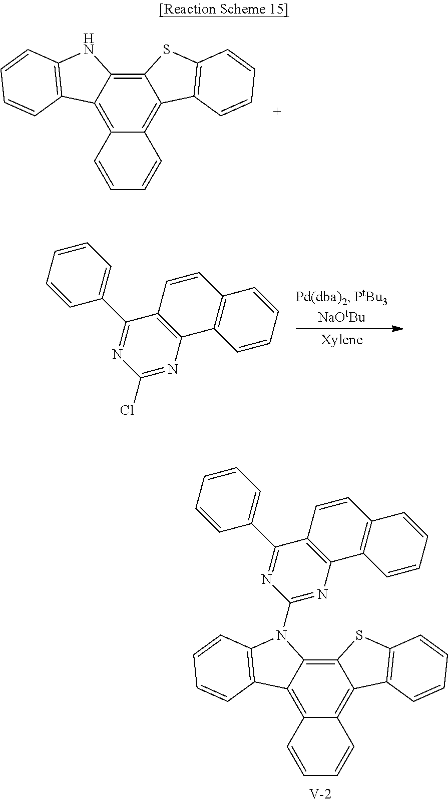

5.7 g (Yield=57%) of Compound V-2 was obtained as shown in Reaction Scheme 15.

### Comparative Synthesis Example 3: Synthesis of Compound V-3

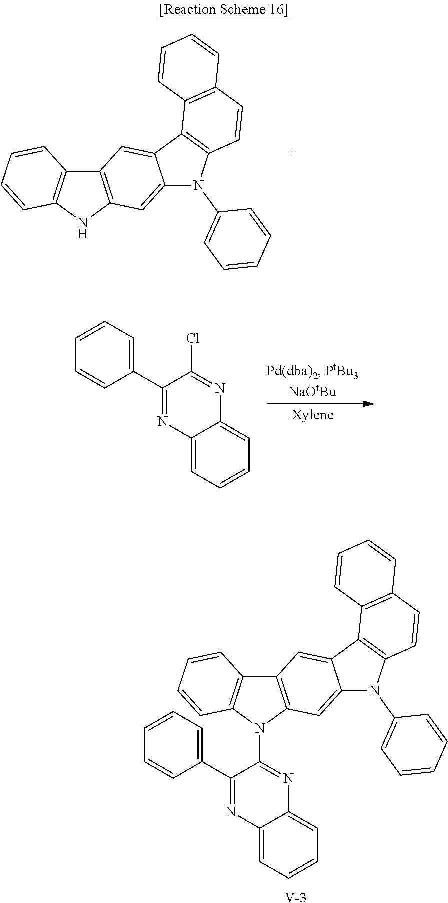

6.4 g (Yield=47%) of Compound V-3 was obtained as shown in Reaction Scheme 16.

(Preparation of Third Compound)

### Synthesis Example 17: Synthesis of Compound E-9

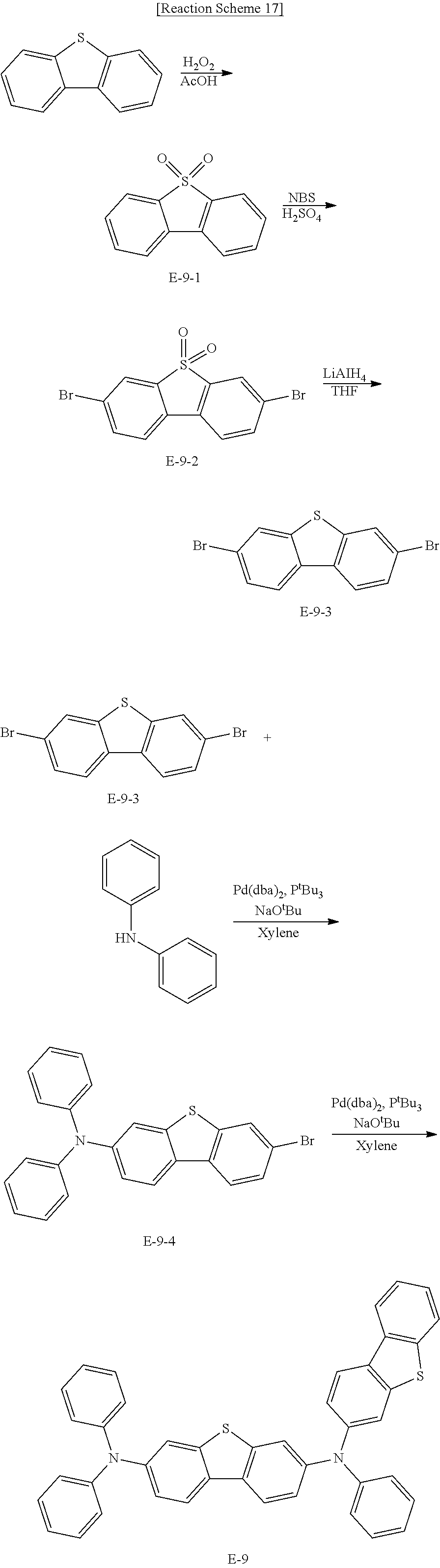

1st Step: Synthesis of Intermediate E-9-1

50 g (271.43 mmol) of dibenzothiophene was put in 500 mL of acetic acid in a 1 L round-bottomed flask, and an internal temperature was set at 0° C. 117 ml (1.36 mol) of hydrogen peroxide was slowly added thereto. Herein, the internal temperature was maintained at 0° C. The flask was heated at 90° C. under a nitrogen atmosphere. After 12 hours, the reaction solution was cooled down, treated with dichloromethane (DCM) for an extraction, and treated with magnesium sulfate anhydrous to remove moisture, and the residue was filtered and concentrated under a reduced pressure to obtain 55 g (Yield=94%) of Intermediate E-9-1.

2nd Step: Synthesis of Intermediate E-9-2

54 g (249.70 mmol) of Intermediate E-9-1 was added to 500 mL of sulfuric acid in a 1 L round-bottomed flask, and an internal temperature was set at 0° C. 90.7 g (499.40 mmol) of NBS was slowly added thereto. Herein, the internal temperature was maintained at 0° C. The reaction solution was stirred under a nitrogen atmosphere at ambient temperature for 4 hours and then, slowly put in ice water, extracted with dichloromethane (DCM), treated with magnesium sulfate anhydrous to remove moisture, filtered, and concentrated under a reduced pressure. The obtained residue was separated and purified through flash column chromatography to obtain 46 g (49%) of Intermediate E-9-2.

3rd Step: Synthesis of Intermediate E-9-3

45 g (120.30 mmol) of Intermediate E-9-2 was added to 500 mL of tetrahydrofuran in a 1 L round-bottomed flask, and an internal temperature thereof was set at 0° C. 10.1 g (252.64 mmol) of lithium aluminum hydride was slowly added thereto. Herein, the internal temperature was maintained at 0° C. The reaction solution was stirred under a nitrogen atmosphere at 75° C. for 3 hours, slowly put in ice water, and then, Celite-filtered. Dichloromethane (DCM) was used for an extraction, moisture was removed by using magnesium sulfate anhydrous, and the residue was filtered and concentrated under a reduced pressure. The obtained residue was separated and purified through flash column chromatography to obtain 28 g (68%) of Intermediate E-9-3.

4th Step: Synthesis of Intermediate E-9-4

15.0 g (43.92 mmol) of Intermediate E-9-3, 6.69 g (39.30 mmol) of diphenylamine, 10.56 g (109.8 mmol) of sodium t-butoxide, and 1.8 g (4.38 mmol) of tri-tert-butylphosphine were dissolved in 300 ml of xylene, and 2.01 g (2.19 mmol) of Pd(dba)₂ was added thereto and then, stirred under a nitrogen atmosphere for 12 hours at 100° C. When a reaction was complete, xylene and distilled water were used for an extraction, an organic layer therefrom was dried with magnesium sulfate anhydrous and filtered, and the filtrate was under reduced pressure concentrate. A product therefrom was purified through silica gel column chromatography with n-hexane/dichloromethane (in a volume ratio of 2:1) to obtain 10.5 g (Yield=56%) of Intermediate E-9-4 as a white solid.

5th Step: Synthesis of Compound E-9

3.5 g (8.15 mmol) of Intermediate E-9-4, 3.3 g (8.96 mmol) of 3-dibenzothiophene-phenylamine, 1.96 g (20.37 mmol) of sodium t-butoxide, and 0.3 g (0.81 mmol) of tri-tert-butylphosphine were dissolved in 50 ml of xylene, 0.37 g (0.41 mmol) of Pd(dba)₂ was added thereto and then, refluxed and stirred under a nitrogen atmosphere for 12 hours. When a reaction was complete, xylene and distilled water were used for an extraction, an organic layer therefrom was dried with magnesium sulfate anhydrous and filtered, and the filtrate was concentrated under a reduced pressure. A product therefrom was purified through silica gel column chromatography with n-hexane/dichloromethane (a volume ratio of 2:1) to obtain 3.8 g (Yield=75%) of Compound E-9 as a white solid.

LC/MS calculated for: C42H28N2S2 Exact Mass: 624.17 found for 625.15 [M+H].

### Synthesis Example 18: Synthesis of Compound E-12

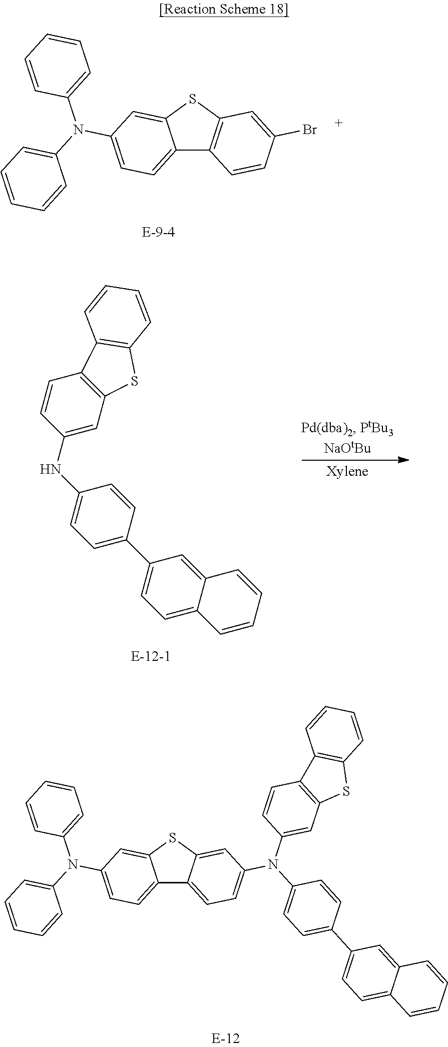 [SMILES: Brc1ccc2c(c1)sc1cc(N(c3ccccc3)c3ccccc3)ccc12 ; c1ccc2cc(-c3ccc(Nc4ccc5c(c4)sc4ccccc45)cc3)ccc2c1 ; c1ccc(N(c2ccccc2)c2ccc3c(c2)sc2cc(N(c4ccc(-c5ccc6ccccc6c5)cc4)c4ccc5c(c4)sc4ccccc45)ccc23)cc1]

4.7 g (Yield of 68%) of Compound E-12 as a white solid was according to the same method as the 5th step of Compound E-9 according to Example 17.

LC/MS calculated for: C52H34N2S2 Exact Mass: 750.22 found for 751.24 [M+H].

### Synthesis Example 19: Synthesis of Compound E-13

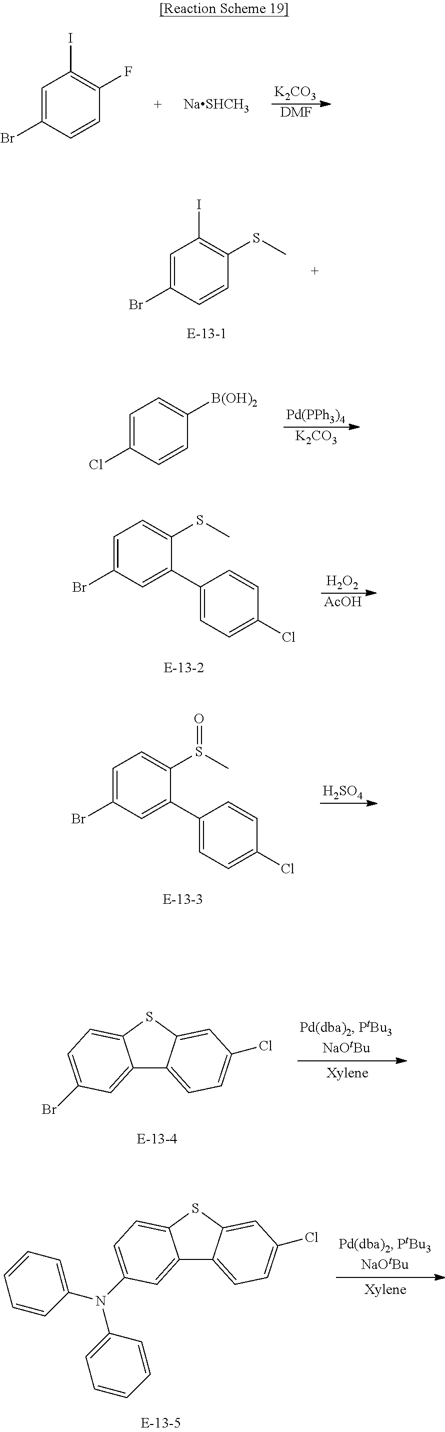

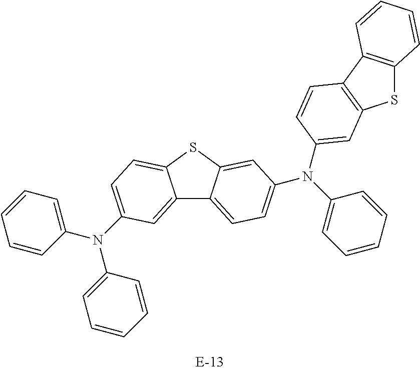 [SMILES: c1ccc(N(c2ccc3c(c2)sc2ccccc23)c2ccc3c(c2)sc2ccc(N(c4ccccc4)c4ccccc4)cc23)cc1]

1st Step: Synthesis of Intermediate E-13-1

150 g (498.5 mmol) of 4-bromo-2-fluoro-1-iodobenzene was added to 1.5 L of N,N-dimethylformamide in a 3 L round-bottomed flask, and an internal temperature thereof was set at 0° C. 35.44 g (498.52 mmol) of sodium thiomethoxide (CAS No.: 5188-07-8) and 103.19 g (747.98 mmol) of potassium carbonate were slowly added thereto. Herein, the internal temperature was maintained at 0° C. The flask was heated at 80° C. under a nitrogen atmosphere. After 6 hours, the reaction solution was cooled down, ethyl acetate and an aqueous layer were added thereto and then, stirred, and an organic layer therefrom was treated through column chromatography under a reduced pressure to obtain 106.61 g (Yield of 65%) of Intermediate E-13-1.

2nd Step: Synthesis of Intermediate E-13-2

Intermediate E-13-1 (106 g, 322 mmol) was dissolved in 1.0 L of tetrahydrofuran (THF), and 4-chlorophenylboronic acid (57.66 g, 322 mmol) and tetrakis(triphenylphosphine) palladium (11.2 g, 9.7 mmol) were added thereto and then stirred. Subsequently, potassium carbonate saturated in water (111.32 g, 805 mmol) was added thereto and then, heated and refluxed at 80° C. for 12 hours. When a reaction was complete, water was added to the reaction solution, dichloromethane (DCM) was used for an extraction, magnesium sulfate anhydrous was used to remove moisture therefrom, and the residue was filtered and concentrated under a reduced pressure. The obtained residue was separated and purified through flash column chromatography to obtain 63.66 g (63%) of Intermediate E-13-2.

3rd Step: Synthesis of Intermediate E-13-3

63 g (200.87 mmol) of Intermediate E-13-2 was added to 600 mL of acetic acid, and an internal temperature thereof was set at 0° C. 20.4 ml of hydrogen peroxide was slowly added thereto. Herein, the internal temperature was maintained at 0° C. The reaction solution was stirred at ambient temperature for 6 hours, put in ice water, extracted with dichloromethane (DCM), treated with magnesium sulfate anhydrous to remove moisture, filtered, and concentrated under a reduced pressure to obtain 61 g (Yield of 92%) of Intermediate E-13-3.

4th Step: Synthesis of Intermediate E-13-4

60 g (182.12 mmol) of Intermediate E-13-3 was added to 400 mL of sulfuric acid and then, stirred at ambient temperature for 6 hours, the reaction solution was put in ice water and then, adjusted to a pH of 9 by using an NaOH aqueous solution. Subsequently, the reaction solution was extracted with dichloromethane (DCM), treated with magnesium sulfate anhydrous to remove moisture therefrom, filtered, and concentrated under a reduced pressure to obtain 38 g (Yield of 70%) of Intermediate E-13-4.

5th Step: Synthesis of Intermediate E-13-5

5.0 g (16.82 mmol) of Intermediate E-13-4, 2.85 g (16.82 mmol) of diphenylamine, 4.04 g (42.04 mmol) of sodium t-butoxide, and 0.7 g (1.69 mmol) of tri-tert-butylphosphine were dissolved in 100 ml of xylene, and 0.77 g (0.84 mmol) of Pd(dba)₂ was added thereto and then, stirred under a nitrogen atmosphere for 12 hours at 100° C. When a reaction was complete, xylene and distilled water were used for an extraction, and an organic layer therefrom was dried with magnesium sulfate anhydrous, filtered, and concentrated under a reduced pressure. A product therefrom was purified through silica gel column chromatography by using n-hexane/dichloromethane (in a volume ratio of 2:1) to obtain 4.7 g (Yield=72%) of a white solid, Intermediate E-13-5.

6th Step: Synthesis of Compound E-13

4.5 g (11.68 mmol) of Intermediate E-13-5, 3.3 g (11.68 mmol) of 3-dibenzothiophene-phenylamine, 2.81 g (29.21 mmol) of sodium t-butoxide, and 1.2 g (1.17 mmol) of tri-tert-butylphosphine were dissolved in 50 ml of xylene, and 0.54 g (0.58 mmol) of Pd(dba)₂ was added thereto and then, refluxed and stirred under a nitrogen atmosphere for 12 hours. When a reaction was complete, xylene and distilled water were used for an extraction, an organic layer therefrom was dried with magnesium sulfate anhydrous and filtered, and the filtrate was concentrated under a reduced pressure. A product therefrom was purified through silica gel column chromatography with n-hexane/dichloromethane (in a volume ratio of 2:1) to obtain 5.5 g (Yield of 75%) of Compound E-13 as a white solid.

LC/MS calculated for: C42H28N2S2 Exact Mass: 624.17 found for 625.15 [M+H].

### Synthesis Example 20: Synthesis of Compound E-16

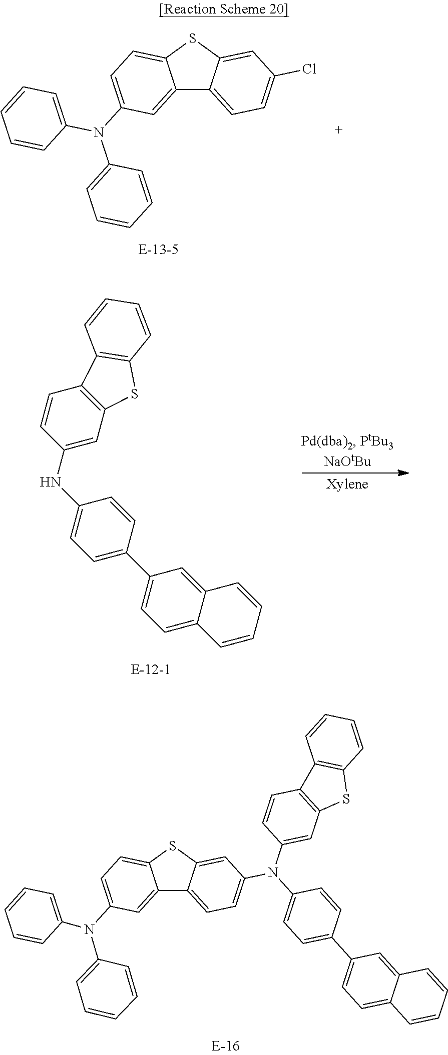 [SMILES: Clc1ccc2c(c1)sc1ccc(N(c3ccccc3)c3ccccc3)cc12 ; c1ccc2cc(-c3ccc(Nc4ccc5c(c4)sc4ccccc45)cc3)ccc2c1 ; c1ccc(N(c2ccccc2)c2ccc3sc4cc(N(c5ccc(-c6ccc7ccccc7c6)cc5)c5ccc6c(c5)sc5ccccc56)ccc4c3c2)cc1]

3.5 g (Yield=70%) of Compound E-16 as a white solid was obtained according to the same synthesis method as Compound E-13 according to the 6th step of Example 19.

LC/MS calculated for: C52H34N2S2 Exact Mass: 750.22 found for 751.23 [M+H].

### Synthesis Example 21: Synthesis of Compound E-33

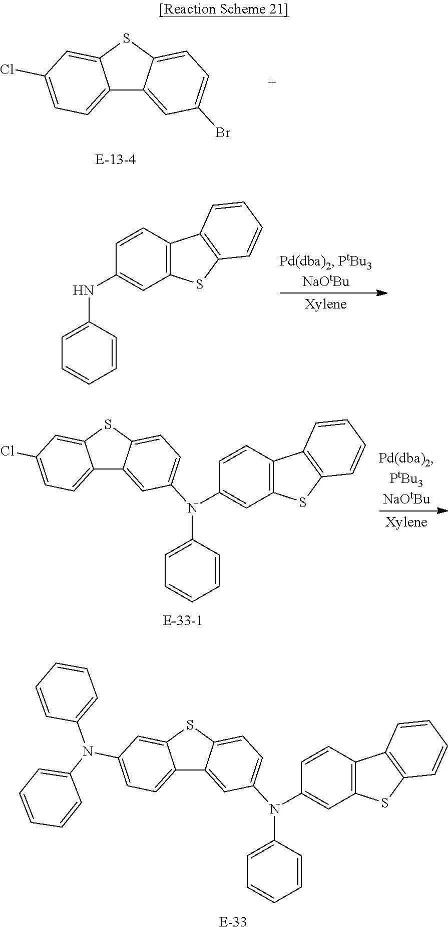

1st Step: Synthesis of Intermediate E-33-1

Intermediate E-33-1 was obtained according to the same method as Intermediate E-13-5 according to the 5th step of Example 19 except that 3-dibenzothiophene-phenylamine was used instead of the diphenylamine.

2nd Step: Synthesis of Compound E-33

5.1 g (Yield of 62%) of Compound E-33 as a white solid was obtained according to the same synthesis method as Compound E-13 according to the 6th step of Example 19 except that Intermediate E-33-1 was used instead of Intermediate E-13-5.

LC/MS calculated for: C42H28N2S2 Exact Mass: 624.17 found for 625.18 [M+H].

### Synthesis Example 22: Synthesis of Compound E-65

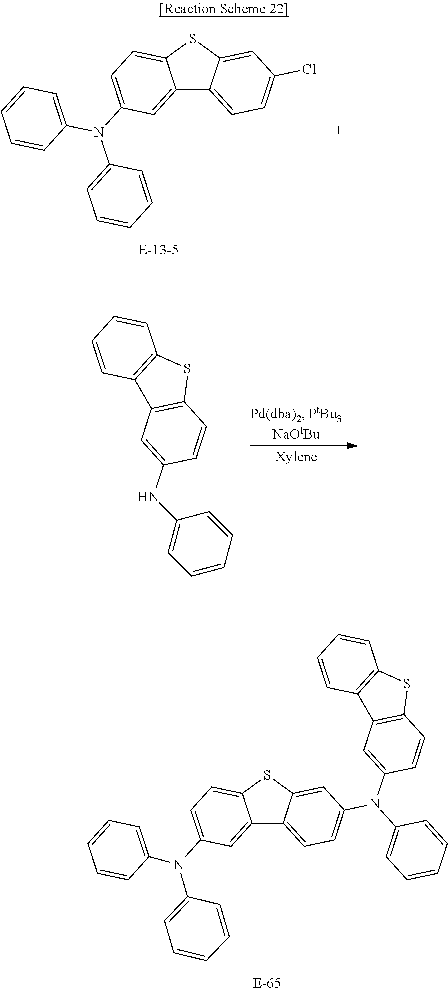 [SMILES: Clc1ccc2c(c1)sc1ccc(N(c3ccccc3)c3ccccc3)cc12 ; c1ccc(Nc2ccc3sc4ccccc4c3c2)cc1 ; c1ccc(N(c2ccc3c(c2)sc2ccc(N(c4ccccc4)c4ccccc4)cc23)c2ccc3sc4ccccc4c3c2)cc1]

3.9 g (Yield of 66%) of Compound E-65 as a white solid was obtained according to the same synthesis method as Compound E-13 according to the 6th step of Example 19 except that 2-dibenzothiophene-phenylamine was used instead of the 3-dibenzothiophene-Phenylamine.

LC/MS calculated for: C42H28N2S2 Exact Mass: 624.17 found for 625.15 [M+H].

### Synthesis Example 23: Synthesis of Compound E-93

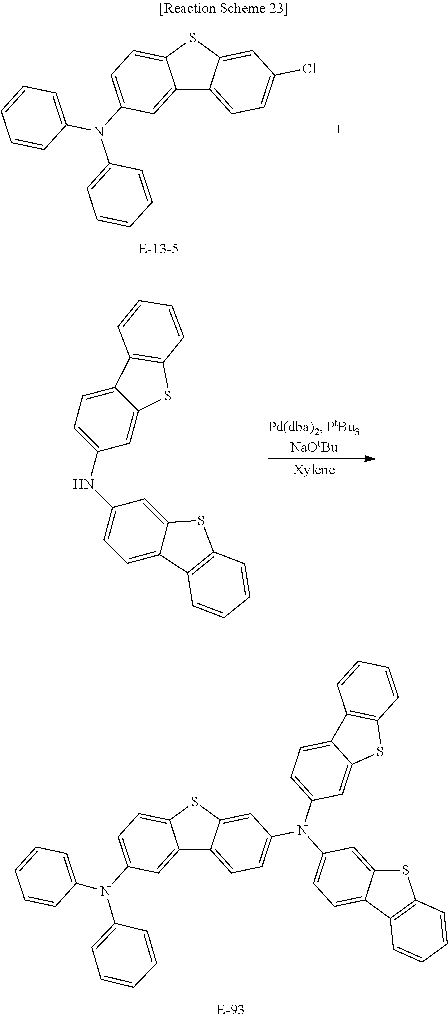 [SMILES: Clc1ccc2c(c1)sc1ccc(N(c3ccccc3)c3ccccc3)cc12 ; c1ccc2c(c1)sc1cc(Nc3ccc4c(c3)sc3ccccc34)ccc12 ; c1ccc(N(c2ccccc2)c2ccc3sc4cc(N(c5ccc6c(c5)sc5ccccc56)c5ccc6c(c5)sc5ccccc56)ccc4c3c2)cc1]

3.4 g (Yield of 64%) of Compound E-93 as a white solid was obtained according to the same synthesis method as Compound E-13 according to the 6th step of Example 19 except that N-3-dibenzothienyl-3-dibenzothiophenamine (CAS num: 1705596-48-0) was used instead of the 3-dibenzothiophene-phenylamine.

LC/MS calculated for: C48H30N2S3 Exact Mass: 730.16 found for 731.15 [M+H].

### Synthesis Example 24: Synthesis of Compound F-17

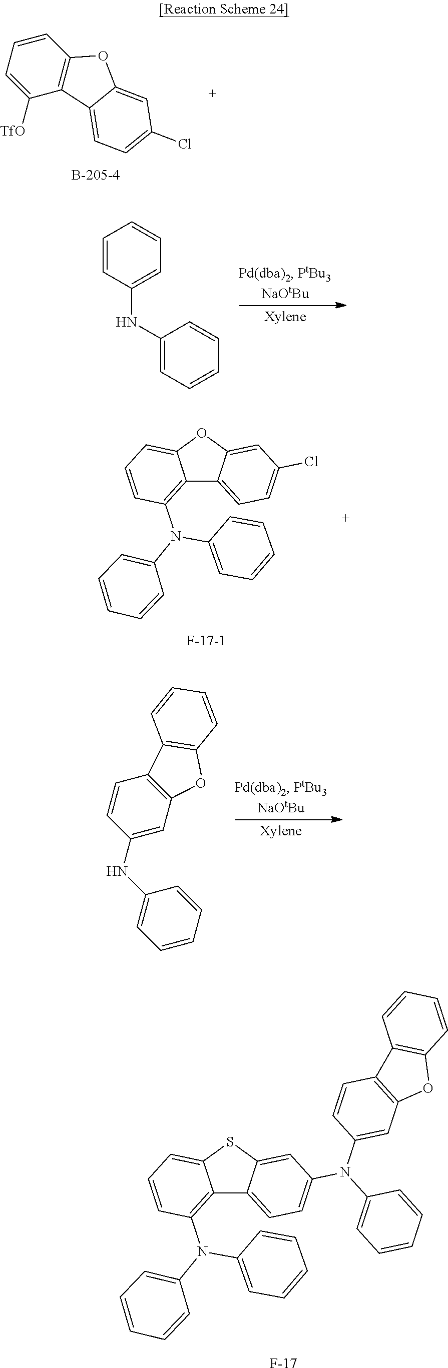

1st step: Synthesis of Intermediate F-17-1

Intermediate F-17-1 was obtained according to the same synthesis method as Intermediate E-13-5 according to the 5th step of Example 19 except that Intermediate B-205-4 was used instead of Intermediate E-13-4.

2nd Step: Synthesis of Compound F-17

6.8 g (Yield of 70%) of Compound F-17 as a white solid was obtained according to the same synthesis method as Compound E-13 according to the 6th step of Example 19 except that Intermediate F-17-1 and 3-dibenzofuran-phenylamine were used.

LC/MS calculated for: C42H28N2O2 Exact Mass: 592.21 found for 593.23 [M+H].

### Synthesis Example 25: Synthesis of Compound F-37

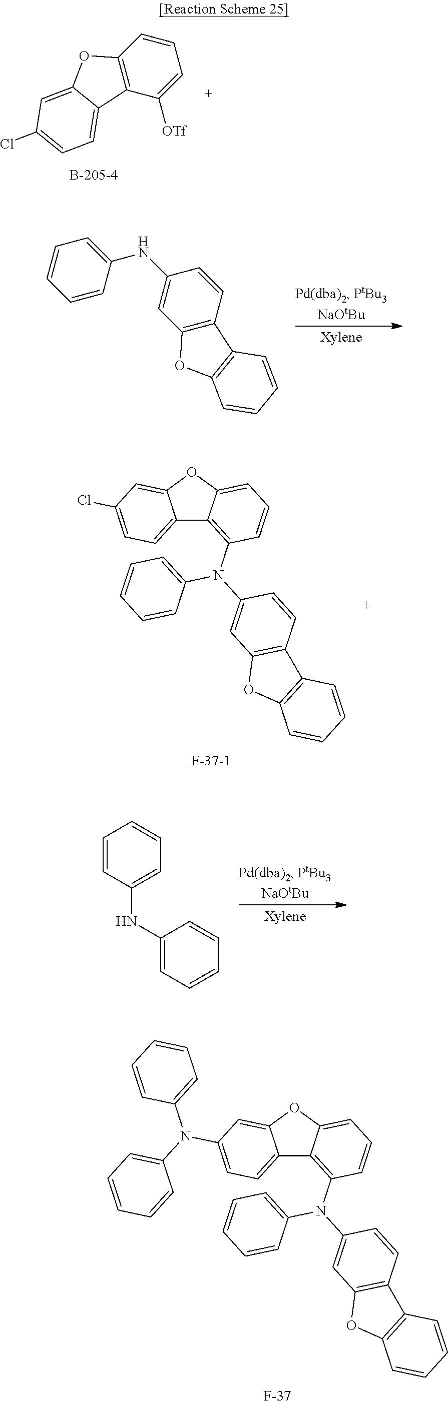

1st Step: Synthesis of Intermediate F-37-1

Intermediate F-37-1 was obtained according to the same synthesis method as Intermediate E-13-5 according to the 5th step of Example 19 except that B-205-4 and 3-dibenzofuran-phenylamine were used.

2nd Step: Synthesis of Compound F-37

6.2 g (Yield=69%) of Intermediate F-37 as a white solid was obtained according to the same synthesis method as Intermediate E-13 according to the 6th step of Example 19 except that Intermediate F-37-1 and diphenylamine were used.

LC/MS calculated for: C421H28N2O2 Exact Mass: 592.21 found for 593.22 [M+H].

### Synthesis Example 26: Synthesis of Compound G-13

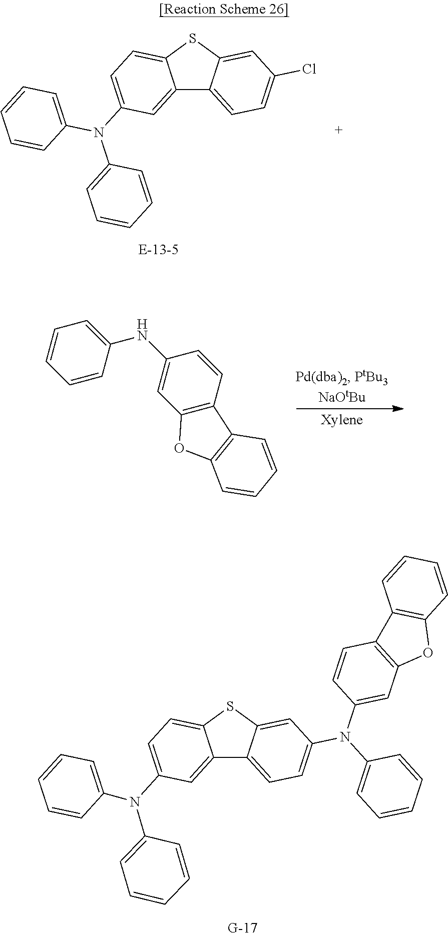 [SMILES: Clc1ccc2c(c1)sc1ccc(N(c3ccccc3)c3ccccc3)cc12 ; c1ccc(Nc2ccc3c(c2)oc2ccccc23)cc1 ; c1ccc(N(c2ccc3c(c2)oc2ccccc23)c2ccc3c(c2)sc2ccc(N(c4ccccc4)c4ccccc4)cc23)cc1 ; "G-17"]

7.0 g (Yield=71%) of Compound G-13 as a white solid was obtained according to the same synthesis method as Intermediate E-13 according to the 6th step of Example 19 except that Intermediate E-13-5 and 3-dibenzofuran-phenylamine were used.

LC/MS calculated for: C42H28N2OS Exact Mass: 608.19 found for 608.20 [M+H].

### Synthesis Example 27: Synthesis of Compound H-17

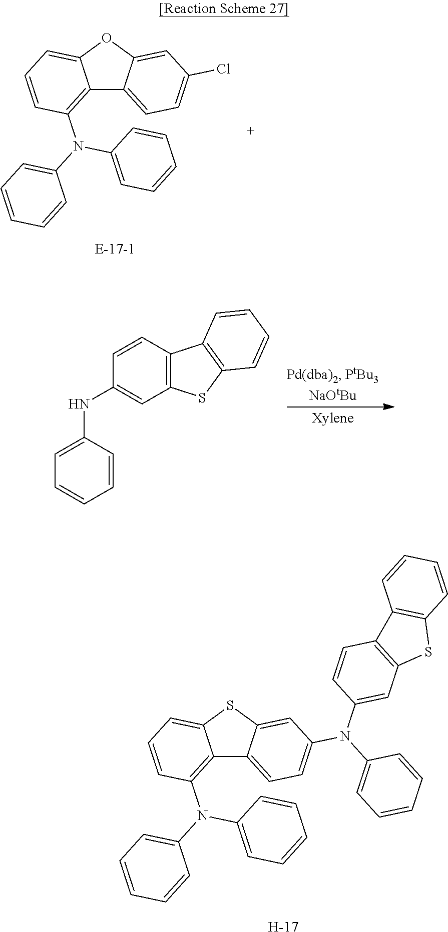

5.7 g (Yield=68%) of Compound H-17 as a white solid was obtained according to the same synthesis method as Intermediate E-13 according to the 6th step of Example 19 except that Intermediate F-17-1 and 3-dibenzothiophene-phenylamine were used.

LC/MS calculated for: C42H28N2OS Exact Mass: 608.19 found for 608.21 [M+H].

(Manufacture of Organic Light Emitting Diode)

### Example 1

A glass substrate coated with ITO (indium tin oxide) as a 1,500 Å-thick film was washed with distilled water. After washing with the distilled water, the glass substrate was ultrasonic wave-washed with a solvent such as isopropyl alcohol, acetone, and methanol, and dried and then, moved to a plasma cleaner, cleaned by using oxygen plasma for 10 minutes, and moved to a vacuum depositor. This obtained ITO transparent electrode was used as an anode, Compound A was vacuum-deposited on the ITO substrate to form a 700 Å-thick hole injection layer, Compound B was deposited to be 50 Å thick on the injection layer, and Compound C was deposited to be 700 Å thick to form a hole transport layer. On the hole transport layer, a 700 Å-thick hole transport auxiliary layer was formed by vacuum-depositing Compound E-13. On the hole transport auxiliary layer, a 400 Å-thick light emitting layer was formed by vacuum-depositing Compounds A-3 and B-135 simultaneously as a host and doping 2 wt % of [Ir(piq)₂acac] as a dopant. Herein, Compound A-3 and Compound B-135 were used in a weight ratio of 6:4, and their ratios in the following Examples were separately provided. Subsequently, on the light emitting layer, a 300 Å-thick electron transport layer was formed by simultaneously vacuum-depositing Compound D and Liq in a ratio of 1:1, and on the electron transport layer, Liq and Al were sequentially vacuum-deposited to be 15 Å thick and 1,200 Å thick, manufacturing an organic light emitting diode.

The organic light emitting diode included a six-layered organic thin layer, and had the following structure.

ITO/Compound A (700 Å)/Compound B (50 Å)/Compound C (700 Å)/Compound E-13 (700 Å)/EML [Compound A-3: B-135: [Ir(piq)₂acac] (2 wt %)](400 Å)/Compound D: Liq (300 Å)/Liq (15 Å)/A1 (1,200 Å).

Compound A: N4,N4′-diphenyl-N4,N4′-bis(9-phenyl-9H-carbazol-3-yl)biphenyl-4,4′-diamine

Compound B: 1,4,5,8,9,11-hexaazatriphenylene-hexacarbonitrile (HAT-CN)

Compound C: N-(biphenyl-4-yl)-9,9-dimethyl-N-(4-(9-phenyl-9H-carbazol-3-yl)phenyl)-9H-fluoren-2-amine

Compound D: 8-(4-(4,6-di(naphthalen-2-yl)-1,3,5-triazin-2-yl)phenyl)quinoline

### Examples 2 to 23

Organic light emitting diodes were manufactured according to the same method as Example 1 except that the composition was changed into compositions shown in Table 1.

### Comparative Examples 1 to 3

Organic light emitting diodes were manufactured according to the same method as Example 1 except that the composition was changed into compositions shown in Table 1. For example, in Comparative Examples 2 and 3, the organic light emitting diodes included only a single compound as the host.

Evaluation

Driving voltages and power efficiency of the organic light emitting diodes according to Examples 1 to 23 and Comparative Examples 1 to 3 were evaluated.

Specific measurement methods are as follows, and the results are shown in Table 1.

(1) Measurement of Driving Voltage

Driving voltages of each device were measured using a current-voltage meter (Keithley 2400).

(2) Measurement of Current Density Change Depending on Voltage Change

The obtained organic light emitting diodes were measured regarding a current value flowing in the unit device, while increasing the voltage from 0 V to 10 V using a current-voltage meter (Keithley 2400), and the measured current value was divided by area to provide the results.

(3) Measurement of Luminance Change Depending on Voltage Change

Luminance was measured by using a luminance meter (Minolta Cs-1000A), while the voltage of the organic light emitting diodes was increased from 0 V to 10 V.

(4) Measurement of Power Efficiency

Power efficiency (lm/w) was calculated by using the luminance, current density, and voltages (V) from the items (2) and (3).

TABLE 1

| | | | Hole transport | | Power |
|---|---|---|---|---|---|
| | Light emitting layer | | auxiliary layer | Driving | effi- |
| | First | Second | Third | voltage | ciency |
| | compound | compound | compound | (V) | (lm/w) |
| Example 1 | A-3 | B-135 | E-13 | 3.86 | 121% |
| Example 2 | A-3 | B-135 | E-16 | 3.76 | 113% |
| Example 3 | A-3 | B-135 | E-33 | 3.84 | 125% |
| Example 4 | A-3 | B-135 | E-65 | 3.83 | 127% |
| Example 5 | A-3 | B-135 | E-93 | 3.83 | 121% |
| Example 6 | A-3 | B-135 | F-17 | 4.30 | 107% |
| Example 7 | A-3 | B-135 | F-37 | 4.22 | 114% |
| Example 8 | A-3 | B-135 | G-13 | 3.82 | 122% |
| Example 9 | A-3 | B-135 | H-17 | 4.33 | 108% |
| Example 10 | A-3 | B-17 | E-13 | 3.93 | 118% |
| Example 11 | A-3 | B-183 | E-13 | 3.74 | 123% |
| Example 12 | A-3 | B-205 | E-13 | 3.78 | 113% |
| Example 13 | A-3 | B-209 | E-13 | 4.13 | 115% |
| Example 14 | A-3 | C-23 | E-13 | 4.05 | 115% |
| Example 15 | A-3 | C-25 | E-13 | 4.05 | 117% |
| Example 16 | A-3 | D-57 | E-13 | 3.97 | 118% |
| Example 17 | A-2 | B-135 | E-13 | 3.93 | 121% |
| Example 18 | A-7 | B-135 | E-13 | 3.86 | 119% |
| Example 19 | A-17 | B-135 | E-13 | 3.90 | 122% |
| Example 20 | A-23 | B-135 | E-13 | 3.82 | 117% |
| Example 21 | A-27 | B-135 | E-13 | 3.97 | 117% |
| Example 22 | A-33 | B-135 | E-13 | 3.97 | 119% |
| Example 23 | A-47 | B-135 | E-13 | 3.90 | 118% |
| Comparative | V-1 | B-135 | E-13 | 4.62 | 77% |
| Example 1 | | | | | |

| | | | | |
|---|---|---|---|---|
| Comparative | V-3 | E-13 | 4.47 | 89% |
| Example 2 | | | | |
| Comparative | V-4 | E-13 | 4.36 | 100% |
| Example 3 | | | | |

Referring to Table 1, the organic light emitting diodes according to Examples 1 to 23 exhibited significantly lower driving voltages and improved power efficiency, compared with the organic light emitting diodes of Comparative Examples 1 to 3.

One or more embodiments may provide an organic optoelectronic device exhibiting high efficiency and a long life-span.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the following claims.

What is claimed is:

1. An organic optoelectronic device, comprising:
an anode and a cathode facing each other,
a light emitting layer between the anode and the cathode,
a hole transport layer between the anode and the light emitting layer, and a hole transport auxiliary layer between the light emitting layer and the hole transport layer,
wherein:
the light emitting layer includes a first compound represented by Chemical Formula 1 and a second compound represented by Chemical Formula 2, and
the hole transport auxiliary layer includes a third compound represented by Chemical Formula 3, 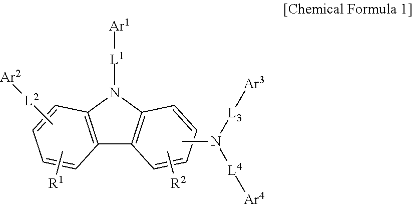
wherein, in Chemical Formula 1,
Ar¹ and Ar² are each independently a substituted or unsubstituted C6 to C12 aryl group,
Ar³ and Ar⁴ are each independently a substituted or unsubstituted C6 to C30 aryl group,
L¹ is a single bond or a substituted or unsubstituted phenylene group,
L² to L⁴ are each independently a single bond or a substituted or unsubstituted C6 to C12 arylene group, and
R¹ and R² are each independently hydrogen, deuterium, a cyano group, or a substituted or unsubstituted C1 to C10 alkyl group; 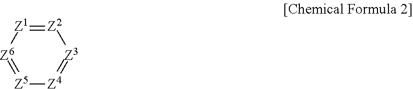
wherein, in Chemical Formula 2,
Z¹ is N or C-L⁵-R³,
Z² is N or C-L⁶-R⁴,
Z³ is N or C-L⁷-R⁵,
Z⁴ is N or C-L¹⁰-R⁶,
Z⁵ is N or C-L⁹-R⁷,
Z⁶ is N or C-L¹⁰-R⁸,
at least two of Z¹ to Z⁶ are N,
L⁵ to L¹⁰ are each independently a single bond, a substituted or unsubstituted C6 to C20 arylene group, a substituted or unsubstituted divalent C2 to C20 heterocyclic group, or a combination thereof,
R³ to R⁸ are each independently hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted silyl group, a substituted or unsubstituted amine group, a halogen, a cyano group, or a combination thereof,
at least one of R³ to R⁸ is a substituted or unsubstituted dibenzofuranyl group, a substituted or unsubstituted dibenzothiophenyl group, a substituted or unsubstituted carbazolyl group, a substituted or unsubstituted benzocarbazolyl group, a substituted or unsubstituted dibenzocarbazolyl group, or a substituted or unsubstituted triphenylene group, and
R³ to R⁸ are each separately present or adjacent groups thereof are linked with each other to form a substituted or unsubstituted aliphatic monocyclic or polycyclic ring, a substituted or unsubstituted aromatic monocyclic or polycyclic ring, or a substituted or unsubstituted heteroaromatic monocyclic or polycyclic ring; 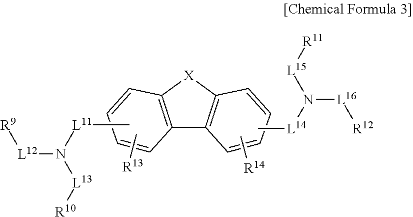
wherein, in Chemical Formula 3,
X is O or S,
L¹¹ to L¹⁶ are each independently a single bond, a substituted or unsubstituted C6 to C20 arylene group, a substituted or unsubstituted divalent C2 to C20 heterocyclic group, or a combination thereof,
R⁹ to R¹² are each independently a substituted or unsubstituted C6 to C30 aryl group, a substituted or unsubstituted C2 to C30 heterocyclic group, or a combination thereof, and
R¹³ and R¹⁴ are each independently hydrogen, deuterium, a cyano group, or a substituted or unsubstituted C1 to C10 alkyl group.

2. The organic optoelectronic device as claimed in claim 1, wherein the first compound is represented by Chemical Formula 1-2 or Chemical Formula 1-3: 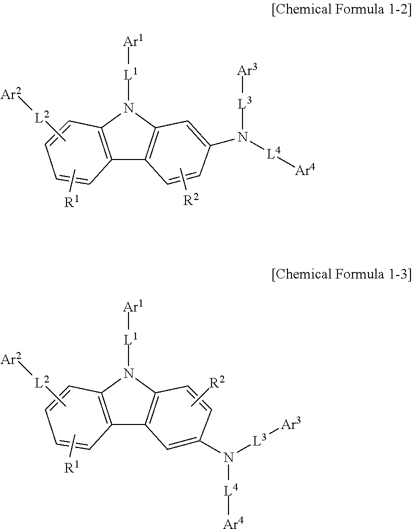
wherein, in Chemical Formula 1-2 and Chemical Formula 1-3,
Ar¹ and Ar² are each independently a substituted or unsubstituted C6 to C12 aryl group,
Ar³ and Ar⁴ are each independently a substituted or unsubstituted C6 to C30 aryl group,
L¹ is a single bond or a substituted or unsubstituted phenylene group,
L² to L⁴ are each independently a single bond or a substituted or unsubstituted C6 to C12 arylene group, and
R¹ and R² are each independently hydrogen, deuterium, a cyano group, or a substituted or unsubstituted C1 to C10 alkyl group.

3. The organic optoelectronic device as claimed in claim 1, wherein the first compound is represented by Chemical Formula 1-2b or Chemical Formula 1-3b: 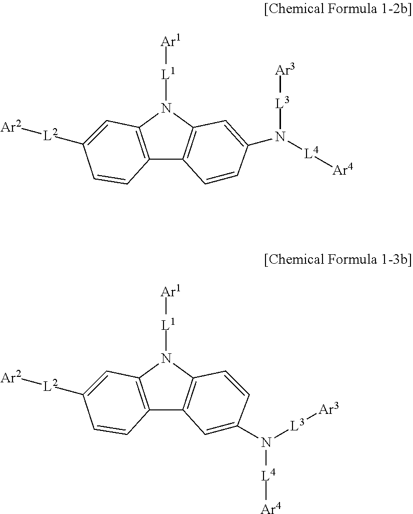
wherein, in Chemical Formula 1-2b and Chemical Formula 1-3b,
Ar¹ and Ar² are each independently a substituted or unsubstituted C6 to C12 aryl group,
Ar³ and Ar⁴ are each independently a substituted or unsubstituted C6 to C30 aryl group,
L¹ is a single bond or a substituted or unsubstituted phenylene group, and
L² to L⁴ are each independently a single bond or a substituted or unsubstituted C6 to C12 arylene group.

4. The organic optoelectronic device as claimed in claim 1, wherein Ar¹ and Ar² are each independently a substituted or unsubstituted phenyl group, a substituted or unsubstituted biphenyl group, or a substituted or unsubstituted naphthyl group.

5. The organic optoelectronic device as claimed in claim 1, wherein Ar³ and Ar⁴ are each independently a substituted or unsubstituted phenyl group, a substituted or unsubstituted biphenyl group, a substituted or unsubstituted naphthyl group, a substituted or unsubstituted terphenyl group, a substituted or unsubstituted anthracenyl group, or a substituted or unsubstituted phenanthrenyl group.

6. The organic optoelectronic device as claimed in claim 1, wherein the second compound is represented by Chemical Formula 2-1 to Chemical Formula 2-3: 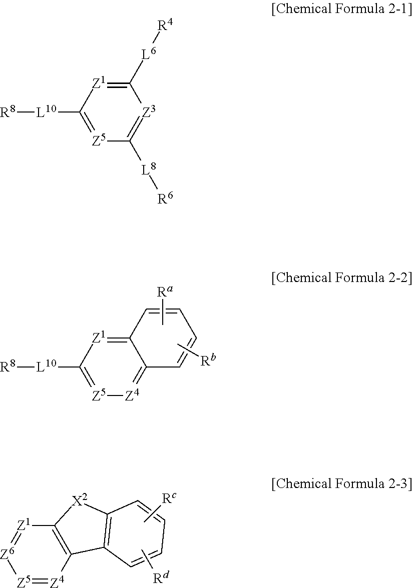
wherein, in Chemical Formula 2-1 to Chemical Formula 2-3,
Z is N or C-L⁵-R³,
Z³ is N or C-L⁷-R⁵,
Z⁴ is N or C-L⁸-R⁶,
Z⁵ is N or C-L⁹-R⁷,
Z⁶ is N or C-L¹⁰-R⁸,
at least two of Z¹ and Z³ to Z⁶ are N,
L⁵ to L¹⁰ are each independently a single bond, a substituted or unsubstituted C6 to C20 arylene group, a substituted or unsubstituted divalent C2 to C20 heterocyclic group, or a combination thereof,
R³ to R⁸ are each independently hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted silyl group, a substituted or unsubstituted amine group, a halogen, a cyano group, or a combination thereof,
at least one of R³ to R⁸ is a substituted or unsubstituted dibenzofuranyl group, a substituted or unsubstituted dibenzothiophenyl group, a substituted or unsubstituted carbazolyl group, a substituted or unsubstituted benzocarbazolyl group, a substituted or unsubstituted dibenzocarbazolyl group, or a substituted or unsubstituted triphenylene group, and
R^{a}, R^{b}, R^{c}, and R^{d} are each independently hydrogen, deuterium, a halogen, a cyano group, a C1 to C20 alkyl group, a C6 to C30 aryl group, a C2 to C30 heteroaryl group, or a combination thereof.

7. The organic optoelectronic device as claimed in claim 1, wherein at least one of R³ to R⁸ is a group of the following Group II: 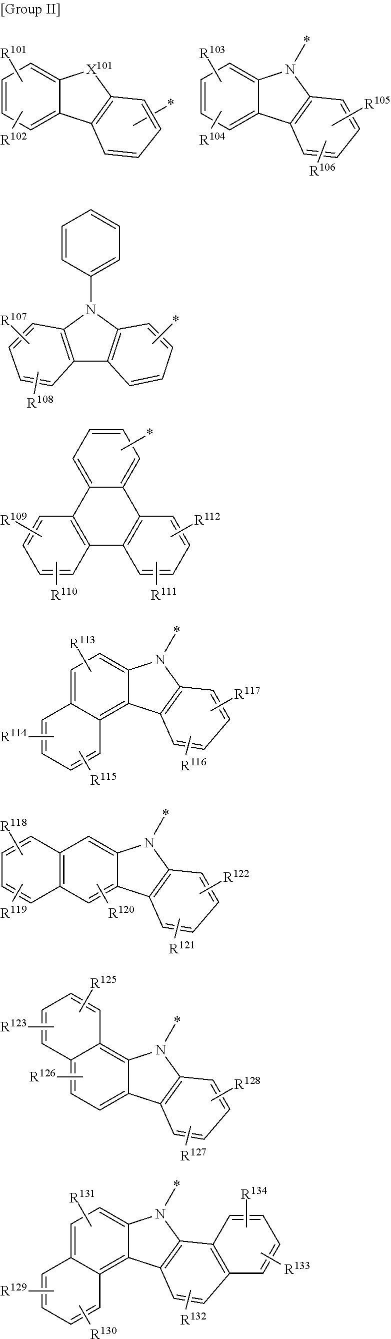 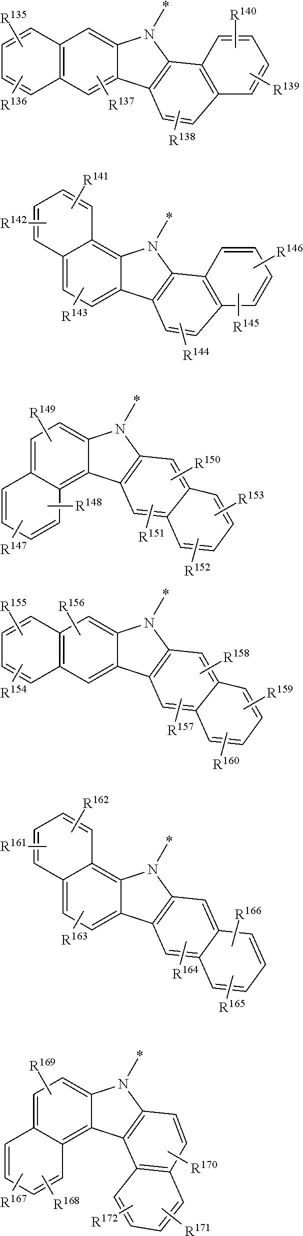 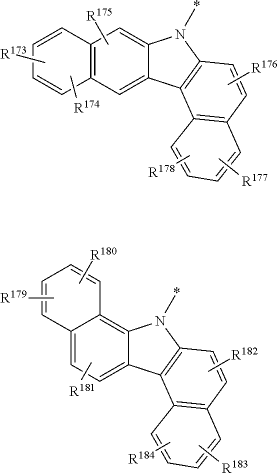
wherein, in Group II,
X¹⁰¹ is O or S,
R¹⁰¹ to R¹²³ and R¹²⁵ to R¹⁸⁴ are each independently hydrogen, deuterium, a halogen, a cyano group, a C1 to C20 alkyl group, a C6 to C30 aryl group, a C2 to C30 heteroaryl group, or a combination thereof, and
is a linking point.

8. The organic optoelectronic device as claimed in claim 1, wherein the second compound is represented by Chemical Formula 2-1a or Chemical Formula 2-3a: 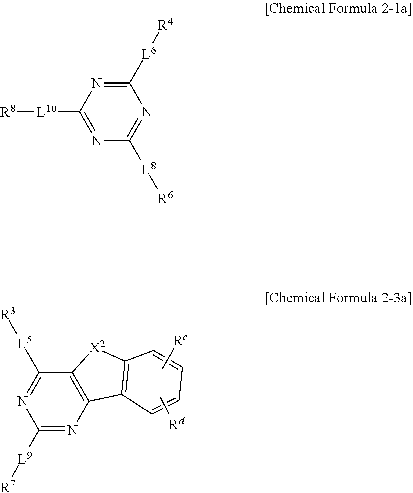
wherein, in Chemical Formula 2-1a,
L⁶, L⁸, and L¹⁰ are each independently a single bond or a substituted or unsubstituted phenylene group,
R⁴, R⁶, and R⁸ are each independently a substituted or unsubstituted phenyl group, a substituted or unsubstituted biphenyl group, a substituted or unsubstituted terphenyl group, a substituted or unsubstituted naphthyl group, a substituted or unsubstituted triphenylene group, a substituted or unsubstituted dibenzofuranyl group, a substituted or unsubstituted dibenzothiophenyl group, or a substituted or unsubstituted carbazolyl group,
at least one of R⁴, R⁶, and R⁸ is a substituted or unsubstituted triphenylene group, a substituted or unsubstituted carbazolyl group, a substituted or unsubstituted dibenzofuranyl group, or a substituted or unsubstituted dibenzothiophenyl group;
wherein, in Chemical Formula 2-3a,
X² is O or S,
L⁵ and L⁹ are each independently a single bond or a substituted or unsubstituted phenylene group,
R³ and R⁷ are each independently a substituted or unsubstituted phenyl group, a substituted or unsubstituted biphenyl group, a substituted or unsubstituted terphenyl group, a substituted or unsubstituted naphthyl group, a substituted or unsubstituted triphenylene group, a substituted or unsubstituted dibenzofuranyl group, a substituted or unsubstituted dibenzothiophenyl group, or a substituted or unsubstituted carbazolyl group,
at least one of R³ and R⁷ is a substituted or unsubstituted carbazolyl group, a substituted or unsubstituted dibenzofuranyl group, or a substituted or unsubstituted dibenzothiophenyl group, and
R^{c} and R^{d} are each independently hydrogen, deuterium, a C1 to C10 alkyl group, a C6 to C12 aryl group, or a combination thereof.

9. The organic optoelectronic device as claimed in claim 8, wherein the first compound is represented by Chemical Formula 1-2b: 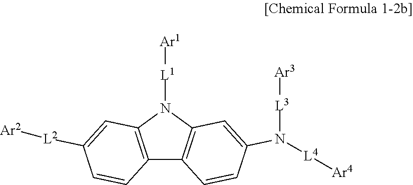
wherein, in Chemical Formula 1-2b,
L¹ to L⁴ are each independently a single bond or a substituted or unsubstituted phenylene group, and
Ar¹ to Ar⁴ are each independently a substituted or unsubstituted phenyl group, a substituted or unsubstituted biphenyl group, or a substituted or unsubstituted naphthyl group.

10. The organic optoelectronic device as claimed in claim 1, wherein the third compound is represented by one of Chemical Formula 3-1 to Chemical Formula 3-4: 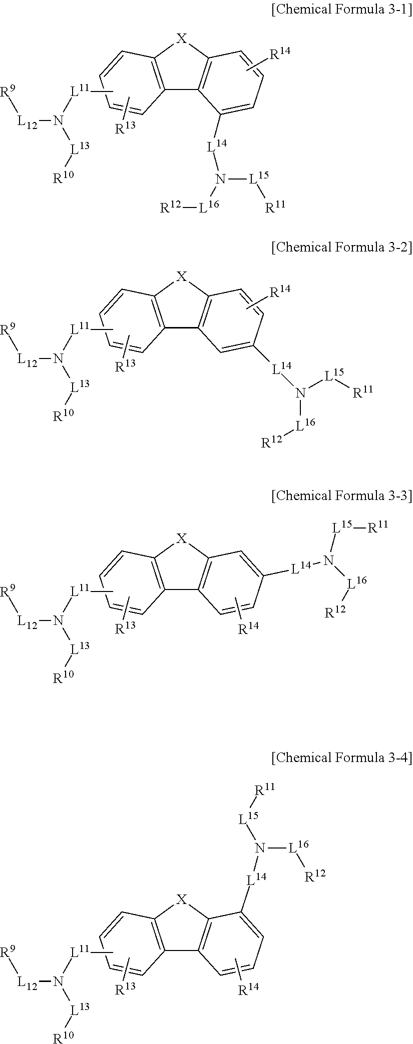
wherein, in Chemical Formula 3-1 to Chemical Formula 3-4,
X is O or S,
L¹¹ to L¹⁶ are each independently a single bond, a substituted or unsubstituted C6 to C20 arylene group, a substituted or unsubstituted divalent C2 to C20 heterocyclic group, or a combination thereof,
R⁹ to R¹² are each independently a substituted or unsubstituted C6 to C30 aryl group, a substituted or unsubstituted C2 to C30 heterocyclic group, or a combination thereof, and
R¹³ and R¹⁴ are each independently hydrogen, deuterium, a cyano group, or a substituted or unsubstituted C1 to C10 alkyl group.

11. The organic optoelectronic device as claimed in claim 1, wherein the third compound is represented by one of Chemical Formula 3-1b, Chemical Formula 3-2b, Chemical Formula 3-2c, Chemical Formula 3-3b, Chemical Formula 3-3c, and 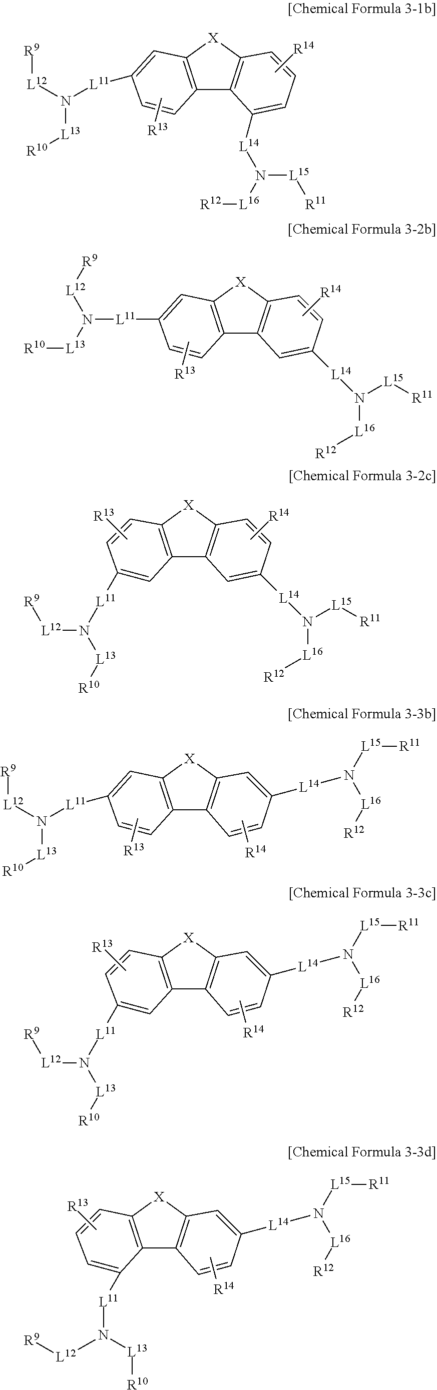
wherein, in Chemical Formula 3-1b, Chemical Formula 3-2b, Chemical Formula 3-2c, Chemical Formula 3-3b, Chemical Formula 3-3c, and Chemical Formula 3-3d,
X is O or S,
L¹¹ to L¹⁶ are each independently a single bond, a substituted or unsubstituted C6 to C20 arylene group, a substituted or unsubstituted divalent C2 to C20 heterocyclic group, or a combination thereof,
R⁹ to R¹² are each independently a substituted or unsubstituted C6 to C30 aryl group, a substituted or unsubstituted C2 to C30 heterocyclic group, or a combination thereof, and
R¹³ and R¹⁴ are each independently hydrogen, deuterium, a cyano group, or a substituted or unsubstituted C1 to C10 alkyl group.

12. The organic optoelectronic device as claimed in claim 11, wherein:
the first compound is represented by Chemical Formula 1-2b, and
the second compound is represented by Chemical Formula 2-1a or Chemical Formula 2-3a, 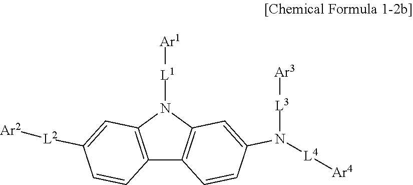
wherein, in Chemical Formula 1-2b,
L¹ to L⁴ are each independently a single bond or a substituted or unsubstituted phenylene group, and
Ar¹ to Ar⁴ are each independently a substituted or unsubstituted phenyl group, a substituted or unsubstituted biphenyl group, or a substituted or unsubstituted naphthyl group; 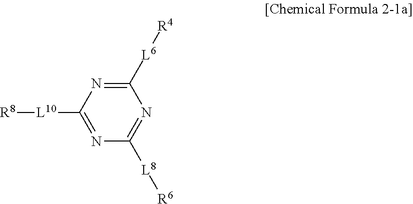
wherein, in Chemical Formula 2-1a,
L⁶, L⁸, and L¹⁰ are each independently a single bond or a substituted or unsubstituted phenylene group,
R⁴, R⁶, and R⁸ are each independently a substituted or unsubstituted phenyl group, a substituted or unsubstituted biphenyl group, a substituted or unsubstituted terphenyl group, a substituted or unsubstituted naphthyl group, a substituted or unsubstituted triphenylene group, a substituted or unsubstituted dibenzofuranyl group, a substituted or unsubstituted dibenzothiophenyl group, or a substituted or unsubstituted carbazolyl group, and
at least one of R⁴, R⁶, and R⁸ is a substituted or unsubstituted triphenylene group, a substituted or unsubstituted carbazolyl group, a substituted or unsubstituted dibenzofuranyl group, or a substituted or unsubstituted dibenzothiophenyl group; 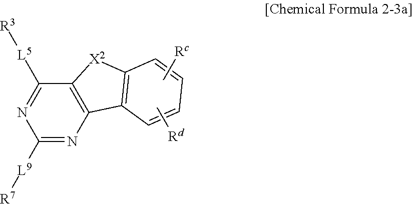
wherein, in Chemical Formula 2-3a,
X² is O or S,
L⁵ and L⁹ are each independently a single bond or a substituted or unsubstituted phenylene group,
R³ and R⁷ are each independently substituted or unsubstituted phenyl group, a substituted or unsubstituted biphenyl group, a substituted or unsubstituted terphenyl group, a substituted or unsubstituted naphthyl group, a substituted or unsubstituted triphenylene group, a substituted or unsubstituted dibenzofuranyl group, a substituted or unsubstituted dibenzothiophenyl group, or a substituted or unsubstituted carbazolyl group,
at least one of R³ and R⁷ is a substituted or unsubstituted carbazolyl group, a substituted or unsubstituted dibenzofuranyl group, or a substituted or unsubstituted dibenzothiophenyl group, and
R^{c} and R^{d} are each independently hydrogen, deuterium, a C1 to C10 alkyl group, a C6 to C12 aryl group, or a combination thereof.

13. The organic optoelectronic device as claimed in claim 1, wherein at least one of R⁹ to R¹² of Chemical Formula 3 is a substituted or unsubstituted dibenzofuranyl group or a substituted or unsubstituted dibenzothiophenyl group.

14. The organic optoelectronic device as claimed in claim 1, wherein the first compound and the second compound are phosphorescent hosts of the light emitting layer.

15. The organic optoelectronic device as claimed in claim 1, wherein the light emitting layer further includes a dopant.

16. A display device comprising the organic optoelectronic device as claimed in claim 1.
